(19) United States
(12) Patent Application Publication
(10) Pub. No.: US 2025/0308265 A1
(43) Pub. Date: Oct. 2, 2025

(54) METHODS AND SYSTEMS FOR CHARACTERIZING MORPHODYNAMIC PROFILES OF OBJECTS

(71) Applicant: The Chancellor, Masters and Scholars of the University of Oxford, Oxford (GB)

(72) Inventors: Felix Zhou, Oxford (GB); Xin Lu, Oxford (GB)

(21) Appl. No.: 19/109,697

(22) PCT Filed: Nov. 9, 2023
(86) PCT No.: PCT/EP2023/081367

§ 371 Date: Mar. 7, 2025

Related U.S. Application Data
(60) Provisional application No. 63/383,087, filed on Nov. 10, 2022.

(51) Int. Cl. G06V 20/69; G06T 7/00; G06T 7/215; G06T 7/246; G06V 10/26; G06V 10/46; G06V 10/762; G06V 10/771; G06V 10/82

(52) U.S. Cl. CPC G06V 20/695; G06T 7/0016; G06T 7/215; G06T 7/246; G06V 10/26; G06V 10/46; G06V 10/7625; G06V 10/763; G06V 10/771; G06V 10/82; G06T 2207/10016; G06T 2207/10056; G06T 2207/10064; G06T 2207/20076; G06T 2207/20084; G06T 2207/30024; G06T 2207/30241

(57) ABSTRACT

This disclosure provides a novel method and system for characterizing morphodynamic profiles of objects, such as biological entities. This disclosure provides a shape, appearance, and motion (SAM) phenotype Observation Tool (SPOT). SPOT establishes a standardized SAM “phenome,” image descriptors resembling single-cell transcriptomes, to comprehensively quantify a cell's instantaneous state without prior knowledge. SPOT also establishes a standardized workflow for temporal analysis. SPOT is a generalist tool, applicable to any live-cell imaging and advances biomedical discovery through its standardized, unbiased, streamlined workflow to quantify phenotypic heterogeneity and predict phenotype-genotype-function coupling.

### CROSS-REFERENCE TO RELATED APPLICATIONS

This application claims priority under 35 U.S.C. § 119 (e) to U.S. Provisional Patent Application No. 63/383,087, filed Nov. 10, 2022. The foregoing application is incorporated by reference herein in its entirety.

### FIELD OF THE INVENTION

This invention relates to methods and systems for characterizing morphodynamic profiles of objects.

### BACKGROUND OF THE INVENTION

With the advent of spatial multiplexing technologies, it is increasingly evident that genetic makeup alone is insufficient to explain cell behavior. Where cells are located in tissue architecture and what other cell types are present in their spatial neighborhood all contribute to shaping cell function. While sequencing can provide in-depth molecular information, it represents only a frozen snapshot in time with no temporal causation. In vivo, cells are phenotypically dynamic and plastic, changing their cell signaling and function in response to genetic, signaling, and environmental perturbations. Moreover, these additional levels of phenotypic complexity are often afforded by the joint regulation of protein translation, post-translational modifications, and metabolism that cannot be captured by measuring RNA levels. Alternatively, morphological and image texture are quantifiable features of cells in complex tissue that have been shown to reflect the sum of interactions between cells, their microenvironment, and genetic makeup, but do not require tissue destruction. Moreover, morphology can be crucial for cell function. Unbiased and comprehensive measurement of dynamical changes in morphology and appearance under live-cell imaging may thus present an inexpensive, high throughput, and label-free assay to monitor phenotypic heterogenicity.

Image-based phenotypic screening has gained momentum in recent years due to its ability to identify the functional consequences of genetic and chemical perturbations in a cost-effective manner. However, most phenotypic studies focus on a limited number of a priori known or predicted phenotypes and biomarkers, such as cellular toxicity, and compare time-averaged measurements or focus on a small number of selected timepoints. Due to these limitations, image-based phenotypic screening is not effective in studying intrinsic heterogeneity, environmental perturbations, and genetic alterations that generate complex, dynamic genotype-phenotype relationships determining cell behavior and cell fate.

With parallels to the pre-molecular sequencing era, quantitative live-cell imaging studies, of organoids in particular, have been largely ad hoc; not only experiment- and assay-specific but limited in scale and scope. Also, there is no standard analysis for temporal imaging and no standardized image descriptors that have been shown to act like a transcriptome to provide a comprehensive description of instantaneous phenome states.

Accordingly, there exists a strong need for technological advancements, similar to that of single-cell transcriptome analysis, to realize the potential of live-cell imaging to unbiasedly and comprehensively capture and quantitatively measure phenotypic heterogeneity overtime.

### SUMMARY OF THE INVENTION

This disclosure addresses the need mentioned above in a number of aspects. In one aspect, this disclosure provides a method for characterizing morphodynamic profiles of one or more objects. In some embodiments, the method comprises: (i) obtaining an image dataset comprising a plurality of images; (ii) detecting a set of objects in each image of the image dataset; (iii) segmenting each image in the image dataset to generate a plurality of image patches, each of the plurality of image patches comprising at least a portion of an object of the set of objects; (iv) determining shape, appearance, and motion (SAM) features for each of the plurality of image patches, wherein the SAM features comprise a set of shape features, a set of appearance features, and a set of motion features; (v) generating SAM descriptors based on the SAM features; and (vi) clustering the set of objects based on the SAM descriptors to provide one or more SAM phenotype clusters of objects having different morphodynamic profiles.

In some embodiments, the method further comprises: after the step of determining the SAM features, performing dimensionality reduction of the SAM features to analyze the SAM features in a reduced- or two-dimensional space. In some embodiments, the dimensionality reduction is performed by Uniform Manifold Approximation and Projection (UMAP).

In some embodiments, the method comprises tracking the detected objects between consecutive images of an image sequence in the image dataset if the image dataset is derived from a video.

In some embodiments, the method comprises pre-processing the SAM features to remove zero-valued, noisy, or non-temporally varying features to select a subset of the SAM features.

In some embodiments, the method comprises computing a SAM phenotype trajectory over a period of time for each of the SAM phenotype clusters or user-defined sub-population of objects in a dataset that has been tagged with the same label to determine temporal evolution of phenotypic diversity in a given object population. In some embodiments, the method comprises determining SAM phenotype frequency over a period of time and/or transition probability between the SAM phenotype clusters. In some embodiments, the method may include determining the cluster transition probability using the categorical hidden markov model (HMM).

In some embodiments, the method comprises automatically grouping the SAM features that exhibit the same covariation into one or more SAM modules. In some embodiments, the method further comprises automatic hierarchical clustering to automatically identifying the one or more SAM modules using a clustering metric.

In some embodiments, the method may include comprising identifying representative image exemplars to visualize a mean of the SAM phenotype clusters, for example, using principal components analysis (PCA). In some embodiments, the method may include finding representative image exemplars and the most important (“driving”) SAM features to visualize and describe respectively what imaging phenotypes the SAM modules are quantifying. In some embodiments, the method may include scoring the relative contribution of shape, appearance or motion or of the spatial scale; global, local-regional or local-distributional, for example using PCA, to describe what types of features are most important in the dataset that has been analyzed.

In some embodiments, the shape features comprise maximum curvature, minimum curvature, mean curvature, mean curvature magnitude, standard deviation curvature, skew curvature, kurtosis curvature, maximum centroid distance, mean centroid distance, standard deviation mean centroid distance ratio, maximum chordal distance, maximum of minimum centroid distance ratio, chordal distance histogram, area, convex hull area, solidity, extent, perimeter, equivalent circular diameter, major axis length, minor axis length, area perimeter aspect ratio, major over minor axis length ratio (eccentricity), moment of eccentricity, Hu moments, Zernike moments, Fourier features, Euler characteristic curves (ecc), shape context, a combination thereof, or transformations thereof.

In some embodiments, the shape features comprise mean global intensity, standard deviation global intensity, mean regional intensity (3 equipartitioned internal regions), standard deviation intensity (3 equipartitioned internal regions), Haralick features, SIFT descriptor, a combination thereof, or transformations thereof.

In some embodiments, the motion features comprise mean global speed, standard deviation global speed, mean global optical flow speed, standard deviation global optical flow speed, mean curl optical flow, standard deviation curl optical flow, mean divergence optical flow, standard deviation divergence optical flow, mean regional optical flow speed (3 equipartitioned internal regions), standard deviation optical flow speed (3 equipartitioned internal regions), histogram regional optical flow speeds (1 for each of the 3 equipartitioned internal regions), sift descriptor of optical flow speed, sift descriptor of curl of optical flow, SIFT descriptor of divergence of optical flow, or a combination and transformations thereof.

In some embodiments, the step of clustering comprises performing k-means clustering for the set of objects. In some embodiments, the step of clustering comprises performing Scikit-learn k-means clustering for the set of objects. In some embodiments, the step of clustering comprises performing hierarchical clustering for the set of objects.

In some embodiments, the step of clustering comprises performing an elbow method to select the number of the one or more SAM phenotype clusters of the objects.

In some embodiments, the step of clustering comprises clustering temporal trajectories of the set of objects. In some embodiments, the method comprises generating a pairwise distance matrix using multidimensional dynamical time warping (DTW).

In some embodiments, the method comprises determining a relationship between the one or more clusters. In some embodiments, the method further comprises determining the relationship between the one or more SAM phenotype clusters using partition-based graph abstraction (PAGA).

In some embodiments, the set of objects comprise biological entities. In some embodiments, the biological entities comprise a cell, an organoid, an organelle, a virus particle, a biopolymer, a polypeptide, a nucleic acid, a lipid, an oligosaccharide, a biomarker, or any combination thereof. In some embodiments, the cell is selected from a eukaryotic cell, a prokaryotic cell, a mammalian cell, a yeast cell, a tumor cell, a circulating tumor cell, a blood cell, a peripheral blood mononuclear cell, a cell of an immune system, a white blood cell, a T cell, a T helper cell, a lymphocyte, a CD4 lymphocyte, a progenitor cell, an endothelial progenitor cell, and a fetal cell.

In some embodiments, the morphodynamic profiles comprise a morphodynamic phenotype of the biological entities. In some embodiments, the morphodynamic phenotype comprises morphodynamic properties of spatially proximal neighboring objects. In some embodiments, the method further comprises correlating the morphodynamic phenotype with a molecular profile of the biological entities.

In some embodiments, the molecular profile is selected from genotype, transcription activity, transcriptomic profile, gene expression activity, genomic profile, protein expression activity, proteomic profile, protein interaction activity, cellular receptor expression activity, lipid profile, lipid activity, carbohydrate profile, microvesicle activity, glucose activity, metabolic profile, and combinations thereof.

In some embodiments, the method comprises correlating the morphodynamic phenotype of the biological entities with gene expression or transcription activities of the biological entities.

In some embodiments, the method comprises correlating the morphodynamic phenotype of the biological entities with a clinical outcome of a treatment.

In some embodiments, the method comprises characterizing a molecular profile of the biological entities. In some embodiments, the method comprises determining the molecular profile by a method selected from DNA analysis, RNA analysis, protein analysis, lipid analysis, metabolite analysis, mass spectrometry, and combinations thereof. In some embodiments, the method comprises determining the molecular profile of the biological entities by single cell RNA sequencing.

In some embodiments, the set of objects are detected by a trained object detection algorithm comprising a convolutional neural network. In some embodiments, the trained object detection algorithm is YOLOv3.

In some embodiments, the set of objects are tracked by a multi-object tracker algorithm. In some embodiments, the tracker algorithm is a frame-by-frame intersection-over-union tracker with optical flow assisted object linking.

In some embodiments, the set of objects are segmented by a trained object detection algorithm comprising a convolutional neural network. In some embodiments, the trained object detection algorithm is attention U-Net.

In some embodiments, the images are obtained by timelapse microscopy. In some embodiments, the images are obtained for the biological entities under different conditions over a period of time. In some embodiments, the images are derived from a video or static images acquired over a period of time. In some embodiments, the images are label free images or fluorescent images.

In some embodiments, the images comprise two-dimensional images, and the method comprises converting three-dimensional z-stack image frames into the two-dimensional images. In some embodiments, the method comprises cropping or rescaling the images. In some embodiments, the method comprises assembling videos of an object acquired from multi-part acquisitions into one long timelapse.

In another aspect, this disclosure provided a system for characterizing morphodynamic profiles of one or more objects. In some embodiments, the system comprises a processor, configured to: (a) obtain an image dataset comprising a plurality of images; (b) detect a set of objects in each image of the image dataset; (c) segment each image in the image dataset to generate a plurality of image patches, each of the plurality of image patches comprising at least a portion of an object of the set of objects; (d) determine shape, appearance, and motion (SAM) features for each of the plurality of image patches, wherein the SAM features comprise a set of shape features, a set of appearance features, and a set of motion features; (e) generate SAM descriptors based on the SAM features; and (f) cluster the set of objects based on the SAM descriptors to provide one or more SAM phenotype clusters of objects having different morphodynamic profiles.

In some embodiments, the processor is further configured to, after the step of determining the SAM features, perform dimensionality reduction for the SAM features to analyze the SAM features in a reduced- or two-dimensional space. In some embodiments, the dimensionality reduction is performed by Uniform Manifold Approximation and Projection (UMAP).

In some embodiments, the processor is configured to track the detected objects between consecutive images of an image sequence in the image dataset if the image dataset is derived from a video.

In some embodiments, the processor is configured to pre-process the SAM features to remove zero-valued, noisy, or non-temporally varying features to select a subset of the SAM features.

In some embodiments, the processor is configured to compute a SAM phenotype trajectory over a period of time for each of the SAM phenotype clusters or user-defined sub-population of objects in a dataset that has been tagged with the same labelto determine temporal evolution of phenotypic diversity in a given object population. In some embodiments, the processor is configured to determine SAM phenotype frequency over a period of time and/or transition probability between the SAM phenotype clusters. In some embodiments, the processor is configured to determine the cluster transition probability using a categorical hidden markov model (HMM).

In some embodiments, the processor is configured to automatically group the SAM features that exhibit the same covariation into one or more SAM modules. In some embodiments, the process is further configured to perform automatic hierarchical clustering to automatically identify the one or more SAM modules using a clustering metric. In some embodiments, the processor is further configured to identify representative image exemplars to visualize a mean of the SAM phenotype clusters, In some embodiments, the processor is further configured to score the relative contribution of shape, appearance or motion or of the spatial scale; global, local-regional or local-distributional to describe what types of features are most important in the dataset that has been analyzed.

In some embodiments, the shape features comprise maximum curvature, minimum curvature, mean curvature, mean curvature magnitude, standard deviation curvature, skew curvature, kurtosis curvature, maximum centroid distance, mean centroid distance, standard deviation mean centroid distance ratio, maximum chordal distance, maximum of minimum centroid distance ratio, chordal distance histogram, area, convex hull area, solidity, extent, perimeter, equivalent circular diameter, major axis length, minor axis length, area perimeter aspect ratio, major over minor axis length ratio (eccentricity), moment of eccentricity, Hu moments, Zernike moments, Fourier features, Euler characteristic curves (ecc), shape context, a combination thereof, or transformations thereof.

In some embodiments, the shape features comprise mean global intensity, standard deviation global intensity, mean regional intensity (3 equipartitioned internal regions), standard deviation intensity (3 equipartitioned internal regions), Haralick features, SIFT descriptor, a combination thereof, or transformations thereof.

In some embodiments, the motion features comprise mean global speed, standard deviation global speed, mean global optical flow speed, standard deviation global optical flow speed, mean curl optical flow, standard deviation curl optical flow, mean divergence optical flow, standard deviation divergence optical flow, mean regional optical flow speed (3 equipartitioned internal regions), standard deviation optical flow speed (3 equipartitioned internal regions), histogram regional optical flow speeds (1 for each of the 3 equipartitioned internal regions), sift descriptor of optical flow speed, sift descriptor of curl of optical flow, SIFT descriptor of divergence of optical flow, a combination thereof, or transformations thereof.

In some embodiments, the step of clustering comprises performing k-means clustering for the set of objects. In some embodiments, the step of clustering comprises performing Scikit-learn k-means clustering for the set of objects. In some embodiments, the step of clustering comprises performing hierarchical clustering for the set of objects.

In some embodiments, the step of clustering comprises performing an elbow system to generate the one or more SAM phenotype clusters of the objects.

In some embodiments, the step of clustering comprises clustering temporal trajectories of the set of objects. In some embodiments, the processor is configured to generate a pairwise distance matrix using multidimensional dynamical time warping (DTW).

In some embodiments, the processor is configured to determine a relationship between the one or more clusters. In some embodiments, the processor is further configured to determine the relationship between the one or more SAM phenotype clusters using partition-based graph abstraction (PAGA).

In some embodiments, the set of objects comprise biological entities. In some embodiments, the biological entities comprise a cell, an organoid, an organelle, a virus particle, a biopolymer, a polypeptide, a nucleic acid, a lipid, an oligosaccharide, a biomarker, or any combination thereof. In some embodiments, the cell is selected from a eukaryotic cell, a prokaryotic cell, a mammalian cell, a yeast cell, a tumor cell, a circulating tumor cell, a blood cell, a peripheral blood mononuclear cell, a cell of an immune system, a white blood cell, a T cell, a T helper cell, a lymphocyte, a CD4 lymphocyte, a progenitor cell, an endothelial progenitor cell, and a fetal cell.

In some embodiments, the morphodynamic profiles comprise a morphodynamic phenotype of the biological entities. In some embodiments, the morphodynamic phenotype comprises morphodynamic properties of spatially proximal neighboring objects. In some embodiments, the processor is further configured to correlate the morphodynamic phenotype with a molecular profile of the biological entities.

In some embodiments, the molecular profile is selected from genotype, transcription activity, transcriptomic profile, gene expression activity, genomic profile, protein expression activity, proteomic profile, protein interaction activity, cellular receptor expression activity, lipid profile, lipid activity, carbohydrate profile, microvesicle activity, glucose activity, metabolic profile, and combinations thereof.

In some embodiments, the processor is configured to correlate the morphodynamic phenotype of the biological entities with gene expression or transcription activities of the biological entities.

In some embodiments, the processor is configured to correlate the morphodynamic phenotype of the biological entities with a clinical outcome of a treatment.

In some embodiments, the processor is further configured to characterize a molecular profile of the biological entities. In some embodiments, the processor is configured to determine the molecular profile by a system selected from DNA analysis, RNA analysis, protein analysis, lipid analysis, metabolite analysis, mass spectrometry, and combinations thereof. In some embodiments, the processor is configured to determine the molecular profile of the biological entities by single cell RNA sequencing.

In some embodiments, the set of objects are detected by a trained object detection algorithm comprising a convolutional neural network. In some embodiments, the trained object detection algorithm is YOLOv3.

In some embodiments, the set of objects are tracked by a multi-object tracker algorithm. In some embodiments, the tracker algorithm is a frame-by-frame intersection-over-union tracker with optical flow assisted object linking.

In some embodiments, the set of objects are segmented by a trained object detection algorithm comprising a convolutional neural network. In some embodiments, the trained object detection algorithm is attention U-Net.

In some embodiments, the images are obtained by timelapse microscopy. In some embodiments, the images are obtained for the biological entities under different conditions over a period of time.

In some embodiments, the images are derived from a video or static images acquired over a period of time. In some embodiments, the images are label free images or fluorescent images.

In some embodiments, the images comprise two-dimensional images, and the processor is configured to convert three-dimensional z-stack image frames into the two-dimensional images. In some embodiments, the processor is configured to crop or rescale the images. In some embodiments, the processor is configured to assemble videos of an object acquired from multi-part acquisitions into one long timelapse.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1a, 1b, 1c, and 1d show that comprehensive shape, appearance, and motion (SAM) characterization enables analysis of phenotypic heterogeneity. FIG. 1a shows that measurement of a standardized high-dimensional library of gene transcripts in single-cell sequencing (scRNA-seq) enables unbiased characterization of the full transcriptome: both that which is known or selected a priori (circle) and unknown (grey ring). Measurement of a standardized high-dimensional set of SAM features is necessary to characterize both known or selected a priori (circle) and unknown (grey ring) imaging phenotypes. FIG. 1b shows an example illustrating how SAM cues provide complementary information and are the minimal three atomic concepts necessary to fully describe the instantaneous state of a dynamic object. FIG. 1c shows categorization of the standardized SAM feature set used in this study with respect to type: shape, appearance or motion (columns) and the spatial scale of detail they measure of an object (rows); global, of the whole object, regional, of a part of the object or distribution, of the object at the level of individual constituent pixels. See Tables 1-3 for the definition of individual features. The number of feature dimensions is given, assuming an object contour of 200 equidistantly spaced points. FIG. 1d shows an overview of the three stages in the SPOT workflow, illustrating the steps in stage 1: video acquisition (orange box), stage 2: computation of SAM phenome for each object instance, and stage 3: the temporal analyses performed using the computed SAM phenomes.

FIGS. 2a, 2b, 2c, 2d, 2e, 2f, 2g, 2h, 2i, and 2j show that standardized SAM features can comprehensively map phenotypic diversity in computer vision datasets. FIG. 2a shows exemplar binary shape images of the MPEG-7 shape computer vision database (Latecki, L. J., and Lakamper, R. (2000). IEEE Transactions on Pattern Analysis and Machine Intelligence 22, 1185-1190) shape classes used to test the informativeness of SAM-S, the shape-only feature set, 1 image per shape category. FIG. 2b shows a shape phenomic landscape constructed by applying 2D UMAP to the full SAM-S(Shape (kernel ECC)) features. Each point is an individual shape image in the MPEG-7 dataset highlighted by its shape category. Similar shapes colocalize to the same local region of the landscape, as shown by zoom-ins 1-4. FIG. 2c shows a visual panel summary of the Normalized Brodatz computer vision texture images (Abdelmounaime, S., and Dong-Chen, H. International Scholarly Research Notices, 2013 (2013)) used to derive a texture dataset for testing the informativeness of SAM-A, the appearance only feature set. FIG. 2d shows an appearance phenomic landscape constructed by applying 2D UMAP to the full SAM-A features. Each shape is a 64×64 image patch cropped from the original 512×512 112 Normalized Brodatz texture image. Images cropped from the same original Brodatz texture image and with similar texture colocalize to the same local region of the landscape as shown by zoom-ins 1-4. FIG. 2e shows selecting 5 representative MPEG-7 shape classes according to eccentricity, the ratio of the length of the longest axis to the shortest axis of an ellipse fitted to the shape (1, top) to create textured MPEG-7 shapes that combine MPEG-7 shape with Normalized Brodatz texture to jointly test SAM-SA (concatenation of shape and appearance features). The textured shape images were generated by multiplication of images of the same size, one MPEG-7 binary image, and one grayscale image crop from a Normalized Brodatz texture image (2, bottom). FIG. 2f shows a shape-appearance phenomic landscape constructed by applying 2D UMAP to the concatenated full SAM-S(Shape (kernel ECC)) and SAM-A feature sets of the textured shapes created from 5 selected MPEG-7 shape categories (see FIGS. 2e) and 5 randomly selected Normalized Brodatz texture images. Each point is a 128×128 image highlighted by its source MPEG-7 shape category (FIG. 2f and FIG. 2g) source Brodatz texture. FIG. 2h shows examples of the object-movement pair annotation in the YouTube-derived A2D dataset (Xu, Chenliang, et al. Proceedings of the IEEE conference on computer vision and pattern recognition. 2015.) used to test the full SAM feature set on “in-the-wild” videos. SAM phenomic landscape of object-motion constructed by applying 2D UMAP to the full SAM feature set concatenating SAM-S (kernel ECC), SAM-A, and SAM-M feature sets highlighted by FIG. 2i object type or FIG. 2j movement class for each A2D annotated object in the test split. Insets: similarity graph between object and motion classes, respectively, based on the pairwise distance between median 2D UMAP coordinates.

FIGS. 3a, 3b, 3c, 3d, 3e, 3f, and 3g show that SPOT characterizes phenotypic heterogeneity of single cell migration. FIG. 3a shows a snapshot of the full field-of-view at timepoint 0 of migrating glioblastoma-astrocytoma U373 cells with the boundary of each cell uniquely highlighted (left) and snapshots of select cells at later timepoints (right). The cells did not divide over the video duration. FIG. 3b shows a SAM phenomic landscape constructed by applying UMAP to SAM phenome and identification of phenotype clusters by k-means clustering of 2D UMAP coordinates using the elbow method. 2×2 image panel shows exemplars of the principal phenotypes in each highlighted cluster (left). Local point density of mapped cell instances, whereby each point, representing a cell instance, is highlighted to indicate low-to-high measured values in global SAM features of shape (eccentricity), appearance (intensity), and motion (speed) (right, first to fourth panel, top-to-bottom). Instances were also highlighted discretely to indicate if it was the first timepoint after cell division (right, fifth panel). As the U373 cells did not divide, no point was highlighted. FIG. 3c shows mapping a single cell tracked over time (left) and all continuously tracked cells (right) into the SAM phenomic landscape of FIG. 3b, and highlighting each temporal instance by the corresponding phenotype cluster. FIG. 3d shows a Stacked barplot showing the relative frequency of each phenotype cluster over discrete time bins (left). Graph showing the Hidden Markov Model inferred transition probability of a cell transitioning to another phenotype cluster in the next timepoint given its phenotype cluster label in the current timepoint. Arrows are highlighted by the source cluster. The more transparent the arrow, the smaller the probability of the transition (right). FIG. 3e shows a single SAM phenotype trajectory summarizing the temporal phenotype dynamics of a single U373 cell (top) and the SAM phenotype trajectory of all cells (bottom) with the starting timepoint highlighted. Black arrow shows the directionality of time. FIG. 3f shows a single population-level SAM phenotype trajectory summarizing the temporal evolution and phenotypic diversity across all cells. The starting timepoint is highlighted. Black arrow shows the directionality of time. FIG. 3g shows a contribution score of shape, appearance, motion, and global, regional and distributional features in explaining the dataset variance defined as the absolute value of the 1^{st} principal component (left). Automated hierarchical clustering of the covariation between SAM features to identify principal SAM feature modules (middle: modules outlined and numbered along the diagonal). Expression of each SAM module in each phenotype cluster (right). Each module is depicted with its top 3 most representative images, its top driving SAM features, and whether the feature is enriched (up arrow) or depleted (down arrow).

FIGS. 4a, 4b, 4c, 4d, and 4e show that SPOT characterizes changes in murine duodenum organoid branching dynamics when treated with HDAC inhibitors. FIG. 4a shows duodenum organoids derived from WT p53^{+/+} and knockout p53^{-/-} mice were grown in 24-well plates. The organoids were untreated or V/C treated (+V/C), a combination of valproic acid (V) and CHIR99021 (C), and filmed every 15 min for 5 days. Media was changed every day, resulting in 5 separate video stacks per well, which were then frame-by-frame registered and concatenated into one video for analysis (top right). Snapshots of an example video from each condition on days 0-5. Scalebar: 50 μm. FIG. 4b shows a UMAP phenomic landscape of filtered SAM phenomes from a total of 80 videos (20 per condition), 1-5 organoids in the field-of-view and carried out in 24-well plates. n=34,121 points, each point=a segmented organoid instance. The landscape is highlighted and numbered in ascending order according to the mean shape eccentricity of each phenotype cluster identified by k-means clustering and the elbow method (top, left). For each cluster, 4 representative images arranged as a 2×2 grid are visualized, using their real size for comparison of scale across clusters (bottom, top row) and after cropping and resizing to a standardized size to show organoid appearance (bottom, bottom row). Illustration of real vs. standardized (std.) size (top right). FIG. 4c shows hierarchically clustered treatment conditions according to UMAP SAM temporal phenotype trajectories with complete linkage (bottom). Row (i): final frame of example videos in FIG. 4a for each condition. Scalebar: 50 μm. Row (ii): smoothed UMAP local point density heatmaps. Row (iii): stacked barplots showing the temporal changes in the phenotype cluster distribution of segmented organoid instances in consecutive 3 h time intervals for each indicated condition. Row (iv): graph showing the Hidden Markov Model inferred transition probability of an organoid transitioning to another phenotype cluster in the next timepoint given its phenotype cluster label in the current timepoint. Arrows are highlighted by the source cluster; the more transparent the arrow, the lower the probability. Row (v): UMAP SAM temporal phenotype trajectories for a density threshold of mean+3 standard deviation. Each highlighted point on a trajectory denotes half day increment from the starting point on each trajectory. Black arrow indicates the arrow of time. FIG. 4d shows a contribution score of shape, appearance, motion, and global, regional and distributional features (left), automated hierarchical clustering to identify SAM feature modules (right) and SAM module expression in each phenotype cluster (lower) constructed as in FIG. 2g. FIG. 4e shows a multidimensional scaling (MDS) plot of the log₂ (CPM) expression of bulk RNA-seq counts from WT p53^{+/+} and knockout p53^{-/-} organoids with no treatment harvested at day 0 or harvested at day 5 after V/C treatment (V/C), where CPM stands for transcript counts per million.

FIGS. 5a, 5b, 5c, and 5d show that SPOT captures the timing of WNT signaling-induced phenotypic dynamics in patient-derived duodenal organoids. FIG. 5a shows patient-derived D2 duodenum organoids grown from endoscopy biopsies and cultured in different medium conditions: human organoid media (HOM), ENR and ENR+WNT3A media. FIG. 5b shows timelines of organoid culture and timelapse video acquisition using brightfield microscopy (left) and temporal snapshots of organoids cultured in HOM, ENR and ENR+WNT3A media after initial 7-day incubation in HOM media (right). Dashed white contour line from day 6 in ENR outlines the flattened organoids, merging into a sheet. Scalebars: 200 μm. FIG. 5c shows the timeline of treatment and image acquisition for progressively delayed WNT3A restoration to ENR cultured organoids. FIG. 5d shows UMAP SAM temporal phenotype trajectory from 0-14 days. Each coloured point denotes one day increments, with the starting timepoint coloured black. Black arrows show the direction of time. Black box indicates the timepoint of WNT3A addition. Average linkage hierarchical clustering of the mean trajectories (right).

FIG. 6 shows an example computing system to implement the disclosed methods according to various embodiments of this disclosure.

### DETAILED DESCRIPTION OF THE INVENTION

This disclosure provides a novel method and system for characterizing morphodynamic profiles of objects, such as biological entities. This disclosure provides a shape, appearance, and motion (SAM) phenotype Observation Tool (SPOT). SPOT establishes a standardized SAM “phenome,” image descriptors resembling single-cell transcriptomes, to comprehensively quantify a cell's instantaneous state without prior knowledge. SPOT also establishes a standardized workflow for temporal analysis. SPOT is a generalist tool applicable to any live-cell imaging and advances biomedical discovery through its standardized, unbiased, streamlined workflow to quantify phenotypic heterogeneity and predict phenotype-genotype-function coupling.

### Methods and Systems for Analyzing Chromatins

Accordingly, in one aspect, this disclosure provides a method for characterizing morphodynamic profiles of one or more objects. In some embodiments, the method may include: (i) obtaining an image dataset comprising a plurality of images; (ii) detecting a set of objects in each image of the image dataset; (iii) segmenting each image in the image dataset to generate a plurality of image patches, each of the plurality of image patches comprising a least a portion of an object of the set of objects; (iv) determining shape, appearance, and motion (SAM) features for each of the plurality of image patches, wherein the SAM features may include a set of shape features, a set of appearance features, and a set of motion features; (v) generating SAM descriptors based on the SAM features; and (vi) clustering the set of objects based on the SAM descriptors to provide one or more clusters of objects having different morphodynamic profiles.

In some embodiments, the method may include tracking the detected objects between consecutive images of an image sequence in the image dataset if the image dataset is derived from a video.

In some embodiments, the method comprises pre-processing the SAM features to remove zero-valued, noisy, or non-temporally varying features to select a subset of the SAM features.

In some embodiments, the method comprises computing a SAM phenotype trajectory over a period of time for each of the SAM phenotype clusters or user-defined sub-population of objects in a dataset that has been tagged with the same label to determine temporal evolution of phenotypic diversity in a given object population. In some embodiments, the method comprises determining SAM phenotype frequency over a period of time and/or transition probability between the SAM phenotype clusters. In some embodiments, the method may include determining the cluster transition probability using the categorical hidden markov model (HMM).

In some embodiments, the method comprises automatically grouping the SAM features that exhibit the same covariation into one or more SAM modules. In some embodiments, the method may further include automatic hierarchical clustering to automatically identifying the one or more SAM modules using a clustering metric, such as the Davies-Bouldin index.

In some embodiments, the method may include finding representative image exemplars to visualize what the phenotype clusters mean/represent, for example, using principal components analysis (PCA). In some embodiments, the method may include finding representative image exemplars and the most important (“driving”) SAM features to visualize and describe respectively what imaging phenotypes the SAM modules are quantifying. In some embodiments, the method may include scoring the relative contribution of shape, appearance or motion or of the spatial scale; global, local-regional or local-distributional, for example using PCA, to describe what types of features are most important in the dataset that has been analyzed.

In some embodiments, the method may include: after the step of determining the SAM features, performing dimensionality reduction for the SAM features to analyze the SAM features in a reduced- or two-dimensional space. Dimensionality reduction approaches are methods for reducing the number of random variables under consideration by identifying a set of principal variables. They are typically used for processes such as feature aggregation, feature selection, and feature extraction. Examples of dimensionality reduction approaches that may be incorporated into machine learning approaches include, but are not limited to, principal component analysis (PCA), multidimensional scaling (MDS), t-distributed stochastic neighbor embedding (t-SNE), autoencoders, and uniform manifold approximation and projection (UMAP).

In some embodiments, the dimensionality reduction is performed by UMAP. UMAP is a machine learning technique for dimension reduction. UMAP is constructed from a theoretical framework based on Riemannian geometry and algebraic topology. The result is a practical, scalable algorithm that applies to real world data. The UMAP algorithm is competitive with t-SNE for visualization quality and, in some cases, preserves more of the global data structure with superior run time performance. Furthermore, UMAP has no computational restrictions on embedding dimension, making it viable as a general purpose dimension reduction technique for machine learning.

### Sam Features-Based Characterization of Morphodynamic Profiles

The existing image-based analytical tools in the art compute only a limited number of imaging features for particular experiments and are not universally applicable across imaging and experimental conditions. The choices of these features are either too small or insufficiently diverse, informed by prior knowledge, or, if machine learning-based, limited to the generation of features that only discriminate between the given experimental conditions or to the ability to encode and reconstruct only the input data. In contrast, the disclosed methods utilize diverse local and global SAM features that comprehensively capture the complexity of the instantaneous phenotypic state of biological entities. Non-limiting examples of local and global SAM features are shown in Tables 1-3.

In some embodiments, the shape features may include maximum curvature, minimum curvature, mean curvature, mean curvature magnitude, standard deviation curvature, skew curvature, kurtosis curvature, maximum centroid distance, mean centroid distance, standard deviation mean centroid distance ratio, maximum chordal distance, maximum of minimum centroid distance ratio, chordal distance histogram, area, convex hull area, solidity, extent, perimeter, equivalent circular diameter, major axis length, minor axis length, area perimeter aspect ratio, major over minor axis length ratio (eccentricity), moment of eccentricity, Hu moments, Zernike moments, Fourier features, Euler characteristic curves (ecc), shape context, a combination thereof, or transformations thereof.

In some embodiments, the shape features may include mean global intensity, standard deviation global intensity, mean regional intensity (3 equipartitioned internal regions), standard deviation intensity (3 equipartitioned internal regions), Haralick features, SIFT descriptor, a combination thereof, or transformations thereof.

In some embodiments, the motion features may include mean global speed, standard deviation global speed, mean global optical flow speed, standard deviation global optical flow speed, mean curl optical flow, standard deviation curl optical flow, mean divergence optical flow, standard deviation divergence optical flow, mean regional optical flow speed (3 equipartitioned internal regions), standard deviation optical flow speed (3 equipartitioned internal regions), histogram regional optical flow speeds (1 for each of the 3 equipartitioned internal regions), sift descriptor of optical flow speed, sift descriptor of curl of optical flow, SIFT descriptor of divergence of optical flow, a combination thereof, or transformations thereof.

In some embodiments, the step of clustering may include performing k-means clustering for the set of objects. In some embodiments, the step of clustering may include performing Scikit-learn k-means clustering for the set of objects. K-means is an iterative clustering algorithm that seeks to partition the given input dataset into K clusters, such that every datapoint is assigned to the cluster it is closest to by distance. The number of clusters, K is provided by the user. In some embodiments, K is chosen by the elbow method. Scikit-learn refers to the specific code library implementation of the K-means clustering algorithm. Scikit-learn refers to the specific code library implementation of the K-means clustering algorithm.

In some embodiments, the step of clustering may include performing hierarchical clustering for the set of objects.

As used herein, “hierarchical clustering” refers to the building (agglomerative) or break up (divisive) of a hierarchy of clusters. The traditional representation of this hierarchy is a dendrogram, with individual elements at one end and a single cluster containing every element at the other. Agglomerative algorithms begin at the leaves of the tree, whereas divisive algorithms begin at the root. Methods for performing hierarchical clustering are well-known in the art. Hierarchical clustering methods have been widely used to cluster biological samples based on their gene expression patterns and derive subgroup structures in populations of samples in biomedical research. “Agglomerative hierarchical clustering” refers to clustering techniques that produce a hierarchical clustering by starting with each point as a singleton cluster and then repeatedly merging the two closest clusters until a single, all-encompassing cluster remains. Agglomerative hierarchical clustering cannot be viewed as globally optimizing an objective function. Instead, agglomerative hierarchical clustering techniques use various criteria to decide locally, at each step, which clusters should be merged (or split for divisive approaches). This approach yields clustering algorithms that avoid the difficulty of attempting to solve a hard combinatorial optimization problem. Furthermore, such approaches do not have problems with local minima or difficulties in choosing initial points. Of course, the time complexity of O(m² log m) and the space complexity of O(m²) are prohibitive in many cases. Agglomerative hierarchical clustering algorithms tend to make good local decisions about combining two clusters since they can use information about the pair-wise similarity of all points. However, once a decision is made to merge two clusters, it cannot be undone at a later time. This approach prevents a local optimization criterion from becoming a global optimization criterion.

In some embodiments, the step of clustering may include performing an elbow method to select the number of one or more clusters of the objects. In cluster analysis, the elbow method is a heuristic used in determining the number of clusters in a data set. The method consists of plotting the explained variation as a function of the number of clusters and picking the elbow of the curve as the number of clusters to use. The elbow method runs k-means clustering on the dataset for a range of values of k, and for each value of k calculate the sum of squared errors (SSE).

In some embodiments, the step of clustering may include clustering temporal trajectories of the set of objects. In some embodiments, the method may include generating a pairwise distance matrix using multidimensional dynamical time warping (DTW). In time series analysis, dynamic time warping (DTW) is an algorithm for measuring similarity between two temporal sequences, which may vary in speed. DTW can be applied to temporal sequences of video, audio, and graphics data.

In some embodiments, the method may include determining a relationship between one or more clusters. In some embodiments, the method may include determining the relationship between one or more clusters using partition-based graph abstraction (PAGA). PAGA provides an interpretable graph-like map of the arising data manifold, based on estimating connectivity of manifold partitions. PAGA maps preserve the global topology of data, allow analyzing data at different resolutions, and result in much higher computational efficiency than the typical exploratory data analysis workflow.

In some embodiments, objects may include biological entities. In some embodiments, the biological entities may include a cell, an organoid, an organelle, a virus particle, a biopolymer, a polypeptide, a nucleic acid, a lipid, an oligosaccharide, a biomarker, or any combination thereof.

In some embodiments, the cell may be selected from a eukaryotic cell, a prokaryotic cell, a mammalian cell, a yeast cell, a tumor cell, a circulating tumor cell, a blood cell, a peripheral blood mononuclear cell, a cell of an immune system, a white blood cell, a T cell, a T helper cell, a lymphocyte, a CD4 lymphocyte, a progenitor cell, an endothelial progenitor cell, and a fetal cell.

Correlation of Morphodynamic Profiles with Other Characteristics

In some embodiments, the morphodynamic profiles may include a morphodynamic phenotype of the biological entities. As used herein, the term “phenotype” refers to an observable physical or biochemical characteristic of a biological entity (e.g., a cell or an organoid). The term “morphodynamic phenotype” refers to morphological and dynamic characteristics, such as shape, appearance, and motion properties of a biological entity.

In some embodiments, the method may include correlating the morphodynamic phenotype with a molecular profile of the biological entity.

In some embodiments, the molecular profile may include, without limitation, genotype, transcription activity, transcriptomic profile, gene expression activity, genomic profile, protein expression activity, proteomic profile, protein interaction activity, cellular receptor expression activity, lipid profile, lipid activity, carbohydrate profile, microvesicle activity, glucose activity, metabolic profile, and combinations thereof.

In some embodiments, the method may include correlating a morphodynamic phenotype of the biological entity with its gene expression or transcription activities. In some embodiments, the method may include correlating a morphodynamic phenotype of the biological entity with its genotype. In some embodiments, the genotype and phenotype descriptors are concatenated together to predict the outcome of treatment (or other desired outcome). The extent of shared information between genotype and phenotype descriptors may be analyzed using canonical correlation analysis (CCA) (Ash et al. Nat Commun. 2021; 12:1609). The similarities and differences in the clustering of conditions may be compared using genotype and phenotype descriptors separately or in combination.

As used herein, the term “genotype” refers to the genetic constitution of a cell or organism. As used herein, the term “genotyping” or “determining the genotype” refers to the process of determining genetic variations among individuals in a species. Single nucleotide polymorphisms (SNPs) are the most common type of genetic variation that are used for genotyping and, by definition, are single-base differences at a specific locus.

In some embodiments, the method may include characterizing a molecular profile of the biological entity. In some embodiments, the method may include determining the molecular profile by a method selected from DNA analysis, RNA analysis, protein analysis, lipid analysis, metabolite analysis (e.g., glucose analysis), mass spectrometry, and combinations thereof.

In some embodiments, the molecular profile may be determined by DNA analysis. In some embodiments, the DNA analysis may include amplification of DNA sequences from one or more identified cells. In some embodiments, the amplification may be carried out by the polymerase chain reaction (PCR).

In some embodiments, the molecular profile may be determined by RNA analysis. In some embodiments, the RNA analysis includes RNA quantification. In some embodiments, the RNA quantification may be carried out by reverse transcription quantitative PCR (RT-qPCR), multiplexed qRT-PCR, fluorescence in situ hybridization (FISH), and combinations thereof.

In some embodiments, the molecular profile may be determined by RNA or DNA sequencing. In some embodiments, the RNA or DNA sequencing may be determined by methods that may include, without limitation, whole transcriptome analysis, whole genome analysis, barcoded sequencing of whole or targeted regions of the genome, and combinations thereof. In some embodiments, the method may include determining the molecular profile of the biological entities by single cell RNA sequencing.

In some embodiments, the microvesicles, exosomes or microparticles secreted by the individual cells or aggregates may be detected by RNA-sequencing or antibody-based methods.

In some embodiments, the molecular profile may be determined by protein analysis. In some embodiments, the protein analysis may be carried out at the proteomic level. In some embodiments, the protein analysis may be carried out by multiplexed fluorescent staining. In some embodiments, the comprehensive metabolic profile of single cells may be determined by using mass spectrometry.

In some embodiments, the method may include correlating the morphodynamic phenotype of the biological entities with a clinical outcome of a treatment. For example, in some embodiments, the correlated information can be utilized for at least one of predicting a clinical outcome of a treatment, predicting or determining efficacy of drugs, screening cells, retrieving cells for further evaluation, facilitating a treatment, diagnosing a disease, monitoring cellular activity, and combinations thereof.

In some embodiments, the correlated information can be utilized to facilitate a treatment. In some embodiments, the treatment includes immunotherapy. For example, in some embodiments, the method may include dynamically profiling interactions between immune cells and tumor cells, and performing subsequent proteomic/transcriptomic profiling on the immune cells allows for engineering better immunotherapies.

In some embodiments, the correlated information can be utilized to monitor cellular activity. In some embodiments, the monitored cellular activity includes an immune response.

In some embodiments, the correlated information can be utilized to screen cells, such as the screening of cells for clinical efficacy. For example, in some embodiments, the screened cells include multi-killer T cells. In some embodiments, the functional and molecular characteristics of the multi-killer T-cells are evaluated before selecting subsets for preclinical and clinical tests.

In some embodiments, the correlated information can be utilized to predict clinical outcome, such as the outcome of an immunotherapy. For example, in some embodiments, the observed ability of a T cell to persist and participate in killing of tumor cells can be utilized as a predictor of the therapeutic success of the identified T-cell in cancer therapy. Likewise, the characterized protein expression activity of the identified T-cell can be utilized to introduce various markers (e.g., immune receptors) onto the T-cell in order to enhance therapeutic success in vivo.

In some embodiments, the images are obtained by timelapse microscopy. In some embodiments, the images are obtained for the biological entities under different conditions over a period of time.

In some embodiments, the images are obtained for cells under different conditions or treatments. For example, the method may include pre-treating a cell population with an agent. In some embodiments, the agent includes, without limitation, small molecules, drugs, antibodies, cytokines, chemokines, growth factors, and combinations thereof.

In some embodiments, the method may include pre-treating a cell population with other biological entities. In some embodiments, the other biological entities may include cells of the same species, pathogens or symbiotes. In some embodiments, other biological entities can include, without limitation, viruses, bacteria, parasites, and combinations thereof.

In some embodiments, the images are derived from a video (e.g., live cell imaging videos) or static images acquired over a period of time. In some embodiments, the images are label free images or fluorescent images. For example, in some embodiments, the images are acquired at sequential intervals for a period of time. In some embodiments, the period of time ranges from about 1 minute to about 30 days. In some embodiments, the period of time ranges from about 1 minute to about 24 hours. In some embodiments, the period of time ranges from about 1 hour to about 24 hours. In some embodiments, the period of time ranges from about 5 hours to about 24 hours. In some embodiments, the period of time ranges from about 12 hours to about 14 hours.

In some embodiments, the sequential intervals range from about 1 minute to about 60 minutes. In some embodiments, the sequential intervals range from about 1 minute to about 10 minutes. In some embodiments, the sequential intervals range from about 5 minutes to about 10 minutes. In some embodiments, the sequential intervals range from about 5 minutes to about 6 minutes.

In some embodiments, the method may include converting three-dimensional z-stack image frames into two-dimensional images.

In some embodiments, the method may include preprocessing an image. In some embodiments, preprocessing may include cropping, resizing, gradation conversion, median filtering, histogram equalization, or size-normalized image processing. In some embodiments, the method may include cropping or rescaling the images. In some embodiments, the method comprises assembling videos of an object acquired from multi-part acquisitions into one long timelapse.

As used herein, the term “image” or “images” refers to single or multiple frames of still or animated images, video clips, video streams, etc. The images may be in any suitable format. Many types of images or formats may be used in the context of the present disclosure, for example, compressed images such as in Joint Photographic Experts Group (JPEG) or Luminance/Chrominance (YUV) formats, compressed images as frames stemming from a compressed video format such as H.264/Advanced Video Coding (AVC) or H.265/High Efficiency Video Coding (HEVC), raw images such as originating from Red Clear Blue (RCCB), Red Clear (RCCC) or other type of imaging sensor. According to various embodiments of the present disclosure, images may be 8-bit RGB images.

In some embodiments, videos of an object can be acquired from multi-part acquisitions and then subsequently spatiotemporally registered and “stitched” together into one long timelapse.

In some embodiments, objects, such as cells, may be detected or identified by a trained object detection algorithm comprising a convolutional neural network. In some embodiments, the trained object detection algorithm is YOLO (e.g., YOLOv3). The YOLO machine learning algorithm uses features learned by a deep convolutional neural network to detect an object. YOLOv3 is a real-time object detection algorithm that identifies specific objects in videos, live feeds, or images. YOLOv3 is a real-time, single-stage object detection model that builds on YOLOv2 with several improvements. Improvements include the use of a new backbone network, Darknet-53 that utilizes residual connections, as well as some improvements to the bounding box prediction step, and use of three different scales from which to extract features.

Cell identification may be carried out by various methods. For example, in some embodiments, one or more cells are identified manually. In some embodiments, cells may be identified automatically. In some embodiments, cells may be identified automatically through the use of algorithms. In some embodiments, cells may be identified through the use of automated segmentation and tracking algorithms.

In another aspect, the disclosed method may be used to characterize various dynamic behaviors of cell populations. For example, in some embodiments, the dynamic behavior includes, without limitation, cellular activation, cellular inhibition, cellular interaction, protein expression, protein secretion, metabolite secretion, changes in lipid profiles, microvesicle secretion, exosome secretion, microparticle secretion, changes in cellular mass, cellular proliferation, changes in cellular morphology, motility, cell death, cell cytotoxicity, cell lysis, cell membrane polarization, establishment of a synapse, dynamic trafficking of proteins, granule polarization, calcium activation, metabolic changes, and combinations thereof.

In some embodiments, the dynamic behavior may include protein secretion. In some embodiments, the dynamic behavior may include motility. In some embodiments, the dynamic behavior may include cell death, such as activation-induced cell death.

In some embodiments, the dynamic behavior may include cellular interaction. In some embodiments, the cellular interaction may include, without limitation, heterologous cellular interaction, homologous cellular interaction, and combinations thereof.

In some embodiments, the dynamic behavior may include a change in cellular morphology. In some embodiments, the change in cellular morphology may include, without limitation, a change in cell shape, a change in cell volume, a change in cell mass, a change in cell size, a change in cell polarization, and combinations thereof.

In some embodiments, the dynamic behavior may include a change in cellular appearance.

In some embodiments, the change in cellular appearance may include, without limitation, a change in image intensity, image contrast, image edges, image intensity gradients, image intensity patterns, image textures, or combinations thereof.

In another aspect, this disclosure provided a system for characterizing morphodynamic profiles of one or more objects. In some embodiments, the system comprises a processor, configured to: (a) obtain an image dataset comprising a plurality of images; (b) detect a set of objects in each image of the image dataset; (c) segment each image in the image dataset to generate a plurality of image patches, each of the plurality of image patches comprising a least a portion of an object of the set of objects; (d) determine shape, appearance, and motion (SAM) features for each of the plurality of image patches, wherein the SAM features comprise a set of shape features, a set of appearance features, and a set of motion features; (e) generate SAM descriptors based on the SAM features; and (f) cluster the set of objects based on the SAM descriptors to provide one or more clusters of objects having different morphodynamic profiles.

In some embodiments, the processor is further configured to: after the step of determining the SAM features, perform dimensionality reduction for the SAM features to analyze the SAM features in a reduced- or two-dimensional space. In some embodiments, the dimensionality reduction is performed by Uniform Manifold Approximation and Projection (UMAP).

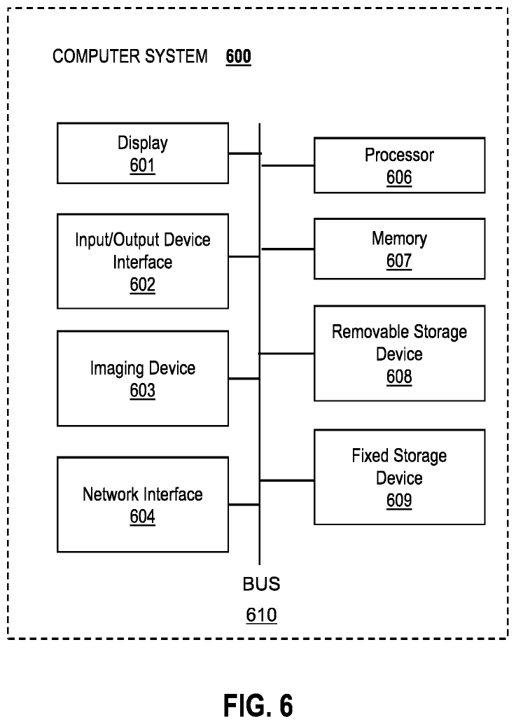

FIG. 6 is a functional diagram illustrating a programmed computer system to implement the disclosed methods in accordance with some embodiments. As will be apparent, other computer system architectures and configurations can be used to perform the described methods. Computer system 600, which includes various subsystems as described below, includes at least one microprocessor subsystem (also referred to as a processor or a central processing unit (CPU) 606). For example, processor 606 can be implemented by a single-chip processor or by multiple processors. In some embodiments, processor 606 is a general purpose digital processor that controls the operation of the computer system 600. In some embodiments, processor 606 also includes one or more coprocessors or special purpose processors (e.g., a graphics processor, a network processor, etc.). Using instructions retrieved from memory 607, processor 606 controls the reception and manipulation of input data received on an input device (e.g., image processing device 603, I/O device interface 602), and the output and display of data on output devices (e.g., display 601).

Processor 606 is coupled bi-directionally with memory 607, which can include, for example, one or more random access memories (RAM) and/or one or more read-only memories (ROM). As is well known in the art, memory 607 can be used as a general storage area, a temporary (e.g., scratchpad) memory, and/or a cache memory. Memory 607 can also be used to store input data and processed data, as well as to store programming instructions and data, in the form of data objects and text objects, in addition to other data and instructions for processes operating on processor 606. Also, as is well known in the art, memory 607 typically includes basic operating instructions, program code, data, and objects used by the processor 606 to perform its functions (e.g., programmed instructions). For example, memory 607 can include any suitable computer-readable storage media described below, depending on whether, for example, data access needs to be bi-directional or uni-directional. For example, processor 606 can also directly and very rapidly retrieve and store frequently needed data in a cache memory included in memory 607.

A removable mass storage device 608 provides additional data storage capacity for the computer system 600, and is optionally coupled either bi-directionally (read/write) or uni-directionally (read-only) to processor 606. A fixed mass storage 609 can also, for example, provide additional data storage capacity. For example, storage devices 608 and/or 609 can include computer-readable media such as magnetic tape, flash memory, PC-CARDS, portable mass storage devices such as hard drives (e.g., magnetic, optical, or solid state drives), holographic storage devices, and other storage devices. Mass storages 608 and/or 609 generally store additional programming instructions, data, and the like that typically are not in active use by the processor 606. It will be appreciated that the information retained within mass storages 608 and 609 can be incorporated, if needed, in a standard fashion as part of memory 607 (e.g., RAM) as virtual memory.

In addition to providing processor 606 access to storage subsystems, bus 610 can be used to provide access to other subsystems and devices as well. As shown, these can include a display 601, a network interface 604, an input/output (I/O) device interface 602, an image processing device 603, as well as other subsystems and devices. For example, image processing device 603 can include a camera, a scanner, etc.; I/O device interface 602 can include a device interface for interacting with a touchscreen (e.g., a capacitive touch sensitive screen that supports gesture interpretation), a microphone, a sound card, a speaker, a keyboard, a pointing device (e.g., a mouse, a stylus, a human finger), a global positioning system (GPS) receiver, a differential global positioning system (DGPS) receiver, an accelerometer, and/or any other appropriate device interface for interacting with system 600. Multiple I/O device interfaces can be used in conjunction with computer system 600. The I/O device interface can include general and customized interfaces that allow the processor 606 to send and, more typically, receive data from other devices such as keyboards, pointing devices, microphones, touchscreens, transducer card readers, tape readers, voice or handwriting recognizers, biometrics readers, cameras, portable mass storage devices, and other computers.

The network interface 604 allows processor 606 to be coupled to another computer, computer network, or telecommunications network using a network connection as shown. For example, through the network interface 604, the processor 606 can receive information (e.g., data objects or program instructions) from another network, or output information to another network in the course of performing method/process steps. Information, often represented as a sequence of instructions to be executed on a processor, can be received from and outputted to another network. An interface card or similar device and appropriate software implemented by (e.g., executed/performed on) processor 606 can be used to connect the computer system 600 to an external network and transfer data according to standard protocols. For example, various process embodiments disclosed herein can be executed on processor 606 or can be performed across a network such as the Internet, intranet networks, or local area networks, in conjunction with a remote processor that shares a portion of the processing. Additional mass storage devices (not shown) can also be connected to processor 606 through network interface 604.

In addition, various embodiments disclosed herein further relate to computer storage products with a computer-readable medium that includes program code for performing various computer-implemented operations. The computer-readable medium includes any data storage device that can store data that can thereafter be read by a computer system. Examples of computer-readable media include, but are not limited to: magnetic media such as disks and magnetic tape; optical media such as CD-ROM disks; magneto-optical media such as optical disks; and specially configured hardware devices such as application-specific integrated circuits (ASICs), programmable logic devices (PLDs), and ROM and RAM devices. Examples of program code include both machine code as produced, for example, by a compiler, or files containing higher level code (e.g., script) that can be executed using an interpreter.

The computer system as shown in FIG. 6 is an example of a computer system suitable for use with the various embodiments disclosed herein. Other computer systems suitable for such use can include additional or fewer subsystems. In some computer systems, subsystems can share components (e.g., for touchscreen-based devices such as smartphones, tablets, etc., I/O device interface 602 and display 601 share the touch-sensitive screen component, which both detects user inputs and displays outputs to the user). In addition, bus 610 is illustrative of any interconnection scheme serving to link the subsystems. Other computer architectures having different configurations of subsystems can also be utilized.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Aspects of the present disclosure are described herein with reference to flowchart illustrations and/or block diagrams of methods, apparatus (systems), and computer program products according to embodiments of the invention. In some embodiments, the flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Unless specifically stated otherwise, it is appreciated that throughout the disclosure, descriptions utilizing terms such as “obtaining,” “performing,” “receiving,” “computing,” “associating,” “assigning,” “traversing,” “calculating,” “determining,” “identifying,” “transforming,” “ranking,” “providing,” “transmitting,” or the like, refer to the action and processes of a computer system, or similar electronic computing device, that manipulates and transforms data represented as physical (or electronic) quantities within the computer system memories or registers or other such information storage, transmission or display devices.

It will be understood that, although the terms “first,” “second,” etc., may be used herein to describe various elements, components, regions, layers and/or sections. These elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

It is noted here that, as used in this specification and the appended claims, the singular forms “a,” “an,” and “the” include plural reference unless the context clearly dictates otherwise. The terms “including,” “comprising,” “containing,” or “having” and variations thereof are meant to encompass the items listed thereafter and equivalents thereof as well as additional subject matter unless otherwise noted.

As used herein, “plurality” means two or more. As used herein, a “set” of items may include one or more of such items.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples or exemplary language (e.g., “such as”) provided herein is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes the materials and methods used in the subsequent EXAMPLES below.

### Human Duodenum Organoids

Duodenum biopsies were collected from outpatients during endoscopy at the John Radcliffe Hospital, Oxford, with written informed consent (through the Oxford Gastrointestinal Illness Biobank, authorized by Yorkshire & The Humber—Sheffield Research Ethics Committee: 16/YH/0247; laboratory research using the samples authorized by South Central—Oxford C Research Ethics Committee: 09/H0606/78).

### Isolation of Endoscopic-Derived Duodenum for Organoid Culture.

Half of the duodenum tissues collected from endoscopy were cut into 2-5 mm² pieces and then washed twice with cold PBS. Tissue fragments were isolated in 5 mM EDTA-PBS (Thermo Fisher Scientific) on a roller at 4° C. for 15 min and digested in TrypLE (Thermo Fisher Scientific) at 37° C. water bath for 30 min (Jung, P., et al. Nat Med. 2011 September). Microcentrifugation was carried out for 15 sec, TrypLE was removed, and fragments were washed with ice-cold PBS twice. Vigorous trituration broke up organoids and facilitated cell release into the PBS. The remaining tissues were left to settle, and the supernatant was collected and filtered through a cell strainer (70 μm, Corning). This was microcentrifuged, and the pellet was resuspended in 100 μL of growth factor reduced (GFR) Matrigel (Corning), then seeded in a well of a pre-warmed 24-well flat bottom cell culture plate (Corning). The Matrigel was then solidified by incubation in a 37° C. and 5% CO₂ cell culture incubator for 10 min and overlaid with 500 μL of human organoid media, which was subsequently replaced every other day. The remaining half of the duodenum tissues were placed in a cryovial and stored at −80° C.

### Passaging the Duodenum Organoids.

Passage was performed every 1-2 weeks with a 1:4 split ratio. Briefly, duodenum organoids were mechanically harvested from Matrigel using ice-cold PBS, then microcentrifuged for 15 s to pellet the organoids. PBS was removed and replaced with 1 mL TrypLE solution for 5 min at room temperature. The solution was microcentrifuged for 15 s to pellet the organoids, and the TrypLE supernatant was removed. The pellet was then resuspended in GFR Matrigel and reseeded in a well of a pre-warmed 24-well flat bottom cell culture plate (Corning). The Matrigel was then solidified by incubation in a 37° C. and 5% CO₂ cell culture incubator for 10 min and overlaid with 500 μL of human organoid media, which was subsequently replaced every other day.

### Organoid Culture Medium.

HEK293T Rspo1-Fc cells (provided by the laboratory of Prof. Gijs van den Brink), HEK293T Nog-Fc cells (provided by the laboratory of Prof. Calvin Kuo, Stanford University) and L Wnt3A (CRL-2647™) cells were used to generate R-Spondin-, Noggin-, and Wnt3A conditioned medium respectively. To produce sufficient quantities of conditioned medium, cells were initially grown to confluency at 37° C., 5% CO₂, in three T175 flasks containing the relevant selective medium and then expanded into 15 T175 flasks containing the relevant non-selective medium. HEK293T Rspo1-Fc cells and HEK293T Nog-Fc cells were grown in 50 mL of non-selective medium for 7 days before collection. L Wnt3A cells were grown in 25 mL of non-selective medium for 5 days, followed by medium collection and the addition of another 25 mL of fresh non-selective medium for a further two days. The two Wnt3A conditioned medium collections were then pooled together. A HEK293T Wnt3A luciferase reporter cell line (provided by the laboratory of Prof. Fiona Powrie, Oxford University) was used to test the quality of the R-Spondin and Wnt3A conditioned medium. Cells were expanded in the relevant selective medium, plated at confluency in a 24-well plate (in a non-selective medium), and allowed to adhere overnight. Cells were then transfected with 0.4 ng/ml of Renilla, using Lipofectamine 2000 (ThermoFisher) and Opti-MEM (ThermoFisher) as the transfection reagents. 5 hours later, 500 μL of conditioned medium was added to each well. The cells were left to incubate overnight, following which the Dual Luciferase® Reporter Assay System (Promega) and GloMax Multi Detection Plate Reader (Promega) were used to determine the luminescence of each well. To test the quality of the Noggin-conditioned medium, 500 μL of organoid medium containing the newly made Noggin-conditioned medium was placed on growing organoids, and the growth was monitored for 7-10 days. If the organoids began to die or grow at a slower rate, the batch was discarded. Once tested, all conditioned medium was aliquoted and stored at −80° C.

### Murine Fluorescent Mutant Colon Organoids

Animal work was approved by local ethical review and licensed by the UK Home Office (PPL 30/3451). Animals were bred and housed in individually ventilated cages at the Wellcome Trust Centre for Human Genetics, Oxford. Unless otherwise stated, 6 to 10 week old C57BL/6 mice were used in all experiments.

Organoids were derived using published protocols (Sato, T. et al. Naturc 459, 262-265 (2009); O'Rourke, K. P., et al. Bio Protoc 6 (2016)). The proximal colon was removed and cut into small (˜5 mm) pieces and washed with ice-cold PBS until the supernatant was clear. Tissue fragments were washed and incubated with cold 5 mM EDTA/PBS (Fisher Scientific, BP2483) and placed on a roller at 4° C. for 15 min. Tissue fragments were washed twice more with ice-cold PBS and incubated in TrypLE (ThermoFisher Scientific, 12605010) at 37° C. for 30 min. Following a further two washes with ice-cold PBS, sedimented tissue fragments were vigorously resuspended in ice-cold PBS and allowed to settle under gravity. The supernatant, enriched with cells released from the tissue fragments, was collected, passed through a 35 μm cell strainer (Corning, 352235), and centrifuged at 200×g for 5 min. The pellets were embedded in growth factor-reduced Matrigel (Corning, 354230) and seeded onto 24-well plates (˜30 μL of Matrigel per well). Matrigel was allowed to polymerize at 37° C. for 5 min, and 500 μL of the murine organoid culture medium (Table 8) was overlaid and replaced every two days. To prevent anoikis, 10 μM ROCK inhibitor (Y-27632) was added to the medium for the first 2 days. Organoids were passaged every 7-10 days at a ratio of 1:4 and kept at 37° C. in an atmosphere of 5% CO₂.

### Murine Mutant Duodenum Organoids

Organoids were derived using the protocol of Sato et al. (Sato, T. et al. Nature 459, 262-265 (2009)). The small intestine was dissected, opened longitudinally, and washed with cold PBS. The isolated tissue was cut into 0.5 cm segments and vigorously washed with ice-cold PBS ˜5 times until the supernatant was clear. Tissue fragments were incubated in 2 μM EDTA/PBS and placed on a roller at 4° C. for 1 h. The EDTA/PBS was removed after the incubation, and the tissue segments were washed once with ice-cold PBS. The tissue was then washed 4× with Advanced DMEM/F12 (ADF, ThermoFisher Scientific, 12634028) by vigorous pipetting. The supernatant obtained from these washes was collected and centrifuged for 5 min at 1,200 rpm. The cell pellet was resuspended in 15 mL ADF and passed through a 70 μm cell strainer. The cells were collected and centrifuged for 2 min at 600 rpm. The pellet, now enriched with intestinal crypts, was embedded in Matrigel, seeded onto a pre-warmed 48-well plate (Greiner Bio-One Ltd) at 20 μL Matrigel per well, and incubated for 10 min at 37° C. 200 μL of organoid culture medium was added to each well, and the plate was incubated at 37° C. in the presence of 5% CO₂. For passaging, organoids were retrieved from Matrigel using ice-cold PBS and broken up mechanically by passing through a 23G, ⅝″ needle. The organoid fragments were centrifuged and washed with ice-cold PBS, and then reseeded in fresh Matrigel. The passage was performed every 7-10 days at a ratio of 1:4.

### Organoid Experiments Details

Chemotherapy Drug Toxicity Screen with Human Duodenum Organoids

Organoids were plated onto 96 well plates as follows. Using ice-cold PBS, the organoids were retrieved from the Matrigel and briefly microcentrifuged. To aid organoid dissociation, the pellet was vigorously resuspended in Trypsin and incubated at 37° C. for 10 min to ensure a high degree of dissociation. The organoid fragments were then briefly microcentrifuged and resuspended in 200 μL of Matrigel. The resuspended organoids were then kept on ice to prevent gelation of the Matrigel. Then, 2 μL of the organoid suspension was seeded inside each well. During this process, the organoid-Matrigel mix was resuspended every 12 wells to ensure an even mix. The Matrigel was allowed to gelate at room temperature for 5 min. Once the Matrigel solidified, 100 μL of human organoid medium, pre-warmed at 37° C. to prevent the detachment of the Matrigel, was then overlaid and replaced every 2 days. Once the organoids had grown for 6 days inside the 96-well plate, the medium was removed by inversion and washed with pre-warmed PBS at 37° C. After removing the PBS by inversion, the wells were filled with 100 μL of human organoid medium (HOM) with a titration of chemotherapy drugs. The maximum drug concentration values used were as follows: Gefitinib, 0.5 μM; Docetaxel, 12.5 nM; Oxaliplatin, 20 μM; Irinotecan, 5 μM; Cisplatin, 6 μM; Mitomycin C, 0.03 μM; 5-Fluorouacil, 20 μM; Epirubicin 4.5 μM. These concentrations were based on the previously published screening concentrations (van de Wetering, M. et al. Cell 161, 933-945 (2015)). All drugs were solubilized and diluted with DMSO. The different conditions were filmed on a Nikon microscope for 120 h at a frequency of 1 image per hour with a 2× objective.

### VPA/CHIR99021 Treatment

VPA and CHIR99021 for the treatment of murine intestinal organoids were purchased from Cambridge Bioscience Ltd, and stock solutions were prepared following the manufacturer's protocols. The working concentrations of VPA and CHIR99021 were 1 mM and 3 μM, respectively.

### Polymerase Chain Reaction (PCR)

PCR was performed to confirm the murine colon organoid genotypes using the primers outlined in Table 10 and GoTaq Green Master Mix (New England BioLabs® Inc.), according to the Manufacturer's instructions, on the ProFlex PCR System (ThermoFisher Scientific). Zymo Research Quick-DNA Miniprep Plus Kit (D4068) was used to extract organoid DNA.

### Lentivirus Production

A lentivirus expressing the mCherry and mBanana fluorescent proteins was produced to fluorescently label individual mutant murine colon organoids. Phoenix cells (CRL-3213™) were seeded onto a 15 cm plate pre-coated with 0.01% poly-L-lysine and allowed to reach confluency. The cells were then transfected with 15 μg of lentiviral vector (CSII pEF mCherry P2A 3F MCS and CSII pEF mBanana P2A 3F MCS), 12 μg of pCD/NL-BH*DDD (HIV-1 Gag/Pol, Tat, Rev) and 9 μg of VSV-G (Vesicular Stomatitis Virus G Glycoprotein) using Lipofcctaminc2000 and Opti-MEM as the transfection reagents. 24 h later, the medium was removed and replaced with 15 mL of fresh medium. After a further 24 h incubation, the medium was collected, passed through a 0.45 μm filter, and stored at 4° C. 15 mL of fresh medium was then placed onto the cells for a further 24 h, followed by the same collection and filtration. The two collections were then pooled, aliquoted, and stored at −80° C.

### Lentiviral Transduction of Organoids

Following in vivo tamoxifen treatment, KRAS^{LSL-G12D/+ CreERT2-FYFP}:p53^{Null} murine colon organoids were confirmed to be EYFP positive by confocal microscopy. To fluorescently label the remaining two mutant organoid populations, APC^{min/+} and p53^{null} organoids were transduced with lentivirus expressing mCherry and mBanana fluorescent proteins, respectively. The medium was removed from at least six confluent wells of the relevant genotype, and the organoids were retrieved from the Matrigel by resuspension with ice-cold PBS followed by brief microcentrifugation. To aid dissociation, the pellet was vigorously resuspended in TrypLE and left at room temperature for 5 min. Organoid fragments were briefly microcentrifuged, and the pellet was resuspended in 5 mL of the relevant lentivirus. The organoid/lentivirus suspension was added to a 6-well plate and centrifuged at 600×g at room temperature for 1 h. The plate was then incubated at 37° C. for 3 h. Labeled organoids were collected, embedded in Matrigel, and seeded in 24-well plates (˜30 μL of Matrigel per well). The Matrigel was allowed to polymerize at 37° C. for 5 min, and 500 μL of the relevant murine organoid medium was overlaid and replaced every 2 days.

To generate a homogenous population of organoids positive for the fluorescent protein of interest, a MA900 Multi-Application Cell Sorter (Sony Biotechnology) was used. Fluorescence was detected within 24 h of lentiviral transduction.

To generate p53^{R172H:R172H} and KRAS^{G12D/+} organoids, p53^{LSI-R172H} homozygous mBanana and KRAS^{LSL-G12D/+ CreERT2-EYFP} organoids were infected with Cre Recombinase Adenovirus (Vector Labs Catalogue No. 1045). Dissociated organoids were infected at a Multiplicity of Infection (MOI) of 50 in 1 mL of organoid culture medium and incubated at 37° C. for 3 h. The infected organoids were then briefly spun down, the pellet embedded in Matrigel, and seeded in 24-well plates (˜30 μL of Matrigel per well). This was repeated until the mutation was homogenously expressed throughout the organoids. To note, treatment of KRAS^{LSL-G12D/+ CreERT2-EYFP} organoids with the Cre Recombinase Adenovirus rendered them EYFP positive. During the culture process, routine PCR was carried out, and it was found that a subset of KRAS^{G12D/+} mutant organoids had additionally acquired a p53^{null} mutation. These were included as an additional genotype in the SPOT analysis.

### Phase-Contrast Timelapse Microscopy of Mouse Duodenum Organoids

Images were acquired using a Nikon TE 2000-E Eclipse inverted microscope, with a 10×/0.3 NA Plan Fluor Ph1 objective. The system was maintained at 37° C. in the presence of 5% CO₂. Z-stack images of 1 mm in depth were acquired at 100 μm intervals (11 steps per acquisition), one frame every 15 min.

### Time-Lapse Confocal Microscopy of Mouse Colon Mutant Organoids Grown in 1 μL Droplets

Fluorescent mutant colon organoids, APC^{min/+} mCherry, KRAS^{G12D/+} EYFP, KRAS^{G12D/+}: p53^{null} EYFP, p53^{null} mBanana and p53^{R172H:R172H} mBanana were seeded as 1 μL droplets into a 384-well plate, overlaid with 40 μL of medium. Organoids were grown for 72 h before being imaged on a Zeiss 710MP confocal microscope using the 10× objective. During the time-lapse experiment, a z-stack was performed every 2 h for 5 days at each of the chosen positions.

### Label-Free Timelapse Microscopy of Organoids

### Murine Colon Organoids.

Organoids were individually seeded onto a 96-well plate and grown for 72 h, then imaged on a Nikon TE-2000e microscope using the 4× objective. During the course of the time-lapse experiment, a z-stack was performed every hour for 5 days at each of the chosen positions.

### Human Duodenum Organoids.

Human duodenum organoids were harvested and dissociated into single cells following the passaging procedure described above. Cell pellets were resuspended in 500 μL of PBS. Cells were filtered through the cell strainer (70 μm) and counted with the automated cell counter (BIO-RAD). The appropriate cell dilutions were made in GFR Matrigel. Then, the cells were seeded in a pre-warmed 96-well black tissue culture-treated (TC-treated) sterile microplate (PerkinElmer), and plates were incubated for 10 min in a cell culture incubator at 37° C. and 5% CO₂ to solidify the Matrigel. Human duodenum organoid culture media (HOM, Table 9) was added to each well (200 μL) and refreshed every other day. For WNT experiments, before performing timelapse video collection, the HOM media was changed to ENR (EGF, Noggin, R-spondin only) for the ENR imaging conditions. Specifically, group HOM organoids were cultured in HOM culture medium for 14 days. Group ENR+WNT3A (day 3) organoids were under ENR treatment for 3 days and then ENR+Wnt3A for 11 days. Group ENR+WNT3A (day 6) organoids were under ENR treatment for 6 days and then ENR+Wnt3a for the next 8 days. Group ENR+Wnt3A (day 9) organoids were under ENR treatment for 9 days and then under ENR+Wnt3A treatment for 5 days. Group ENR organoids were under ENR treatment for 14 days. All images were acquired using a Nikon TE 2000-E Eclipse inverted microscope, with a 10×/0.3 NA Plan Fluor Ph1 objective. The system was maintained at 37° C. in the presence of 5% CO₂. Z-stack images 1000 μm in depth were acquired at 100 μm intervals (11 steps per acquisition) approximately every 60 min for 5 days (chemotherapy drug screen) and 14 days (WNT addition experiments).

### RNA Isolation and Bulk RNA Sequencing and Analysis

For bulk RNA sequencing, intestinal organoids were retrieved from Matrigel using ice-cold PBS, washed 2 to 3 times to remove dead cells, and collected using a benchtop centrifuge. Total RNA was extracted with the RNeasy Plus Micro Kit. First-strand cDNA was generated following the poly(A) enrichment protocol, and the resulting cDNA libraries were sequenced as 100 bp paired-end reads on the HiSeq 2500 System (Illumina). Library prep and sequencing were performed by the Oxford Genomics Centre based at the Wellcome Centre for Human Genetics. Sequence alignment used STAR and counts generated by Samtools. Counts per million (cpm) were computed from the raw counts using edgeR (version 3.16.5) and R (version 3.3.3) using default parameters to remove any transcripts that did not have at least 2 samples with cpm>2. Default parameters of calcNormFactors were used to compute the normalization library size factors for the reduced count matrix and were used with a prior.count=3 to compute the log 2 (CPM) values. Multidimensional scaling (MDS, using cmdscale) was then applied to the sample pairwise Euclidean distance matrix of log₂(CPM) to generate the two-dimensional MDS plotting coordinates.

### Single-Cell RNA Sequencing

scRNA-seq was performed on the murine wild type, mutant fluorescent colon organoids, and human duodenum organoids in order to determine the differences in their gene expression profiles.

### Murine Organoids.

Organoids were individually seeded in a 384-well plate and cultured for 7 days before processing. On day 7, the organoids were retrieved from the Matrigel using ice-cold PBS, centrifuged at 300×g for 5 min, and incubated in TrypLE for 15 min. The dissociated organoids were then passed through a 35 μm cell strainer, and the flow-through was centrifuged at 300×g for 5 min. The pellet was resuspended in 1 mL Cell Recovery Solution (Sigma Aldrich, DLW354253) and incubated for 2 h at 4° C. under constant rotation. Samples were then centrifuged at 300×g for 5 min, and the pellet was resuspended in 1 mL 0.04% BSA (in PBS). The pellet was resuspended in 100 μL 0.04% BSA, and dissociated organoids were passed through a 35 μm cell strainer. Filtered cell suspensions were then counted using an automated cell counter, diluted to a concentration of 1 million total cells/mL in 0.04% BSA, and kept on ice until encapsulation.

scRNA-seq was conducted using a 5′ scRNA-seq gene expression workflow (Chromium Single Cell Immune Profiling, Solution v1.1, 10× Genomics). Following encapsulation of cells using the Chromium Controller, GEM-RT, cDNA amplification, and construction of final libraries were conducted following manufacturer's instructions. Size profile and concentration of final libraries were assessed by the Agilent 2100 Bioanalyzer (High Sensitivity DNA Kit, Agilent, 5067-4626) and Qubit (BR DNA Assay, ThermoFisher, Q32853), respectively. Sequencing of final libraries was conducted on the Illumina NextSeq 550 10× Genomics recommendations (26 cycles read 1, 8 cycles i7 index, 98 cycles read 2, targeting a minimum depth of 20,000 reads/cell).

### Human Organoids.

Organoids were individually seeded onto a 24-well plate and cultured for 7 days before treatment as described above.

For scRNA-seq, Group HOM was collected after the initial 7 days' culture before further treatment, that is day 0 of the timelapse acquisition. Group ENR+WNT3A (day 3) organoids were under ENR treatment for 3 days, ENR+Wnt3A for 11 days, then collected. Group ENR+WNT3A (day 6) organoids were under ENR treatment for 6 days, ENR+Wnt3a for the next 8 days, and then collected. Group ENR organoids were under ENR treatment for a total of 3, 6, and 9 days, respectively, then collected. After being harvested, the organoids were processed in the same manner as for murine organoids.

### Example 2

### Validation Datasets

### Computer Vision Datasets for Testing SAM Phenome

Computer vision datasets with manually curated reference annotation were used to validate that the SAM phenome was able to capture and discriminate heterogeneity and be universally applicable as-is without having to refine or define new features. Performance was assessed quantitatively by comparing the result of k-means clustering on the computed respective SAM feature set, where the number of clusters, k was set to the known number of classes in the given dataset. Two standard quality clustering metrics were used. Adjusted mutual information (AMI, 0-1, scikit-learn metrics.adjusted_mutual_info_score) is an adjustment of the Mutual Information (MI) metric which measures the similarity between two clusterings to account for chance. A value of 1 is perfect concordant clustering. Adjusted rand index (ARI, −0.5-1, scikit-learn metrics.adjusted_rand_score) is an adjustment of the Rand Index metric, which computes a similarity measure between two clusterings by considering all pairs of samples and counting pairs that are assigned in the same or different clusters in the predicted and true clusterings, to account for chance. It is 1 for perfect concordant clustering and lower bound by −0.5 for particularly discordant clusterings. For A2D dataset, the performance of training a supervised classifier on the SAM features was also assessed. Performance was reported using the balanced accuracy score (labeled only as ‘accuracy’ in figures), which is the average of the recall obtained on each class to account for imbalanced classes, and F-score (or F1-score), defined as 2*precision*recall/(precision+recall) for a single class. The mean across all classes weighted by the number of truc instances in each class to account for class imbalance was reported.

### MPEG-7 Shape Dataset to Test Shape

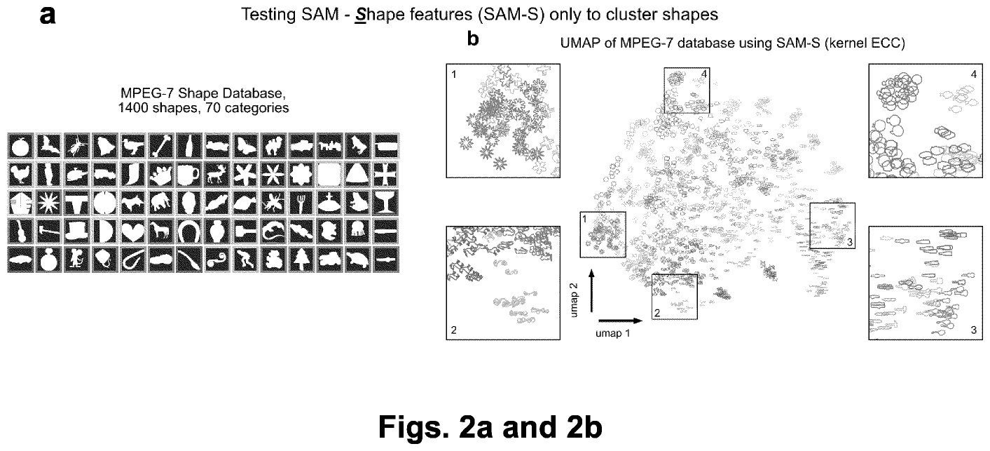

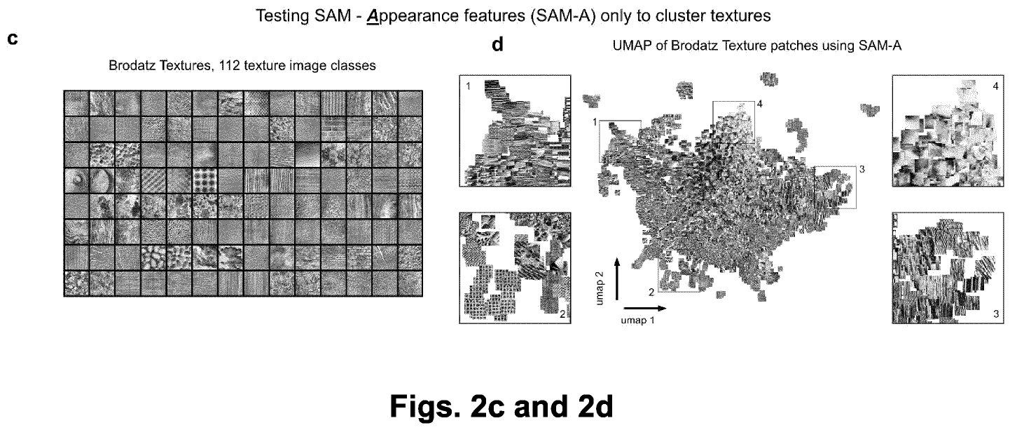

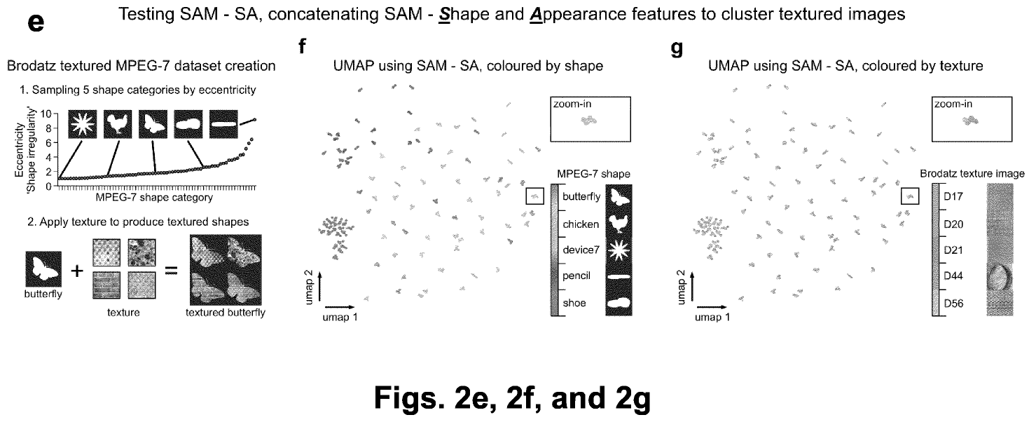

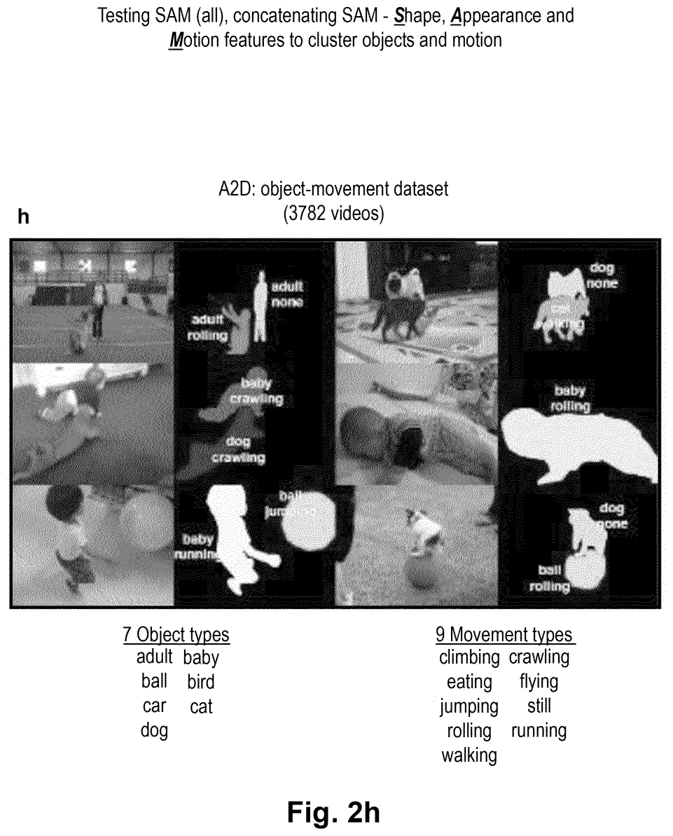

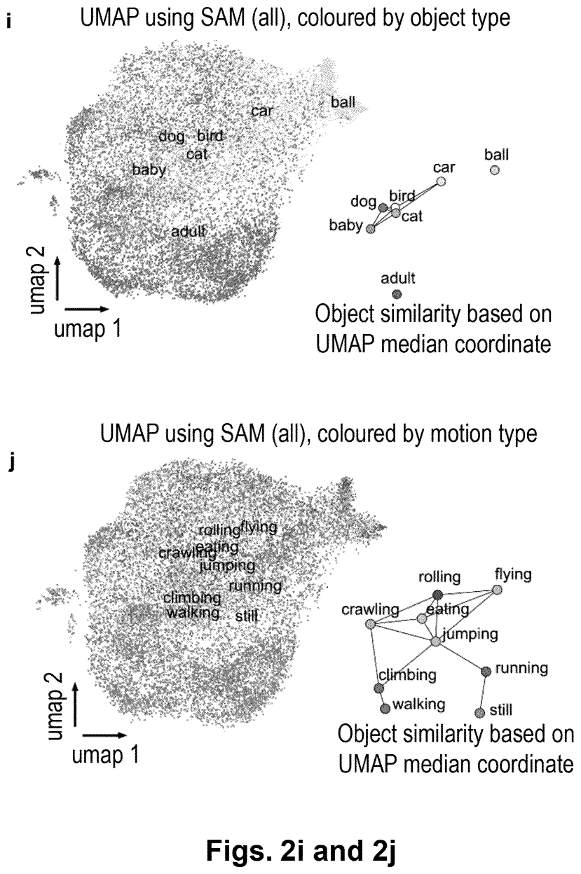

The MPEG-7 Core Experiment CE-Shape-1 (shortened to MPEG-7) database (Latecki, L. J. & Lakamper, IEEE Transactions on Pattern Analysis and Machine Intelligence 22, 1185-1190) is a standard shape dataset comprising a total of 1400 shapes as binary images; 20 unique image examples each of 70 different shape classes representing different unique everyday objects such as bone, bat, heart, horseshoe, octopus, turtle captured with different sizes, rotations, and poses (FIG. 2). Each image was binary dilated by 3 pixels, and topologically simplified by binary-infilling holes and extracting the external boundary of the shape as a contour of (x,y coordinates) using marching squares (scikit-image find_contours). The number of contour coordinates varies across shapes. To ensure the same dimension of SAM phenome is computed, the boundary contour of each shape was resampled to 200 equidistantly spaced boundary points. All SAM-S features were computed and were used to conduct the analysis in FIG. 2 with no other filtering of features except for kernel map dimensionality reduction of ECC features.

### Brodatz Texture Database to Test Appearance

The Brodatz texture database (Hersey, I. Leonardo 1, 91-92 (1968)) is a standard dataset for testing appearance descriptors in computer vision. It comprises 112 unique 512×512 pixels images representing different grayscale patterns. Here, the normalized Brodatz texture variant (Abdelmounaime, S. & Dong-Chen, International Scholarly Research Notices 2013 (2013)), an improved and more challenging dataset, was used. The dataset uses an intensity normalization process to eliminate grayscale background effect of the original. To use the Brodatz textures for testing, a 14,336 image dataset was derived by cropping each of the 512×512 texture images into 64 non-overlapping 64×64 patches and using both the patch and the patch rotated by 90°. SAM-A features were computed directly using the patches, treating the whole patch as an object with a square boundary contour, and used to conduct the analysis in FIG. 2 with no other filtering of features. Features were power normalized and z-scored as described for SPOT, stage 3, step i below.

### Brodatz Textured MPEG-7 Shapes to Test Shape and Appearance

To test the combined shape and appearance features, a dataset of normalized Brodatz texture MPEG-7 shapes was created. Five shape classes from MPEG-7 were chosen equally, sampling the measured eccentricity used as a proxy measure of shape complexity, and five normalized 512×512 Brodatz texture images were chosen randomly. To texture the MPEG-7 shapes, their images were all standardized by rescaling to 128×128 pixels. Brodatz texture images were rescaled to 192×192 images. Then, for each of the 20 unique images in each MPEG-7 class, each 192×192 Brodatz texture image was taken, 20 random 128×128 cropped patches were sampled, and multiplied with the MPEG-7 image to create 20 different textured variants of the same basic texture and shape. This gives a total dataset of 10,000 image patches (5 shape classes×20 images per shape class×5 texture classes×20 per texture class). SAM-SA features were obtained by concatenating computed SAM-S(SAM-S (kernel ECC)) and SAM-A features and used to conduct the analysis in FIG. 2 with no other filtering of features. Features were, however, power normalized and z-scored as described for SPOT, stage 3, step i below.

### A2D Object-Motion Video Dataset to Test SAM Features in the Wild

To simultaneously detect joint variations in shape, appearance, and motion (SAM) in a setting reminiscent of real application to a heterogeneous dataset, the A2D dataset (Hersey, I. Leonardo 1, 91-92 (1968)) was used. This dataset comprises 3782 videos sourced from YouTube, depicting seven classes of moving objects (adult, baby, bird, cat, dog, ball, and car) performing nine different movements (still, labeled for no movement), climbing, crawling, eating, flying, jumping, rolling, running, and walking (FIG. 2h). No object performs all actions. There are 43 unique object-movement pairs, the frequency of which are unequally distributed. A video may contain more than one instance of an object-movement pair or instances of different object-movement pairs. For each video, 3-5 non-contiguous frames are annotated.

### Generating Consecutive Frame Object Segmentation Instances to Compute SAM Phenomes.

To compute the full SAM features for each annotated object instance, the Segment Anything model (Kirillov, A. et al. arXiv preprint arXiv:2304.02643 (2023)) and the bounding box of its annotated contour to resegment the object were used. Then, optical flow (Farnebäck, G. in Scandinavian conference on Image analysis. 363-370 (Springer)) was used to predict the object's bounding box in the immediate next frame and use it as the prompt to segment its outline using the Segment Anything model, to ensure that objects were segmented in the same way across frames.

### Evaluating Clustering and Classification Performance on Test Dataset Split.

The A2D dataset has 43 unique object-movement pairs and was pre-split into 3036 training videos and 746 testing videos. For both clustering and classification analysis, object instances in the training videos were used to set the parameters of the k-means and classifiers and report performance only on objects in the testing videos. Full SAM features were defined by concatenating computed SAM-S(SAM-S(kernel ECC)), SAM-A, and SAM-M features, and were used to conduct the analysis in FIG. 2 with no other filtering of features. Features were power normalized and z-scored as described for SPOT, stage 3, step i below.

Object and Movement Type Similarity Graph Analysis from UMAP Median Coordinate.

Using the median 2D UMAP coordinates of each object, movement or object-movement type, compute the pairwise affinity matrix between types defined as affinity=e^{-D/μ(D)}, where D is the pairwise Euclidean distance matrix between coordinates and μ(D) is the mean value of the D matrix. The final similarity graph plots all connections between type i and type j if the affinity is greater than the mean value of the affinity matrix (after excluding the main diagonal self-connections).

### Single-Cell Tracking Datasets for Testing SPOT Analysis

Two datasets (U373 and HL60) were chosen from the 2D single cell tracking challenge (Ulman, V. et al. Nature Methods, 14, 1141-1152 (2017).) with distinct and easily interpretable phenotypes (distinct cell morphologies as U373 cells migrate and distinct appearance changes as HL60 cells divide) to test SPOT analysis (stage 3). Each dataset comprised two unique videos, and each video has provided outline annotation and temporal tracking information available for all timepoints used to avoid introducing additional errors in segmentation or tracking in SPOT, stage 2. Full SAM features were computed and compiled for all annotated cell instances that were fully present in the imaged field-of-view. Cells on the border and partially in-view were removed prior to SAM feature computation, and the corresponding single-cell tracks were truncated correspondingly into separate contiguous tracklets. The compiled SAM phenomes were filtered and processed as described below, and SPOT analysis (stage 3) was applied to both datasets, in the same manner, using all the data instead of setting a percentile cutoff to exclude potential outliers when computing the representative phenotype cluster exemplar images.

### CellProfiler Features Computation, Processing, and Comparison

### Computation of CellProfiler Shape-Related Metrics

All 1400 image files in the MPEG-7 dataset were uploaded to CellProfiler 4.2.6. The images were processed using CellProfiler's ‘ConvertImageToObject’ and ‘MaskObject’ modules in succession. The ‘MeasureObjectSizeShape’ module was then used to generate shape-related metrics derived from object boundaries. These metrics were exported using the ‘ExportToSpreadsheet’ module, and filtered to remove irrelevant columns such as centroid measurements and non-numerical columns prior to analysis.

### Computation of CellProfiler Appearance-Related Metrics

All 22,400 of the data augmented Normalized Brodatz dataset were uploaded to CellProfiler 4.2.6 (112 Brodatz image classes×200 patches per Brodatz class). Generation of appearance-related metrics was completed by grouping the images into 10 batches to prevent crashing. Within each batch, patches were grouped based on their image of origin. The “MeasureGranularity,” “MeasureTexture,” “MeasureImageIntensity,” “MeasureImageQuality,” and “MeasureImageSkeleton” modules were run in succession, selecting for all metrics available to be computed within each module's settings, wherever relevant. These metrics were exported using the ‘ExportToSpreadsheet’ module, and filtered to remove duplicated, irrelevant (such as execution time related), and non-numerical columns prior to analysis.

### Computation of CellProfiler Shape and Appearance-Related Metrics

The 10,000 images of the Brodatz textured MPEG-7 shapes were cropped using the tightest bounding box around the shape, and then uploaded to CellProfiler. The appearance-related metrics were computed at the whole-image level for each crop, a design choice made due to inadequate performance of the ‘ConvertImageToObject’ module on this dataset. The shape-related metrics are identical to those calculated for the entire MPEG-7 dataset and, thus, were not recomputed. All appearance-related metrics were computed by running the “MeasureGranularity,” “MeasureTexture,” “MeasurcImageIntensity,” “MeasurcImageQuality,” and “MeasureImageSkeleton” modules in succession. These metrics were exported using the ‘ExportToSpreadsheet’ module, and filtered to remove duplicate, irrelevant, and non-numerical columns prior to analysis. The shape-related metrics and appearance-related metrics were concatenated to form the combined CellProfiler shape-appearance descriptor.

### Analysis of CellProfiler Computed Features

To ensure like-for-like comparison, CellProfiler computed features were handled and processed in the exact same manner as SPOT's SAM features for each relevant validation dataset.

### Shape, Appearance and Motion Phenotype Observation Tool (SPOT)

### Stage 1: Video Acquisition

### Extended Focus 2D Video Assembly of Stacked Timelapse Acquisition for Analysis

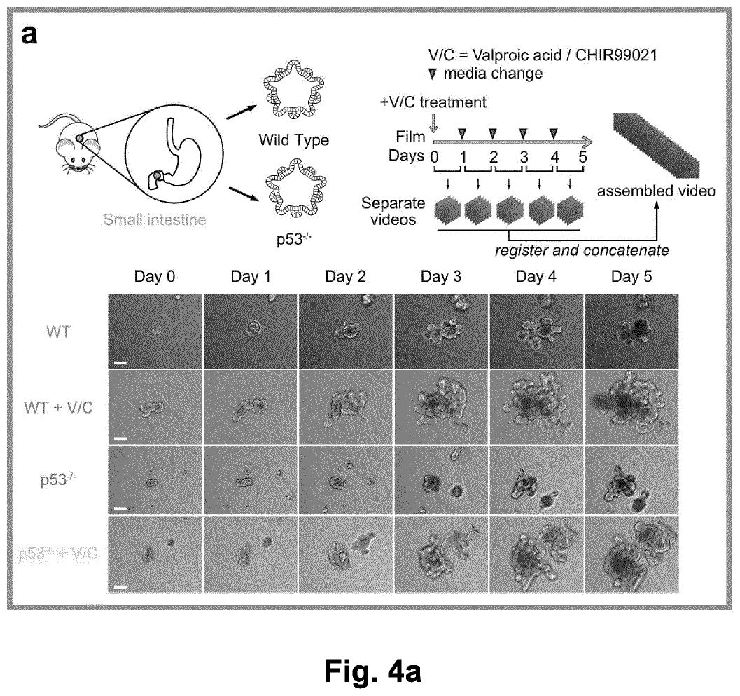

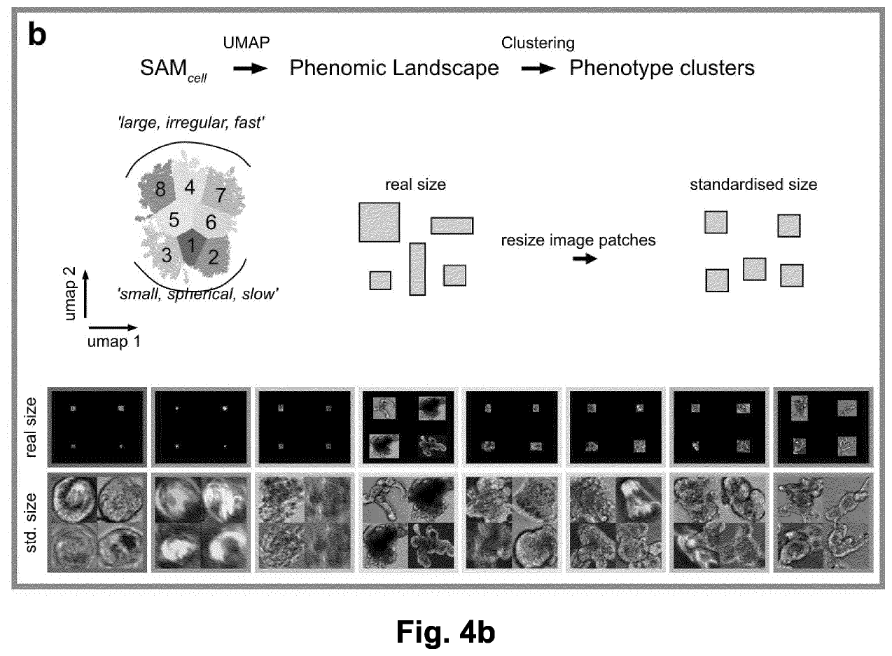

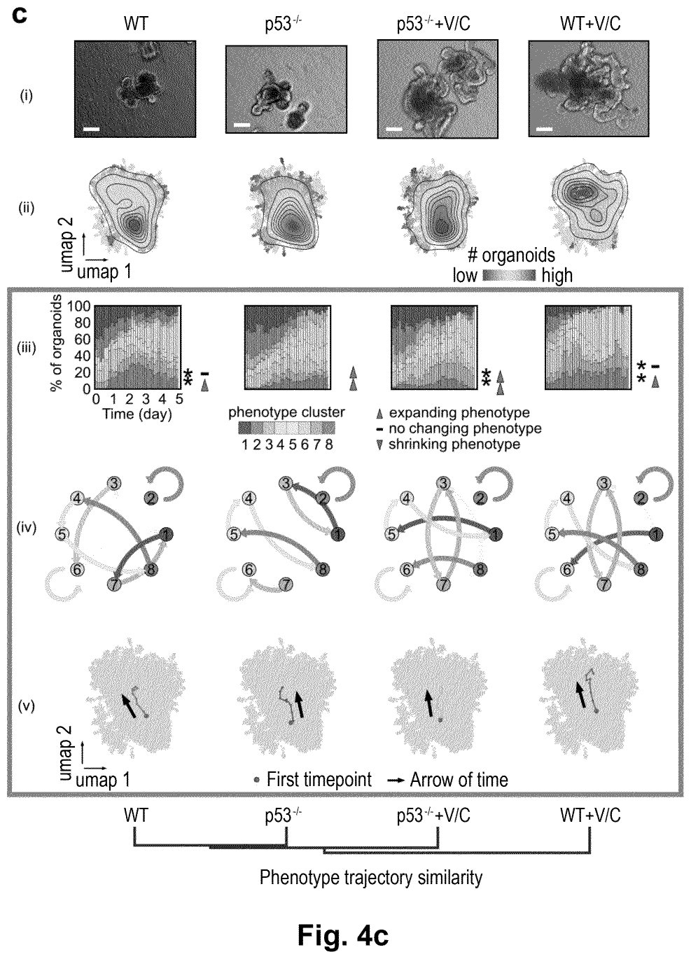

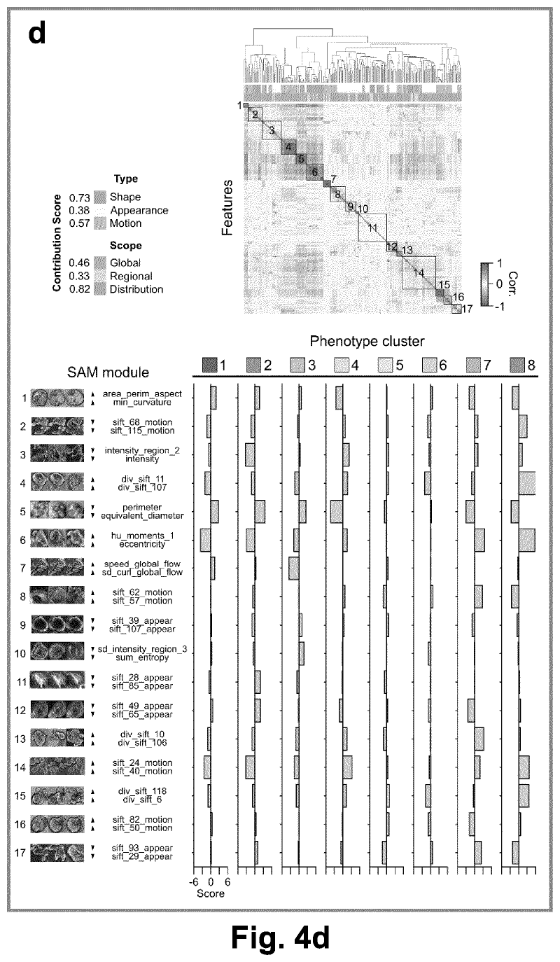

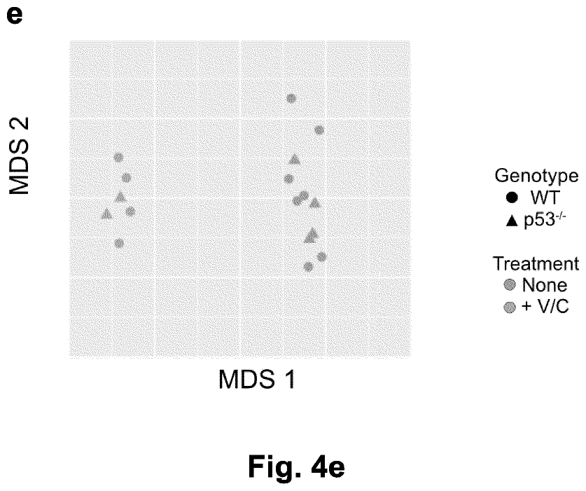

To capture in-focus organoids in a well plate, multiple z-planes are acquired per timepoint. For each timepoint, the z-stack is then projected into a single 2D image where the majority of organoids are in focus using the ImageJ Stack_Focuser plugin. For extended long-time timelapse data that were acquired in multiple stages due to the need for regular medium change (FIG. 4), Fourier transform cross-correlation methods (Kuglin, C. D. in IEEE Int. Conf. on Cybernetics and Society, 1975. 163-165) was applied sequentially to register and stitch together the extended focused images from different time periods while minimizing for potential translational shift due to stage shift. Blurry frames were automatically detected as those with a mean edge magnitude one standard deviation less than the mean over all video frames and were removed prior to the registration. For more robust registration, the absolute magnitude of difference of Gaussian filtered (img—gaussian (img, sigma=15)) frames was used as ‘edge enhanced’ images to obtain the correct translational correction. This correction was then applied to the raw video frames.

Organoid Detection, Tracking, and Segmentation from Timelapse Microscopy

YOLOv3 (Redmon, J. & Farhadi, arXiv:1804.02767 (2018)) (from http://github.com/AlexeyAB/darknet) was used to train an organoid bounding box detector operating on 512×512 pixel images on an in-house curated organoid image dataset comprising label-free and confocal microscopy imaging. A YOLOv3 bounding box detection gives, for each box, a (x,y) centroid coordinate, width, height, and a detection confidence score (0-1). For a given video, the trained detector was applied to individual color channels independently on a frame-by-frame basis to detect all organoids. A custom optical-flow-based predictive tracker tracks and links the detected bounding boxes into tracklets and imputes missing detections. Briefly, starting from the initial bounding boxes in frame 0, which form individual tracklets, the coordinates of the last box in the tracklet in the next frame were predicted by estimating a rigid motion model (scale, translation, rotation) from the local optical flow within the box. Using the predicted coordinates, the boxes are matched to detected boxes from the next frame, frame 1, via linear assignment (c.f. Scipy linear_sum_assignment), using the pairwise cost matrix constructed from 1-intersection-over-union (IoU) between individual boxes. A cut-off is used to determine a successful pairing. The tracklets of successfully matched boxes are extended. Tracklets with boxes that were unsuccessfully matched in frame 0 are extended opportunistically with the predicted coordinates. Boxes unsuccessfully matched in frame 1 start new tracklets, adding to the pool of tracklets. The process is repeated until the end of the video.

Running tracklets that have not been successfully matched to a ‘real’ organoid box detected by the trained YOLOv3 detector after a specified number of frames are ‘terminated’ and no longer considered. This tracking process was designed to ensure coverage of all detected organoids throughout the length of the video. The final collection of tracklets was obtained by retaining individual tracklets filtering for both having a minimal lifetime coverage (measured as a fraction of tracklet length) and the mean YOLOv3 confidence detection score of boxes within the tracklet. An attention UNet (Oktay, O. et al. arXiv:1804.03999 (2018)) operating at 64×64 pixels were separately trained on a custom in-house label-free and confocal microscopy organoid image patches cropped from randomly sampled video frames to segment and produce individual organoid boundaries from the detected organoid tracklets. Extracted organoid boundaries at 64×64 pixels were resampled to the desired number of points using periodic spline interpolation (Scipy splprep) and transformed back to real image size coordinates for analysis.

### Postprocessing Organoid Segmentation Boundaries for Analysis

Organoid segmentations were post-processed to remove potential duplicate detections in dense organoid areas and, for confocal fluorescence microscopy images, bleed-through across channels. This was done on a tracklet basis. All tracklets from all image channels were jointly considered. For each pair of tracklets, the mean intersection over union (IoU) bounding box overlap restricted to the times for which they co-exist was computed. Tracklets were deemed to overlap the same tracked organoid if the computed IoU score was greater than a user-specified cutoff, here 0.25. Overlapped pairs of tracklets were grouped into unique cliques. Within each clique, only one set of tracklets was retained—that which was longest and with the best consistency. All other tracklets were removed. Next, individual organoids in tracklets lying on the border of the field-of-view that are only partially captured (defined as having a percentage of border points greater than a pre-specified cutoff within a number of pixels from the image border) were removed from the analysis. Tracklets were subsequently temporally filtered using centered moving averaging to improve the downstream extraction of more temporally consistent measurements. In the final step, each tracklet was checked, pre-terminating and creating new tracklets to ensure a minimum frame-to-frame IoU consistency for each tracklet. This optional step minimizes cases in which the tracker may ‘identity switch’ to a nearby, but different, organoid during the track.

### Stage 2: Computation of Shape, Appearance, and Motion (SAM) Features

SAM features were computed with custom code using, where possible, standard implementations available in established Python libraries such as scikit-image and opencv (see the GitHub code available at https://github.com/fyz11/SPOT). Detailed descriptions and relevant references for each feature are provided in Tables 1-3. Motion features are only computable for an object instance if it is present in at least the successive image frame. Most features are dimensionless. Where a measurement is not dimensionless, all measurements were extracted in standard physical units of micrometer (μm) for length and hours (h) for time.

### Stage 3: Discovery and Analysis of Dynamic Phenotypic Heterogeneity

### Step i: Filtering and Preprocessing of SAM Phenomes for Analysis

Raw computed SAM phenomes require filtering to remove those features that are too small or zero-valued and those that exhibit too high a variation across the dataset to be analyzed. Specifically, all features that are zero across all organoids (from possibly different experiments) were removed to be compared in the same analysis. High variance features (defined typically >2 standard deviations of the global mean) were also removed. The remaining features require normalization so that the values are comparable across features (for example, to enable shape area to be considered with equal weighting to shape curvature, where the raw values differ greatly in magnitude).

First, all curvature-associated features with units of length⁻¹ were multiplied by the equivalent diameter to correct for the inherent bias of curvature decreasing across organoids of the same shape but of different sizes. Second, a radial basis function (RBF) kernel was applied using the Nystroem approximation (c.f. scikit-learn Nystroem) to compress the raw, sparse, and discrete Euler characteristic curve (ECC) features to 100 dimensions which yielded improved clustering performance, and made the inclusion of ECC features more beneficial than its exclusion (see Tables 1-3, SAM-S (ECC) vs. SAM-S (kernel ECC)). Third, the feature values were correct to remove “batch” variation using linear regression to enable comparative analysis. Here, “batch” refers to the primary experimental source of variation; for example, for fluorescent murine mutant organoids, it is the plates acquired on different days, and for human duodenum organoids, it is the patient. Finally, the batch-corrected feature values were normalized by applying standard scaling (scikit-learn StandardScaler) followed by a power transformation (scikit-learn PowerTransformer, method=‘yeo-johnson’). The normalized feature values or SAM scores are plotted as ‘Score’ in figures. For label-free organoid imaging, an additional ridge regression (scikit-learn Ridge, alpha=1) with the frame number was applied as the dependent variable, and the SAM score of each feature was applied as independent variables to further select only those features that exhibit temporal variation, defined as those with an absolute regression coefficient greater than the mean absolute regression coefficient of all features.

### Step ii: Construction of SAM Phenomic Landscape

The SAM phenomic landscape aims to capture and visualize all variations in SAM at every timepoint and across time in 2D. As such, while any dimensionality reduction works, for best results, the dimensionality reduction method should ‘spread’ the SAM variation in 2D and not collapse object instances into branches or singular points. Here, Uniform Maniform Approximation and Projection (UMAP) (McInnes, L., Healy, J. & Melville, arXiv preprint arXiv:1802.03426 (2018)) was used through the Python umap-learn implementation to project preprocessed SAM features onto 2-dimensions for plotting and analysis using the following parameters throughout for organoids: n_neighbors=100, random_state=0, spread=0.5, min_dist=0.5, metric=‘euclidean’. PCA analysis yields a similar phenomic landscape but with greater compaction of points (data not shown). For very large datasets (>1 million points), or if it is desired to add new organoid points after initial mapping, a dimensionality reduction method is used to support batch-based evaluation such as parametric UMAP or deep neural autoencoders.

Step iii: Partitioning the Phenomic Landscape into Discrete Phenotype Clusters

Clustering was used to partition the phenomic landscape into a discrete number of phenotype clusters such that the object instances within each phenotype cluster share similar SAM features. For example, k-means clustering was used to generate approximately equal-sized phenotype clusters. However, if it is known or hypothesized that phenotype clusters should exhibit different abundances, for example, corresponding to a rare phenotype, alternative clustering methods such as density-based clustering and Leiden may be more suitable and can be used instead. Scikit-learn k-means clustering (random_state=0) was applied to the UMAP projected 2-dimensional coordinates to cluster and classify individual organoid datapoints as a small set of prototypical ‘phenotype clusters.’ The choice of number of clusters was made from a minimum of 2 clusters to a maximum of 20 clusters guided by the elbow method applied to the k-means inertia loss. Using these phenotype clusters, an arbitrary collection of organoid instances can be described with a histogram recording the occurrence frequency of each prototype. This description approach naturally reconciles multi-parametric phenotypic heterogeneity and is motivated by the success of bag of words/features approaches in computer vision.

### Auto-Generating Exemplar Object Instances for Each Phenotype Cluster.

The first principal component (PC) obtained by applying PCA to all object instances within a phenotype cluster provides a measure of the contribution of each object instance to the variance within the cluster. Object instances within a cluster were ranked in descending order of the first PC value, and the top rankings were visualized as exemplars and aligned with the first PC. As PCA is sensitive to outliers caused by errors in the initial object segmentation or tracking, ranking was performed only for instances for which the values of the first PC were less than the 90^{th} percentile. For single-cell tracking datasets, where segmentations and tracking were manually created, all object instances were used without exclusion (i.e., the 100^{th} percentile was used as the cutoff).

### Step iv) Monitoring Phenotype Cluster Dynamics

### Temporal Phenotype Cluster Frequency Stacked Barplots.

Each object instance was assigned the phenotype cluster into which its 2D UMAP coordinates fall. The total temporal duration of video acquisition (e.g., 14 days) was partitioned into equal time intervals (e.g., 1 day). For each time interval, a normalized histogram of the number of object instances within the interval was constructed where (the frequency of phenotype cluster i)=(#object instances in a time interval of cluster id i)/(total #object instances in that time interval). The normalized histograms for all time intervals were concatenated chronologically to form a stacked barplot of phenotype cluster frequency over time.

### Hidden Markov Model (HMM) Phenotype Cluster Transition Graph.

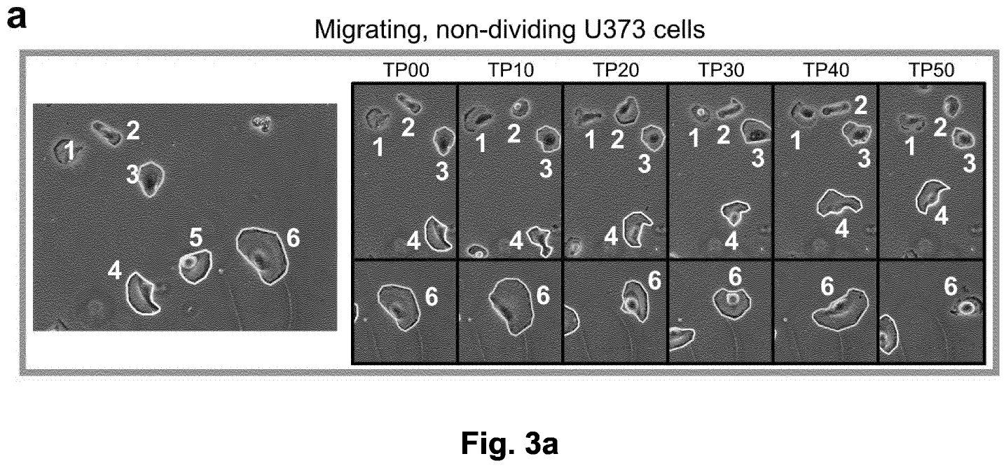

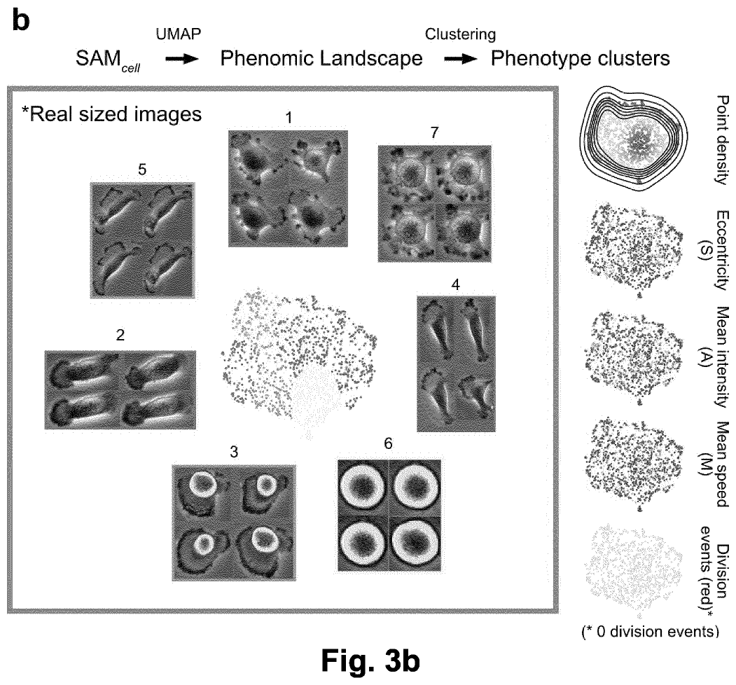

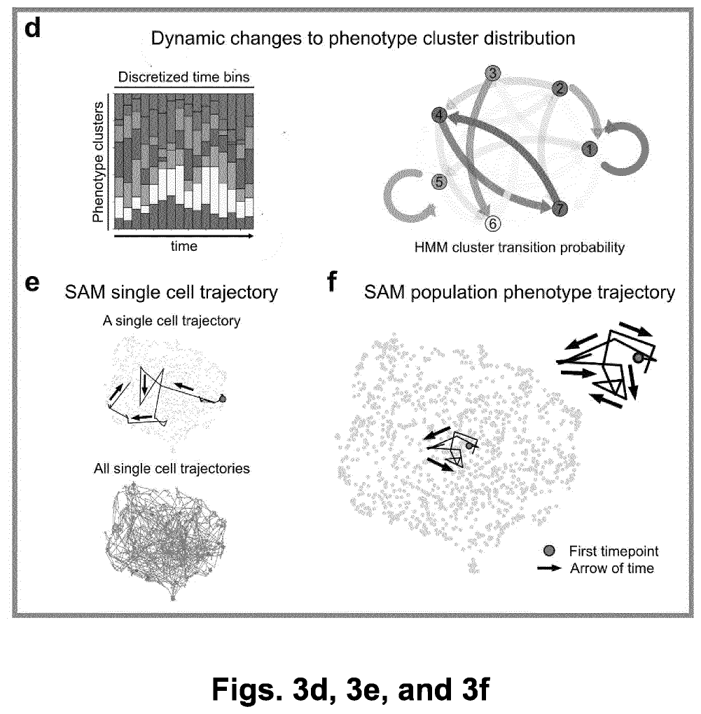

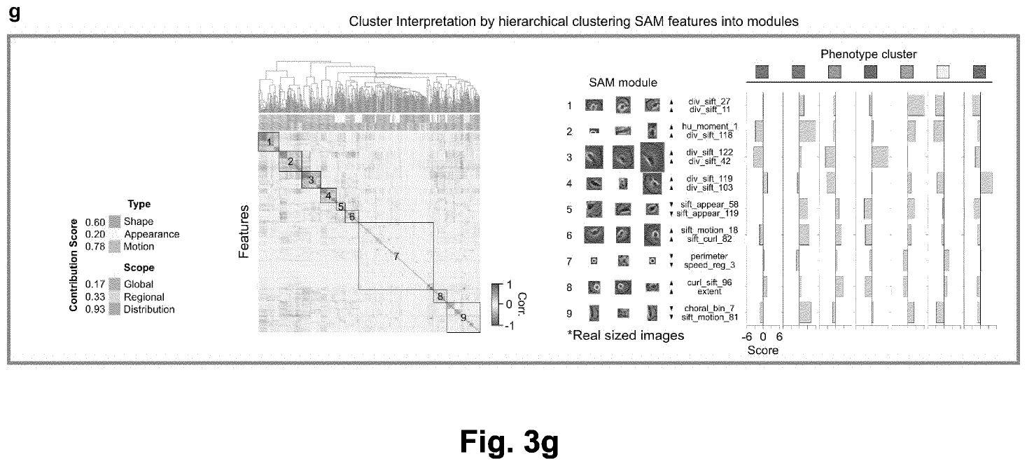

The HMM phenotype cluster transition graph requires the objects-of-interest to be tracked over time. Each object instance is assigned the phenotype cluster into which its 2D UMAP coordinate falls. Using the object tracks from SPOT, Stage 2, the phenotype cluster label time series were reconstructed for each tracked object (FIG. 3c). The phenotype cluster labeled cell tracks were modeled as individual observed sequences of a hidden Markov process, whereby the probability of a phenotype cluster transitioning to another cluster was fixed and independent of its historical state. To infer the transition probability matrix, the categoricalHMM model in the Python hmmlearn package was used. The number of hidden states was specified to be the same as the number of phenotype clusters, and the identity of each hidden state was matched to the phenotype cluster by comparing the labeling of predicted cell tracks with the fitted HMM and the observed labeled cell tracks. The cluster transition graph was visualized using the Python hmmviz package.

### Step v) Computation of the SAM Phenotype Trajectory

To construct temporal SAM phenotype trajectories in phenomic landscape, the total temporal duration of video acquisition (e.g., 14 days) was partitioned into equal time intervals (e.g., 1 day). For each time interval, a heatmap density image was computed using only the individual object instances observed during that time interval. The heatmap image is thresholded into high- and low-density regions using the mean density (±2 or 3 standard deviations) as a cutoff. The mean UMAP coordinate of the high-density regions was used as the phenomic landscape coordinate that best captures the majority phenotype during this time interval. The full phenotype trajectory was formed by chronologically linking together the mean UMAP coordinates of high-density regions across all time intervals.

Step vi) Automatic Grouping of Filtered SAM Phenome Features into SAM Modules

SAM modules were defined by grouping features together such that there is lower variation between these features within a module than between different modules. All the compiled SAM phenomes in the dataset were analyzed across all conditions. Following filtering and preprocessing above (Stage 3, step i), the (#features)×(#features) pairwise Pearson correlation coefficient matrix between the retained and transformed SAM features was computed using all object instances as “samples.” To automatically group features into SAM modules and determine the number of SAM modules, hierarchical clustering, with clustering quality metrics, was used. Hierarchical clustering generates a dendrogram whereby the root node groups all features as one, and progressive branch splitting corresponds to splitting of the larger parental grouping into unique, smaller, mutually exclusive groupings. At the bottommost leaves of the dendrogram, the individual SAM modules are the individual SAM features. To determine the optimal module number, the Davies-Bouldin (DB) (Davies, D. L. & Bouldin, D. W. IEEE Transactions on Pattern Analysis and Machine Intelligence, 224-227 (1979)) score, defined as the average similarity measure of each cluster with its most similar cluster, was used, where similarity is the ratio of within-cluster to between-cluster distances. The lower the DB score, the better the clustering, with the minimum DB score as 0. In computing the DB score as a function of the increasing number of modules with progressive dendrogram branch splits, the DB score increases then decreases with typically multiple peaks. The number of modules was set to correspond to the first peak in the DB score. This strikes a balance between having too few and too many modules to describe phenotypic heterogeneity.

Step vii) Finding Exemplar Object Instances and Evaluating SAM Module Expression in Individual Phenotype Clusters

Principal component analysis (PCA) finds a linear transformation of the data such that the first coordinate or first principal component corresponds to the direction of greatest variance of the data. The expression of SAM module i for each object instance is defined as the 1^{st} principal component after applying PCA to the preprocessed and normalized SAM features from SPOT Stage 3, step i (i.e., the SAM scores of final features) that form SAM module i. As the normalized SAM features have zero mean and the same standard deviation and as PCA without whitening preserves this property for SAM expression, a score of how much an object instance expresses SAM module i was defined as the expression of SAM module i minus the maximum expression for any other SAM module. For each SAM module, object instances were ranked in descending order with this score, and the top instances were visualized as exemplars.

### Scoring the Contribution of Feature Type and Spatial Scope

Principal component analysis (PCA) finds a linear transformation of the given input data such that the first coordinate or first principal component corresponds to the direction of greatest variance of the data. This property was used to derive a score of the contribution of feature type; shape, appearance or motion, and spatial scope; global, regional or distribution to the phenotypic variation in a given dataset. Each of SPOT's SAM features has an associated feature type and spatial scope (see Tables 1-3). To compute the contribution of feature type, the ‘expression’ of shape, appearance, and motion of each object was first derived, defined by the first principal component after applying PCA to the subset of pre-processed and normalized SAM features from SPOT Stage 3, step i, categorized as shape, appearance, and motion respectively. PCA was applied again to the object's shape, appearance, and motion expression. The absolute value of the coefficients of the first principal axis, which is associated with the first principal component, is defined as a score of the contribution of shape, appearance, and motion, respectively, to the data variance. The contribution of spatial scope was computed in the same manner.

### Phenomic Landscape Density Heatmaps

To produce flow cytometry-like density heatmaps of 2D UMAP projected points efficiently, the Gaussian kernel density of points was estimated using an image-based approximation. The UMAP plot was rasterized as an image of a desired resolution (e.g., 2000×2000 pixels). Individual 2D UMAP coordinates (umapₓ, umap_{y}) were transformed to integer image coordinates using

$$( {image}_{x} , {image}_{y} ) = ( ⌊ resolution \times \frac{{umap}_{x} - min ( {umap}_{x} , {umap}_{y} )}{max ( {umap}_{x} , {umap}_{y} ) - min ( {umap}_{x} , {umap}_{y} )} ⌋ , ⌊ resolution \times \frac{{umap}_{y} - min ( {umap}_{x} , {umap}_{y} )}{max ( {umap}_{x} , {umap}_{y} ) - min ( {umap}_{x} , {umap}_{y} )} ⌋ ) ,$$

where └ ┘ is the floor operator, max is the maximum value of UMAP (x, y) coordinates plus a padding value (in one example, 1 was used), and min is the minimum of UMAP (x, y) coordinates minus a padding value (in one example, 1 was used). Then, each UMAP point for the condition under consideration contributes +1 to the image pixel value at the computed corresponding image coordinate, producing a count of the number of individual points that map to a particular image pixel. The resultant image was finally Gaussian smoothed with an automatically determined sigma,

$$σ = \frac{1}{4} ( mean ( Euclidean pairwise distance between UMAP image coordinates ) ) .$$

The full pairwise distance matrix between all points does not need to be used for computation. It was found that a random sample of 1000 points was sufficient to obtain a smoothly varying density heatmap.

### Clustering of SAM Temporal Trajectories

To cluster the temporal trajectories, computed as described above, of organoids grown under different conditions, a pairwise distance matrix was constructed using multidimensional dynamical time warping (DTW) (through the dtaidistance Python package) to measure the distance between any two trajectories. Compared to pairwise Euclidean distances, the use of DTW accounts for variations in trajectory speed. The resulting distance matrix, D, is unbounded in magnitude; for clustering (or dimension reduction) D is transformed into an affinity matrix, A with entries in the range from 0-1 using the formula,

$${A}_{i j} = exp ( - \frac{{D}_{i j}}{s t d ( D )} )$$

where std( ) is the standard deviation of distances in D. The rows of the affinity matrix were used as the feature descriptor describing the trajectory of each condition. By default, average linkage and the Euclidean metric were used to hierarchically cluster conditions. For clustering of drug responses and mouse organoids in FIG. 4, the complete linkage was used.
scRNA-seq Analysis
scRNA-Seq Pre-Processing and Quality Control Filtering

Pre-processing of scRNA-seq data was conducted using Cell Ranger (v3.1.0). Briefly, FASTQ files were demultiplexed from raw BCL format using cellranger mkfastq. A custom Cell Ranger reference was then prepared (cellranger mkref) using a concatenation of the default Cell Ranger mouse genome sequence and annotation files (mm 10-2020-A: GENCODE vM23/Ensembl 98) and custom sequence and annotation files for the fluorescent markers used in this study. Alignment to this custom reference, identification of true cells, and tabulation of UMI counts for each sample was performed using cellranger count. The UMI counts for each sample were then combined into a single matrix using cellranger aggr without normalization.

Initial downstream processing was conducted using Seurat (v3.9.9.9038). As an initial filter for low-quality cells, cellular barcodes with less than 2000 genes detected, or more than 10% mitochondrial reads were removed. Normalization, variance-stabilizing transformation (VST), and scaling were conducted on the remaining cells using Seurat's implementation of sctransform (v0.3.2.9002), and the scaled VST counts for the 3000 most variable features were extracted from the Seurat object (scale.data slot).

scRNA-seq Selection of Variable Genes and Clustering

The scaled variance-stabilized counts of the 3000 most variable genes were used to cluster single cells using the graph-based Louvain algorithm (scanpy Louvain function) applied to the k nearest neighbor adjacency matrix (scanpy.pp.neighbors) using 20 principal components and k=15 for murine colorectal organoid scRNA-seq.

scRNA-Seq Cluster Connectivity and Dimensionality Reduction

To identify relationships between clusters (‘cluster connectivity’), Partition-based Graph Abstraction (PAGA) with connectivity model v1.2 was used, with Louvain clustering at resolution=1. Based on the PAGA connectivity, murine colon organoid scRNA-seq was visualized in 2D using the ForceAtlas2, ‘fa’ layout positions through scanpy.pl.draw_graph. For human duodenum scRNA-seq, 2D UMAP coordinates were computed using the ForceAtlas2 PAGA as initial embedding positions. Results of the ForceAtlas2 and UMAP gave the same interpretative results. UMAP was chosen for figure visualization as it spread the points better.

### Combined Visualization of Multiple Genes

The combined expression of signatures was plotted on the PAGA graph or UMAP coordinates as the mean z-score expression of the selected marker genes. The z-score of each gene was computed after log transformation, In (raw count+1) using scanpy.

### Data and Code Availability

Sequencing datasets used in this study are available from GEO: mutant mouse colonoid scRNA-seq, accession number GSE218339, mouse small intestinal bulk RNA-seq, accession number GSE218337, and human small intestinal scRNA-seq.

The developed SPOT Python library used to detect, track, and segment individual organoids and computation of SAM features to generate the results is available at GitHub, https://github.com/fyz11/SPOT.

### Example 3

Shape, Appearance, and Motion (SAM) are three major properties that characterize dynamic objects. Simultancous SAM assessment of a moving object, such as a worm, an organoid or a cell, can be used to predict behavior and reflect underlying cell-cell interactions and interactions between cells and their surrounding environment. It was hypothesized that an ability to measure SAM simultaneously and quantitatively could provide a comprehensive and dynamic read-out of the complex genetic and environmental interactions that control cell behavior.

As described in this example, an advanced and universal quantitative phenotypic imaging analysis framework, SPOT (SAM Phenotype Observation Tool), applicable to any object and both label-free and fluorescent images, was developed. SPOT operates by establishing a standardized set of SAM features, termed the SAM phenome, to describe the instantaneous object state. SPOT then establishes a standardized workflow to analyze the temporal evolution of SAM phenomes based upon the construction of a shared phenomic landscape, the partitioning of the landscape into phenotype clusters to describe phenotypic diversity, and the construction of phenotype trajectories to capture evolution. Finally, SPOT establishes an automatic procedure to group correlated SAM features into SAM modules, which can then be used to unbiasedly interpret the discovered phenotype clusters. SPOT is developed in the spirit of generalist or foundational models from machine learning, such as chatGPT or Segment Anything Model. Notably, however, SPOT is not learning-based. Consequently, it does not suffer from data-bias, and operates on data in a fully automated, agnostic manner, requiring no fine-tuning, user supervision or prior knowledge.

Using computer vision, single-cell tracking, and organoid imaging datasets, it was demonstrated that the proposed SAM phenome serves as a general imaging-based “transcriptome” that enables unbiased and comprehensive measurements of phenotypical diversity without prior-knowledge. Using SPOT, quantitative and dynamic measurement and monitoring of SAM can distinguish subtle differences in organoid growth between treatment conditions and predict phenotype-genotype-function coupling as supported by independent single-cell RNA sequencing (scRNA-seq). When combined with high-content organoid cultures and longitudinal live cell imaging, SPOT will advance the ability to identify and manipulate phenotypic heterogeneity and cellular plasticity through large scale genetic and drug screens.

### Development of Spot to Detect and Measure Sam Features

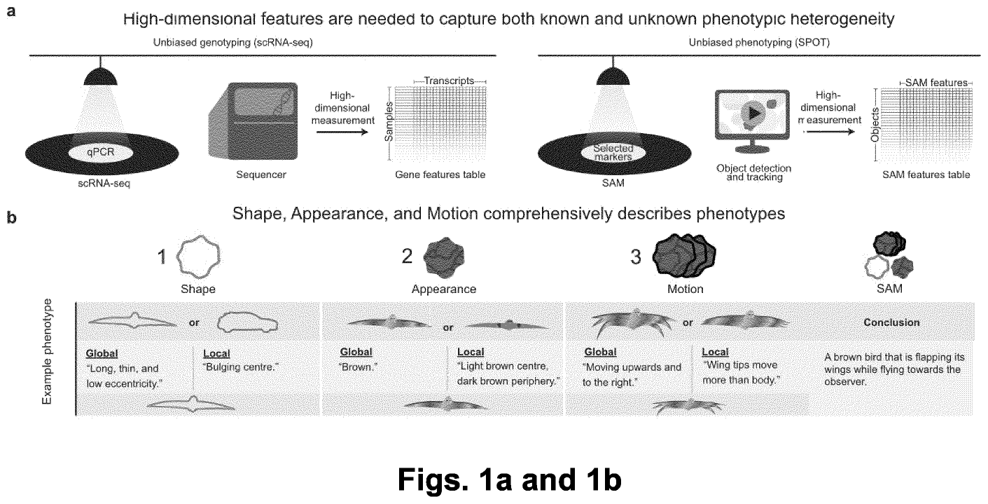

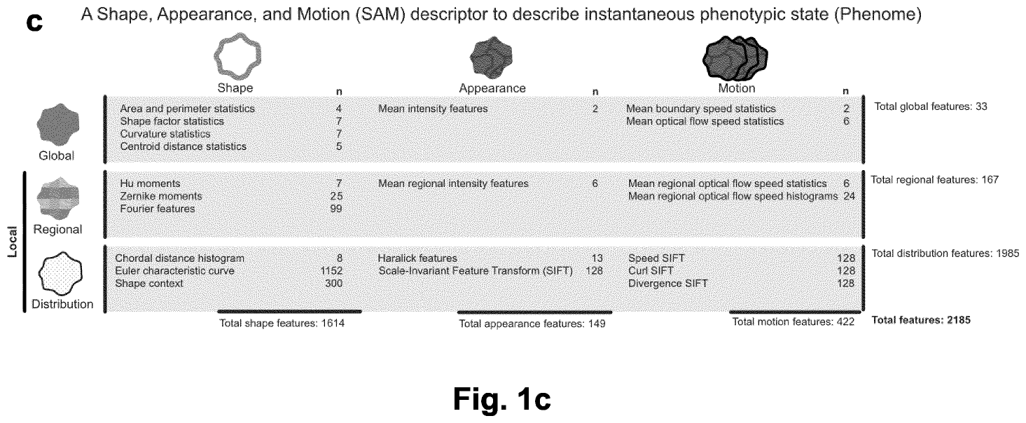

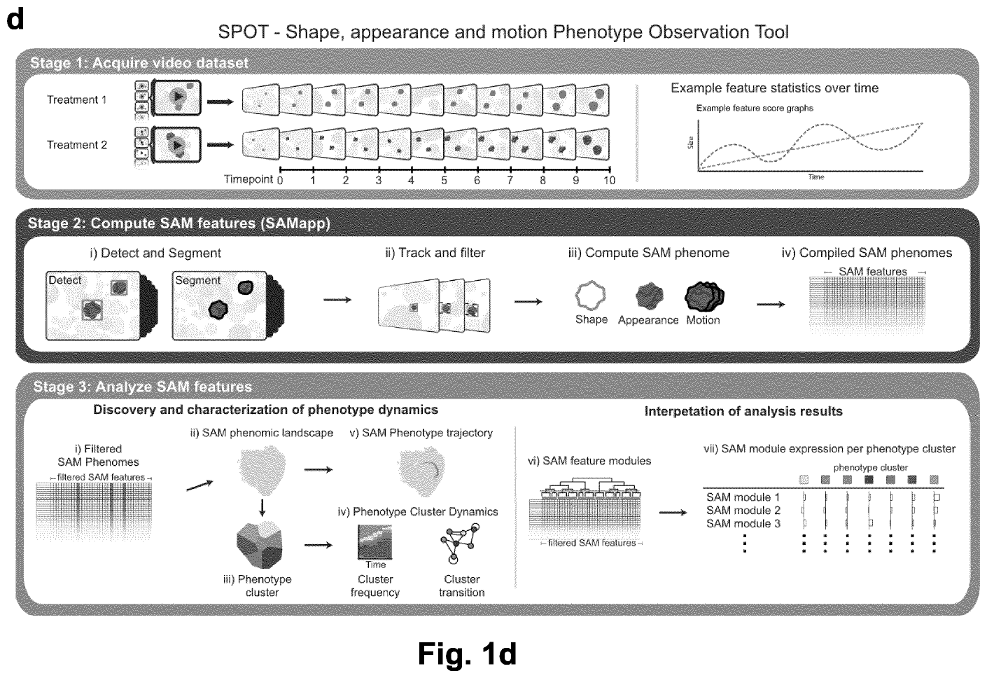

Single-cell sequencing, at scale with scRNA-seq, has transformed the ability to identify previously unknown and rare cell types. This is because the transcriptome of a cell can be comprehensively and unbiasedly measured by simultaneous sequencing of all detectable transcripts (average 200-6000 transcripts/cell). The transcriptomes of thousands or millions of individual cells can then be simultaneously analyzed, using dimensionality reduction and clustering of a filtered subset of the most informative, or relevant transcripts, to assess the transcriptional associations among cells and to define consistent molecular subtypes of cell clusters (FIG. 1a, left). Similarly, harnessing the full potential of high-content timelapse videos to detect unknown and complex phenotypes requires the construction of a set of object-agnostic, high-dimensional imaging features that comprehensively capture the imaging phenome. These features should moreover be easy to compute and scalable, so that hundreds of thousands of individual moving objects can be characterized, regardless of the imaged object, acquisition frequency, and time (FIG. 1a, right).

SAM constitutes a minimal set of image properties to quantify the instantaneous phenotypic state of an object. Shape is defined as the external border outline of an object and excludes internal composition. Appearance corresponds to the pattern of image intensity internal to the object. Motion is the difference in the object's shape and appearance between consecutive timepoints. For example, as shown in FIG. 1b, 1) combined global and local shape characteristics distinguish a bird-like from a car-like silhouette; 2) global and local appearance cues such as the difference in color hue, saturation, and patterning exclude car while consolidating the notion of bird vs plane; and 3) global and local motion determines whether or not the bird is currently flapping its wings and flying. While there will inevitably be some conceptual overlap between the three properties, crucially, when taken separately, they provide complementary information to quantify the object and its instantaneous action state.

An ideal quantitative SAM descriptor must be comprehensive and necessarily capture measurements at multiple spatial scales; global, regional, and pixel-level distribution. These scales correspond to the measurement of SAM features of the whole object (global), within parts of the whole object (local-regional), and the pattern of pixel-level SAM features within any given region of the object (local-distribution). Additionally, in order to be computable from videos of different objects, the measured SAM features must capture the distinctive aspects of a single object while also capturing a sufficiently broad range of additional features to distinguish this object from any other yet-to-be-measured object. To achieve this, the shape of segmented objects was discretized using 200 equally spaced boundary points, and the object area was partitioned into 3 regions of equal distance from its boundary, representing its border, middle, and inner core to compute regional SAM features. For consistent presentation throughout the paper, every object was treated as grayscale, converting any color images first to grayscale, and one set of appearance and motion was computed for the single color channel. These choices produce a 2185-D SAM descriptor per object instance (one object, one timepoint) (FIG. 1c, Tables 1-3). In practice, if all videos have the same number of image channels, separate appearance and motion descriptors for each channel can be computed and concatenated to form a more comprehensive SAM descriptor. The choice of 200 boundary points was chosen to minimize memory usage while still capturing the shape complexity.

To simultaneously detect and analyze SAM features over time in an unsupervised and data-driven manner, the SAM Phenotype Observation Tool (SPOT) was developed. The three main stages of the SPOT workflow are summarized in FIG. 1d. Stage 1 is video data acquisition. Videos of the objects are recorded over time with microscope modality, temporal sampling, and a spatial resolution relevant to the phenotype dynamics, e.g., cell migration, to be studied. Stage 2 is SAM feature computation. At each timepoint, for all identified objects, SPOT i) detects and segments; ii) tracks “genuine” objects by filtering out noise; iii) computes a set of SAM image features (Tables 1-3) termed the objects' instantaneous phenome (serving as a quantitative fingerprint of the instantaneous state of each object) and iv) compiles the multiple SAM phenomes of each object at multiple timepoints for all objects across all videos in the dataset into a single table for combined analysis. Stage 2 can be facilitated through the SPOTapp, which detects, segments, and tracks objects at every timepoint for every video through a graphical user interface. SPOTapp also allows users to directly use the results of external programs for the detection, segmentation, and tracking of objects (steps i, ii). SPOT filters out noise and identifies ‘genuine’ object instances after step ii, before SAM phenome computation. A ‘genuine’ instance is one that can be tracked consistently for a minimum number of consecutive frames as set by the user.

Stage 3 is SAM temporal analysis. SPOT exploits dimensionality reduction to analyze all measured SAM phenomes in the video dataset in a manner analogous to scRNA-seq workflows, where each SAM feature is treated as a genetic transcript equivalent. Specifically, SPOT i) pre-processes SAM phenomes, removing zero-valued, noisy, and non-temporally varying features to retain the subset of measured SAM features most informative to the timelapse experiment; ii) applies dimensionality reduction to the filtered SAM phenomes to construct a reduced 2D coordinate space, which maps the spatiotemporal phenomic variation between objects—the SAM phenomic landscape. SPOT uses this shared SAM phenomic landscape to jointly discover and analyze dynamic phenotypes across all conditions through iii) identifying SAM phenotype clusters, and iv) monitoring phenotype cluster dynamics (the cluster frequency over time and transition probability between clusters). The shared landscape is also used to v) construct SAM phenotype trajectories that capture the temporal evolution of phenotypic diversity in a given object population and can be used for comparative analysis. Lastly, to enable data interpretation, SPOT can vi) automatically group individual SAM features that exhibit the same covariation in the dataset into distinct sets or SAM modules. The expression pattern of SAM modules (vii) can subsequently be used to unbiasedly interpret the different phenotype clusters identified from iii). Henceforth, in all figures, all figure panels associated with a particular SPOT stage were outlined and color-coded. By treating every temporal instance of an object as an independent observation, SPOT amplifies an object by the number of temporal instances and increases the ability of analyses to detect under-represented and rare phenotypes. This feature enables SPOT to map and visualize the full phenotypic variation across space and time into a single 2D coordinate space (SAM phenomic landscape) that preserves the global and local similarity between objects and can be used as a world atlas to develop simplified analytics (SPOT Stage 3) to monitor and summarize dynamic phenotypic heterogeneity for each treatment condition.

### Validating SPOT's SAM Phenomes on Computer Vision Datasets

To validate and demonstrate the effectiveness of SPOT's SAM phenomes as discriminative descriptors that can identify unique SAM phenotypes across different complex datasets, three publicly available computer vision datasets with reference annotation were used, and the performance was also compared to similar features extracted using CellProfiler (Stirling, D. R. et al. BMC bioinformatics 22, 1-11 (2021))—the most popular software to measure shape and appearance features for biological applications. The terminology of SAM ‘descriptors’ was used to refer to the ensemble of SAM features used, and ‘feature set’ to refer to a specific group of SAM features such as shape (SAM-S), appearance (SAM-A), and motion (SAM-M) features. Dimension (n) refers to the number of SAM features used in an analysis.

The MPEG-7 database (Latecki, L. J. & Lakamper, R. IEEE Transactions on Pattern Analysis and Machine Intelligence 22, 1185-1190, doi: 10.1109/34.879802 (2000)) is an established computer vision benchmark shape classification dataset containing 1400 shapes equally sampled from 70 different categories (FIG. 2). Using this dataset, the ability of the SAM shape feature set (SAM-S) to correctly cluster objects into their shape categories using unsupervised k-means clustering (Lloyd, S. P. IEEE T Inform Theory 28, 129-137 (1982)) was tested. Clustering performance was measured based on adjusted mutual information (AMI) and adjusted rand index (ARI) (these scores range between 0-1, with 1 being the best performing score). Both the full SAM-S as well as its component feature sets were tested (Tables 1-3). The Euler characteristic curve (ECC) shape feature set has been demonstrated to perform better after dimensional reduction using kernel maps (Amézquita, E. J. et al. in silico Plants 4, diab033 (2022); Marsh, L. et al. arXiv preprint arXiv: 2212.10883 (2022)). Both variants were tested as separate feature subsets and as part of the full SAM-S. The full SAM-S using ECC features without reduction was referred to as SAM-S (ECC), n=1614, and full SAM-S with dimensionality reduction was referred to as SAM-S (kernel ECC), n=562, (in FIG. 2b). SAM-S (kernel ECC) (AMI=0.71, ARI=0.52) not only gave the best clustering performance compared to any subset of shape features but was also better than CellProfiler computed shape features (AMI=0.68, ARI=0.47) (Tables 1-3). Henceforth, unless otherwise specified, SAM-S is synonymous with SAM-S (Kernel-ECC). 2D-UMAP analysis applied to SAM-S (Kernel-ECC) displays the MPEG-7 shape variation in an ordered manner, from round and symmetrical (left-side of UMAP) to elongated (right-side of UMAP), while maintaining clustering amongst shape categories (FIG. 2b). 2D-UMAP analysis applied to CellProfiler could also similarly order shapes but visibly showed reduced ability to cluster similar shapes, resulting in the emergence of two distinct point clouds (FIGS. 2a-b).

The Normalized Brodatz database is an established computer vision benchmark texture (appearance) classification dataset, containing 112 unique texture images. Treating these 112 images as independent texture classes, cropping and rotation were applied to construct an augmented dataset of >10,000 image patches to test the ability of the SAM appearance feature set (SAM-A) (FIG. 2c) to correctly cluster patches into their appearance category using unsupervised k-means clustering. As for the shape analysis, the full SAM-A (AMI=0.46, ARI=0.14) outperformed all other component feature sets at classification and competitive to CellProfiler computed appearance features (AMI=0.49, ARI=0.14) (Tables 1-7). Despite the similar performance, UMAP analysis of SAM-A and CellProfiler appearance exhibited different behavior. Whereas SAM-A organized image patches with an emphasis on textural pattern similarity in 2D UMAP space (FIG. 2d), CellProfiler emphasizes more similarities in global brightness (FIGS. 2c-d).

To test the ability to measure shape and appearance simultaneously, 5 different Brodatz textures and 5 MPEG-7 shape classes were combined, where for each Brodatz class, 20 random crops were extracted and applied to each and every of the 20 unique shape images in each MPEG-7 class, to construct a synthetic dataset of 10,000 images (5 shapes×20 images per shape×5 textures×20 images per texture) with joint shape and appearance variations (FIG. 2c). Applying SPOT to compute combined all shape and appearance descriptors (SAM-SA) and using UMAP to display the results in 2D, it was found that unique shape images were separated as individual tightly grouped highlighted point clouds (FIG. 2f). Each point cloud was further subdivided into 5 smaller point-clouds, each corresponding to a Brodatz texture (FIGS. 2f and 2g and zoom-ins). Thus SAM-SA could reverse-engineer the data generation process. Globally, there is also broad separation of the five primary shape categories representing the main variation of difference. In contrast, UMAP applied to CellProfiler combined shape-appearance features finds point clouds that cluster at the coarse level of MPEG-7 shape and Brodatz texture classes but cannot resolve individual unique shape images (FIGS. 2e-g). Together, these analyses of synthetic datasets validate the ability of SPOT's SAM phenome to accurately and simultaneously classify objects based on their shape and appearance.

Finally, the ability of the full set of SAM descriptors was tested to simultaneously detect combined variations in shape, appearance, and motion (SAM) using the A2D dataset (Xu, C., et al. in Proceedings of the IEEE Conference on Computer Vision and Pattern Recognition. 2264-2273). This dataset comprises 3782 videos collected from YouTube and reflects unconstrained ‘real-life’ scenarios. The videos depict seven classes of moving objects (adult, baby, bird, cat, dog, ball, and car) performing nine different movements (still, labeled for no movement), climbing, crawling, eating, flying, jumping, rolling, running, and walking (FIG. 2h). A single movement class can be performed by different objects, but no object can perform all eight movements. Altogether, there are 43 unique action-movement pairings. The dataset is provided as 3036 training videos and 746 testing videos. For each video, only 3-5 non-contiguous frames are annotated. Optical flow and the segment anything model were used to generate consistent segmentations of each object for the annotated frame and the immediate next frame in order to compute SAM descriptors for Shape, Appearance or Motion individually or combined (SAM-S, n=562, SAM-A, n=149, SAM-M, n=422, or full SAM, n=1133). K-means clustering analysis with cluster centers learned on the training videos, then applied to the testing videos revealed again that the full SAM descriptor (SAM) outperformed in both AMI and ARI metrics compared to SAM-S, SAM-A, and SAM-M (Tables 1-3). Whether SAM descriptors could be used for supervised machine learning was also tested, and improved classification on the testing videos was found. Again, the full SAM descriptor significantly outperformed either SAM-S, SAM-A, and SAM-M features alone in the classification of object, movement, and object-movement pairings (scores ranging 0-1, the higher, the better). Notably, the results are competitive with those previously reported using more sophisticated machine learning techniques that model probabilistically the object-movement pairing and use specialized features that exploit long-time tracking of objects over the full duration of the video, while here, only a consecutive frame pair was used.

Applying UMAP to the full SAM features and coloring by object (FIG. 2i) and movement class separately (FIG. 2j), object-movement (actor-action) pairings that are more commonly associated are also more commonly co-clustered in the UMAP. For example, the ‘ball’ object maps to the top right of the UMAP and strongly colocalizes with the ‘flying’ movement. Alternatively, the ‘adult’ object maps to the bottom of the UMAP and is strongly associated with the movements; running, climbing, and walking—two-footed or ‘upright’ activities that are unlikely to be filmed being carried out by objects such as ‘baby’, ‘dog’ or ‘cat’. Next, whether a similarity graph could be inferred between actors and actions by binary thresholding on the pairwise distance between the 2D UMAP median coordinate of each actor or action was tested (FIG. 2i-j inset). The resulting graphs reveal a clique that groups together 4-“legged” objects, including baby, cat, and dog, co-localized with actions more frequently observed and associated with these objects; crawling, jumping, eating, and rolling. This observation was further verified by constructing and visualizing an additional similarity graph based on unique object-motion pairings. Moreover, similar conclusions could be inferred using different dimensionality reduction techniques (FIG. 4). These results verify that the SAM UMAP 2D phenomic landscape is semantically meaningful. This landscape can simultaneously distinguish between object and motion phenotypes and co-localize object-motion associations in a manner that allows for the application of cartography. In summary, the results demonstrate that SPOT's SAM phenome is general, informative, and inherently takes into account the relative importance of shape, appearance, and motion.

### SPOT Detects Phenotype Clusters and Maps Temporal Phenotype Trajectory

When cells grow and/or migrate, they exhibit diverse morphological changes globally and locally. Global morphological changes include transitions from round to elongated and division from one cell to two daughter cells. Local changes include subcellular protrusions such as blebs, lamellipodia, ruffles, and filopodia. Cells may also exhibit different types of movement, such as amoeboidal, mesenchymal, and neuronal. Standard analytical approaches are based on tracking individual cells over time. The resultant single-cell trajectories are ideal for monitoring the phenotypic change of the unique cell over time. However, this trajectory representation becomes increasingly challenging when behavior across cells should be compared, where the number of frames that each cell is tracked for differs, and the cells may be in different phenotypic states. Harnessing the ability of SPOT's standardized and comprehensive SAM, SPOT stage 3 presents a streamlined temporal analysis framework utilizing the constructed shared 2D SAM phenomic landscape as a global positioning coordinate system.

The ability of SPOT's simplified temporal analysis framework was tested by detecting phenotypic variations of single cells within small datasets by applying Stages 2 and 3 of the SPOT workflow to two publicly available single cell tracked videos, with known reference segmentation and single-cell tracks from the cell tracking challenge (Maska, M. et al. Bioinformatics 30, 1609-1617 (2014)). The first dataset records the cell migration of glioblastoma-astrocytoma U373 cells without cell division (FIG. 3a). The second dataset records the division of simulated acute promyelocytic leukemia HL60 cells HL60 with negligible cell migration. The U373 cells were first analyzed using the provided segmentations and single cell tracks, computing the SAM phenomes of each cell for every timepoint it was tracked and compiling all SAM phenomes (SPOT stage 2: SAM computation). Stage 3 of the SPOT workflow was then used to analyze the data generated from stage 2 by i) filtering out non-informative SAM features and generating filtered SAM phenomes that retain the most temporally changing features that are most likely to drive the temporal behavior (scc Methods); ii) dimensionality reduction using UMAP to map all cell instances into a single 2D SAM phenomic landscape. The 2D UMAP coordinates of an individual cell timepoint position its multidimensional SAM ‘expression’ profile relative to all instances in the full dataset of this phenomic landscape; iii) clustering phenotypes and iv) phenotype dynamics (SPOT Stage 3, FIG. 2d). Using k-means clustering, with the elbow method to select the number of clusters, SPOT partitioned the phenomic landscape into seven phenotypic clusters (FIG. 3b, left, Methods). To aid cluster interpretation, the phenomic landscape was highlighted using selected measurements (FIG. 3b, right). The local point density shows that most cell instances map to the center (FIG. 3b, right panel, first plot), covered by clusters 1,6,7, while the left and right extremities (cluster 2, 5, and 4, respectively) are composed of eccentric, elongated cell shapes (FIG. 3b, right panel, second plot). In this case, the mean intensity and mean speed exhibit no clustering, indicating that global brightness and speed are not distinguishing features of U373 migration (FIG. 3b, right panel, third and fourth plots).

Lastly, as expected, no cell division events were detected (FIG. 3b, right panel, fifth plot). To further illustrate what is represented by each SAM phenotype cluster, the top 4 cell instances per cluster identified by SPOT were visualized (FIG. 3b, left). Specifically, cluster 1 represents the majority phenotype whereby most cells are approximately spherical, have a lamellipodium, and exhibit asymmetric ruffling. This cluster overlaps with the region of the highest density in the UMAP SAM phenome. Cluster 2 cells are elongated, with symmetric, similar-sized ruffles at both tips. Cluster 3 cells have a bright, round nucleus, an asymmetric lamellipodium, and small, uniform ruffles along their perimeters. Cluster 4 cells are elongated, with larger lamellipodia at one tip. Cluster 5 cells possess wide, polarized lamellipodia with ruffles characteristic of mesenchymal migration. Cluster 6 cells have a single bright, round nucleus and no lamellipodia. Finally, cluster 7 cells have nuclei that are centrally black and textured, small lamellipodia, and typically star-shaped, with finger-like protrusions.

The phenotype clusters act like a dictionary, and the cluster label categorizes the SAM state of every cell instance, which can be determined from its 2D UMAP coordinate. For a single cell, this labeled trajectory reveals how phenotype switching evolves over time (FIG. 3c, left). However, the labeled trajectories of all cells are difficult to compare: not only are they of different temporal lengths, but state transitions may be stochastic. The same phenotype cluster transition in one cell occurs after different amounts of time and may not be preceded nor succeeded by the same cluster transitions in another cell. This makes it difficult to extract any consensus patterns of behavior. Furthermore, any alignment across cell trajectories is challenging or unscalable (FIG. 3c, right). In SPOT, the concept of pseudotime and clonotypes were adapted from sequencing analysis and developed dynamic, population-level measurements of SAM phenotype cluster frequency, plotted as stacked barplots (Step 3: iv, FIG. 1d). The resulting histograms show how the proportion of cell instances assigned to each phenotype cluster change over time (FIG. 3d, left). The expansion or contraction of a phenotype cluster reveals time-dependent treatment effects. Notably, all U373 phenotype clusters exhibited periodic expansion and contraction, and this is most visible for cluster 6, consistent with these cells undergoing regular phases of migration and complete contraction of their lamellipodia before expanding their ruffles and migrating again. Hidden Markov models (HMM) (Eddy, S. R. Nature biotechnology 22, 1315-1316 (2004); Held, M. et al. Nature Methods 7, 747-754 (2010)) was used to estimate the probability of transitioning between phenotype clusters from the cell tracking information generated by step 2 of SPOT (FIG. 3d, right; the more transparent the arrow color, the lower the transition probability). Using HMM, several dominant recurring phenotype transitions were observed: a nucleus-only cell (cluster 6) evolves to have large, spreading lamellipodia (cluster 3) and vice-versa; symmetric cells with multiple pseudopodia (cluster 7) transition to become polarized, with large lamellipodia and ruffling (cluster 4) and vice-versa; the preference of an elongated, polarized and ruffling cell (cluster 2) transitioning towards a more contracted lamellipodium with multiple pseudopodia and ruffles (cluster 1), which appears self-persistent; and a self-persistent state of cells with polarized large, ruffling lamellipodia (cluster 5).

For high-throughput screening, it is necessary to have a single readout that preserves both temporal dynamics and phenotype diversity, and that can be used to compare behaviors across conditions. An alternative way to quantify a cell's phenotypic evolution over time is to concatenate individual cell instances chronologically and map its position in the UMAP SAM phenomic landscape to generate a SAM phenotype trajectory (FIG. 3e, top). Computing the phenotype trajectories for all 29 tracked cells, comprising a total of 1189 cell instances from the two U373 videos, significant heterogeneity in the dynamics of individual cells was found (FIG. 3e, bottom). To describe the phenotypic behavior of a given cell population or cluster, the SAM temporal phenotype trajectory was developed. All cell instances within each temporal bin were mapped onto the global UMAP SAM phenomic landscape, and a single average coordinate per bin was derived and integrated into a chronological trajectory. The shape and direction of the resultant trajectory captures the dominant behavior of the population over time. If there is no change in phenotypic state across all cells between time intervals, then the trajectory is static. If a proportion of cells changes phenotypic state, the direction of the trajectory captures the transition towards a different part of the phenomic landscape. The ‘length’ of the trajectory movement between timepoints reflects the phenotypic difference before and after transition. The further apart the two phenotypes are in the landscape, the longer the trajectory movement. The length also takes into account the diversity across cells undergoing phenotype switching. The more cells that undergo phenotype transitions between different phenotype clusters within a particular time interval, the shorter the net trajectory movement. For U373, the population phenotype trajectory indicates an overall cyclic transition, starting from cluster 1 towards 5, towards 2, towards 3, towards 6, back to 1. However, the movement of the trajectory is constrained to the center of the phenomic landscape (FIG. 3f). This pattern indicates that individual cell phenotypic switches are asynchronous and involve different phenotype clusters.

SPOT's SAM phenome descriptors were used for unbiased data interpretation. The absolute value of the first principal components of the SAM-S(Shape), SAM-A (Appearance), and SAM-M (Motion) feature sets defines a series of contribution scores, indicating the relative importance of shape, appearance, and motion to the total phenomic variation across the whole dataset. Likewise, the absolute value of the first principal component of features grouped by spatial scope indicates the contribution of global, regional, and distributional SAM features. In this dataset, motion and distribution are the largest sources of variation, with contribution scores of 0.78 and 0.93, respectively (FIG. 3g, left). For further interpretation of individual phenotype clusters, SPOT can automatically cluster the SAM features into independent SAM modules by applying hierarchical clustering to the feature correlation matrix (FIG. 3g middle). The mean ‘expression’ of each SAM module in each phenotype cluster can then be quantified as the mean of the first principal component score when principal components analysis is applied to the component SAM features (denoted as just ‘score’ in FIG. 3g, right, barplots, Methods). This score reflects the extent to which a SAM module is enriched across phenotype clusters and can be used to interpret phenotypic differences across clusters. To interpret each SAM module, SPOT automatically generates the most relevant image examples and SAM features, and indicates whether a feature is increasing (up arrowhead) or decreasing (down arrowhead). Here, the top three images and two features were used (FIG. 3g, right, images and text).

Stages 2 and 3 of SPOT were also applied to analyze the video of the simulated fluorescence labeled HL60 cells, using the same workflow as described above for U373 cells. HL60 cells are grown in suspension culture and show high levels of cell division, with little cell movement. SPOT identified six phenotype clusters. Coloring the SAM phenomic landscape based on the local mapped point density, shape eccentricity, mean intensity, mean speed, and division events and visualizing the top 4 cell instances per cluster, clusters 1, 2, 4, and 5 were associated with cell division as chromosome segregation is strongly associated with cells that have elongated, eccentric shapes. By contrast, clusters 3 and 6 correspond to non-dividing cell clusters. Of the cell division clusters, clusters 2 and 4 have relatively lower fluorescent image intensity, while clusters 1 and 5 have higher fluorescent image intensity. Similarly, of the non-dividing cell clusters, cluster 6 has low fluorescence image intensity, whereas cluster 3 has high fluorescent image intensity. As HL60 cells are fluorescently labeled, when cells divide, they lose fluorescence. Therefore, clusters with low intensity are likely to derive from those with high intensity. As for U373 cells, it was difficult to identify consensus behavior from the SAM phenotype labeling of individual cells, though cell divisions were synchronized and identified 4 cell cycles. Nevertheless, SPOT was able to detect the changes in fluorescence intensity, and the cluster dynamics associated with these changes were captured by the temporal stacked barplot. Early dominance of clusters 1 and 3, and the transient cluster 5 were typically superseded by clusters 2, 4, and 6. The prevalent time-dependent transitions between clusters were captured by HMM. Cluster 1 transits to cluster 3 and vice versa; cluster 2 transits to cluster 6 and vice versa; cluster 4 transits to cluster 2; while cluster 5 is largely self-persistent.

As for the U373 cells, individual phenotype trajectories are heterogeneous, and it is difficult to draw conclusions from them. The SAM population phenotype trajectory shows clear phenotypic evolution of the population, starting from cluster 3, and moving towards cluster 6 (dominated by the loss of fluorescent intensity), then towards cluster 4 as cells divide, back through cluster 6 as daughter cells grow, and towards cluster 2 as cells divide again. In particular, the trajectory has 4 ‘loop-like’ directional changes, predicting 4 cell cycles, which corroborates with the division pattern over all the cells.

Plotting the top 3 images for each SAM module and using first principal components of SAM-S, SAM-A, and SAM-M revealed that the major SAM score contributors are appearance-associated features such as ‘mean_intensity,’ ‘sum_entropy’ as well as more global features like ‘eccentricity’ and ‘curvature’. This is reflected in the contribution score; appearance is the largest contributor (0.78) and motion the smallest (0.22). Although distributional SAM features (0.87) were best suited to capture the speckled patterns of chromosome segregation prior to cell division, in this dataset, global features (0.37) are significantly more important than for U373 cells, being on a par with regional features (0.34).

Consistent with the U373 videos mainly exhibiting cell migration and the HL60 videos exhibiting cell division, the primary SAM contributors differed significantly between the two video datasets. For the U373 cell videos, motion and distribution-associated features were the main SAM score contributors, whereas for the HL60 videos, appearance and global and regional associated features contributed the most to the cell variation. In summary, these results confirm that the workflow of steps 2 and 3 of SPOT successfully automatically detects and extracts informative and interpretable SAM features and can cluster objects based on their SAM phenotypes. Moreover, SPOT can generate concise readouts of dynamic phenotypic heterogeneity as stacked barplots of phenotype cluster frequency and phenotype trajectory maps that characterize cellular behavior.

### SPOTting the Spatiotemporal Dynamics of Complex Organoids

Multicellular structures, such as organoids, have emerged as attractive experimental models. Further, the complex spatiotemporal dynamics of organoids are underlined by the cells' genetic makeup, cell/cell interactions, and the constituent cells' responses to environmental perturbations. SPOT's potential was therefore tested to measure the spatiotemporal dynamics of organoids using normal patient-derived duodenal organoids grown in a 96-well plate and imaged label-free. The entire SPOT workflow was applied to measure organoid response to eight chemotherapy drugs at three different concentrations. 60-100 organoids were seeded per well, and all organoids were filmed every hour for 5 days (stage 1). In total, 516,503 organoid instances were segmented from the 96 videos. For each organoid instance, the same 2185-D SAM descriptor was generated for single-cell tracking (stage 2). The SAM descriptors associated with each organoid instance over all timepoints were then compiled and filtered. UMAP dimensionality reduction was applied to construct the 2D SAM UMAP phenomic landscape (stage 3).

As with the single-cell tracking datasets in FIG. 3, k-means clustering with the elbow method identified seven clusters that capture the phenotypic heterogeneity. The clusters group organoids with similar multidimensional SAM characteristics. The clusters are numbered 1 to 7 and sorted in order of increasing mean eccentricity, a measure of the deviation from the normal, spherical morphology of intestinal organoids cultured in human organoid medium (HOM), a culture condition adopted consistently for organoids throughout this study. For each cluster, the most representative four images were selected as exemplars to depict the variability in scale amongst clusters. To emphasize differences in appearance, the exemplar images were resized to generate standardized images of the same image size. Qualitatively, cluster 1 and 2 organoids are spherical and have bright lumens, while cluster 7 organoids are dark.

As for single cell tracking, SPOT was applied to identify the temporal phenotypic behavior across conditions without a priori ascribing meaning to the identified phenotype clusters. The results from the final timepoints, at maximum concentration across all drugs, indicate that, under the conditions used, dimethyl sulfoxide (DMSO, representing control), cisplatin, oxaliplatin, mitomycin C, 5-fluorouracil and gefitinib have lower relative toxicity than irinotecan, docetaxel and epirubicin. This is supported by visual inspection of the density heatmaps, which show the total number of organoid instances treated with the indicated drugs across all three concentrations mapped to local regions of the phenomic landscape. While the exact location of heatmap hotspots varies, the results for cisplatin, oxaliplatin, mitomycin C, and 5-fluorouracil were similar to those for the DMSO control and appeared to have limited impact on organoid morphology, even at the highest concentrations (6, 20, 0.03 and 20 μM, respectively). However, treatment with gefitinib (up to 0.5 μM) resulted in smaller organoids which clustered at the bottom of the UMAP, compared to DMSO control. Interestingly, organoids treated with Irinotecan (up to 5 μM), docetaxel (up to 0.0125 μM) or epirubicin (up to 4.5 μM) were enriched in both the lower and left areas of the UMAP (clusters 4,7), where the organoids appear small, with dark lumen and irregular morphology, phenotypic features associated with cell death. Consistent with this analysis, the final frame images depict high levels of cell death for treatments with these drugs.

Next, the SPOT temporal SAM analysis was applied to monitor dynamic changes in phenotypic heterogeneity (Stage 3, steps iv,v). The stacked barplots of phenotype cluster frequency over time show substantial expansion of the more toxic-associated phenotype clusters 4 and 7 from day 2-3 onwards. In contrast, the low-toxicity drugs show expansion of clusters 1, 2, 6, and mildly toxic 5-fluorouracil and gefitinib approximately maintain the fraction of the cluster 2 large spherical organoid phenotype. The HMM cluster transition analysis shows that the dominant cluster transitions are preserved across non-toxic drugs, but reveals differences in cluster transitions amongst mildly toxic 5-fluorouracil and gefitinib. Amongst the three toxic drugs, docetaxel and epirubicin appear to have a conserved cluster transition pattern, while irinotecan loses the autoregulatory loop of cluster 2. Notably, this transition pattern is most similar to that of 5-fluorouracil, whose mechanism of action has been reported to involve double-strand DNA breaks.

Next, the temporal phenotype trajectories were used to summarize the net phenotype behavior for each drug. Predominant organoid growth is indicated by a trajectory from bottom to top in DMSO, cisplatin, oxaliplatin, and mitomycin C-treated organoids. Increasing toxicity leads to deviation of this trajectory path, with the start moving right-to-left and developing into an opposing trajectory from top to bottom, as for organoids treated with irinotecan and epirubicin. The more toxic, the faster the deviation. 5-fluorouracil and gefitinib showed deviations from the DMSO trajectory, initially going upwards and then coming back down, implying that a longer time was required to generate toxicity, and thus, there is milder toxicity that was not evident from either the final frame snapshots or the heatmap. The trajectories were hierarchically clustered to compare their similarity, and the resulting dendrogram correlates with the drugs' principal mechanisms of action. Notably, organoids treated with chemotherapeutic drugs affecting DNA synthesis (cisplatin, oxaliplatin, mitomycin C) and inhibiting mitosis (irinotecan, docetaxel or epirubicin) cluster into two distinct groups. It was further found that 5-fluorouracil and gefitinib were functionally similar but had different mechanisms of action, as detected by the HMM transition graph analysis.

Lastly, SPOT's SAM module analysis (stage 3, step vi) was used to automatically interpret the phenotypic clusters. As expected for toxicity, the contribution scores identified shape and appearance as the dominant SAM features. Further, in contrast to single-cell tracking, which involves local morphological changes, global features (0.67) were almost equally as informative as distributional SAM features (0.72). 13 SAM modules summarize the phenotypic variation. Impressively, modules 1-4, corresponding to the first 4 partitions of the dendrogram, automatically discovered the notions of area, eccentricity, brightness, and mean speed without any user supervision. Specifically, SAM module 2 ranks clusters 1-7 in increasing eccentricity. From the SAM module expression table, clusters 2 and 6 were identified as normal growth phenotypes based on large size (module 1), relatively bright lumen (module 3), and low moving speed (module 4) in contrast to clusters 4 and 7, which are the smallest (module 1), darkest (module 3) and exhibit the highest moving speed (module 4) and also have morphodynamics of non-growing organoids.

SPOT, therefore, enables fully automated discovery and evaluation of the similarity of organoid phenotypes under different treatment conditions. Moreover, the phenotype trajectories can be used to hierarchically cluster conditions analogous to the comparison of transcriptional similarity between single cell subpopulations in scRNA-seq analysis. Together with the stacked barplots and HMM cluster transitions, these results demonstrate how SPOT can measure, fingerprint, and interpret the underlying spatiotemporal dynamics in phenotypically heterogeneous organoid imaging datasets in a manner applicable to drug screening.

To further validate SPOT on a more morphologically complex dataset, label-free live-cell imaging of mouse small intestinal organoid cultures that carry defined genetic alterations were analyzed. Wild type (WT, p53^{+/+}) and p53 null (p53^{-/-}) organoids were grown in normal mouse organoid culture medium with or without inhibitors and imaged every 15 mins over 5 days (FIG. 4a). Valproic acid (V) inhibits histone deacetylase, an enzyme that regulates chromatin function, whereas CHIR99021 inhibits GSK-3 beta, a serinc/thrconine kinase that inhibits the WNT signaling pathway. Consistent with a previous report (Yin, X. et al. Nat Methods 11, 106-112 (2014)), treatment with V/C induced enhanced growth and branching morphology in organoids. This effect is largely due to V/C's ability to alter key signaling pathways, including epigenetic alteration and WNT activation, to induce stem cell expansion and differentiation.

SPOT identified three phenotypically distinct clusters, clusters 4, 7, and 8 of large area and branching morphologies, as depicted by representative image exemplars (FIG. 4b). The UMAP density heatmaps of all organoids (WT and p53^{-/-}, with and without V/C) show a milder and stronger effect of V/C in p53^{-/-} and WT organoids, respectively, with larger and more branched organoids in both cases (FIG. 4c i and ii). This is reflected in the stacked barplots of phenotype cluster frequency over time, which show pronounced increases in the proportion of branched organoid phenotypes, particularly clusters 7 and 8 (FIG. 4c iii). The HMM cluster transition analysis reveals large differences among all four conditions but also a conserved cluster 3-to-7 bi-directional transition upon V/C addition (FIG. 4c iv). The cluster 3-to-7 transition is consistent with accelerated growth and branching induced by V/C. The cluster 7-to-3 transition is likely due to the fact that, when the imaged organoid is sufficiently large, the branches appear to ‘buckle’ or fold in, and there is slowed growth such that its state is no longer well described by any of clusters 4, 7, and 8. Again, the phenotype trajectory summarizes the phenotypic heterogeneity and dynamics with respect to the phenomic landscape (FIG. 4c v). All four conditions show a bottom-to-top temporal trajectory, consistent with growth and increased irregularity, with changes occurring most dramatically for WT+V/C, which have the longest and most directional trajectories. Hierarchical clustering of trajectory similarity demonstrates the global effect of V/C treatment, and its stronger effect on WT.

Applying SPOT's SAM module analysis (stage 3, step vi), it was found that the contribution scores now reflect the fact that branching is more strongly associated with shape (0.73) than motion (0.57), with appearance features (0.38) contributing least (FIG. 4d). Further, branching is best characterized by distributional (0.82) rather than global (0.46) or regional (0.33) features. 17 SAM modules summarize the phenotypic variation. Using the top representative exemplar images and top SAM features, and recording whether a feature is enriched (up arrow) or depleted (down arrow) in a given module, it was found that module 1 automatically captures the reduced solidity and increased curvature associated with branching clusters 4,7, and 8. By contrast, module 2 appears to have discovered specific distributional motion pattern features (sift_motion features) that score the clusters according to the extent of growth and branching. As with the drug screen, it was again automatically discovered that modules 5, 6, 3, and 7 correspond to area (equivalent_diameter), eccentricity (eccentricity), brightness (intensity), and mean speed (speed_global_flow), respectively. This indicates that these SAM characteristics can be used as a consistent set of informative features to monitor intestinal organoid morphodynamics. Examining the SAM expression profiles across clusters, it was verified that clusters 4, 7, and 8 indeed share similar SAM traits and that cluster 3 exhibits a profile associated with reduced growth and branching. These findings are consistent with the HMM-predicted cluster 7-to-3 transition, where organoid growth and branching significantly slow at later timepoints. Henceforth, area, eccentricity, intensity, and mean speed were used as targeted SAM features to profile intestinal organoid phenotype clusters in order to simplify presentation.

To explore a potential genotype-phenotype connection, bulk RNA-seq was performed on organoid populations, harvested after 5 days of treatment. Dimensionality reduction plotting of the transcriptomes using multidimensional scaling (MDS) indicates a clear separation of treated vs untreated organoids (FIG. 4c). SPOT resolved both the global phenotypic effect of V/C treatment and its differential potency on WT and p53^{-/-} organoids (FIG. 4c and dendrogram). This demonstrates that SPOT provides a complementary analysis to molecular sequencing. SPOT not only captures the endpoint outcome but, thanks to the additional incorporation of time, can offer additional insight into how the phenotypes developed.

### SPOT Reveals a Previously Unrecognized Phenotype-Genotype Connection

To showcase the ability of SPOT to detect cellular plasticity using SAM features, detailed phenotyping of mouse intestinal organoids was performed with defined, disease-relevant genetic alterations. Mutant APC, KRAS, and TP53 are well-known drivers of colorectal cancer (CRC) tumorigenesis. Inactivating mutations in APC are present in 50-83% of sporadic CRC cases and lead to enhanced WNT signaling and stemness of intestinal epithelial cells. APC-deficient stem cells acquire growth advantages that facilitate their clonal expansion in intestinal crypts in vivo. Activating mutations in KRAS, an essential mediator of the MAPK pathway, are present in 35-45% of sporadic CRCs and lead to the constitutive activation of downstream signaling pathways, promoting cell survival and proliferation. TP53 is mutated in 40-50% of sporadic CRCs, most commonly by missense mutations that lead to p53 dysfunction. Mutant TP53 (deletion or missense mutation) carrying cells are often resistant to cell death. Further, mutations in tumor suppressors APC and TP53 and the oncogene KRAS are known inducers of cellular plasticity in 2D cell cultures. Therefore, it was hypothesized that organoids carrying mutant APC, TP53, and KRAS cither alone or in combination might exhibit distinctive morphodynamics that reflect dysregulated signaling pathways and their oncogenic potential. SAM features detected by SPOT in this set of organoids may provide new insights into genotype-phenotype connections.

Organoids were derived from the proximal colon of mice carrying mutations in Apc, Tp53 or Kras either alone or in combination. Organoid genotypes were confirmed by PCR, labeled with fluorescent proteins and artificially highlighted for imaging as indicated: APC^{min/+} (mCherry-red); p53^{null} (mBanana-green); p53^{R172H:R172H} (mBanana-yellow); KRAS^{G12D/+} (EYFP-blue) and KRAS^{G12D/+}:p53^{null} (EYFP-purple). Organoids with the above-mentioned genotypes were first cultured for 72 h and imaged with confocal microscopy every 2 h for 4.5 days. A total of 79,662 organoid instances pooled from 5 independent experiments across all timepoints, and all genotypes were segmented and measured. KRAS^{G12D/+}, APC^{min/+}, and KRAS^{G12D/+}:p53^{null} organoids grew to larger areas than p53^{null} and p53^{R172H:R172H} organoids throughout the 4 days of filming. In contrast, all genotypes showed similar entropy—an appearance-based feature that measures the roughness of the image texture, at day 4 despite, a higher initial value for APC^{min/+} organoids. Interestingly, APC^{min/+} organoids exhibited higher eccentricity and mean speed during day 1 and day 2 of filming, but the double mutant KRAS^{G12D/+}:p53^{null} organoids caught up by day 4.

These results demonstrate that the choice of a single statistic or timepoint cannot adequately quantify the subtle and dynamic, multidimensional phenotypic differences between organoids with different genotypes. Using SPOT (Stages 1,2,3 steps i-ii), the same 2185-D SAM phenome for all organoids from all genotypes, and all timepoints were computed. UMAP was then used to project the compiled and filtered SAM phenomes onto a 2D phenomic landscape, which identified 8 phenotypic clusters, ordered by increasing eccentricity. As expected, the stacked barplot of phenotype cluster frequency over time for each genotype showed that the KRAS and p53 genotypes expectedly exhibit similar patterns of phenotype cluster dynamics while APC^{min/+} is different. Over time, the most shape eccentric clusters expanded the most in APC^{min/+} organoids compared to organoids of other genotypes. Specifically, ˜15-20% of APC^{min/+} organoids have features identified in orange clusters during the early stages of filming, with the number of organoids in these two clusters expanding to 30-40% of the total organoids measured at the end of the videos. HMM cluster transition analysis revealed different cluster transition dynamics across all genotypes, with consensus between APC^{min/+} and KRAS^{G12D/+}:p53^{null}. The temporal phenotype trajectories highlight the early deviation of the APC^{min/+} trajectory leftwards from p53^{null}, p53^{R172H:R172H}, KRAS^{G12D/+} and KRAS^{G12D/+}:p53^{null} genotypes. Moreover, hierarchical clustering of trajectories confirmed that APC^{min/+} clustered separately from all other genotypes. Notably, and in agreement with the HMM predicted cluster transitions, at later timepoints, the KRAS^{G12D/+}:p53^{null} trajectory deviates towards irregular morphologies; a similar profile to the APC^{min/+} trajectory.

To ensure these observations were not caused by phototoxicity from prolonged laser exposure, and brightfield microscopy-acquired timelapse videos of the genotypes show the same expansion of shape eccentric clusters in APC^{min/+} was verified.

Together, the results demonstrate that SPOT can detect phenotypic changes caused by single-gene perturbations and produce consistent results irrespective of whether videos were acquired with fluorescent confocal microscopy or label-free under brightfield microscopy.

### Wnt Depletion Sensitizes Human Duodenum Organoids to Morphological Changes Similar to That Observed in Apc^{min/+} Organoids

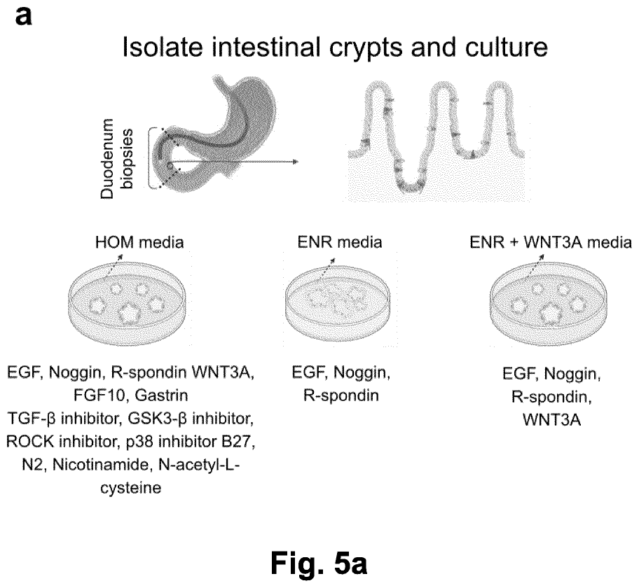

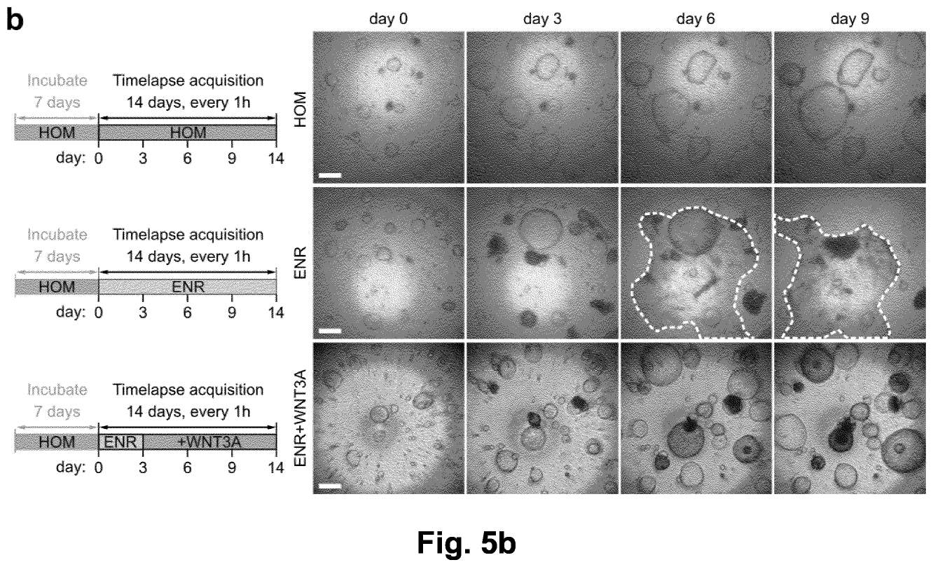

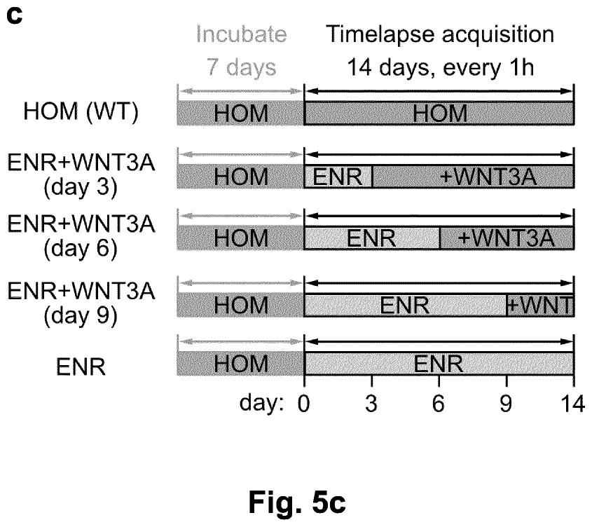

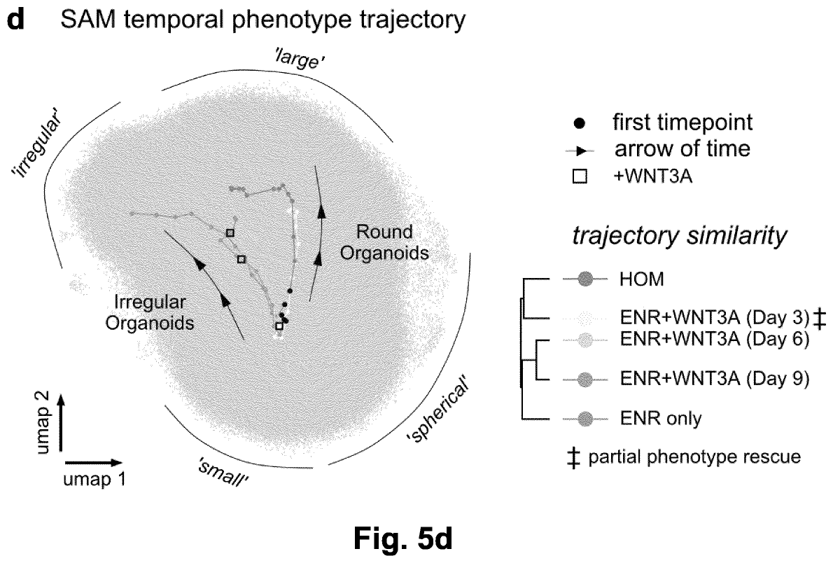

APC deletion leads to elevated WNT signaling, and this regulation is evolutionarily conserved from drosophila to humans. This presents a conundrum. If WNT signaling is elevated in Apc^{min/+} mouse organoids, then more spherical morphologies are expected. Instead, more irregular, flattened organoid morphology was observed. If the observed genotype-phenotype connection is evolutionarily conserved, an alteration in WNT signaling should have a similar impact on the spatiotemporal dynamics of both mouse and human organoids. This hypothesis was tested using human organoids derived from endoscopic biopsies of non-cancerous D2 duodenum from three individuals. To support the expansion and maintenance of Lgr5+CBC stem cells, these organoids were maintained in conventional HOM, containing>12 growth factors, including WNT and inhibitors and activators of various signaling pathways (FIG. 5a). All organoids cultured in HOM increased in size and retained a spherical morphology (FIG. 5b, top panel). Notably, when organoids were transferred from HOM to an ingredient-reduced culture medium containing only EGF, Noggin and R-spondin (ENR), a dramatic morphological change was observed. Organoids grown in ENR medium developed eccentric, flattened morphologies and often ‘merged’ with neighboring organoids (FIG. 5b, middle panel). The ‘flattened’ sheet-like organoid morphology was observed consistently in organoids derived from all 3 individuals and arose 3-5 days following their transfer to ENR medium. This phenomenon was cell density-dependent and required a sufficiently high spatial organoid density.

The observed flattening phenotype is visually reminiscent of Apc^{min/+} murine colon organoids. However, the role of WNT on the morphodynamic changes of human organoids was unexpected. Whereas Apc^{min/+} organoids should have high WNT signaling, ENR medium lacks WNT. To test the direct impact of WNT on the morphological changes of human duodenum organoids, WNT3A, a potent inducer of WNT signaling, was added to the ENR medium at day 3, 6, and 9 after the initial transfer of human duodenum organoids to ENR. Remarkably, restoring WNT3A to ENR medium at day 3 largely prevented the formation of morphologically flattened irregular organoids (FIG. 5b, bottom panel). To quantify the impact of WNT3A restoration, all organoids were grown in 96-well plates for 7 days, and live-cell images were captured every hour for the next 14 days (FIG. 5c). The spatiotemporal dynamics of a library of >500,000 segmented organoid instances over all timepoints and videos were analyzed by SPOT in the same manner as mouse colon organoids, and the organoids were subsequently classified into one of 7 phenotype clusters. Quantifying the expression of representative SAM features, as expected, organoids cultured in HOM were large and spherical, shown by enriched distribution of yellow cluster 6. Conversely, ENR organoids exhibited suppressed growth and were substantially enriched in the irregular-shaped orange cluster 7.

In line with qualitative observations, restoring WNT3A on day 3 substantially reduced the percentage of irregularly shaped organoids (cluster 7) and increased their growth (cluster 6) as observed in HOM. The timing of WNT3A restoration post transfer to ENR medium had a significant impact on phenotype rescue, with day 3 having the biggest impact, day 6 having little impact, and day 9 having minimal impact on reducing the relative proportion of irregularly shaped organoids to spherical organoids, as visualized by the local point density heatmap. This is further supported by stacked barplots of the phenotype cluster distribution over time. Restoring WNT3A on day 3 uniquely led to enhanced proliferation (cluster 6 expansion) and suppressed the expansion of irregularly shaped organoids (cluster 7), from day 4 onwards. On days 1-3, prior to restoration, there was little evidence of growth, and irregular organoids were seen to expand, in contrast to the rapid growth in HOM conditions. Notably, in all other ENR conditions, no expansion of cluster 6 was observed, and no regression of cluster 7. The HMM cluster transition analysis reflects the gradual departure from wild type HOM as the transitions become ‘rewired’. Notably, the transition graph of restoring WNT3A on day 6 (ENR+WNT3A (Day 6)) best reflects the ENR-only condition. Restoring WNT3A on day 9 introduces two new transitions, cluster 5-to-3 and cluster 4-to-2. These transitions are associated with a large-to-small organoid area change, which may reflect additional cell death. The ‘rescue’ potential is also succinctly captured in the temporal phenotype trajectories and its clustering, which show a close overlap between day 3 WNT3A restoration and HOM conditions (FIG. 5d, right two trajectories), this overlap being distinct from the overlap of the other three conditions: WNT3A restoration on day 6 and 9 and ENR only (FIG. 5d, left three trajectories). All these demonstrate that reduced WNT signaling predisposes human intestine organoids to morphodynamic changes.

scRNA-Seq Highlights the Potential of SPOT to Couple Morphodynamics to Molecular Signaling

To investigate the question why irregularly shaped, flattened organoids are enriched in two seemingly opposing conditions: high WNT-containing mouse APC^{min/+} organoids and WNT-deprived human duodenum organoids, single cell RNA sequencing (scRNA-seq) was performed. scRNA-seq was first performed on mouse organoids carrying Apc, Kras or Tp53 mutations and grown under the same culture conditions as those used for SPOT analysis. In total, 4737 single cell transcriptomes were generated. In agreement with SPOT analysis, cells derived from APC^{min/+} organoids show a unique clustering following dimensionality reduction, separated from all other indicated genotypes. PAGA analysis (Wolf, F. A. et al. Genome Biol 20, 59, (2019)) of all single cell transcriptomes (n=4737 cells) identified 12 interconnected clusters.

Gene expression profiles define cell types with known biological functions and differentiation lineages. Cells expressing high WNT targets and adult stem cell signatures are considered cryptbase columnar cells (CBCs), whereas cells expressing high YAP targets, fetal, and RSC signatures are considered regenerative stem cells (RSCs). As expected, high proportions of cells expressing gene signatures of CBC stem cells also strongly express WNT targets, whereas fetal gene markers expressing RSCs strongly express YAP targets.

Under steady-state intestinal homeostasis, WNT-dependent Lgr5+/Olfm4/Axin2 CBC stem cells dominate, whereas RSC populations are key for epithelial repair under stress and injury. RSCs are Lgr5-, YAP-dependent, and express Ly6a, Anxa1, and Clu. As expected, high WNT target-expressing cells are enriched in cell clusters derived from APC^{min/+} and to some extent in p53^{R172H:R17211} organoids. Notably, however, the expression levels of WNT targets differ among APC^{min/+} PAGA subclusters (6, 7, and 8), with cluster 6 and 8 expressing the lowest and highest WNT target, respectively. In contrast to WNT targets, the highest and lowest YAP target expression cells were found in cluster 6 and 8, respectively. Detailed analysis of APC^{min/+} clusters shows that WNT and YAP targets act antagonistically, with the RSC signature correlating strongly to YAP target expression, and weak anti-correlation to WNT targets.

To understand the molecular basis of WNT depletion-induced morphodynamic changes, scRNA-seq was also performed on the human duodenum organoids cultured under conditions and timescales identical to those used for the SPOT analysis. In total, 8763 single cell transcriptomes were generated. UMAP analysis of transcriptional similarity showed two large global groups, corresponding to organoid-derived cells grown in HOM for 7 days before exposure to ENR, or cultured in ENR for 3-9 days. Examining the expression of WNT, YAP targets, fetal and RSC signatures in organoid-derived cells as a function of the time of WNT3A rescue, it is evident that there is an inverse association between high WNT and high YAP target-expressing cells. Also, the kinetics of RSC signature expression levels coincide with the extent of phenotypic changes detected by SPOT. Thus, WNT-deprived ENR medium induces RSCs that lead to the development of morphologically irregular and flattened organoids. This effect can be prevented by restoring WNT3A, and is detected in phenotypic morphodynamic changes by SPOT.

Altogether, these results indicate that the observed ‘flattened’ irregularly shaped organoids in both human duodenum and mouse Apc^{min/+} organoids are regulated by the same low WNT and high YAP signaling pathways. This is consistent with YAP as a mechanical sensor in response to environmental perturbation and a key regulator of cell fate. The fact that similar SAM features detected by SPOT are underlined by the same signaling pathways under two seemingly contradicting culture conditions highlights the potential of SPOT as a powerful tool to detect the coupling between SAM phenotype dynamics and molecular signaling.

### DISCUSSION

Inspired by advances in single-cell sequencing, in particular single-cell transcriptome studies, this disclosure addresses the key technological challenge of how to unleash the power of live-cell imaging for quantifying and tracking phenotypic heterogeneity to advance biomedical discovery. By systematically constructing a standardized set of Shape, Appearance, and Motion (SAM) features as an image-based “phenome” akin to a scRNAseq transcriptome, the SAM Phenotype Observation Tool (SPOT) reported here acts as a “sequencer” that measures>2000 SAM features (transcripts) which together contains sufficient information to construct an image-based “transcriptome”: a SAM “phenome.” The input to SPOT is a collection of objects that have been provided by the user or, alternatively, identified by using SPOT to track and segment all individual objects over time in each video. SPOT measures and generates standardized output metrics to detect, map, and monitor known and unknown phenotypic heterogeneity. SPOT was designed to be a robust and generalized tool that operates on data in a fully automated manner, requiring no user supervision or prior knowledge. It can, therefore, ideally fill the missing gap of an unbiased, frontline data-mining and exploration tool.

Prior to this study, most previous reports used features that are readily available under regionprops function in Matlab or Python scikit-image packages, which primarily measure global properties, including various area or eccentricity properties that are far from comprehensive. Features detected by programs like CellProfiler and PerkinElmer Harmony are geared towards shape and appearance only, with a bias towards high-throughput fluorescent fixed imaging screens. With the advent of deep learning, most studies now seek to ‘learn’ the features necessary to describe phenotype through self-supervision or classification tasks. However, as the representativeness of the input data, task formulation, and architectural design will always limit the learning, any features learned may unintentionally reflect the data biases and assumptions used for training. Lastly, none of these studies demonstrate that a single set of imaging features, equipped with a standardized workflow, can be applied in a broad setting to study diverse datasets from multiple fields, such as computer vision and biology. All these argue for the development of a general, translatable, universal feature encoder, which was developed here in the form of SPOT and the SAM phenome.

The SAM phenome was designed to contain universal features that are not subject to dataset biases. This SAM phenome captures a conceptual matrix of shape, appearance, motion vs. global, regional and distributional scope, enabling a comprehensive description of imaging phenotypes. Through extensive analysis of datasets generated on general computer vision, including>350 YouTube videos, single-cell tracking, and videos of >1.5 million organoids, the universality of the SAM phenome was demonstrated. By exploiting this universality in combination with dimensionality reduction techniques, in all cases, a comprehensive space-time phenomic landscape can be generated to globally position phenotypic heterogeneity. The very simple techniques of temporal phenotype cluster stacked barplots, and SAM phenotype trajectories can be developed to extract powerful insights from these extensive video collections. Individual SAM features can also be automatically grouped into SAM modules for data-informed interpretation. The complete SPOT workflow effectively democratizes the complexities of video analysis to the wider community, enabling dynamic imaging phenotypes to be analyzed as readily as scRNA-seq.

SPOT operates on arbitrarily labeled fluorescent and label-free live-cell imaging, ensuring maximum applicability for cost-effective unbiased phenotyping without the need for specialized microscopes. SAM phenomes are efficiently computed with standard software libraries without code optimization. SPOT can extract>500,000 organoid instances and image features from >9000 organoids (100 organoids per well of a 96-well plate) in less than 12 h on a single PC (Intel i7-5820K CPU, 64 GB RAM, NVIDIA GTX TITAN Black GPU). SPOT is modular and can be readily customized and extended. For example, the SPOT SAM phenome can be augmented with additional features, both SAM-based and from different modalities, e.g., genetics-based. Lastly, the SAM phenome output of SPOT analysis is exportable in tabular form and can be analyzed by external software, such as CellProfiler and Perkin Elmer Harmony, and included in custom analytical workflows.

SPOT was able to assess phenotype-genotype coupling effectively and efficiently. Among all organoids examined (>1.5 million human and mouse organoids), only a small percentage of organoids underwent morphological changes to irregular and flattened organoids. Such phenomena occurred in APC^{min/+} mouse organoid cultures, and only at a low frequency. The relative fraction of these irregular organoids increases over time and with organoid density, but never becomes the majority population. Without SPOT's ability to unbiasedly and quantitatively measure universal image features comprehensively over time, such time-dependent multidimensional morphodynamic changes are likely to be missed or ignored using static snapshots or statistical testing based on single feature timeseries. The scRNA-seq results indicate that many high YAP target-expressing cells are likely destined to become RSCs, and RSC cells are mainly detected in genotypes and conditions that produced flattened organoids (APC^{min/+} mouse colon organoids or human duodenum organoids cultured in WNT-deprived ENR for 9 days). In contrast, few RSCs nor flattened organoids were detected in those conditions where proliferation was promoted, such as culturing in a HOM medium.

Detecting the presence of RSCs in APC^{min/+} mouse intestine organoids was unexpected. APC deletion stabilizes nuclear β-catenin, leading to elevated WNT signaling and an enhanced number of CBC stem cells. This led us to ask the question: what could cause a small percentage of APC^{min/+} cells to downregulate WNT signaling and upregulate YAP and RSC signatures to expand RSCs? Standard organoid formation assays can only measure the growth potential and growth rate of expanding organoids, whereas SPOT can measure the temporal changes in imaging features resulting from alterations in dynamic signaling pathway interactions that cause functional consequences. For example, SPOT revealed that some APC^{min/+} mouse intestinal organoids develop very irregular shapes, low proliferation, and high motility compared to organoids of WT, mutant KRAS or mutant TP53 genotypes. A possible explanation for this would be that high motility speed and morphological plasticity enable APC^{min/+} organoids to reach the edge of the Matrigel or well bottom more rapidly, where they then experience differential mechanical forces. YAP is known to translocate from the cytoplasm to the nucleus, where it forms a complex with TEAD, a transcription factor, turning on YAP targets and activating YAP signaling. High YAP target expression is a signature of RSC-expressing cells. The observed strong association between flattened organoid phenotype and the presence of RSC cell cluster thus highlights SPOT's potential as a proxy read out for phenotype-genotype and phenotype-signaling coupling.

Together, this example shows that SPOT is a highly robust and versatile high throughput and high-content image tool that will enhance the ability to use patient-derived primary cultures, including organoids, for hypothesis generation with little to no assumptions, operating under label-free conditions. Inspired by the ability of single-cell sequencing to detect many previously unknown cell clusters from a highly heterogeneous cell population, SPOT will unleash the power of live-cell imaging-based studies to advance biomedical research.

The present disclosure is not to be limited in scope by the specific embodiments described herein. Indeed, various modifications of the invention, in addition to those described herein, will become apparent to those skilled in the art from the foregoing description and the accompanying figures. Such modifications are intended to fall within the scope of the appended claims.

TABLE 1
SAM - S (Shape phenome (raw))

| | | | | # of | | Mea- | | | |
|---|---|---|---|---|---|---|---|---|---|
| | | | | Features | | surement | Metric | | |
| Feature/Descriptor | Feature Names | Category | Feature set | (dimension) | Scope | Type | Unit | Description | References |
| Maximum curvature | max_curvature | curvature_statistics | Geometrical | 1 | Global | scalar | 1/μm | Maximum line curvature of the object contour | GitHub |
| Minimum curvature | min_curvature | curvature_statistics | Geometrical | 1 | Global | scalar | 1/μm | Minimum line curvature of the object contour | GitHub |
| Mean curvature | mean_curvature | curvature_statistics | Gcomctrical | 1 | Global | scalar | 1/μm | Mean line curvature of the object contour | GitHub |
| Mean curvature magnitude | mean_abs_curvature | curvature_statistics | Geometrical | 1 | Global | scalar | 1/μm | Mean absolute line curvature of the object | GitHub |
| | | | | | | | | contour | |
| Standard deviation | std_curvature | curvature_statistics | Geometrical | 1 | Global | scalar | 1/μm | Standard deviation of the line curvature of | GitHub |
| curvature | | | | | | | | the object contour | |
| Skew curvature | mean_skew_curvature | curvature_statistics | Geometrical | 1 | Global | scalar | | Skewness of the line curvature of the organoid | GitHub |
| | | | | | | | | contour | |
| Kurtosis curvature | mean_kurtosis_{—} | curvature_statistics | Geometrical | 1 | Global | scalar | | Kurtosis of the line curvature of the organoid | GitHub |
| | curvature | | | | | | | contour | |
| Maximum centroid distance | max_centroid_distance | centroid distance | Centroid statistics | 1 | Global | scalar | μm | Maximum of the distance between organoid contour | GitHub |
| | | statistics | | | | | | points to organoid centroid | |
| Mean centroid distance | mean_centroid_distance | centroid distance | Centroid statistics | 1 | Global | scalar | μm | Mean of the distance between organoid contour | GitHub |
| | | statistics | | | | | | points to organoid centroid | |
| Standard deviation mean | std_mean_centroid_{—} | centroid distance | Centroid statistics | 1 | Global | scalar | μm | Standard deviation of the distance between | GitHub |
| centroid distance ratio | distance_ratio | statistics | | | | | | organoid contour points to organoid centroid | |
| Maximum chordal distance | max_chordal_distance | centroid distance | Centroid statistics | 1 | Global | scalar | μm | Maximum euclidean distance between two organoid | GitHub |
| | | statistics | | | | | | contour points | |
| Maximum of minimum | max_min_centroid_{—} | centroid distance | Centroid statistics | 1 | Global | scalar | μm | Maximum centroid distance of organoid contour | GitHub |
| centroid distance ratio | distance_ratio | statistics | | | | | | point/minimum centroid distance of organoid | |
| | | | | | | | | contour point | |
| Chordal distance histogram | chordal_hist_bin_1 | chordal distance | Chordal distance | 8 | Distri- | histogram | | Histogram of all pairwise Euclidean distance | GitHub |
| | | histogram | histogram | | bution | | | between organoid contour points | |
| | chordal_hist_bin_2 | chordal distance | Chordal distance | | | | | | GitHub |
| | | histogram | histogram | | | | | | |
| | chordal_hist_bin_3 | chordal distance | Chordal distance | | | | | | GitHub |
| | | histogram | histogram | | | | | | |
| | chordal_hist_bin_4 | chordal distance | Chordal distance | | | | | | GitHub |
| | | histogram | histogram | | | | | | |
| | chordal_hist_bin_5 | chordal distance | Chordal distance | | | | | | GitHub |
| | | histogram | histogram | | | | | | |
| | chordal_hist_bin_6 | chordal distance | Chordal distance | | | | | | GitHub |
| | | histogram | histogram | | | | | | |
| | chordal_hist_bin_7 | chordal distance | Chordal distance | | | | | | GitHub |
| | | histogram | histogram | | | | | | |
| | chordal_hist_bin_8 | chordal distance | Chordal distance | | | | | | GitHub |
| | | histogram | histogram | | | | | | |
| Area | area | area and perimeter | Geometrical | 1 | Global | scalar | μm\hat{ }2 | Number of pixels within the organoid contour | skimage.mea- |
| | | statistics | | | | | | scaled by per pixel-arca | sure.regionprops |
| Convex hull area | convex_area | area and perimeter | Geometrical | 1 | Global | scalar | μm\hat{ }2 | Number of pixels of organoid contour bounding | skimage.mea- |
| | | statistics | | | | | | box scaled by per pixel-arca | sure.regionprops |
| Perimeter | perimeter | area and perimeter | Geometrical | 1 | Global | scalar | μm | Total Euclidean distance length of the organoid | skimage.mea- |
| | | statistics | | | | | | contour | sure.regionprops |
| Equivalent circular | equivalent_diameter | area and perimeter | Geometrical | 1 | Global | scalar | μm | The diameter of circle with the same area as | skimage.mea- |
| diameter | | statistics | | | | | | the number of pixels in the organoid contour | sure.regionprops |
| Major axis length | major_axis_length | shape factor | Geometrical | 1 | Global | scalar | μm | The length of the major axis of the ellipse | skimage.mea- |
| | | statistics | | | | | | with the same normalized second central | sure.regionprops |
| | | | | | | | | moment as the organoid region | |
| Minor axis length | minor_axis_length | shape factor | Geometrical | 1 | Global | scalar | μm | The length of the minor axis of the ellipse | skimage.mea- |
| | | statistics | | | | | | with the same normalized second central | sure.regionprops |
| | | | | | | | | moment as the organoid region | |
| Area perimeter | area_perim_{—} | shape factor | Geometrical | 1 | Global | scalar | | 4π × area/(perimeter\hat{ }2) | skimage.mea- |
| aspect ratio | aspect_ratio | statistics | | | | | | | sure.regionprops |
| Major over minor | major_minor_{—} | shape factor | Geometrical | 1 | Global | scalar | | Major axis length/minor axis length | skimage.mea- |
| axis length | axis_ratio | statistics | | | | | | | sure.regionprops |
| ratio (eccentricity) | | | | | | | | | |
| Moment of | moment_eccentricity | shape factor | Geometrical | 1 | Global | scalar | | Distance between focal points of a fitted | skimage.mea- |
| eccentricity | | statistics | | | | | | ellipse/major axis length. Value in the interval | sure.regionprops |
| | | | | | | | | [0, 1). When 0, the ellipse is a circle | |
| Solidity | solidity | shape factor | Geometrical | 1 | Global | scalar | | Number of pixels in the organoid contour/number | skimage.mea- |
| | | statistics | | | | | | of pixels in the convex hull of the organoid | sure.regionprops |
| | | | | | | | | contour | |
| Extent | extent | shape factor | Geometrical | 1 | Global | scalar | | Number of pixels in the organoid contour/number | skimage.mea- |
| | | statistics | | | | | | of pixels in the bounding box of the organoid | sure.regionprops |
| | | | | | | | | contour | |
| Hu moments | hu_moments_1 | Hu_moments | Hu moment | 7 | Regional | vector | | Weighted average of image pixel intensities | Hu, M. K., |
| | hu_moments_2 | Hu_moments | Hu moment | | | | | invariant to image transformations; translation, | 1962. Visual |
| | hu_moments_3 | Hu_moments | Hu moment | | | | | scale, rotation and reflection | pattern |
| | hu_moments_4 | Hu_moments | Hu moment | | | | | | recognition |
| | hu_moments_5 | Hu_moments | Hu moment | | | | | | by moment |
| | hu_moments_6 | Hu_moments | Hu moment | | | | | | invariants. |
| | hu_moments_7 | Hu_moments | Hu moment | | | | | | IRE |
| Zernike moments | zernike_moments_1 | Zernike_moments | Zernike moment | 25 | Regional | vector | | Fit coefficients when region is fitted by | Teague, M R. |
| | zernike_moments_2 | Zernike_moments | Zernike moment | | | | | Zernike polynomials, a family of orthogonal | (1980). |
| | zernike_moments_3 | Zernike_moments | Zernike moment | | | | | radial polynomials defined on a disk | Image |
| | zernike_moments_4 | Zernike_moments | Zernike moment | | | | | | Analysis via |
| | zernike_moments_5 | Zernike_moments | Zernike moment | | | | | | the General |
| | zernike_moments_6 | Zernike_moments | Zernike moment | | | | | | Theory of |
| | zernike_moments_7 | Zernike_moments | Zernike moment | | | | | | Moments. J. |
| | zernike_moments_8 | Zernike_moments | Zernike moment | | | | | | Opt. Soc. |
| | zernike_moments_9 | Zernike_moments | Zernike moment | | | | | | Am. |
| | zernike_moments_10 | Zernike_moments | Zernike moment | | | | | | 70(8): 920- |
| | zernike_moments_11 | Zernike_moments | Zernike moment | | | | | | 930. |
| | zernike_moments_12 | Zernike_moments | Zernike moment | | | | | | |
| | zernike_moments_13 | Zernike_moments | Zernike moment | | | | | | |
| | zernike_moments_14 | Zernike_moments | Zernike moment | | | | | | |
| | zernike_moments_15 | Zernike_moments | Zernike moment | | | | | | |
| | zernike_moments_16 | Zernike_moments | Zernike moment | | | | | | |
| | zernike_moments_17 | Zernike_moments | Zernike moment | | | | | | |
| | zernike_moments_18 | Zernike_moments | Zernike moment | | | | | | |
| | zernike_moments_19 | Zernike_moments | Zernike moment | | | | | | |
| | zernike_moments_20 | Zernike_moments | Zernike moment | | | | | | |
| | zernike_moments_21 | Zernike_moments | Zernike moment | | | | | | |
| | zernike_moments_22 | Zernike_moments | Zernike moment | | | | | | |
| | zernike_moments_23 | Zernike_moments | Zernike moment | | | | | | |
| | zernike_moments_24 | Zernike_moments | Zernike moment | | | | | | |
| | zernike_moments_25 | Zernike_moments | Zernike moment | | | | | | |
| Fourier features | Fourier_1 | Fourier Features | Fourier Features | 99 | Regional | vector | | Absolute magnitude of the Fourier transform | Zhang, D. |
| | Fourier_2 | Fourier Features | Fourier Features | | | | | of the object boundary contour specified as | and Lu, G., |
| | Fourier_3 | Fourier Features | Fourier Features | | | | | the complex geometric vector displacement, | 2005. Study |
| | Fourier_4 | Fourier Features | Fourier Features | | | | | Δx + jΔy relative to the object centroid | and |
| | Fourier_5 | Fourier Features | Fourier Features | | | | | | evaluation of |
| | Fourier_6 | Fourier Features | Fourier Features | | | | | | different |
| | Fourier_7 | Fourier Features | Fourier Features | | | | | | Fourier |
| | Fourier_8 | Fourier Features | Fourier Features | | | | | | methods for |
| | Fourier_9 | Fourier Features | Fourier Features | | | | | | image |
| | Fourier_10 | Fourier Features | Fourier Features | | | | | | retrieval. |
| | Fourier_11 | Fourier Features | Fourier Features | | | | | | Image and |
| | Fourier_12 | Fourier Features | Fourier Features | | | | | | vision |
| | Fourier_13 | Fourier Features | Fourier Features | | | | | | computing, |
| | Fourier_14 | Fourier Features | Fourier Features | | | | | | 23(1), pp. 33-49. |
| | Fourier_15 | Fourier Features | Fourier Features | | | | | | |
| | Fourier_16 | Fourier Features | Fourier Features | | | | | | |
| | Fourier_17 | Fourier Features | Fourier Features | | | | | | |
| | Fourier_18 | Fourier Features | Fourier Features | | | | | | |
| | Fourier_19 | Fourier Features | Fourier Features | | | | | | |
| | Fourier_20 | Fourier Features | Fourier Features | | | | | | |
| | Fourier_21 | Fourier Features | Fourier Features | | | | | | |
| | Fourier_22 | Fourier Features | Fourier Features | | | | | | |
| | Fourier_23 | Fourier Features | Fourier Features | | | | | | |
| | Fourier_24 | Fourier Features | Fourier Features | | | | | | |
| | Fourier_25 | Fourier Features | Fourier Features | | | | | | |
| | Fourier_26 | Fourier Features | Fourier Features | | | | | | |
| | Fourier_27 | Fourier Features | Fourier Features | | | | | | |
| | Fourier_28 | Fourier Features | Fourier Features | | | | | | |
| | Fourier_29 | Fourier Features | Fourier Features | | | | | | |
| | Fourier_30 | Fourier Features | Fourier Features | | | | | | |
| | Fourier_31 | Fourier Features | Fourier Features | | | | | | |
| | Fourier_32 | Fourier Features | Fourier Features | | | | | | |
| | Fourier_33 | Fourier Features | Fourier Features | | | | | | |
| | Fourier_34 | Fourier Features | Fourier Features | | | | | | |
| | Fourier_35 | Fourier Features | Fourier Features | | | | | | |
| | Fourier_36 | Fourier Features | Fourier Features | | | | | | |
| | Fourier_37 | Fourier Features | Fourier Features | | | | | | |
| | Fourier_38 | Fourier Features | Fourier Features | | | | | | |
| | Fourier_39 | Fourier Features | Fourier Features | | | | | | |
| | Fourier_40 | Fourier Features | Fourier Features | | | | | | |
| | Fourier_41 | Fourier Features | Fourier Features | | | | | | |
| | Fourier_42 | Fourier Features | Fourier Features | | | | | | |
| | Fourier_43 | Fourier Features | Fourier Features | | | | | | |
| | Fourier_44 | Fourier Features | Fourier Features | | | | | | |
| | Fourier_45 | Fourier Features | Fourier Features | | | | | | |
| | Fourier_46 | Fourier Features | Fourier Features | | | | | | |
| | Fourier_47 | Fourier Features | Fourier Features | | | | | | |
| | Fourier_48 | Fourier Features | Fourier Features | | | | | | |
| | Fourier_49 | Fourier Features | Fourier Features | | | | | | |
| | Fourier_50 | Fourier Features | Fourier Features | | | | | | |
| | Fourier_51 | Fourier Features | Fourier Features | | | | | | |
| | Fourier_52 | Fourier Features | Fourier Features | | | | | | |
| | Fourier_53 | Fourier Features | Fourier Features | | | | | | |
| | Fourier_54 | Fourier Features | Fourier Features | | | | | | |
| | Fourier_55 | Fourier Features | Fourier Features | | | | | | |
| | Fourier_56 | Fourier Features | Fourier Features | | | | | | |
| | Fourier_57 | Fourier Features | Fourier Features | | | | | | |
| | Fourier_58 | Fourier Features | Fourier Features | | | | | | |
| | Fourier_59 | Fourier Features | Fourier Features | | | | | | |
| | Fourier_60 | Fourier Features | Fourier Features | | | | | | |
| | Fourier_61 | Fourier Features | Fourier Features | | | | | | |
| | Fourier_62 | Fourier Features | Fourier Features | | | | | | |
| | Fourier_63 | Fourier Features | Fourier Features | | | | | | |
| | Fourier_64 | Fourier Features | Fourier Features | | | | | | |
| | Fourier_65 | Fourier Features | Fourier Features | | | | | | |
| | Fourier_66 | Fourier Features | Fourier Features | | | | | | |
| | Fourier_67 | Fourier Features | Fourier Features | | | | | | |
| | Fourier_68 | Fourier Features | Fourier Features | | | | | | |
| | Fourier_69 | Fourier Features | Fourier Features | | | | | | |
| | Fourier_70 | Fourier Features | Fourier Features | | | | | | |
| | Fourier_71 | Fourier Features | Fourier Features | | | | | | |
| | Fourier_72 | Fourier Features | Fourier Features | | | | | | |
| | Fourier_73 | Fourier Features | Fourier Features | | | | | | |
| | Fourier_74 | Fourier Features | Fourier Features | | | | | | |
| | Fourier_75 | Fourier Features | Fourier Features | | | | | | |
| | Fourier_76 | Fourier Features | Fourier Features | | | | | | |
| | Fourier_77 | Fourier Features | Fourier Features | | | | | | |
| | Fourier_78 | Fourier Features | Fourier Features | | | | | | |
| | Fourier_79 | Fourier Features | Fourier Features | | | | | | |
| | Fourier_80 | Fourier Features | Fourier Features | | | | | | |
| | Fourier_81 | Fourier Features | Fourier Features | | | | | | |
| | Fourier_82 | Fourier Features | Fourier Features | | | | | | |
| | Fourier_83 | Fourier Features | Fourier Features | | | | | | |
| | Fourier_84 | Fourier Features | Fourier Features | | | | | | |
| | Fourier_85 | Fourier Features | Fourier Features | | | | | | |
| | Fourier_86 | Fourier Features | Fourier Features | | | | | | |
| | Fourier_87 | Fourier Features | Fourier Features | | | | | | |
| | Fourier_88 | Fourier Features | Fourier Features | | | | | | |
| | Fourier_89 | Fourier Features | Fourier Features | | | | | | |
| | Fourier_90 | Fourier Features | Fourier Features | | | | | | |
| | Fourier_91 | Fourier Features | Fourier Features | | | | | | |
| | Fourier_92 | Fourier Features | Fourier Features | | | | | | |
| | Fourier_93 | Fourier Features | Fourier Features | | | | | | |
| | Fourier_94 | Fourier Features | Fourier Features | | | | | | |
| | Fourier_95 | Fourier Features | Fourier Features | | | | | | |
| | Fourier_96 | Fourier Features | Fourier Features | | | | | | |
| | Fourier_97 | Fourier Features | Fourier Features | | | | | | |
| | Fourier_98 | Fourier Features | Fourier Features | | | | | | |
| | Fourier_99 | Fourier Features | Fourier Features | | | | | | |
| Euler | ECT_1 | Euler characteristic | Euler characteristic | 1152 | Distri- | Count- | | A descriptor of the local topology, | Amézquita, |
| characteristic | | curve (ECC) | curve (ECC) | | bution | based | | created by computing the Euler | E. J., Quigley |
| curves (ECC) | ECT_2 | Euler characteristic | Euler characteristic | | | | | characteristic, a topological invatiant | M. Y., |
| | | curve (ECC) | curve (ECC) | | | | | | Ophelders, |
| | ECT_3 | Euler characteristic | Euler characteristic | | | | | | T., Landis |
| | | curve (ECC) | curve (ECC) | | | | | | J. B., Koenig, |
| | ECT_4 | Euler characteristic | Euler characteristic | | | | | | D., Munch, |
| | | curve (ECC) | curve (ECC) | | | | | | E. and |
| | ECT_5 | Euler characteristic | Euler characteristic | | | | | | Chitwood, |
| | | curve (ECC) | curve (ECC) | | | | | | D. H., 2022. |
| | ECT_6 | Euler characteristic | Euler characteristic | | | | | | Measuring |
| | | curve (ECC) | curve (ECC) | | | | | | hidden |
| | ECT_7 | Euler characteristic | Euler characteristic | | | | | | phenotype: |
| | | curve (ECC) | curve (ECC) | | | | | | Quantifying |
| | ECT_8 | Euler characteristic | Euler characteristic | | | | | | the shape of |
| | | curve (ECC) | curve (ECC) | | | | | | barley seeds |
| | ECT_9 | Euler characteristic | Euler characteristic | | | | | | using the |
| | | curve (ECC) | curve (ECC) | | | | | | Euler |
| | ECT_10 | Euler characteristic | Euler characteristic | | | | | | Characteristic |
| | | curve (ECC) | curve (ECC) | | | | | | Transform. |
| | ECT_11 | Euler characteristic | Euler characteristic | | | | | | in silico |
| | | curve (ECC) | curve (ECC) | | | | | | Plants, 4(1) |
| | ECT_12 | Euler characteristic | Euler characteristic | | | | | | |
| | | curve (ECC) | curve (ECC) | | | | | | |
| | ECT_13 | Euler characteristic | Euler characteristic | | | | | | |
| | | curve (ECC) | curve (ECC) | | | | | | |
| | ECT_14 | Euler characteristic | Euler characteristic | | | | | | |
| | | curve (ECC) | curve (ECC) | | | | | | |
| | ECT_15 | Euler characteristic | Euler characteristic | | | | | | |
| | | curve (ECC) | curve (ECC) | | | | | | |
| | ECT_16 | Euler characteristic | Euler characteristic | | | | | | |
| | | curve (ECC) | curve (ECC) | | | | | | |
| | ECT_17 | Euler characteristic | Euler characteristic | | | | | | |
| | | curve (ECC) | curve (ECC) | | | | | | |
| | ECT_18 | Euler characteristic | Euler characteristic | | | | | | |
| | | curve (ECC) | curve (ECC) | | | | | | |
| | ECT_19 | Euler characteristic | Euler characteristic | | | | | | |
| | | curve (ECC) | curve (ECC) | | | | | | |
| | ECT_20 | Euler characteristic | Euler characteristic | | | | | | |
| | | curve (ECC) | curve (ECC) | | | | | | |
| | ECT_21 | Euler characteristic | Euler characteristic | | | | | | |
| | | curve (ECC) | curve (ECC) | | | | | | |
| | ECT_22 | Euler characteristic | Euler characteristic | | | | | | |
| | | curve (ECC) | curve (ECC) | | | | | | |
| | ECT_23 | Euler characteristic | Euler characteristic | | | | | | |
| | | curve (ECC) | curve (ECC) | | | | | | |
| | ECT_24 | Euler characteristic | Euler characteristic | | | | | | |
| | | curve (ECC) | curve (ECC) | | | | | | |
| | ECT_25 | Euler characteristic | Euler characteristic | | | | | | |
| | | curve (ECC) | curve (ECC) | | | | | | |
| | ECT_26 | Euler characteristic | Euler characteristic | | | | | | |
| | | curve (ECC) | curve (ECC) | | | | | | |
| | ECT_27 | Euler characteristic | Euler characteristic | | | | | | |
| | | curve (ECC) | curve (ECC) | | | | | | |
| | ECT_28 | Euler characteristic | Euler characteristic | | | | | | |
| | | curve (ECC) | curve (ECC) | | | | | | |
| | ECT_29 | Euler characteristic | Euler characteristic | | | | | | |
| | | curve (ECC) | curve (ECC) | | | | | | |
| | ECT_30 | Euler characteristic | Euler characteristic | | | | | | |
| | | curve (ECC) | curve (ECC) | | | | | | |
| | ECT_31 | Euler characteristic | Euler characteristic | | | | | | |
| | | curve (ECC) | curve (ECC) | | | | | | |
| | ECT_32 | Euler characteristic | Euler characteristic | | | | | | |
| | | curve (ECC) | curve (ECC) | | | | | | |
| | ECT_33 | Euler characteristic | Euler characteristic | | | | | | |
| | | curve (ECC) | curve (ECC) | | | | | | |
| | ECT_34 | Euler characteristic | Euler characteristic | | | | | | |
| | | curve (ECC) | curve (ECC) | | | | | | |
| | ECT_35 | Euler characteristic | Euler characteristic | | | | | | |
| | | curve (ECC) | curve (ECC) | | | | | | |
| | ECT_36 | Euler characteristic | Euler characteristic | | | | | | |
| | | curve (ECC) | curve (ECC) | | | | | | |
| | ECT_37 | Euler characteristic | Euler characteristic | | | | | | |
| | | curve (ECC) | curve (ECC) | | | | | | |
| | ECT_38 | Euler characteristic | Euler characteristic | | | | | | |
| | | curve (ECC) | curve (ECC) | | | | | | |
| | ECT_39 | Euler characteristic | Euler characteristic | | | | | | |
| | | curve (ECC) | curve (ECC) | | | | | | |
| | ECT_40 | Euler characteristic | Euler characteristic | | | | | | |
| | | curve (ECC) | curve (ECC) | | | | | | |
| | ECT_41 | Euler characteristic | Euler characteristic | | | | | | |
| | | curve (ECC) | curve (ECC) | | | | | | |
| | ECT_42 | Euler characteristic | Euler characteristic | | | | | | |
| | | curve (ECC) | curve (ECC) | | | | | | |
| | ECT_43 | Euler characteristic | Euler characteristic | | | | | | |
| | | curve (ECC) | curve (ECC) | | | | | | |
| | ECT_44 | Euler characteristic | Euler characteristic | | | | | | |
| | | curve (ECC) | curve (ECC) | | | | | | |
| | ECT_45 | Euler characteristic | Euler characteristic | | | | | | |
| | | curve (ECC) | curve (ECC) | | | | | | |
| | ECT_46 | Euler characteristic | Euler characteristic | | | | | | |
| | | curve (ECC) | curve (ECC) | | | | | | |
| | ECT_47 | Euler characteristic | Euler characteristic | | | | | | |
| | | curve (ECC) | curve (ECC) | | | | | | |
| | ECT_48 | Euler characteristic | Euler characteristic | | | | | | |
| | | curve (ECC) | curve (ECC) | | | | | | |
| | ECT_49 | Euler characteristic | Euler characteristic | | | | | | |
| | | curve (ECC) | curve (ECC) | | | | | | |
| | ECT_50 | Euler characteristic | Euler characteristic | | | | | | |
| | | curve (ECC) | curve (ECC) | | | | | | |
| | ECT_51 | Euler characteristic | Euler characteristic | | | | | | |
| | | curve (ECC) | curve (ECC) | | | | | | |
| | ECT_52 | Euler characteristic | Euler characteristic | | | | | | |
| | | curve (ECC) | curve (ECC) | | | | | | |
| | ECT_53 | Euler characteristic | Euler characteristic | | | | | | |
| | | curve (ECC) | curve (ECC) | | | | | | |
| | ECT_54 | Euler characteristic | Euler characteristic | | | | | | |
| | | curve (ECC) | curve (ECC) | | | | | | |
| | ECT_55 | Euler characteristic | Euler characteristic | | | | | | |
| | | curve (ECC) | curve (ECC) | | | | | | |
| | ECT_56 | Euler characteristic | Euler characteristic | | | | | | |
| | | curve (ECC) | curve (ECC) | | | | | | |
| | ECT_57 | Euler characteristic | Euler characteristic | | | | | | |
| | | curve (ECC) | curve (ECC) | | | | | | |
| | ECT_58 | Euler characteristic | Euler characteristic | | | | | | |
| | | curve (ECC) | curve (ECC) | | | | | | |
| | ECT_59 | Euler characteristic | Euler characteristic | | | | | | |
| | | curve (ECC) | curve (ECC) | | | | | | |
| | ECT_60 | Euler characteristic | Euler characteristic | | | | | | |
| | | curve (ECC) | curve (ECC) | | | | | | |
| | ECT_61 | Euler characteristic | Euler characteristic | | | | | | |
| | | curve (ECC) | curve (ECC) | | | | | | |
| | ECT_62 | Euler characteristic | Euler characteristic | | | | | | |
| | | curve (ECC) | curve (ECC) | | | | | | |
| | ECT_63 | Euler characteristic | Euler characteristic | | | | | | |
| | | curve (ECC) | curve (ECC) | | | | | | |
| | ECT_64 | Euler characteristic | Euler characteristic | | | | | | |
| | | curve (ECC) | curve (ECC) | | | | | | |
| | ECT_65 | Euler characteristic | Euler characteristic | | | | | | |
| | | curve (ECC) | curve (ECC) | | | | | | |
| | ECT_66 | Euler characteristic | Euler characteristic | | | | | | |
| | | curve (ECC) | curve (ECC) | | | | | | |
| | ECT_67 | Euler characteristic | Euler characteristic | | | | | | |
| | | curve (ECC) | curve (ECC) | | | | | | |
| | ECT_68 | Euler characteristic | Euler characteristic | | | | | | |
| | | curve (ECC) | curve (ECC) | | | | | | |
| | ECT_69 | Euler characteristic | Euler characteristic | | | | | | |
| | | curve (ECC) | curve (ECC) | | | | | | |
| | ECT_70 | Euler characteristic | Euler characteristic | | | | | | |
| | | curve (ECC) | curve (ECC) | | | | | | |
| | ECT_71 | Euler characteristic | Euler characteristic | | | | | | |
| | | curve (ECC) | curve (ECC) | | | | | | |
| | ECT_72 | Euler characteristic | Euler characteristic | | | | | | |
| | | curve (ECC) | curve (ECC) | | | | | | |
| | ECT_73 | Euler characteristic | Euler characteristic | | | | | | |
| | | curve (ECC) | curve (ECC) | | | | | | |
| | ECT_74 | Euler characteristic | Euler characteristic | | | | | | |
| | | curve (ECC) | curve (ECC) | | | | | | |
| | ECT_75 | Euler characteristic | Euler characteristic | | | | | | |
| | | curve (ECC) | curve (ECC) | | | | | | |
| | ECT_76 | Euler characteristic | Euler characteristic | | | | | | |
| | | curve (ECC) | curve (ECC) | | | | | | |
| | ECT_77 | Euler characteristic | Euler characteristic | | | | | | |
| | | curve (ECC) | curve (ECC) | | | | | | |
| | ECT_78 | Euler characteristic | Euler characteristic | | | | | | |
| | | curve (ECC) | curve (ECC) | | | | | | |
| | ECT_79 | Euler characteristic | Euler characteristic | | | | | | |
| | | curve (ECC) | curve (ECC) | | | | | | |
| | ECT_80 | Euler characteristic | Euler characteristic | | | | | | |
| | | curve (ECC) | curve (ECC) | | | | | | |
| | ECT_81 | Euler characteristic | Euler characteristic | | | | | | |
| | | curve (ECC) | curve (ECC) | | | | | | |
| | ECT_82 | Euler characteristic | Euler characteristic | | | | | | |
| | | curve (ECC) | curve (ECC) | | | | | | |
| | ECT_83 | Euler characteristic | Euler characteristic | | | | | | |
| | | curve (ECC) | curve (ECC) | | | | | | |
| | ECT_84 | Euler characteristic | Euler characteristic | | | | | | |
| | | curve (ECC) | curve (ECC) | | | | | | |
| | ECT_85 | Euler characteristic | Euler characteristic | | | | | | |
| | | curve (ECC) | curve (ECC) | | | | | | |
| | ECT_86 | Euler characteristic | Euler characteristic | | | | | | |
| | | curve (ECC) | curve (ECC) | | | | | | |
| | ECT_87 | Euler characteristic | Euler characteristic | | | | | | |
| | | curve (ECC) | curve (ECC) | | | | | | |
| | ECT_88 | Euler characteristic | Euler characteristic | | | | | | |
| | | curve (ECC) | curve (ECC) | | | | | | |
| | ECT_89 | Euler characteristic | Euler characteristic | | | | | | |
| | | curve (ECC) | curve (ECC) | | | | | | |
| | ECT_90 | Euler characteristic | Euler characteristic | | | | | | |
| | | curve (ECC) | curve (ECC) | | | | | | |
| | ECT_91 | Euler characteristic | Euler characteristic | | | | | | |
| | | curve (ECC) | curve (ECC) | | | | | | |
| | ECT_92 | Euler characteristic | Euler characteristic | | | | | | |
| | | curve (ECC) | curve (ECC) | | | | | | |
| | ECT_93 | Euler characteristic | Euler characteristic | | | | | | |
| | | curve (ECC) | curve (ECC) | | | | | | |
| | ECT_94 | Euler characteristic | Euler characteristic | | | | | | |
| | | curve (ECC) | curve (ECC) | | | | | | |
| | ECT_95 | Euler characteristic | Euler characteristic | | | | | | |
| | | curve (ECC) | curve (ECC) | | | | | | |
| | ECT_96 | Euler characteristic | Euler characteristic | | | | | | |
| | | curve (ECC) | curve (ECC) | | | | | | |
| | ECT_97 | Euler characteristic | Euler characteristic | | | | | | |
| | | curve (ECC) | curve (ECC) | | | | | | |
| | ECT_98 | Euler characteristic | Euler characteristic | | | | | | |
| | | curve (ECC) | curve (ECC) | | | | | | |
| | ECT_99 | Euler characteristic | Euler characteristic | | | | | | |
| | | curve (ECC) | curve (ECC) | | | | | | |
| | ECT_100 | Euler characteristic | Euler characteristic | | | | | | |
| | | curve (ECC) | curve (ECC) | | | | | | |
| | ECT_101 | Euler characteristic | Euler characteristic | | | | | | |
| | | curve (ECC) | curve (ECC) | | | | | | |
| | ECT_102 | Euler characteristic | Euler characteristic | | | | | | |
| | | curve (ECC) | curve (ECC) | | | | | | |
| | ECT_103 | Euler characteristic | Euler characteristic | | | | | | |
| | | curve (ECC) | curve (ECC) | | | | | | |
| | ECT_104 | Euler characteristic | Euler characteristic | | | | | | |
| | | curve (ECC) | curve (ECC) | | | | | | |
| | ECT_105 | Euler characteristic | Euler characteristic | | | | | | |
| | | curve (ECC) | curve (ECC) | | | | | | |
| | ECT_106 | Euler characteristic | Euler characteristic | | | | | | |
| | | curve (ECC) | curve (ECC) | | | | | | |
| | ECT_107 | Euler characteristic | Euler characteristic | | | | | | |
| | | curve (ECC) | curve (ECC) | | | | | | |
| | ECT_108 | Euler characteristic | Euler characteristic | | | | | | |
| | | curve (ECC) | curve (ECC) | | | | | | |
| | ECT_109 | Euler characteristic | Euler characteristic | | | | | | |
| | | curve (ECC) | curve (ECC) | | | | | | |
| | ECT_110 | Euler characteristic | Euler characteristic | | | | | | |
| | | curve (ECC) | curve (ECC) | | | | | | |
| | ECT_111 | Euler characteristic | Euler characteristic | | | | | | |
| | | curve (ECC) | curve (ECC) | | | | | | |
| | ECT_112 | Euler characteristic | Euler characteristic | | | | | | |
| | | curve (ECC) | curve (ECC) | | | | | | |
| | ECT_113 | Euler characteristic | Euler characteristic | | | | | | |
| | | curve (ECC) | curve (ECC) | | | | | | |
| | ECT_114 | Euler characteristic | Euler characteristic | | | | | | |
| | | curve (ECC) | curve (ECC) | | | | | | |
| | ECT_115 | Euler characteristic | Euler characteristic | | | | | | |
| | | curve (ECC) | curve (ECC) | | | | | | |
| | ECT_116 | Euler characteristic | Euler characteristic | | | | | | |
| | | curve (ECC) | curve (ECC) | | | | | | |
| | ECT_117 | Euler characteristic | Euler characteristic | | | | | | |
| | | curve (ECC) | curve (ECC) | | | | | | |
| | ECT_118 | Euler characteristic | Euler characteristic | | | | | | |
| | | curve (ECC) | curve (ECC) | | | | | | |
| | ECT_119 | Euler characteristic | Euler characteristic | | | | | | |
| | | curve (ECC) | curve (ECC) | | | | | | |
| | ECT_120 | Euler characteristic | Euler characteristic | | | | | | |
| | | curve (ECC) | curve (ECC) | | | | | | |
| | ECT_121 | Euler characteristic | Euler characteristic | | | | | | |
| | | curve (ECC) | curve (ECC) | | | | | | |
| | ECT_122 | Euler characteristic | Euler characteristic | | | | | | |
| | | curve (ECC) | curve (ECC) | | | | | | |
| | ECT_123 | Euler characteristic | Euler characteristic | | | | | | |
| | | curve (ECC) | curve (ECC) | | | | | | |
| | ECT_124 | Euler characteristic | Euler characteristic | | | | | | |
| | | curve (ECC) | curve (ECC) | | | | | | |
| | ECT_125 | Euler characteristic | Euler characteristic | | | | | | |
| | | curve (ECC) | curve (ECC) | | | | | | |
| | ECT_126 | Euler characteristic | Euler characteristic | | | | | | |
| | | curve (ECC) | curve (ECC) | | | | | | |
| | ECT_127 | Euler characteristic | Euler characteristic | | | | | | |
| | | curve (ECC) | curve (ECC) | | | | | | |
| | ECT_128 | Euler characteristic | Euler characteristic | | | | | | |
| | | curve (ECC) | curve (ECC) | | | | | | |
| | ECT_129 | Euler characteristic | Euler characteristic | | | | | | |
| | | curve (ECC) | curve (ECC) | | | | | | |
| | ECT_130 | Euler characteristic | Euler characteristic | | | | | | |
| | | curve (ECC) | curve (ECC) | | | | | | |
| | ECT_131 | Euler characteristic | Euler characteristic | | | | | | |
| | | curve (ECC) | curve (ECC) | | | | | | |
| | ECT_132 | Euler characteristic | Euler characteristic | | | | | | |
| | | curve (ECC) | curve (ECC) | | | | | | |
| | ECT_133 | Euler characteristic | Euler characteristic | | | | | | |
| | | curve (ECC) | curve (ECC) | | | | | | |
| | ECT_134 | Euler characteristic | Euler characteristic | | | | | | |
| | | curve (ECC) | curve (ECC) | | | | | | |
| | ECT_135 | Euler characteristic | Euler characteristic | | | | | | |
| | | curve (ECC) | curve (ECC) | | | | | | |
| | ECT_136 | Euler characteristic | Euler characteristic | | | | | | |
| | | curve (ECC) | curve (ECC) | | | | | | |
| | ECT_137 | Euler characteristic | Euler characteristic | | | | | | |
| | | curve (ECC) | curve (ECC) | | | | | | |
| | ECT_138 | Euler characteristic | Euler characteristic | | | | | | |
| | | curve (ECC) | curve (ECC) | | | | | | |
| | ECT_139 | Euler characteristic | Euler characteristic | | | | | | |
| | | curve (ECC) | curve (ECC) | | | | | | |
| | ECT_140 | Euler characteristic | Euler characteristic | | | | | | |
| | | curve (ECC) | curve (ECC) | | | | | | |
| | ECT_141 | Euler characteristic | Euler characteristic | | | | | | |
| | | curve (ECC) | curve (ECC) | | | | | | |
| | ECT_142 | Euler characteristic | Euler characteristic | | | | | | |
| | | curve (ECC) | curve (ECC) | | | | | | |
| | ECT_143 | Euler characteristic | Euler characteristic | | | | | | |
| | | curve (ECC) | curve (ECC) | | | | | | |
| | ECT_144 | Euler characteristic | Euler characteristic | | | | | | |
| | | curve (ECC) | curve (ECC) | | | | | | |
| | ECT_145 | Euler characteristic | Euler characteristic | | | | | | |
| | | curve (ECC) | curve (ECC) | | | | | | |
| | ECT_146 | Euler characteristic | Euler characteristic | | | | | | |
| | | curve (ECC) | curve (ECC) | | | | | | |
| | ECT_147 | Euler characteristic | Euler characteristic | | | | | | |
| | | curve (ECC) | curve (ECC) | | | | | | |
| | ECT_148 | Euler characteristic | Euler characteristic | | | | | | |
| | | curve (ECC) | curve (ECC) | | | | | | |
| | ECT_149 | Euler characteristic | Euler characteristic | | | | | | |
| | | curve (ECC) | curve (ECC) | | | | | | |
| | ECT_150 | Euler characteristic | Euler characteristic | | | | | | |
| | | curve (ECC) | curve (ECC) | | | | | | |
| | ECT_151 | Euler characteristic | Euler characteristic | | | | | | |
| | | curve (ECC) | curve (ECC) | | | | | | |
| | ECT_152 | Euler characteristic | Euler characteristic | | | | | | |
| | | curve (ECC) | curve (ECC) | | | | | | |
| | ECT_153 | Euler characteristic | Euler characteristic | | | | | | |
| | | curve (ECC) | curve (ECC) | | | | | | |
| | ECT_154 | Euler characteristic | Euler characteristic | | | | | | |
| | | curve (ECC) | curve (ECC) | | | | | | |
| | ECT_155 | Euler characteristic | Euler characteristic | | | | | | |
| | | curve (ECC) | curve (ECC) | | | | | | |
| | ECT_156 | Euler characteristic | Euler characteristic | | | | | | |
| | | curve (ECC) | curve (ECC) | | | | | | |
| | ECT_157 | Euler characteristic | Euler characteristic | | | | | | |
| | | curve (ECC) | curve (ECC) | | | | | | |
| | ECT_158 | Euler characteristic | Euler characteristic | | | | | | |
| | | curve (ECC) | curve (ECC) | | | | | | |
| | ECT_159 | Euler characteristic | Euler characteristic | | | | | | |
| | | curve (ECC) | curve (ECC) | | | | | | |
| | ECT_160 | Euler characteristic | Euler characteristic | | | | | | |
| | | curve (ECC) | curve (ECC) | | | | | | |
| | ECT_161 | Euler characteristic | Euler characteristic | | | | | | |
| | | curve (ECC) | curve (ECC) | | | | | | |
| | ECT_162 | Euler characteristic | Euler characteristic | | | | | | |
| | | curve (ECC) | curve (ECC) | | | | | | |
| | ECT_163 | Euler characteristic | Euler characteristic | | | | | | |
| | | curve (ECC) | curve (ECC) | | | | | | |
| | ECT_164 | Euler characteristic | Euler characteristic | | | | | | |
| | | curve (ECC) | curve (ECC) | | | | | | |
| | ECT_165 | Euler characteristic | Euler characteristic | | | | | | |
| | | curve (ECC) | curve (ECC) | | | | | | |
| | ECT_166 | Euler characteristic | Euler characteristic | | | | | | |
| | | curve (ECC) | curve (ECC) | | | | | | |
| | ECT_167 | Euler characteristic | Euler characteristic | | | | | | |
| | | curve (ECC) | curve (ECC) | | | | | | |
| | ECT_168 | Euler characteristic | Euler characteristic | | | | | | |
| | | curve (ECC) | curve (ECC) | | | | | | |
| | ECT_169 | Euler characteristic | Euler characteristic | | | | | | |
| | | curve (ECC) | curve (ECC) | | | | | | |
| | ECT_170 | Euler characteristic | Euler characteristic | | | | | | |
| | | curve (ECC) | curve (ECC) | | | | | | |
| | ECT_171 | Euler characteristic | Euler characteristic | | | | | | |
| | | curve (ECC) | curve (ECC) | | | | | | |
| | ECT_172 | Euler characteristic | Euler characteristic | | | | | | |
| | | curve (ECC) | curve (ECC) | | | | | | |
| | ECT_173 | Euler characteristic | Euler characteristic | | | | | | |
| | | curve (ECC) | curve (ECC) | | | | | | |
| | ECT_174 | Euler characteristic | Euler characteristic | | | | | | |
| | | curve (ECC) | curve (ECC) | | | | | | |
| | ECT_175 | Euler characteristic | Euler characteristic | | | | | | |
| | | curve (ECC) | curve (ECC) | | | | | | |
| | ECT_176 | Euler characteristic | Euler characteristic | | | | | | |
| | | curve (ECC) | curve (ECC) | | | | | | |
| | ECT_177 | Euler characteristic | Euler characteristic | | | | | | |
| | | curve (ECC) | curve (ECC) | | | | | | |
| | ECT_178 | Euler characteristic | Euler characteristic | | | | | | |
| | | curve (ECC) | curve (ECC) | | | | | | |
| | ECT_179 | Euler characteristic | Euler characteristic | | | | | | |
| | | curve (ECC) | curve (ECC) | | | | | | |
| | ECT_180 | Euler characteristic | Euler characteristic | | | | | | |
| | | curve (ECC) | curve (ECC) | | | | | | |
| | ECT_181 | Euler characteristic | Euler characteristic | | | | | | |
| | | curve (ECC) | curve (ECC) | | | | | | |
| | ECT_182 | Euler characteristic | Euler characteristic | | | | | | |
| | | curve (ECC) | curve (ECC) | | | | | | |
| | ECT_183 | Euler characteristic | Euler characteristic | | | | | | |
| | | curve (ECC) | curve (ECC) | | | | | | |
| | ECT_184 | Euler characteristic | Euler characteristic | | | | | | |
| | | curve (ECC) | curve (ECC) | | | | | | |
| | ECT_185 | Euler characteristic | Euler characteristic | | | | | | |
| | | curve (ECC) | curve (ECC) | | | | | | |
| | ECT_186 | Euler characteristic | Euler characteristic | | | | | | |
| | | curve (ECC) | curve (ECC) | | | | | | |
| | ECT_187 | Euler characteristic | Euler characteristic | | | | | | |
| | | curve (ECC) | curve (ECC) | | | | | | |
| | ECT_188 | Euler characteristic | Euler characteristic | | | | | | |
| | | curve (ECC) | curve (ECC) | | | | | | |
| | ECT_189 | Euler characteristic | Euler characteristic | | | | | | |
| | | curve (ECC) | curve (ECC) | | | | | | |
| | ECT_190 | Euler characteristic | Euler characteristic | | | | | | |
| | | curve (ECC) | curve (ECC) | | | | | | |
| | ECT_191 | Euler characteristic | Euler characteristic | | | | | | |
| | | curve (ECC) | curve (ECC) | | | | | | |
| | ECT_192 | Euler characteristic | Euler characteristic | | | | | | |
| | | curve (ECC) | curve (ECC) | | | | | | |
| | ECT_193 | Euler characteristic | Euler characteristic | | | | | | |
| | | curve (ECC) | curve (ECC) | | | | | | |
| | ECT_194 | Euler characteristic | Euler characteristic | | | | | | |
| | | curve (ECC) | curve (ECC) | | | | | | |
| | ECT_195 | Euler characteristic | Euler characteristic | | | | | | |
| | | curve (ECC) | curve (ECC) | | | | | | |
| | ECT_196 | Euler characteristic | Euler characteristic | | | | | | |
| | | curve (ECC) | curve (ECC) | | | | | | |
| | ECT_197 | Euler characteristic | Euler characteristic | | | | | | |
| | | curve (ECC) | curve (ECC) | | | | | | |
| | ECT_198 | Euler characteristic | Euler characteristic | | | | | | |
| | | curve (ECC) | curve (ECC) | | | | | | |
| | ECT_199 | Euler characteristic | Euler characteristic | | | | | | |
| | | curve (ECC) | curve (ECC) | | | | | | |
| | ECT_200 | Euler characteristic | Euler characteristic | | | | | | |
| | | curve (ECC) | curve (ECC) | | | | | | |
| | ECT_201 | Euler characteristic | Euler characteristic | | | | | | |
| | | curve (ECC) | curve (ECC) | | | | | | |
| | ECT_202 | Euler characteristic | Euler characteristic | | | | | | |
| | | curve (ECC) | curve (ECC) | | | | | | |
| | ECT_203 | Euler characteristic | Euler characteristic | | | | | | |
| | | curve (ECC) | curve (ECC) | | | | | | |
| | ECT_204 | Euler characteristic | Euler characteristic | | | | | | |
| | | curve (ECC) | curve (ECC) | | | | | | |
| | ECT_205 | Euler characteristic | Euler characteristic | | | | | | |
| | | curve (ECC) | curve (ECC) | | | | | | |
| | ECT_206 | Euler characteristic | Euler characteristic | | | | | | |
| | | curve (ECC) | curve (ECC) | | | | | | |
| | ECT_207 | Euler characteristic | Euler characteristic | | | | | | |
| | | curve (ECC) | curve (ECC) | | | | | | |
| | ECT_208 | Euler characteristic | Euler characteristic | | | | | | |
| | | curve (ECC) | curve (ECC) | | | | | | |
| | ECT_209 | Euler characteristic | Euler characteristic | | | | | | |
| | | curve (ECC) | curve (ECC) | | | | | | |
| | ECT_210 | Euler characteristic | Euler characteristic | | | | | | |
| | | curve (ECC) | curve (ECC) | | | | | | |
| | ECT_211 | Euler characteristic | Euler characteristic | | | | | | |
| | | curve (ECC) | curve (ECC) | | | | | | |
| | ECT_212 | Euler characteristic | Euler characteristic | | | | | | |
| | | curve (ECC) | curve (ECC) | | | | | | |
| | ECT_213 | Euler characteristic | Euler characteristic | | | | | | |
| | | curve (ECC) | curve (ECC) | | | | | | |
| | ECT_214 | Euler characteristic | Euler characteristic | | | | | | |
| | | curve (ECC) | curve (ECC) | | | | | | |
| | ECT_215 | Euler characteristic | Euler characteristic | | | | | | |
| | | curve (ECC) | curve (ECC) | | | | | | |
| | ECT_216 | Euler characteristic | Euler characteristic | | | | | | |
| | | curve (ECC) | curve (ECC) | | | | | | |
| | ECT_217 | Euler characteristic | Euler characteristic | | | | | | |
| | | curve (ECC) | curve (ECC) | | | | | | |
| | ECT_218 | Euler characteristic | Euler characteristic | | | | | | |
| | | curve (ECC) | curve (ECC) | | | | | | |
| | ECT_219 | Euler characteristic | Euler characteristic | | | | | | |
| | | curve (ECC) | curve (ECC) | | | | | | |
| | ECT_220 | Euler characteristic | Euler characteristic | | | | | | |
| | | curve (ECC) | curve (ECC) | | | | | | |
| | ECT_221 | Euler characteristic | Euler characteristic | | | | | | |
| | | curve (ECC) | curve (ECC) | | | | | | |
| | ECT_222 | Euler characteristic | Euler characteristic | | | | | | |
| | | curve (ECC) | curve (ECC) | | | | | | |
| | ECT_223 | Euler characteristic | Euler characteristic | | | | | | |
| | | curve (ECC) | curve (ECC) | | | | | | |
| | ECT_224 | Euler characteristic | Euler characteristic | | | | | | |
| | | curve (ECC) | curve (ECC) | | | | | | |
| | ECT_225 | Euler characteristic | Euler characteristic | | | | | | |
| | | curve (ECC) | curve (ECC) | | | | | | |
| | ECT_226 | Euler characteristic | Euler characteristic | | | | | | |
| | | curve (ECC) | curve (ECC) | | | | | | |
| | ECT_227 | Euler characteristic | Euler characteristic | | | | | | |
| | | curve (ECC) | curve (ECC) | | | | | | |
| | ECT_228 | Euler characteristic | Euler characteristic | | | | | | |
| | | curve (ECC) | curve (ECC) | | | | | | |
| | ECT_229 | Euler characteristic | Euler characteristic | | | | | | |
| | | curve (ECC) | curve (ECC) | | | | | | |
| | ECT_230 | Euler characteristic | Euler characteristic | | | | | | |
| | | curve (ECC) | curve (ECC) | | | | | | |
| | ECT_231 | Euler characteristic | Euler characteristic | | | | | | |
| | | curve (ECC) | curve (ECC) | | | | | | |
| | ECT_232 | Euler characteristic | Euler characteristic | | | | | | |
| | | curve (ECC) | curve (ECC) | | | | | | |
| | ECT_233 | Euler characteristic | Euler characteristic | | | | | | |
| | | curve (ECC) | curve (ECC) | | | | | | |
| | ECT_234 | Euler characteristic | Euler characteristic | | | | | | |
| | | curve (ECC) | curve (ECC) | | | | | | |
| | ECT_235 | Euler characteristic | Euler characteristic | | | | | | |
| | | curve (ECC) | curve (ECC) | | | | | | |
| | ECT_236 | Euler characteristic | Euler characteristic | | | | | | |
| | | curve (ECC) | curve (ECC) | | | | | | |
| | ECT_237 | Euler characteristic | Euler characteristic | | | | | | |
| | | curve (ECC) | curve (ECC) | | | | | | |
| | ECT_238 | Euler characteristic | Euler characteristic | | | | | | |
| | | curve (ECC) | curve (ECC) | | | | | | |
| | ECT_239 | Euler characteristic | Euler characteristic | | | | | | |
| | | curve (ECC) | curve (ECC) | | | | | | |
| | ECT_240 | Euler characteristic | Euler characteristic | | | | | | |
| | | curve (ECC) | curve (ECC) | | | | | | |
| | ECT_241 | Euler characteristic | Euler characteristic | | | | | | |
| | | curve (ECC) | curve (ECC) | | | | | | |
| | ECT_242 | Euler characteristic | Euler characteristic | | | | | | |
| | | curve (ECC) | curve (ECC) | | | | | | |
| | ECT_243 | Euler characteristic | Euler characteristic | | | | | | |
| | | curve (ECC) | curve (ECC) | | | | | | |
| | ECT_244 | Euler characteristic | Euler characteristic | | | | | | |
| | | curve (ECC) | curve (ECC) | | | | | | |
| | ECT_245 | Euler characteristic | Euler characteristic | | | | | | |
| | | curve (ECC) | curve (ECC) | | | | | | |
| | ECT_246 | Euler characteristic | Euler characteristic | | | | | | |
| | | curve (ECC) | curve (ECC) | | | | | | |
| | ECT_247 | Euler characteristic | Euler characteristic | | | | | | |
| | | curve (ECC) | curve (ECC) | | | | | | |
| | ECT_248 | Euler characteristic | Euler characteristic | | | | | | |
| | | curve (ECC) | curve (ECC) | | | | | | |
| | ECT_249 | Euler characteristic | Euler characteristic | | | | | | |
| | | curve (ECC) | curve (ECC) | | | | | | |
| | ECT_250 | Euler characteristic | Euler characteristic | | | | | | |
| | | curve (ECC) | curve (ECC) | | | | | | |
| | ECT_251 | Euler characteristic | Euler characteristic | | | | | | |
| | | curve (ECC) | curve (ECC) | | | | | | |
| | ECT_252 | Euler characteristic | Euler characteristic | | | | | | |
| | | curve (ECC) | curve (ECC) | | | | | | |
| | ECT_253 | Euler characteristic | Euler characteristic | | | | | | |
| | | curve (ECC) | curve (ECC) | | | | | | |
| | ECT_254 | Euler characteristic | Euler characteristic | | | | | | |
| | | curve (ECC) | curve (ECC) | | | | | | |
| | ECT_255 | Euler characteristic | Euler characteristic | | | | | | |
| | | curve (ECC) | curve (ECC) | | | | | | |
| | ECT_256 | Euler characteristic | Euler characteristic | | | | | | |
| | | curve (ECC) | curve (ECC) | | | | | | |
| | ECT_257 | Euler characteristic | Euler characteristic | | | | | | |
| | | curve (ECC) | curve (ECC) | | | | | | |
| | ECT_258 | Euler characteristic | Euler characteristic | | | | | | |
| | | curve (ECC) | curve (ECC) | | | | | | |
| | ECT_259 | Euler characteristic | Euler characteristic | | | | | | |
| | | curve (ECC) | curve (ECC) | | | | | | |
| | ECT_260 | Euler characteristic | Euler characteristic | | | | | | |
| | | curve (ECC) | curve (ECC) | | | | | | |
| | ECT_261 | Euler characteristic | Euler characteristic | | | | | | |
| | | curve (ECC) | curve (ECC) | | | | | | |
| | ECT_262 | Euler characteristic | Euler characteristic | | | | | | |
| | | curve (ECC) | curve (ECC) | | | | | | |
| | ECT_263 | Euler characteristic | Euler characteristic | | | | | | |
| | | curve (ECC) | curve (ECC) | | | | | | |
| | ECT_264 | Euler characteristic | Euler characteristic | | | | | | |
| | | curve (ECC) | curve (ECC) | | | | | | |
| | ECT_265 | Euler characteristic | Euler characteristic | | | | | | |
| | | curve (ECC) | curve (ECC) | | | | | | |
| | ECT_266 | Euler characteristic | Euler characteristic | | | | | | |
| | | curve (ECC) | curve (ECC) | | | | | | |
| | ECT_267 | Euler characteristic | Euler characteristic | | | | | | |
| | | curve (ECC) | curve (ECC) | | | | | | |
| | ECT_268 | Euler characteristic | Euler characteristic | | | | | | |
| | | curve (ECC) | curve (ECC) | | | | | | |
| | ECT_269 | Euler characteristic | Euler characteristic | | | | | | |
| | | curve (ECC) | curve (ECC) | | | | | | |
| | ECT_270 | Euler characteristic | Euler characteristic | | | | | | |
| | | curve (ECC) | curve (ECC) | | | | | | |
| | ECT_271 | Euler characteristic | Euler characteristic | | | | | | |
| | | curve (ECC) | curve (ECC) | | | | | | |
| | ECT_272 | Euler characteristic | Euler characteristic | | | | | | |
| | | curve (ECC) | curve (ECC) | | | | | | |
| | ECT_273 | Euler characteristic | Euler characteristic | | | | | | |
| | | curve (ECC) | curve (ECC) | | | | | | |
| | ECT_274 | Euler characteristic | Euler characteristic | | | | | | |
| | | curve (ECC) | curve (ECC) | | | | | | |
| | ECT_275 | Euler characteristic | Euler characteristic | | | | | | |
| | | curve (ECC) | curve (ECC) | | | | | | |
| | ECT_276 | Euler characteristic | Euler characteristic | | | | | | |
| | | curve (ECC) | curve (ECC) | | | | | | |
| | ECT_277 | Euler characteristic | Euler characteristic | | | | | | |
| | | curve (ECC) | curve (ECC) | | | | | | |
| | ECT_278 | Euler characteristic | Euler characteristic | | | | | | |
| | | curve (ECC) | curve (ECC) | | | | | | |
| | ECT_279 | Euler characteristic | Euler characteristic | | | | | | |
| | | curve (ECC) | curve (ECC) | | | | | | |
| | ECT_280 | Euler characteristic | Euler characteristic | | | | | | |
| | | curve (ECC) | curve (ECC) | | | | | | |
| | ECT_281 | Euler characteristic | Euler characteristic | | | | | | |
| | | curve (ECC) | curve (ECC) | | | | | | |
| | ECT_282 | Euler characteristic | Euler characteristic | | | | | | |
| | | curve (ECC) | curve (ECC) | | | | | | |
| | ECT_283 | Euler characteristic | Euler characteristic | | | | | | |
| | | curve (ECC) | curve (ECC) | | | | | | |
| | ECT_284 | Euler characteristic | Euler characteristic | | | | | | |
| | | curve (ECC) | curve (ECC) | | | | | | |
| | ECT_285 | Euler characteristic | Euler characteristic | | | | | | |
| | | curve (ECC) | curve (ECC) | | | | | | |
| | ECT_286 | Euler characteristic | Euler characteristic | | | | | | |
| | | curve (ECC) | curve (ECC) | | | | | | |
| | ECT_287 | Euler characteristic | Euler characteristic | | | | | | |
| | | curve (ECC) | curve (ECC) | | | | | | |
| | ECT_288 | Euler characteristic | Euler characteristic | | | | | | |
| | | curve (ECC) | curve (ECC) | | | | | | |
| | ECT_289 | Euler characteristic | Euler characteristic | | | | | | |
| | | curve (ECC) | curve (ECC) | | | | | | |
| | ECT_290 | Euler characteristic | Euler characteristic | | | | | | |
| | | curve (ECC) | curve (ECC) | | | | | | |
| | ECT_291 | Euler characteristic | Euler characteristic | | | | | | |
| | | curve (ECC) | curve (ECC) | | | | | | |
| | ECT_292 | Euler characteristic | Euler characteristic | | | | | | |
| | | curve (ECC) | curve (ECC) | | | | | | |
| | ECT_293 | Euler characteristic | Euler characteristic | | | | | | |
| | | curve (ECC) | curve (ECC) | | | | | | |
| | ECT_294 | Euler characteristic | Euler characteristic | | | | | | |
| | | curve (ECC) | curve (ECC) | | | | | | |
| | ECT_295 | Euler characteristic | Euler characteristic | | | | | | |
| | | curve (ECC) | curve (ECC) | | | | | | |
| | ECT_296 | Euler characteristic | Euler characteristic | | | | | | |
| | | curve (ECC) | curve (ECC) | | | | | | |
| | ECT_297 | Euler characteristic | Euler characteristic | | | | | | |
| | | curve (ECC) | curve (ECC) | | | | | | |
| | ECT_298 | Euler characteristic | Euler characteristic | | | | | | |
| | | curve (ECC) | curve (ECC) | | | | | | |
| | ECT_299 | Euler characteristic | Euler characteristic | | | | | | |
| | | curve (ECC) | curve (ECC) | | | | | | |
| | ECT_300 | Euler characteristic | Euler characteristic | | | | | | |
| | | curve (ECC) | curve (ECC) | | | | | | |
| | ECT_301 | Euler characteristic | Euler characteristic | | | | | | |
| | | curve (ECC) | curve (ECC) | | | | | | |
| | ECT_302 | Euler characteristic | Euler characteristic | | | | | | |
| | | curve (ECC) | curve (ECC) | | | | | | |
| | ECT_303 | Euler characteristic | Euler characteristic | | | | | | |
| | | curve (ECC) | curve (ECC) | | | | | | |
| | ECT_304 | Euler characteristic | Euler characteristic | | | | | | |
| | | curve (ECC) | curve (ECC) | | | | | | |
| | ECT_305 | Euler characteristic | Euler characteristic | | | | | | |
| | | curve (ECC) | curve (ECC) | | | | | | |
| | ECT_306 | Euler characteristic | Euler characteristic | | | | | | |
| | | curve (ECC) | curve (ECC) | | | | | | |
| | ECT_307 | Euler characteristic | Euler characteristic | | | | | | |
| | | curve (ECC) | curve (ECC) | | | | | | |
| | ECT_308 | Euler characteristic | Euler characteristic | | | | | | |
| | | curve (ECC) | curve (ECC) | | | | | | |
| | ECT_309 | Euler characteristic | Euler characteristic | | | | | | |
| | | curve (ECC) | curve (ECC) | | | | | | |
| | ECT_310 | Euler characteristic | Euler characteristic | | | | | | |
| | | curve (ECC) | curve (ECC) | | | | | | |
| | ECT_311 | Euler characteristic | Euler characteristic | | | | | | |
| | | curve (ECC) | curve (ECC) | | | | | | |
| | ECT_312 | Euler characteristic | Euler characteristic | | | | | | |
| | | curve (ECC) | curve (ECC) | | | | | | |
| | ECT_313 | Euler characteristic | Euler characteristic | | | | | | |
| | | curve (ECC) | curve (ECC) | | | | | | |
| | ECT_314 | Euler characteristic | Euler characteristic | | | | | | |
| | | curve (ECC) | curve (ECC) | | | | | | |
| | ECT_315 | Euler characteristic | Euler characteristic | | | | | | |
| | | curve (ECC) | curve (ECC) | | | | | | |
| | ECT_316 | Euler characteristic | Euler characteristic | | | | | | |
| | | curve (ECC) | curve (ECC) | | | | | | |
| | ECT_317 | Euler characteristic | Euler characteristic | | | | | | |
| | | curve (ECC) | curve (ECC) | | | | | | |
| | ECT_318 | Euler characteristic | Euler characteristic | | | | | | |
| | | curve (ECC) | curve (ECC) | | | | | | |
| | ECT_319 | Euler characteristic | Euler characteristic | | | | | | |
| | | curve (ECC) | curve (ECC) | | | | | | |
| | ECT_320 | Euler characteristic | Euler characteristic | | | | | | |
| | | curve (ECC) | curve (ECC) | | | | | | |
| | ECT_321 | Euler characteristic | Euler characteristic | | | | | | |
| | | curve (ECC) | curve (ECC) | | | | | | |
| | ECT_322 | Euler characteristic | Euler characteristic | | | | | | |
| | | curve (ECC) | curve (ECC) | | | | | | |
| | ECT_323 | Euler characteristic | Euler characteristic | | | | | | |
| | | curve (ECC) | curve (ECC) | | | | | | |
| | ECT_324 | Euler characteristic | Euler characteristic | | | | | | |
| | | curve (ECC) | curve (ECC) | | | | | | |
| | ECT_325 | Euler characteristic | Euler characteristic | | | | | | |
| | | curve (ECC) | curve (ECC) | | | | | | |
| | ECT_326 | Euler characteristic | Euler characteristic | | | | | | |
| | | curve (ECC) | curve (ECC) | | | | | | |
| | ECT_327 | Euler characteristic | Euler characteristic | | | | | | |
| | | curve (ECC) | curve (ECC) | | | | | | |
| | ECT_328 | Euler characteristic | Euler characteristic | | | | | | |
| | | curve (ECC) | curve (ECC) | | | | | | |
| | ECT_329 | Euler characteristic | Euler characteristic | | | | | | |
| | | curve (ECC) | curve (ECC) | | | | | | |
| | ECT_330 | Euler characteristic | Euler characteristic | | | | | | |
| | | curve (ECC) | curve (ECC) | | | | | | |
| | ECT_331 | Euler characteristic | Euler characteristic | | | | | | |
| | | curve (ECC) | curve (ECC) | | | | | | |
| | ECT_332 | Euler characteristic | Euler characteristic | | | | | | |
| | | curve (ECC) | curve (ECC) | | | | | | |
| | ECT_333 | Euler characteristic | Euler characteristic | | | | | | |
| | | curve (ECC) | curve (ECC) | | | | | | |
| | ECT_334 | Euler characteristic | Euler characteristic | | | | | | |
| | | curve (ECC) | curve (ECC) | | | | | | |
| | ECT_335 | Euler characteristic | Euler characteristic | | | | | | |
| | | curve (ECC) | curve (ECC) | | | | | | |
| | ECT_336 | Euler characteristic | Euler characteristic | | | | | | |
| | | curve (ECC) | curve (ECC) | | | | | | |
| | ECT_337 | Euler characteristic | Euler characteristic | | | | | | |
| | | curve (ECC) | curve (ECC) | | | | | | |
| | ECT_338 | Euler characteristic | Euler characteristic | | | | | | |
| | | curve (ECC) | curve (ECC) | | | | | | |
| | ECT_339 | Euler characteristic | Euler characteristic | | | | | | |
| | | curve (ECC) | curve (ECC) | | | | | | |
| | ECT_340 | Euler characteristic | Euler characteristic | | | | | | |
| | | curve (ECC) | curve (ECC) | | | | | | |
| | ECT_341 | Euler characteristic | Euler characteristic | | | | | | |
| | | curve (ECC) | curve (ECC) | | | | | | |
| | ECT_342 | Euler characteristic | Euler characteristic | | | | | | |
| | | curve (ECC) | curve (ECC) | | | | | | |
| | ECT_343 | Euler characteristic | Euler characteristic | | | | | | |
| | | curve (ECC) | curve (ECC) | | | | | | |
| | ECT_344 | Euler characteristic | Euler characteristic | | | | | | |
| | | curve (ECC) | curve (ECC) | | | | | | |
| | ECT_345 | Euler characteristic | Euler characteristic | | | | | | |
| | | curve (ECC) | curve (ECC) | | | | | | |
| | ECT_346 | Euler characteristic | Euler characteristic | | | | | | |
| | | curve (ECC) | curve (ECC) | | | | | | |
| | ECT_347 | Euler characteristic | Euler characteristic | | | | | | |
| | | curve (ECC) | curve (ECC) | | | | | | |
| | ECT_348 | Euler characteristic | Euler characteristic | | | | | | |
| | | curve (ECC) | curve (ECC) | | | | | | |
| | ECT_349 | Euler characteristic | Euler characteristic | | | | | | |
| | | curve (ECC) | curve (ECC) | | | | | | |
| | ECT_350 | Euler characteristic | Euler characteristic | | | | | | |
| | | curve (ECC) | curve (ECC) | | | | | | |
| | ECT_351 | Euler characteristic | Euler characteristic | | | | | | |
| | | curve (ECC) | curve (ECC) | | | | | | |
| | ECT_352 | Euler characteristic | Euler characteristic | | | | | | |
| | | curve (ECC) | curve (ECC) | | | | | | |
| | ECT_353 | Euler characteristic | Euler characteristic | | | | | | |
| | | curve (ECC) | curve (ECC) | | | | | | |
| | ECT_354 | Euler characteristic | Euler characteristic | | | | | | |
| | | curve (ECC) | curve (ECC) | | | | | | |
| | ECT_355 | Euler characteristic | Euler characteristic | | | | | | |
| | | curve (ECC) | curve (ECC) | | | | | | |
| | ECT_356 | Euler characteristic | Euler characteristic | | | | | | |
| | | curve (ECC) | curve (ECC) | | | | | | |
| | ECT_357 | Euler characteristic | Euler characteristic | | | | | | |
| | | curve (ECC) | curve (ECC) | | | | | | |
| | ECT_358 | Euler characteristic | Euler characteristic | | | | | | |
| | | curve (ECC) | curve (ECC) | | | | | | |
| | ECT_359 | Euler characteristic | Euler characteristic | | | | | | |
| | | curve (ECC) | curve (ECC) | | | | | | |
| | ECT_360 | Euler characteristic | Euler characteristic | | | | | | |
| | | curve (ECC) | curve (ECC) | | | | | | |
| | ECT_361 | Euler characteristic | Euler characteristic | | | | | | |
| | | curve (ECC) | curve (ECC) | | | | | | |
| | ECT_362 | Euler characteristic | Euler characteristic | | | | | | |
| | | curve (ECC) | curve (ECC) | | | | | | |
| | ECT_363 | Euler characteristic | Euler characteristic | | | | | | |
| | | curve (ECC) | curve (ECC) | | | | | | |
| | ECT_364 | Euler characteristic | Euler characteristic | | | | | | |
| | | curve (ECC) | curve (ECC) | | | | | | |
| | ECT_365 | Euler characteristic | Euler characteristic | | | | | | |
| | | curve (ECC) | curve (ECC) | | | | | | |
| | ECT_366 | Euler characteristic | Euler characteristic | | | | | | |
| | | curve (ECC) | curve (ECC) | | | | | | |
| | ECT_367 | Euler characteristic | Euler characteristic | | | | | | |
| | | curve (ECC) | curve (ECC) | | | | | | |
| | ECT_368 | Euler characteristic | Euler characteristic | | | | | | |
| | | curve (ECC) | curve (ECC) | | | | | | |
| | ECT_369 | Euler characteristic | Euler characteristic | | | | | | |
| | | curve (ECC) | curve (ECC) | | | | | | |
| | ECT_370 | Euler characteristic | Euler characteristic | | | | | | |
| | | curve (ECC) | curve (ECC) | | | | | | |
| | ECT_371 | Euler characteristic | Euler characteristic | | | | | | |
| | | curve (ECC) | curve (ECC) | | | | | | |
| | ECT_372 | Euler characteristic | Euler characteristic | | | | | | |
| | | curve (ECC) | curve (ECC) | | | | | | |
| | ECT_373 | Euler characteristic | Euler characteristic | | | | | | |
| | | curve (ECC) | curve (ECC) | | | | | | |
| | ECT_374 | Euler characteristic | Euler characteristic | | | | | | |
| | | curve (ECC) | curve (ECC) | | | | | | |
| | ECT_375 | Euler characteristic | Euler characteristic | | | | | | |
| | | curve (ECC) | curve (ECC) | | | | | | |
| | ECT_376 | Euler characteristic | Euler characteristic | | | | | | |
| | | curve (ECC) | curve (ECC) | | | | | | |
| | ECT_377 | Euler characteristic | Euler characteristic | | | | | | |
| | | curve (ECC) | curve (ECC) | | | | | | |
| | ECT_378 | Euler characteristic | Euler characteristic | | | | | | |
| | | curve (ECC) | curve (ECC) | | | | | | |
| | ECT_379 | Euler characteristic | Euler characteristic | | | | | | |
| | | curve (ECC) | curve (ECC) | | | | | | |
| | ECT_380 | Euler characteristic | Euler characteristic | | | | | | |
| | | curve (ECC) | curve (ECC) | | | | | | |
| | ECT_381 | Euler characteristic | Euler characteristic | | | | | | |
| | | curve (ECC) | curve (ECC) | | | | | | |
| | ECT_382 | Euler characteristic | Euler characteristic | | | | | | |
| | | curve (ECC) | curve (ECC) | | | | | | |
| | ECT_383 | Euler characteristic | Euler characteristic | | | | | | |
| | | curve (ECC) | curve (ECC) | | | | | | |
| | ECT_384 | Euler characteristic | Euler characteristic | | | | | | |
| | | curve (ECC) | curve (ECC) | | | | | | |
| | ECT_385 | Euler characteristic | Euler characteristic | | | | | | |
| | | curve (ECC) | curve (ECC) | | | | | | |
| | ECT_386 | Euler characteristic | Euler characteristic | | | | | | |
| | | curve (ECC) | curve (ECC) | | | | | | |
| | ECT_387 | Euler characteristic | Euler characteristic | | | | | | |
| | | curve (ECC) | curve (ECC) | | | | | | |
| | ECT_388 | Euler characteristic | Euler characteristic | | | | | | |
| | | curve (ECC) | curve (ECC) | | | | | | |
| | ECT_389 | Euler characteristic | Euler characteristic | | | | | | |
| | | curve (ECC) | curve (ECC) | | | | | | |
| | ECT_390 | Euler characteristic | Euler characteristic | | | | | | |
| | | curve (ECC) | curve (ECC) | | | | | | |
| | ECT_391 | Euler characteristic | Euler characteristic | | | | | | |
| | | curve (ECC) | curve (ECC) | | | | | | |
| | ECT_392 | Euler characteristic | Euler characteristic | | | | | | |
| | | curve (ECC) | curve (ECC) | | | | | | |
| | ECT_393 | Euler characteristic | Euler characteristic | | | | | | |
| | | curve (ECC) | curve (ECC) | | | | | | |
| | ECT_394 | Euler characteristic | Euler characteristic | | | | | | |
| | | curve (ECC) | curve (ECC) | | | | | | |
| | ECT_395 | Euler characteristic | Euler characteristic | | | | | | |
| | | curve (ECC) | curve (ECC) | | | | | | |
| | ECT_396 | Euler characteristic | Euler characteristic | | | | | | |
| | | curve (ECC) | curve (ECC) | | | | | | |
| | ECT_397 | Euler characteristic | Euler characteristic | | | | | | |
| | | curve (ECC) | curve (ECC) | | | | | | |
| | ECT_398 | Euler characteristic | Euler characteristic | | | | | | |
| | | curve (ECC) | curve (ECC) | | | | | | |
| | ECT_399 | Euler characteristic | Euler characteristic | | | | | | |
| | | curve (ECC) | curve (ECC) | | | | | | |
| | ECT_400 | Euler characteristic | Euler characteristic | | | | | | |
| | | curve (ECC) | curve (ECC) | | | | | | |
| | ECT_401 | Euler characteristic | Euler characteristic | | | | | | |
| | | curve (ECC) | curve (ECC) | | | | | | |
| | ECT_402 | Euler characteristic | Euler characteristic | | | | | | |
| | | curve (ECC) | curve (ECC) | | | | | | |
| | ECT_403 | Euler characteristic | Euler characteristic | | | | | | |
| | | curve (ECC) | curve (ECC) | | | | | | |
| | ECT_404 | Euler characteristic | Euler characteristic | | | | | | |
| | | curve (ECC) | curve (ECC) | | | | | | |
| | ECT_405 | Euler characteristic | Euler characteristic | | | | | | |
| | | curve (ECC) | curve (ECC) | | | | | | |
| | ECT_406 | Euler characteristic | Euler characteristic | | | | | | |
| | | curve (ECC) | curve (ECC) | | | | | | |
| | ECT_407 | Euler characteristic | Euler characteristic | | | | | | |
| | | curve (ECC) | curve (ECC) | | | | | | |
| | ECT_408 | Euler characteristic | Euler characteristic | | | | | | |
| | | curve (ECC) | curve (ECC) | | | | | | |
| | ECT_409 | Euler characteristic | Euler characteristic | | | | | | |
| | | curve (ECC) | curve (ECC) | | | | | | |
| | ECT_410 | Euler characteristic | Euler characteristic | | | | | | |
| | | curve (ECC) | curve (ECC) | | | | | | |
| | ECT_411 | Euler characteristic | Euler characteristic | | | | | | |
| | | curve (ECC) | curve (ECC) | | | | | | |
| | ECT_412 | Euler characteristic | Euler characteristic | | | | | | |
| | | curve (ECC) | curve (ECC) | | | | | | |
| | ECT_413 | Euler characteristic | Euler characteristic | | | | | | |
| | | curve (ECC) | curve (ECC) | | | | | | |
| | ECT_414 | Euler characteristic | Euler characteristic | | | | | | |
| | | curve (ECC) | curve (ECC) | | | | | | |
| | ECT_415 | Euler characteristic | Euler characteristic | | | | | | |
| | | curve (ECC) | curve (ECC) | | | | | | |
| | ECT_416 | Euler characteristic | Euler characteristic | | | | | | |
| | | curve (ECC) | curve (ECC) | | | | | | |
| | ECT_417 | Euler characteristic | Euler characteristic | | | | | | |
| | | curve (ECC) | curve (ECC) | | | | | | |
| | ECT_418 | Euler characteristic | Euler characteristic | | | | | | |
| | | curve (ECC) | curve (ECC) | | | | | | |
| | ECT_419 | Euler characteristic | Euler characteristic | | | | | | |
| | | curve (ECC) | curve (ECC) | | | | | | |
| | ECT_420 | Euler characteristic | Euler characteristic | | | | | | |
| | | curve (ECC) | curve (ECC) | | | | | | |
| | ECT_421 | Euler characteristic | Euler characteristic | | | | | | |
| | | curve (ECC) | curve (ECC) | | | | | | |
| | ECT_422 | Euler characteristic | Euler characteristic | | | | | | |
| | | curve (ECC) | curve (ECC) | | | | | | |
| | ECT_423 | Euler characteristic | Euler characteristic | | | | | | |
| | | curve (ECC) | curve (ECC) | | | | | | |
| | ECT_424 | Euler characteristic | Euler characteristic | | | | | | |
| | | curve (ECC) | curve (ECC) | | | | | | |
| | ECT_425 | Euler characteristic | Euler characteristic | | | | | | |
| | | curve (ECC) | curve (ECC) | | | | | | |
| | ECT_426 | Euler characteristic | Euler characteristic | | | | | | |
| | | curve (ECC) | curve (ECC) | | | | | | |
| | ECT_427 | Euler characteristic | Euler characteristic | | | | | | |
| | | curve (ECC) | curve (ECC) | | | | | | |
| | ECT_428 | Euler characteristic | Euler characteristic | | | | | | |
| | | curve (ECC) | curve (ECC) | | | | | | |
| | ECT_429 | Euler characteristic | Euler characteristic | | | | | | |
| | | curve (ECC) | curve (ECC) | | | | | | |
| | ECT_430 | Euler characteristic | Euler characteristic | | | | | | |
| | | curve (ECC) | curve (ECC) | | | | | | |
| | ECT_431 | Euler characteristic | Euler characteristic | | | | | | |
| | | curve (ECC) | curve (ECC) | | | | | | |
| | ECT_432 | Euler characteristic | Euler characteristic | | | | | | |
| | | curve (ECC) | curve (ECC) | | | | | | |
| | ECT_433 | Euler characteristic | Euler characteristic | | | | | | |
| | | curve (ECC) | curve (ECC) | | | | | | |
| | ECT_434 | Euler characteristic | Euler characteristic | | | | | | |
| | | curve (ECC) | curve (ECC) | | | | | | |
| | ECT_435 | Euler characteristic | Euler characteristic | | | | | | |
| | | curve (ECC) | curve (ECC) | | | | | | |
| | ECT_436 | Euler characteristic | Euler characteristic | | | | | | |
| | | curve (ECC) | curve (ECC) | | | | | | |
| | ECT_437 | Euler characteristic | Euler characteristic | | | | | | |
| | | curve (ECC) | curve (ECC) | | | | | | |
| | ECT_438 | Euler characteristic | Euler characteristic | | | | | | |
| | | curve (ECC) | curve (ECC) | | | | | | |
| | ECT_439 | Euler characteristic | Euler characteristic | | | | | | |
| | | curve (ECC) | curve (ECC) | | | | | | |
| | ECT_440 | Euler characteristic | Euler characteristic | | | | | | |
| | | curve (ECC) | curve (ECC) | | | | | | |
| | ECT_441 | Euler characteristic | Euler characteristic | | | | | | |
| | | curve (ECC) | curve (ECC) | | | | | | |
| | ECT_442 | Euler characteristic | Euler characteristic | | | | | | |
| | | curve (ECC) | curve (ECC) | | | | | | |
| | ECT_443 | Euler characteristic | Euler characteristic | | | | | | |
| | | curve (ECC) | curve (ECC) | | | | | | |
| | ECT_444 | Euler characteristic | Euler characteristic | | | | | | |
| | | curve (ECC) | curve (ECC) | | | | | | |
| | ECT_445 | Euler characteristic | Euler characteristic | | | | | | |
| | | curve (ECC) | curve (ECC) | | | | | | |
| | ECT_446 | Euler characteristic | Euler characteristic | | | | | | |
| | | curve (ECC) | curve (ECC) | | | | | | |
| | ECT_447 | Euler characteristic | Euler characteristic | | | | | | |
| | | curve (ECC) | curve (ECC) | | | | | | |
| | ECT_448 | Euler characteristic | Euler characteristic | | | | | | |
| | | curve (ECC) | curve (ECC) | | | | | | |
| | ECT_449 | Euler characteristic | Euler characteristic | | | | | | |
| | | curve (ECC) | curve (ECC) | | | | | | |
| | ECT_450 | Euler characteristic | Euler characteristic | | | | | | |
| | | curve (ECC) | curve (ECC) | | | | | | |
| | ECT_451 | Euler characteristic | Euler characteristic | | | | | | |
| | | curve (ECC) | curve (ECC) | | | | | | |
| | ECT_452 | Euler characteristic | Euler characteristic | | | | | | |
| | | curve (ECC) | curve (ECC) | | | | | | |
| | ECT_453 | Euler characteristic | Euler characteristic | | | | | | |
| | | curve (ECC) | curve (ECC) | | | | | | |
| | ECT_454 | Euler characteristic | Euler characteristic | | | | | | |
| | | curve (ECC) | curve (ECC) | | | | | | |
| | ECT_455 | Euler characteristic | Euler characteristic | | | | | | |
| | | curve (ECC) | curve (ECC) | | | | | | |
| | ECT_456 | Euler characteristic | Euler characteristic | | | | | | |
| | | curve (ECC) | curve (ECC) | | | | | | |
| | ECT_457 | Euler characteristic | Euler characteristic | | | | | | |
| | | curve (ECC) | curve (ECC) | | | | | | |
| | ECT_458 | Euler characteristic | Euler characteristic | | | | | | |
| | | curve (ECC) | curve (ECC) | | | | | | |
| | ECT_459 | Euler characteristic | Euler characteristic | | | | | | |
| | | curve (ECC) | curve (ECC) | | | | | | |
| | ECT_460 | Euler characteristic | Euler characteristic | | | | | | |
| | | curve (ECC) | curve (ECC) | | | | | | |
| | ECT_461 | Euler characteristic | Euler characteristic | | | | | | |
| | | curve (ECC) | curve (ECC) | | | | | | |
| | ECT_462 | Euler characteristic | Euler characteristic | | | | | | |
| | | curve (ECC) | curve (ECC) | | | | | | |
| | ECT_463 | Euler characteristic | Euler characteristic | | | | | | |
| | | curve (ECC) | curve (ECC) | | | | | | |
| | ECT_464 | Euler characteristic | Euler characteristic | | | | | | |
| | | curve (ECC) | curve (ECC) | | | | | | |
| | ECT_465 | Euler characteristic | Euler characteristic | | | | | | |
| | | curve (ECC) | curve (ECC) | | | | | | |
| | ECT_466 | Euler characteristic | Euler characteristic | | | | | | |
| | | curve (ECC) | curve (ECC) | | | | | | |
| | ECT_467 | Euler characteristic | Euler characteristic | | | | | | |
| | | curve (ECC) | curve (ECC) | | | | | | |
| | ECT_468 | Euler characteristic | Euler characteristic | | | | | | |
| | | curve (ECC) | curve (ECC) | | | | | | |
| | ECT_469 | Euler characteristic | Euler characteristic | | | | | | |
| | | curve (ECC) | curve (ECC) | | | | | | |
| | ECT_470 | Euler characteristic | Euler characteristic | | | | | | |
| | | curve (ECC) | curve (ECC) | | | | | | |
| | ECT_471 | Euler characteristic | Euler characteristic | | | | | | |
| | | curve (ECC) | curve (ECC) | | | | | | |
| | ECT_472 | Euler characteristic | Euler characteristic | | | | | | |
| | | curve (ECC) | curve (ECC) | | | | | | |
| | ECT_473 | Euler characteristic | Euler characteristic | | | | | | |
| | | curve (ECC) | curve (ECC) | | | | | | |
| | ECT_474 | Euler characteristic | Euler characteristic | | | | | | |
| | | curve (ECC) | curve (ECC) | | | | | | |
| | ECT_475 | Euler characteristic | Euler characteristic | | | | | | |
| | | curve (ECC) | curve (ECC) | | | | | | |
| | ECT_476 | Euler characteristic | Euler characteristic | | | | | | |
| | | curve (ECC) | curve (ECC) | | | | | | |
| | ECT_477 | Euler characteristic | Euler characteristic | | | | | | |
| | | curve (ECC) | curve (ECC) | | | | | | |
| | ECT_478 | Euler characteristic | Euler characteristic | | | | | | |
| | | curve (ECC) | curve (ECC) | | | | | | |
| | ECT_479 | Euler characteristic | Euler characteristic | | | | | | |
| | | curve (ECC) | curve (ECC) | | | | | | |
| | ECT_480 | Euler characteristic | Euler characteristic | | | | | | |
| | | curve (ECC) | curve (ECC) | | | | | | |
| | ECT_481 | Euler characteristic | Euler characteristic | | | | | | |
| | | curve (ECC) | curve (ECC) | | | | | | |
| | ECT_482 | Euler characteristic | Euler characteristic | | | | | | |
| | | curve (ECC) | curve (ECC) | | | | | | |
| | ECT_483 | Euler characteristic | Euler characteristic | | | | | | |
| | | curve (ECC) | curve (ECC) | | | | | | |
| | ECT_484 | Euler characteristic | Euler characteristic | | | | | | |
| | | curve (ECC) | curve (ECC) | | | | | | |
| | ECT_485 | Euler characteristic | Euler characteristic | | | | | | |
| | | curve (ECC) | curve (ECC) | | | | | | |
| | ECT_486 | Euler characteristic | Euler characteristic | | | | | | |
| | | curve (ECC) | curve (ECC) | | | | | | |
| | ECT_487 | Euler characteristic | Euler characteristic | | | | | | |
| | | curve (ECC) | curve (ECC) | | | | | | |
| | ECT_488 | Euler characteristic | Euler characteristic | | | | | | |
| | | curve (ECC) | curve (ECC) | | | | | | |
| | ECT_489 | Euler characteristic | Euler characteristic | | | | | | |
| | | curve (ECC) | curve (ECC) | | | | | | |
| | ECT_490 | Euler characteristic | Euler characteristic | | | | | | |
| | | curve (ECC) | curve (ECC) | | | | | | |
| | ECT_491 | Euler characteristic | Euler characteristic | | | | | | |
| | | curve (ECC) | curve (ECC) | | | | | | |
| | ECT_492 | Euler characteristic | Euler characteristic | | | | | | |
| | | curve (ECC) | curve (ECC) | | | | | | |
| | ECT_493 | Euler characteristic | Euler characteristic | | | | | | |
| | | curve (ECC) | curve (ECC) | | | | | | |
| | ECT_494 | Euler characteristic | Euler characteristic | | | | | | |
| | | curve (ECC) | curve (ECC) | | | | | | |
| | ECT_495 | Euler characteristic | Euler characteristic | | | | | | |
| | | curve (ECC) | curve (ECC) | | | | | | |
| | ECT_496 | Euler characteristic | Euler characteristic | | | | | | |
| | | curve (ECC) | curve (ECC) | | | | | | |
| | ECT_497 | Euler characteristic | Euler characteristic | | | | | | |
| | | curve (ECC) | curve (ECC) | | | | | | |
| | ECT_498 | Euler characteristic | Euler characteristic | | | | | | |
| | | curve (ECC) | curve (ECC) | | | | | | |
| | ECT_499 | Euler characteristic | Euler characteristic | | | | | | |
| | | curve (ECC) | curve (ECC) | | | | | | |
| | ECT_500 | Euler characteristic | Euler characteristic | | | | | | |
| | | curve (ECC) | curve (ECC) | | | | | | |
| | ECT_501 | Euler characteristic | Euler characteristic | | | | | | |
| | | curve (ECC) | curve (ECC) | | | | | | |
| | ECT_502 | Euler characteristic | Euler characteristic | | | | | | |
| | | curve (ECC) | curve (ECC) | | | | | | |
| | ECT_503 | Euler characteristic | Euler characteristic | | | | | | |
| | | curve (ECC) | curve (ECC) | | | | | | |
| | ECT_504 | Euler characteristic | Euler characteristic | | | | | | |
| | | curve (ECC) | curve (ECC) | | | | | | |
| | ECT_505 | Euler characteristic | Euler characteristic | | | | | | |
| | | curve (ECC) | curve (ECC) | | | | | | |
| | ECT_506 | Euler characteristic | Euler characteristic | | | | | | |
| | | curve (ECC) | curve (ECC) | | | | | | |
| | ECT_507 | Euler characteristic | Euler characteristic | | | | | | |
| | | curve (ECC) | curve (ECC) | | | | | | |
| | ECT_508 | Euler characteristic | Euler characteristic | | | | | | |
| | | curve (ECC) | curve (ECC) | | | | | | |
| | ECT_509 | Euler characteristic | Euler characteristic | | | | | | |
| | | curve (ECC) | curve (ECC) | | | | | | |
| | ECT_510 | Euler characteristic | Euler characteristic | | | | | | |
| | | curve (ECC) | curve (ECC) | | | | | | |
| | ECT_511 | Euler characteristic | Euler characteristic | | | | | | |
| | | curve (ECC) | curve (ECC) | | | | | | |
| | ECT_512 | Euler characteristic | Euler characteristic | | | | | | |
| | | curve (ECC) | curve (ECC) | | | | | | |
| | ECT_513 | Euler characteristic | Euler characteristic | | | | | | |
| | | curve (ECC) | curve (ECC) | | | | | | |
| | ECT_514 | Euler characteristic | Euler characteristic | | | | | | |
| | | curve (ECC) | curve (ECC) | | | | | | |
| | ECT_515 | Euler characteristic | Euler characteristic | | | | | | |
| | | curve (ECC) | curve (ECC) | | | | | | |
| | ECT_516 | Euler characteristic | Euler characteristic | | | | | | |
| | | curve (ECC) | curve (ECC) | | | | | | |
| | ECT_517 | Euler characteristic | Euler characteristic | | | | | | |
| | | curve (ECC) | curve (ECC) | | | | | | |
| | ECT_518 | Euler characteristic | Euler characteristic | | | | | | |
| | | curve (ECC) | curve (ECC) | | | | | | |
| | ECT_519 | Euler characteristic | Euler characteristic | | | | | | |
| | | curve (ECC) | curve (ECC) | | | | | | |
| | ECT_520 | Euler characteristic | Euler characteristic | | | | | | |
| | | curve (ECC) | curve (ECC) | | | | | | |
| | ECT_521 | Euler characteristic | Euler characteristic | | | | | | |
| | | curve (ECC) | curve (ECC) | | | | | | |
| | ECT_522 | Euler characteristic | Euler characteristic | | | | | | |
| | | curve (ECC) | curve (ECC) | | | | | | |
| | ECT_523 | Euler characteristic | Euler characteristic | | | | | | |
| | | curve (ECC) | curve (ECC) | | | | | | |
| | ECT_524 | Euler characteristic | Euler characteristic | | | | | | |
| | | curve (ECC) | curve (ECC) | | | | | | |
| | ECT_525 | Euler characteristic | Euler characteristic | | | | | | |
| | | curve (ECC) | curve (ECC) | | | | | | |
| | ECT_526 | Euler characteristic | Euler characteristic | | | | | | |
| | | curve (ECC) | curve (ECC) | | | | | | |
| | ECT_527 | Euler characteristic | Euler characteristic | | | | | | |
| | | curve (ECC) | curve (ECC) | | | | | | |
| | ECT_528 | Euler characteristic | Euler characteristic | | | | | | |
| | | curve (ECC) | curve (ECC) | | | | | | |
| | ECT_529 | Euler characteristic | Euler characteristic | | | | | | |
| | | curve (ECC) | curve (ECC) | | | | | | |
| | ECT_530 | Euler characteristic | Euler characteristic | | | | | | |
| | | curve (ECC) | curve (ECC) | | | | | | |
| | ECT_531 | Euler characteristic | Euler characteristic | | | | | | |
| | | curve (ECC) | curve (ECC) | | | | | | |
| | ECT_532 | Euler characteristic | Euler characteristic | | | | | | |
| | | curve (ECC) | curve (ECC) | | | | | | |
| | ECT_533 | Euler characteristic | Euler characteristic | | | | | | |
| | | curve (ECC) | curve (ECC) | | | | | | |
| | ECT_534 | Euler characteristic | Euler characteristic | | | | | | |
| | | curve (ECC) | curve (ECC) | | | | | | |
| | ECT_535 | Euler characteristic | Euler characteristic | | | | | | |
| | | curve (ECC) | curve (ECC) | | | | | | |
| | ECT_536 | Euler characteristic | Euler characteristic | | | | | | |
| | | curve (ECC) | curve (ECC) | | | | | | |
| | ECT_537 | Euler characteristic | Euler characteristic | | | | | | |
| | | curve (ECC) | curve (ECC) | | | | | | |
| | ECT_538 | Euler characteristic | Euler characteristic | | | | | | |
| | | curve (ECC) | curve (ECC) | | | | | | |
| | ECT_539 | Euler characteristic | Euler characteristic | | | | | | |
| | | curve (ECC) | curve (ECC) | | | | | | |
| | ECT_540 | Euler characteristic | Euler characteristic | | | | | | |
| | | curve (ECC) | curve (ECC) | | | | | | |
| | ECT_541 | Euler characteristic | Euler characteristic | | | | | | |
| | | curve (ECC) | curve (ECC) | | | | | | |
| | ECT_542 | Euler characteristic | Euler characteristic | | | | | | |
| | | curve (ECC) | curve (ECC) | | | | | | |
| | ECT_543 | Euler characteristic | Euler characteristic | | | | | | |
| | | curve (ECC) | curve (ECC) | | | | | | |
| | ECT_544 | Euler characteristic | Euler characteristic | | | | | | |
| | | curve (ECC) | curve (ECC) | | | | | | |
| | ECT_545 | Euler characteristic | Euler characteristic | | | | | | |
| | | curve (ECC) | curve (ECC) | | | | | | |
| | ECT_546 | Euler characteristic | Euler characteristic | | | | | | |
| | | curve (ECC) | curve (ECC) | | | | | | |
| | ECT_547 | Euler characteristic | Euler characteristic | | | | | | |
| | | curve (ECC) | curve (ECC) | | | | | | |
| | ECT_548 | Euler characteristic | Euler characteristic | | | | | | |
| | | curve (ECC) | curve (ECC) | | | | | | |
| | ECT_549 | Euler characteristic | Euler characteristic | | | | | | |
| | | curve (ECC) | curve (ECC) | | | | | | |
| | ECT_550 | Euler characteristic | Euler characteristic | | | | | | |
| | | curve (ECC) | curve (ECC) | | | | | | |
| | ECT_551 | Euler characteristic | Euler characteristic | | | | | | |
| | | curve (ECC) | curve (ECC) | | | | | | |
| | ECT_552 | Euler characteristic | Euler characteristic | | | | | | |
| | | curve (ECC) | curve (ECC) | | | | | | |
| | ECT_553 | Euler characteristic | Euler characteristic | | | | | | |
| | | curve (ECC) | curve (ECC) | | | | | | |
| | ECT_554 | Euler characteristic | Euler characteristic | | | | | | |
| | | curve (ECC) | curve (ECC) | | | | | | |
| | ECT_555 | Euler characteristic | Euler characteristic | | | | | | |
| | | curve (ECC) | curve (ECC) | | | | | | |
| | ECT_556 | Euler characteristic | Euler characteristic | | | | | | |
| | | curve (ECC) | curve (ECC) | | | | | | |
| | ECT_557 | Euler characteristic | Euler characteristic | | | | | | |
| | | curve (ECC) | curve (ECC) | | | | | | |
| | ECT_558 | Euler characteristic | Euler characteristic | | | | | | |
| | | curve (ECC) | curve (ECC) | | | | | | |
| | ECT_559 | Euler characteristic | Euler characteristic | | | | | | |
| | | curve (ECC) | curve (ECC) | | | | | | |
| | ECT_560 | Euler characteristic | Euler characteristic | | | | | | |
| | | curve (ECC) | curve (ECC) | | | | | | |
| | ECT_561 | Euler characteristic | Euler characteristic | | | | | | |
| | | curve (ECC) | curve (ECC) | | | | | | |
| | ECT_562 | Euler characteristic | Euler characteristic | | | | | | |
| | | curve (ECC) | curve (ECC) | | | | | | |
| | ECT_563 | Euler characteristic | Euler characteristic | | | | | | |
| | | curve (ECC) | curve (ECC) | | | | | | |
| | ECT_564 | Euler characteristic | Euler characteristic | | | | | | |
| | | curve (ECC) | curve (ECC) | | | | | | |
| | ECT_565 | Euler characteristic | Euler characteristic | | | | | | |
| | | curve (ECC) | curve (ECC) | | | | | | |
| | ECT_566 | Euler characteristic | Euler characteristic | | | | | | |
| | | curve (ECC) | curve (ECC) | | | | | | |
| | ECT_567 | Euler characteristic | Euler characteristic | | | | | | |
| | | curve (ECC) | curve (ECC) | | | | | | |
| | ECT_568 | Euler characteristic | Euler characteristic | | | | | | |
| | | curve (ECC) | curve (ECC) | | | | | | |
| | ECT_569 | Euler characteristic | Euler characteristic | | | | | | |
| | | curve (ECC) | curve (ECC) | | | | | | |
| | ECT_570 | Euler characteristic | Euler characteristic | | | | | | |
| | | curve (ECC) | curve (ECC) | | | | | | |
| | ECT_571 | Euler characteristic | Euler characteristic | | | | | | |
| | | curve (ECC) | curve (ECC) | | | | | | |
| | ECT_572 | Euler characteristic | Euler characteristic | | | | | | |
| | | curve (ECC) | curve (ECC) | | | | | | |
| | ECT_573 | Euler characteristic | Euler characteristic | | | | | | |
| | | curve (ECC) | curve (ECC) | | | | | | |
| | ECT_574 | Euler characteristic | Euler characteristic | | | | | | |
| | | curve (ECC) | curve (ECC) | | | | | | |
| | ECT_575 | Euler characteristic | Euler characteristic | | | | | | |
| | | curve (ECC) | curve (ECC) | | | | | | |
| | ECT_576 | Euler characteristic | Euler characteristic | | | | | | |
| | | curve (ECC) | curve (ECC) | | | | | | |
| | ECT_577 | Euler characteristic | Euler characteristic | | | | | | |
| | | curve (ECC) | curve (ECC) | | | | | | |
| | ECT_578 | Euler characteristic | Euler characteristic | | | | | | |
| | | curve (ECC) | curve (ECC) | | | | | | |
| | ECT_579 | Euler characteristic | Euler characteristic | | | | | | |
| | | curve (ECC) | curve (ECC) | | | | | | |
| | ECT_580 | Euler characteristic | Euler characteristic | | | | | | |
| | | curve (ECC) | curve (ECC) | | | | | | |
| | ECT_581 | Euler characteristic | Euler characteristic | | | | | | |
| | | curve (ECC) | curve (ECC) | | | | | | |
| | ECT_582 | Euler characteristic | Euler characteristic | | | | | | |
| | | curve (ECC) | curve (ECC) | | | | | | |
| | ECT_583 | Euler characteristic | Euler characteristic | | | | | | |
| | | curve (ECC) | curve (ECC) | | | | | | |
| | ECT_584 | Euler characteristic | Euler characteristic | | | | | | |
| | | curve (ECC) | curve (ECC) | | | | | | |
| | ECT_585 | Euler characteristic | Euler characteristic | | | | | | |
| | | curve (ECC) | curve (ECC) | | | | | | |
| | ECT_586 | Euler characteristic | Euler characteristic | | | | | | |
| | | curve (ECC) | curve (ECC) | | | | | | |
| | ECT_587 | Euler characteristic | Euler characteristic | | | | | | |
| | | curve (ECC) | curve (ECC) | | | | | | |
| | ECT_588 | Euler characteristic | Euler characteristic | | | | | | |
| | | curve (ECC) | curve (ECC) | | | | | | |
| | ECT_589 | Euler characteristic | Euler characteristic | | | | | | |
| | | curve (ECC) | curve (ECC) | | | | | | |
| | ECT_590 | Euler characteristic | Euler characteristic | | | | | | |
| | | curve (ECC) | curve (ECC) | | | | | | |
| | ECT_591 | Euler characteristic | Euler characteristic | | | | | | |
| | | curve (ECC) | curve (ECC) | | | | | | |
| | ECT_592 | Euler characteristic | Euler characteristic | | | | | | |
| | | curve (ECC) | curve (ECC) | | | | | | |
| | ECT_593 | Euler characteristic | Euler characteristic | | | | | | |
| | | curve (ECC) | curve (ECC) | | | | | | |
| | ECT_594 | Euler characteristic | Euler characteristic | | | | | | |
| | | curve (ECC) | curve (ECC) | | | | | | |
| | ECT_595 | Euler characteristic | Euler characteristic | | | | | | |
| | | curve (ECC) | curve (ECC) | | | | | | |
| | ECT_596 | Euler characteristic | Euler characteristic | | | | | | |
| | | curve (ECC) | curve (ECC) | | | | | | |
| | ECT_597 | Euler characteristic | Euler characteristic | | | | | | |
| | | curve (ECC) | curve (ECC) | | | | | | |
| | ECT_598 | Euler characteristic | Euler characteristic | | | | | | |
| | | curve (ECC) | curve (ECC) | | | | | | |
| | ECT_599 | Euler characteristic | Euler characteristic | | | | | | |
| | | curve (ECC) | curve (ECC) | | | | | | |
| | ECT_600 | Euler characteristic | Euler characteristic | | | | | | |
| | | curve (ECC) | curve (ECC) | | | | | | |
| | ECT_601 | Euler characteristic | Euler characteristic | | | | | | |
| | | curve (ECC) | curve (ECC) | | | | | | |
| | ECT_602 | Euler characteristic | Euler characteristic | | | | | | |
| | | curve (ECC) | curve (ECC) | | | | | | |
| | ECT_603 | Euler characteristic | Euler characteristic | | | | | | |
| | | curve (ECC) | curve (ECC) | | | | | | |
| | ECT_604 | Euler characteristic | Euler characteristic | | | | | | |
| | | curve (ECC) | curve (ECC) | | | | | | |
| | ECT_605 | Euler characteristic | Euler characteristic | | | | | | |
| | | curve (ECC) | curve (ECC) | | | | | | |
| | ECT_606 | Euler characteristic | Euler characteristic | | | | | | |
| | | curve (ECC) | curve (ECC) | | | | | | |
| | ECT_607 | Euler characteristic | Euler characteristic | | | | | | |
| | | curve (ECC) | curve (ECC) | | | | | | |
| | ECT_608 | Euler characteristic | Euler characteristic | | | | | | |
| | | curve (ECC) | curve (ECC) | | | | | | |
| | ECT_609 | Euler characteristic | Euler characteristic | | | | | | |
| | | curve (ECC) | curve (ECC) | | | | | | |
| | ECT_610 | Euler characteristic | Euler characteristic | | | | | | |
| | | curve (ECC) | curve (ECC) | | | | | | |
| | ECT_611 | Euler characteristic | Euler characteristic | | | | | | |
| | | curve (ECC) | curve (ECC) | | | | | | |
| | ECT_612 | Euler characteristic | Euler characteristic | | | | | | |
| | | curve (ECC) | curve (ECC) | | | | | | |
| | ECT_613 | Euler characteristic | Euler characteristic | | | | | | |
| | | curve (ECC) | curve (ECC) | | | | | | |
| | ECT_614 | Euler characteristic | Euler characteristic | | | | | | |
| | | curve (ECC) | curve (ECC) | | | | | | |
| | ECT_615 | Euler characteristic | Euler characteristic | | | | | | |
| | | curve (ECC) | curve (ECC) | | | | | | |
| | ECT_616 | Euler characteristic | Euler characteristic | | | | | | |
| | | curve (ECC) | curve (ECC) | | | | | | |
| | ECT_617 | Euler characteristic | Euler characteristic | | | | | | |
| | | curve (ECC) | curve (ECC) | | | | | | |
| | ECT_618 | Euler characteristic | Euler characteristic | | | | | | |
| | | curve (ECC) | curve (ECC) | | | | | | |
| | ECT_619 | Euler characteristic | Euler characteristic | | | | | | |
| | | curve (ECC) | curve (ECC) | | | | | | |
| | ECT_620 | Euler characteristic | Euler characteristic | | | | | | |
| | | curve (ECC) | curve (ECC) | | | | | | |
| | ECT_621 | Euler characteristic | Euler characteristic | | | | | | |
| | | curve (ECC) | curve (ECC) | | | | | | |
| | ECT_622 | Euler characteristic | Euler characteristic | | | | | | |
| | | curve (ECC) | curve (ECC) | | | | | | |
| | ECT_623 | Euler characteristic | Euler characteristic | | | | | | |
| | | curve (ECC) | curve (ECC) | | | | | | |
| | ECT_624 | Euler characteristic | Euler characteristic | | | | | | |
| | | curve (ECC) | curve (ECC) | | | | | | |
| | ECT_625 | Euler characteristic | Euler characteristic | | | | | | |
| | | curve (ECC) | curve (ECC) | | | | | | |
| | ECT_626 | Euler characteristic | Euler characteristic | | | | | | |
| | | curve (ECC) | curve (ECC) | | | | | | |
| | ECT_627 | Euler characteristic | Euler characteristic | | | | | | |
| | | curve (ECC) | curve (ECC) | | | | | | |
| | ECT_628 | Euler characteristic | Euler characteristic | | | | | | |
| | | curve (ECC) | curve (ECC) | | | | | | |
| | ECT_629 | Euler characteristic | Euler characteristic | | | | | | |
| | | curve (ECC) | curve (ECC) | | | | | | |
| | ECT_630 | Euler characteristic | Euler characteristic | | | | | | |
| | | curve (ECC) | curve (ECC) | | | | | | |
| | ECT_631 | Euler characteristic | Euler characteristic | | | | | | |
| | | curve (ECC) | curve (ECC) | | | | | | |
| | ECT_632 | Euler characteristic | Euler characteristic | | | | | | |
| | | curve (ECC) | curve (ECC) | | | | | | |
| | ECT_633 | Euler characteristic | Euler characteristic | | | | | | |
| | | curve (ECC) | curve (ECC) | | | | | | |
| | ECT_634 | Euler characteristic | Euler characteristic | | | | | | |
| | | curve (ECC) | curve (ECC) | | | | | | |
| | ECT_635 | Euler characteristic | Euler characteristic | | | | | | |
| | | curve (ECC) | curve (ECC) | | | | | | |
| | ECT_636 | Euler characteristic | Euler characteristic | | | | | | |
| | | curve (ECC) | curve (ECC) | | | | | | |
| | ECT_637 | Euler characteristic | Euler characteristic | | | | | | |
| | | curve (ECC) | curve (ECC) | | | | | | |
| | ECT_638 | Euler characteristic | Euler characteristic | | | | | | |
| | | curve (ECC) | curve (ECC) | | | | | | |
| | ECT_639 | Euler characteristic | Euler characteristic | | | | | | |
| | | curve (ECC) | curve (ECC) | | | | | | |
| | ECT_640 | Euler characteristic | Euler characteristic | | | | | | |
| | | curve (ECC) | curve (ECC) | | | | | | |
| | ECT_641 | Euler characteristic | Euler characteristic | | | | | | |
| | | curve (ECC) | curve (ECC) | | | | | | |
| | ECT_642 | Euler characteristic | Euler characteristic | | | | | | |
| | | curve (ECC) | curve (ECC) | | | | | | |
| | ECT_643 | Euler characteristic | Euler characteristic | | | | | | |
| | | curve (ECC) | curve (ECC) | | | | | | |
| | ECT_644 | Euler characteristic | Euler characteristic | | | | | | |
| | | curve (ECC) | curve (ECC) | | | | | | |
| | ECT_645 | Euler characteristic | Euler characteristic | | | | | | |
| | | curve (ECC) | curve (ECC) | | | | | | |
| | ECT_646 | Euler characteristic | Euler characteristic | | | | | | |
| | | curve (ECC) | curve (ECC) | | | | | | |
| | ECT_647 | Euler characteristic | Euler characteristic | | | | | | |
| | | curve (ECC) | curve (ECC) | | | | | | |
| | ECT_648 | Euler characteristic | Euler characteristic | | | | | | |
| | | curve (ECC) | curve (ECC) | | | | | | |
| | ECT_649 | Euler characteristic | Euler characteristic | | | | | | |
| | | curve (ECC) | curve (ECC) | | | | | | |
| | ECT_650 | Euler characteristic | Euler characteristic | | | | | | |
| | | curve (ECC) | curve (ECC) | | | | | | |
| | ECT_651 | Euler characteristic | Euler characteristic | | | | | | |
| | | curve (ECC) | curve (ECC) | | | | | | |
| | ECT_652 | Euler characteristic | Euler characteristic | | | | | | |
| | | curve (ECC) | curve (ECC) | | | | | | |
| | ECT_653 | Euler characteristic | Euler characteristic | | | | | | |
| | | curve (ECC) | curve (ECC) | | | | | | |
| | ECT_654 | Euler characteristic | Euler characteristic | | | | | | |
| | | curve (ECC) | curve (ECC) | | | | | | |
| | ECT_655 | Euler characteristic | Euler characteristic | | | | | | |
| | | curve (ECC) | curve (ECC) | | | | | | |
| | ECT_656 | Euler characteristic | Euler characteristic | | | | | | |
| | | curve (ECC) | curve (ECC) | | | | | | |
| | ECT_657 | Euler characteristic | Euler characteristic | | | | | | |
| | | curve (ECC) | curve (ECC) | | | | | | |
| | ECT_658 | Euler characteristic | Euler characteristic | | | | | | |
| | | curve (ECC) | curve (ECC) | | | | | | |
| | ECT_659 | Euler characteristic | Euler characteristic | | | | | | |
| | | curve (ECC) | curve (ECC) | | | | | | |
| | ECT_660 | Euler characteristic | Euler characteristic | | | | | | |
| | | curve (ECC) | curve (ECC) | | | | | | |
| | ECT_661 | Euler characteristic | Euler characteristic | | | | | | |
| | | curve (ECC) | curve (ECC) | | | | | | |
| | ECT_662 | Euler characteristic | Euler characteristic | | | | | | |
| | | curve (ECC) | curve (ECC) | | | | | | |
| | ECT_663 | Euler characteristic | Euler characteristic | | | | | | |
| | | curve (ECC) | curve (ECC) | | | | | | |
| | ECT_664 | Euler characteristic | Euler characteristic | | | | | | |
| | | curve (ECC) | curve (ECC) | | | | | | |
| | ECT_665 | Euler characteristic | Euler characteristic | | | | | | |
| | | curve (ECC) | curve (ECC) | | | | | | |
| | ECT_666 | Euler characteristic | Euler characteristic | | | | | | |
| | | curve (ECC) | curve (ECC) | | | | | | |
| | ECT_667 | Euler characteristic | Euler characteristic | | | | | | |
| | | curve (ECC) | curve (ECC) | | | | | | |
| | ECT_668 | Euler characteristic | Euler characteristic | | | | | | |
| | | curve (ECC) | curve (ECC) | | | | | | |
| | ECT_669 | Euler characteristic | Euler characteristic | | | | | | |
| | | curve (ECC) | curve (ECC) | | | | | | |
| | ECT_670 | Euler characteristic | Euler characteristic | | | | | | |
| | | curve (ECC) | curve (ECC) | | | | | | |
| | ECT_671 | Euler characteristic | Euler characteristic | | | | | | |
| | | curve (ECC) | curve (ECC) | | | | | | |
| | ECT_672 | Euler characteristic | Euler characteristic | | | | | | |
| | | curve (ECC) | curve (ECC) | | | | | | |
| | ECT_673 | Euler characteristic | Euler characteristic | | | | | | |
| | | curve (ECC) | curve (ECC) | | | | | | |
| | ECT_674 | Euler characteristic | Euler characteristic | | | | | | |
| | | curve (ECC) | curve (ECC) | | | | | | |
| | ECT_675 | Euler characteristic | Euler characteristic | | | | | | |
| | | curve (ECC) | curve (ECC) | | | | | | |
| | ECT_676 | Euler characteristic | Euler characteristic | | | | | | |
| | | curve (ECC) | curve (ECC) | | | | | | |
| | ECT_677 | Euler characteristic | Euler characteristic | | | | | | |
| | | curve (ECC) | curve (ECC) | | | | | | |
| | ECT_678 | Euler characteristic | Euler characteristic | | | | | | |
| | | curve (ECC) | curve (ECC) | | | | | | |
| | ECT_679 | Euler characteristic | Euler characteristic | | | | | | |
| | | curve (ECC) | curve (ECC) | | | | | | |
| | ECT_680 | Euler characteristic | Euler characteristic | | | | | | |
| | | curve (ECC) | curve (ECC) | | | | | | |
| | ECT_681 | Euler characteristic | Euler characteristic | | | | | | |
| | | curve (ECC) | curve (ECC) | | | | | | |
| | ECT_682 | Euler characteristic | Euler characteristic | | | | | | |
| | | curve (ECC) | curve (ECC) | | | | | | |
| | ECT_683 | Euler characteristic | Euler characteristic | | | | | | |
| | | curve (ECC) | curve (ECC) | | | | | | |
| | ECT_684 | Euler characteristic | Euler characteristic | | | | | | |
| | | curve (ECC) | curve (ECC) | | | | | | |
| | ECT_685 | Euler characteristic | Euler characteristic | | | | | | |
| | | curve (ECC) | curve (ECC) | | | | | | |
| | ECT_686 | Euler characteristic | Euler characteristic | | | | | | |
| | | curve (ECC) | curve (ECC) | | | | | | |
| | ECT_687 | Euler characteristic | Euler characteristic | | | | | | |
| | | curve (ECC) | curve (ECC) | | | | | | |
| | ECT_688 | Euler characteristic | Euler characteristic | | | | | | |
| | | curve (ECC) | curve (ECC) | | | | | | |
| | ECT_689 | Euler characteristic | Euler characteristic | | | | | | |
| | | curve (ECC) | curve (ECC) | | | | | | |
| | ECT_690 | Euler characteristic | Euler characteristic | | | | | | |
| | | curve (ECC) | curve (ECC) | | | | | | |
| | ECT_691 | Euler characteristic | Euler characteristic | | | | | | |
| | | curve (ECC) | curve (ECC) | | | | | | |
| | ECT_692 | Euler characteristic | Euler characteristic | | | | | | |
| | | curve (ECC) | curve (ECC) | | | | | | |
| | ECT_693 | Euler characteristic | Euler characteristic | | | | | | |
| | | curve (ECC) | curve (ECC) | | | | | | |
| | ECT_694 | Euler characteristic | Euler characteristic | | | | | | |
| | | curve (ECC) | curve (ECC) | | | | | | |
| | ECT_695 | Euler characteristic | Euler characteristic | | | | | | |
| | | curve (ECC) | curve (ECC) | | | | | | |
| | ECT_696 | Euler characteristic | Euler characteristic | | | | | | |
| | | curve (ECC) | curve (ECC) | | | | | | |
| | ECT_697 | Euler characteristic | Euler characteristic | | | | | | |
| | | curve (ECC) | curve (ECC) | | | | | | |
| | ECT_698 | Euler characteristic | Euler characteristic | | | | | | |
| | | curve (ECC) | curve (ECC) | | | | | | |
| | ECT_699 | Euler characteristic | Euler characteristic | | | | | | |
| | | curve (ECC) | curve (ECC) | | | | | | |
| | ECT_700 | Euler characteristic | Euler characteristic | | | | | | |
| | | curve (ECC) | curve (ECC) | | | | | | |
| | ECT_701 | Euler characteristic | Euler characteristic | | | | | | |
| | | curve (ECC) | curve (ECC) | | | | | | |
| | ECT_702 | Euler characteristic | Euler characteristic | | | | | | |
| | | curve (ECC) | curve (ECC) | | | | | | |
| | ECT_703 | Euler characteristic | Euler characteristic | | | | | | |
| | | curve (ECC) | curve (ECC) | | | | | | |
| | ECT_704 | Euler characteristic | Euler characteristic | | | | | | |
| | | curve (ECC) | curve (ECC) | | | | | | |
| | ECT_705 | Euler characteristic | Euler characteristic | | | | | | |
| | | curve (ECC) | curve (ECC) | | | | | | |
| | ECT_706 | Euler characteristic | Euler characteristic | | | | | | |
| | | curve (ECC) | curve (ECC) | | | | | | |
| | ECT_707 | Euler characteristic | Euler characteristic | | | | | | |
| | | curve (ECC) | curve (ECC) | | | | | | |
| | ECT_708 | Euler characteristic | Euler characteristic | | | | | | |
| | | curve (ECC) | curve (ECC) | | | | | | |
| | ECT_709 | Euler characteristic | Euler characteristic | | | | | | |
| | | curve (ECC) | curve (ECC) | | | | | | |
| | ECT_710 | Euler characteristic | Euler characteristic | | | | | | |
| | | curve (ECC) | curve (ECC) | | | | | | |
| | ECT_711 | Euler characteristic | Euler characteristic | | | | | | |
| | | curve (ECC) | curve (ECC) | | | | | | |
| | ECT_712 | Euler characteristic | Euler characteristic | | | | | | |
| | | curve (ECC) | curve (ECC) | | | | | | |
| | ECT_713 | Euler characteristic | Euler characteristic | | | | | | |
| | | curve (ECC) | curve (ECC) | | | | | | |
| | ECT_714 | Euler characteristic | Euler characteristic | | | | | | |
| | | curve (ECC) | curve (ECC) | | | | | | |
| | ECT_715 | Euler characteristic | Euler characteristic | | | | | | |
| | | curve (ECC) | curve (ECC) | | | | | | |
| | ECT_716 | Euler characteristic | Euler characteristic | | | | | | |
| | | curve (ECC) | curve (ECC) | | | | | | |
| | ECT_717 | Euler characteristic | Euler characteristic | | | | | | |
| | | curve (ECC) | curve (ECC) | | | | | | |
| | ECT_718 | Euler characteristic | Euler characteristic | | | | | | |
| | | curve (ECC) | curve (ECC) | | | | | | |
| | ECT_719 | Euler characteristic | Euler characteristic | | | | | | |
| | | curve (ECC) | curve (ECC) | | | | | | |
| | ECT_720 | Euler characteristic | Euler characteristic | | | | | | |
| | | curve (ECC) | curve (ECC) | | | | | | |
| | ECT_721 | Euler characteristic | Euler characteristic | | | | | | |
| | | curve (ECC) | curve (ECC) | | | | | | |
| | ECT_722 | Euler characteristic | Euler characteristic | | | | | | |
| | | curve (ECC) | curve (ECC) | | | | | | |
| | ECT_723 | Euler characteristic | Euler characteristic | | | | | | |
| | | curve (ECC) | curve (ECC) | | | | | | |
| | ECT_724 | Euler characteristic | Euler characteristic | | | | | | |
| | | curve (ECC) | curve (ECC) | | | | | | |
| | ECT_725 | Euler characteristic | Euler characteristic | | | | | | |
| | | curve (ECC) | curve (ECC) | | | | | | |
| | ECT_726 | Euler characteristic | Euler characteristic | | | | | | |
| | | curve (ECC) | curve (ECC) | | | | | | |
| | ECT_727 | Euler characteristic | Euler characteristic | | | | | | |
| | | curve (ECC) | curve (ECC) | | | | | | |
| | ECT_728 | Euler characteristic | Euler characteristic | | | | | | |
| | | curve (ECC) | curve (ECC) | | | | | | |
| | ECT_729 | Euler characteristic | Euler characteristic | | | | | | |
| | | curve (ECC) | curve (ECC) | | | | | | |
| | ECT_730 | Euler characteristic | Euler characteristic | | | | | | |
| | | curve (ECC) | curve (ECC) | | | | | | |
| | ECT_731 | Euler characteristic | Euler characteristic | | | | | | |
| | | curve (ECC) | curve (ECC) | | | | | | |
| | ECT_732 | Euler characteristic | Euler characteristic | | | | | | |
| | | curve (ECC) | curve (ECC) | | | | | | |
| | ECT_733 | Euler characteristic | Euler characteristic | | | | | | |
| | | curve (ECC) | curve (ECC) | | | | | | |
| | ECT_734 | Euler characteristic | Euler characteristic | | | | | | |
| | | curve (ECC) | curve (ECC) | | | | | | |
| | ECT_735 | Euler characteristic | Euler characteristic | | | | | | |
| | | curve (ECC) | curve (ECC) | | | | | | |
| | ECT_736 | Euler characteristic | Euler characteristic | | | | | | |
| | | curve (ECC) | curve (ECC) | | | | | | |
| | ECT_737 | Euler characteristic | Euler characteristic | | | | | | |
| | | curve (ECC) | curve (ECC) | | | | | | |
| | ECT_738 | Euler characteristic | Euler characteristic | | | | | | |
| | | curve (ECC) | curve (ECC) | | | | | | |
| | ECT_739 | Euler characteristic | Euler characteristic | | | | | | |
| | | curve (ECC) | curve (ECC) | | | | | | |
| | ECT_740 | Euler characteristic | Euler characteristic | | | | | | |
| | | curve (ECC) | curve (ECC) | | | | | | |
| | ECT_741 | Euler characteristic | Euler characteristic | | | | | | |
| | | curve (ECC) | curve (ECC) | | | | | | |
| | ECT_742 | Euler characteristic | Euler characteristic | | | | | | |
| | | curve (ECC) | curve (ECC) | | | | | | |
| | ECT_743 | Euler characteristic | Euler characteristic | | | | | | |
| | | curve (ECC) | curve (ECC) | | | | | | |
| | ECT_744 | Euler characteristic | Euler characteristic | | | | | | |
| | | curve (ECC) | curve (ECC) | | | | | | |
| | ECT_745 | Euler characteristic | Euler characteristic | | | | | | |
| | | curve (ECC) | curve (ECC) | | | | | | |
| | ECT_746 | Euler characteristic | Euler characteristic | | | | | | |
| | | curve (ECC) | curve (ECC) | | | | | | |
| | ECT_747 | Euler characteristic | Euler characteristic | | | | | | |
| | | curve (ECC) | curve (ECC) | | | | | | |
| | ECT_748 | Euler characteristic | Euler characteristic | | | | | | |
| | | curve (ECC) | curve (ECC) | | | | | | |
| | ECT_749 | Euler characteristic | Euler characteristic | | | | | | |
| | | curve (ECC) | curve (ECC) | | | | | | |
| | ECT_750 | Euler characteristic | Euler characteristic | | | | | | |
| | | curve (ECC) | curve (ECC) | | | | | | |
| | ECT_751 | Euler characteristic | Euler characteristic | | | | | | |
| | | curve (ECC) | curve (ECC) | | | | | | |
| | ECT_752 | Euler characteristic | Euler characteristic | | | | | | |
| | | curve (ECC) | curve (ECC) | | | | | | |
| | ECT_753 | Euler characteristic | Euler characteristic | | | | | | |
| | | curve (ECC) | curve (ECC) | | | | | | |
| | ECT_754 | Euler characteristic | Euler characteristic | | | | | | |
| | | curve (ECC) | curve (ECC) | | | | | | |
| | ECT_755 | Euler characteristic | Euler characteristic | | | | | | |
| | | curve (ECC) | curve (ECC) | | | | | | |
| | ECT_756 | Euler characteristic | Euler characteristic | | | | | | |
| | | curve (ECC) | curve (ECC) | | | | | | |
| | ECT_757 | Euler characteristic | Euler characteristic | | | | | | |
| | | curve (ECC) | curve (ECC) | | | | | | |
| | ECT_758 | Euler characteristic | Euler characteristic | | | | | | |
| | | curve (ECC) | curve (ECC) | | | | | | |
| | ECT_759 | Euler characteristic | Euler characteristic | | | | | | |
| | | curve (ECC) | curve (ECC) | | | | | | |
| | ECT_760 | Euler characteristic | Euler characteristic | | | | | | |
| | | curve (ECC) | curve (ECC) | | | | | | |
| | ECT_761 | Euler characteristic | Euler characteristic | | | | | | |
| | | curve (ECC) | curve (ECC) | | | | | | |
| | ECT_762 | Euler characteristic | Euler characteristic | | | | | | |
| | | curve (ECC) | curve (ECC) | | | | | | |
| | ECT_763 | Euler characteristic | Euler characteristic | | | | | | |
| | | curve (ECC) | curve (ECC) | | | | | | |
| | ECT_764 | Euler characteristic | Euler characteristic | | | | | | |
| | | curve (ECC) | curve (ECC) | | | | | | |
| | ECT_765 | Euler characteristic | Euler characteristic | | | | | | |
| | | curve (ECC) | curve (ECC) | | | | | | |
| | ECT_766 | Euler characteristic | Euler characteristic | | | | | | |
| | | curve (ECC) | curve (ECC) | | | | | | |
| | ECT_767 | Euler characteristic | Euler characteristic | | | | | | |
| | | curve (ECC) | curve (ECC) | | | | | | |
| | ECT_768 | Euler characteristic | Euler characteristic | | | | | | |
| | | curve (ECC) | curve (ECC) | | | | | | |
| | ECT_769 | Euler characteristic | Euler characteristic | | | | | | |
| | | curve (ECC) | curve (ECC) | | | | | | |
| | ECT_770 | Euler characteristic | Euler characteristic | | | | | | |
| | | curve (ECC) | curve (ECC) | | | | | | |
| | ECT_771 | Euler characteristic | Euler characteristic | | | | | | |
| | | curve (ECC) | curve (ECC) | | | | | | |
| | ECT_772 | Euler characteristic | Euler characteristic | | | | | | |
| | | curve (ECC) | curve (ECC) | | | | | | |
| | ECT_773 | Euler characteristic | Euler characteristic | | | | | | |
| | | curve (ECC) | curve (ECC) | | | | | | |
| | ECT_774 | Euler characteristic | Euler characteristic | | | | | | |
| | | curve (ECC) | curve (ECC) | | | | | | |
| | ECT_775 | Euler characteristic | Euler characteristic | | | | | | |
| | | curve (ECC) | curve (ECC) | | | | | | |
| | ECT_776 | Euler characteristic | Euler characteristic | | | | | | |
| | | curve (ECC) | curve (ECC) | | | | | | |
| | ECT_777 | Euler characteristic | Euler characteristic | | | | | | |
| | | curve (ECC) | curve (ECC) | | | | | | |
| | ECT_778 | Euler characteristic | Euler characteristic | | | | | | |
| | | curve (ECC) | curve (ECC) | | | | | | |
| | ECT_779 | Euler characteristic | Euler characteristic | | | | | | |
| | | curve (ECC) | curve (ECC) | | | | | | |
| | ECT_780 | Euler characteristic | Euler characteristic | | | | | | |
| | | curve (ECC) | curve (ECC) | | | | | | |
| | ECT_781 | Euler characteristic | Euler characteristic | | | | | | |
| | | curve (ECC) | curve (ECC) | | | | | | |
| | ECT_782 | Euler characteristic | Euler characteristic | | | | | | |
| | | curve (ECC) | curve (ECC) | | | | | | |
| | ECT_783 | Euler characteristic | Euler characteristic | | | | | | |
| | | curve (ECC) | curve (ECC) | | | | | | |
| | ECT_784 | Euler characteristic | Euler characteristic | | | | | | |
| | | curve (ECC) | curve (ECC) | | | | | | |
| | ECT_785 | Euler characteristic | Euler characteristic | | | | | | |
| | | curve (ECC) | curve (ECC) | | | | | | |
| | ECT_786 | Euler characteristic | Euler characteristic | | | | | | |
| | | curve (ECC) | curve (ECC) | | | | | | |
| | ECT_787 | Euler characteristic | Euler characteristic | | | | | | |
| | | curve (ECC) | curve (ECC) | | | | | | |
| | ECT_788 | Euler characteristic | Euler characteristic | | | | | | |
| | | curve (ECC) | curve (ECC) | | | | | | |
| | ECT_789 | Euler characteristic | Euler characteristic | | | | | | |
| | | curve (ECC) | curve (ECC) | | | | | | |
| | ECT_790 | Euler characteristic | Euler characteristic | | | | | | |
| | | curve (ECC) | curve (ECC) | | | | | | |
| | ECT_791 | Euler characteristic | Euler characteristic | | | | | | |
| | | curve (ECC) | curve (ECC) | | | | | | |
| | ECT_792 | Euler characteristic | Euler characteristic | | | | | | |
| | | curve (ECC) | curve (ECC) | | | | | | |
| | ECT_793 | Euler characteristic | Euler characteristic | | | | | | |
| | | curve (ECC) | curve (ECC) | | | | | | |
| | ECT_794 | Euler characteristic | Euler characteristic | | | | | | |
| | | curve (ECC) | curve (ECC) | | | | | | |
| | ECT_795 | Euler characteristic | Euler characteristic | | | | | | |
| | | curve (ECC) | curve (ECC) | | | | | | |
| | ECT_796 | Euler characteristic | Euler characteristic | | | | | | |
| | | curve (ECC) | curve (ECC) | | | | | | |
| | ECT_797 | Euler characteristic | Euler characteristic | | | | | | |
| | | curve (ECC) | curve (ECC) | | | | | | |
| | ECT_798 | Euler characteristic | Euler characteristic | | | | | | |
| | | curve (ECC) | curve (ECC) | | | | | | |
| | ECT_799 | Euler characteristic | Euler characteristic | | | | | | |
| | | curve (ECC) | curve (ECC) | | | | | | |
| | ECT_800 | Euler characteristic | Euler characteristic | | | | | | |
| | | curve (ECC) | curve (ECC) | | | | | | |
| | ECT_801 | Euler characteristic | Euler characteristic | | | | | | |
| | | curve (ECC) | curve (ECC) | | | | | | |
| | ECT_802 | Euler characteristic | Euler characteristic | | | | | | |
| | | curve (ECC) | curve (ECC) | | | | | | |
| | ECT_803 | Euler characteristic | Euler characteristic | | | | | | |
| | | curve (ECC) | curve (ECC) | | | | | | |
| | ECT_804 | Euler characteristic | Euler characteristic | | | | | | |
| | | curve (ECC) | curve (ECC) | | | | | | |
| | ECT_805 | Euler characteristic | Euler characteristic | | | | | | |
| | | curve (ECC) | curve (ECC) | | | | | | |
| | ECT_806 | Euler characteristic | Euler characteristic | | | | | | |
| | | curve (ECC) | curve (ECC) | | | | | | |
| | ECT_807 | Euler characteristic | Euler characteristic | | | | | | |
| | | curve (ECC) | curve (ECC) | | | | | | |
| | ECT_808 | Euler characteristic | Euler characteristic | | | | | | |
| | | curve (ECC) | curve (ECC) | | | | | | |
| | ECT_809 | Euler characteristic | Euler characteristic | | | | | | |
| | | curve (ECC) | curve (ECC) | | | | | | |
| | ECT_810 | Euler characteristic | Euler characteristic | | | | | | |
| | | curve (ECC) | curve (ECC) | | | | | | |
| | ECT_811 | Euler characteristic | Euler characteristic | | | | | | |
| | | curve (ECC) | curve (ECC) | | | | | | |
| | ECT_812 | Euler characteristic | Euler characteristic | | | | | | |
| | | curve (ECC) | curve (ECC) | | | | | | |
| | ECT_813 | Euler characteristic | Euler characteristic | | | | | | |
| | | curve (ECC) | curve (ECC) | | | | | | |
| | ECT_814 | Euler characteristic | Euler characteristic | | | | | | |
| | | curve (ECC) | curve (ECC) | | | | | | |
| | ECT_815 | Euler characteristic | Euler characteristic | | | | | | |
| | | curve (ECC) | curve (ECC) | | | | | | |
| | ECT_816 | Euler characteristic | Euler characteristic | | | | | | |
| | | curve (ECC) | curve (ECC) | | | | | | |
| | ECT_817 | Euler characteristic | Euler characteristic | | | | | | |
| | | curve (ECC) | curve (ECC) | | | | | | |
| | ECT_818 | Euler characteristic | Euler characteristic | | | | | | |
| | | curve (ECC) | curve (ECC) | | | | | | |
| | ECT_819 | Euler characteristic | Euler characteristic | | | | | | |
| | | curve (ECC) | curve (ECC) | | | | | | |
| | ECT_820 | Euler characteristic | Euler characteristic | | | | | | |
| | | curve (ECC) | curve (ECC) | | | | | | |
| | ECT_821 | Euler characteristic | Euler characteristic | | | | | | |
| | | curve (ECC) | curve (ECC) | | | | | | |
| | ECT_822 | Euler characteristic | Euler characteristic | | | | | | |
| | | curve (ECC) | curve (ECC) | | | | | | |
| | ECT_823 | Euler characteristic | Euler characteristic | | | | | | |
| | | curve (ECC) | curve (ECC) | | | | | | |
| | ECT_824 | Euler characteristic | Euler characteristic | | | | | | |
| | | curve (ECC) | curve (ECC) | | | | | | |
| | ECT_825 | Euler characteristic | Euler characteristic | | | | | | |
| | | curve (ECC) | curve (ECC) | | | | | | |
| | ECT_826 | Euler characteristic | Euler characteristic | | | | | | |
| | | curve (ECC) | curve (ECC) | | | | | | |
| | ECT_827 | Euler characteristic | Euler characteristic | | | | | | |
| | | curve (ECC) | curve (ECC) | | | | | | |
| | ECT_828 | Euler characteristic | Euler characteristic | | | | | | |
| | | curve (ECC) | curve (ECC) | | | | | | |
| | ECT_829 | Euler characteristic | Euler characteristic | | | | | | |
| | | curve (ECC) | curve (ECC) | | | | | | |
| | ECT_830 | Euler characteristic | Euler characteristic | | | | | | |
| | | curve (ECC) | curve (ECC) | | | | | | |
| | ECT_831 | Euler characteristic | Euler characteristic | | | | | | |
| | | curve (ECC) | curve (ECC) | | | | | | |
| | ECT_832 | Euler characteristic | Euler characteristic | | | | | | |
| | | curve (ECC) | curve (ECC) | | | | | | |
| | ECT_833 | Euler characteristic | Euler characteristic | | | | | | |
| | | curve (ECC) | curve (ECC) | | | | | | |
| | ECT_834 | Euler characteristic | Euler characteristic | | | | | | |
| | | curve (ECC) | curve (ECC) | | | | | | |
| | ECT_835 | Euler characteristic | Euler characteristic | | | | | | |
| | | curve (ECC) | curve (ECC) | | | | | | |
| | ECT_836 | Euler characteristic | Euler characteristic | | | | | | |
| | | curve (ECC) | curve (ECC) | | | | | | |
| | ECT_837 | Euler characteristic | Euler characteristic | | | | | | |
| | | curve (ECC) | curve (ECC) | | | | | | |
| | ECT_838 | Euler characteristic | Euler characteristic | | | | | | |
| | | curve (ECC) | curve (ECC) | | | | | | |
| | ECT_839 | Euler characteristic | Euler characteristic | | | | | | |
| | | curve (ECC) | curve (ECC) | | | | | | |
| | ECT_840 | Euler characteristic | Euler characteristic | | | | | | |
| | | curve (ECC) | curve (ECC) | | | | | | |
| | ECT_841 | Euler characteristic | Euler characteristic | | | | | | |
| | | curve (ECC) | curve (ECC) | | | | | | |
| | ECT_842 | Euler characteristic | Euler characteristic | | | | | | |
| | | curve (ECC) | curve (ECC) | | | | | | |
| | ECT_843 | Euler characteristic | Euler characteristic | | | | | | |
| | | curve (ECC) | curve (ECC) | | | | | | |
| | ECT_844 | Euler characteristic | Euler characteristic | | | | | | |
| | | curve (ECC) | curve (ECC) | | | | | | |
| | ECT_845 | Euler characteristic | Euler characteristic | | | | | | |
| | | curve (ECC) | curve (ECC) | | | | | | |
| | ECT_846 | Euler characteristic | Euler characteristic | | | | | | |
| | | curve (ECC) | curve (ECC) | | | | | | |
| | ECT_847 | Euler characteristic | Euler characteristic | | | | | | |
| | | curve (ECC) | curve (ECC) | | | | | | |
| | ECT_848 | Euler characteristic | Euler characteristic | | | | | | |
| | | curve (ECC) | curve (ECC) | | | | | | |
| | ECT_849 | Euler characteristic | Euler characteristic | | | | | | |
| | | curve (ECC) | curve (ECC) | | | | | | |
| | ECT_850 | Euler characteristic | Euler characteristic | | | | | | |
| | | curve (ECC) | curve (ECC) | | | | | | |
| | ECT_851 | Euler characteristic | Euler characteristic | | | | | | |
| | | curve (ECC) | curve (ECC) | | | | | | |
| | ECT_852 | Euler characteristic | Euler characteristic | | | | | | |
| | | curve (ECC) | curve (ECC) | | | | | | |
| | ECT_853 | Euler characteristic | Euler characteristic | | | | | | |
| | | curve (ECC) | curve (ECC) | | | | | | |
| | ECT_854 | Euler characteristic | Euler characteristic | | | | | | |
| | | curve (ECC) | curve (ECC) | | | | | | |
| | ECT_855 | Euler characteristic | Euler characteristic | | | | | | |
| | | curve (ECC) | curve (ECC) | | | | | | |
| | ECT_856 | Euler characteristic | Euler characteristic | | | | | | |
| | | curve (ECC) | curve (ECC) | | | | | | |
| | ECT_857 | Euler characteristic | Euler characteristic | | | | | | |
| | | curve (ECC) | curve (ECC) | | | | | | |
| | ECT_858 | Euler characteristic | Euler characteristic | | | | | | |
| | | curve (ECC) | curve (ECC) | | | | | | |
| | ECT_859 | Euler characteristic | Euler characteristic | | | | | | |
| | | curve (ECC) | curve (ECC) | | | | | | |
| | ECT_860 | Euler characteristic | Euler characteristic | | | | | | |
| | | curve (ECC) | curve (ECC) | | | | | | |
| | ECT_861 | Euler characteristic | Euler characteristic | | | | | | |
| | | curve (ECC) | curve (ECC) | | | | | | |
| | ECT_862 | Euler characteristic | Euler characteristic | | | | | | |
| | | curve (ECC) | curve (ECC) | | | | | | |
| | ECT_863 | Euler characteristic | Euler characteristic | | | | | | |
| | | curve (ECC) | curve (ECC) | | | | | | |
| | ECT_864 | Euler characteristic | Euler characteristic | | | | | | |
| | | curve (ECC) | curve (ECC) | | | | | | |
| | ECT_865 | Euler characteristic | Euler characteristic | | | | | | |
| | | curve (ECC) | curve (ECC) | | | | | | |
| | ECT_866 | Euler characteristic | Euler characteristic | | | | | | |
| | | curve (ECC) | curve (ECC) | | | | | | |
| | ECT_867 | Euler characteristic | Euler characteristic | | | | | | |
| | | curve (ECC) | curve (ECC) | | | | | | |
| | ECT_868 | Euler characteristic | Euler characteristic | | | | | | |
| | | curve (ECC) | curve (ECC) | | | | | | |
| | ECT_869 | Euler characteristic | Euler characteristic | | | | | | |
| | | curve (ECC) | curve (ECC) | | | | | | |
| | ECT_870 | Euler characteristic | Euler characteristic | | | | | | |
| | | curve (ECC) | curve (ECC) | | | | | | |
| | ECT_871 | Euler characteristic | Euler characteristic | | | | | | |
| | | curve (ECC) | curve (ECC) | | | | | | |
| | ECT_872 | Euler characteristic | Euler characteristic | | | | | | |
| | | curve (ECC) | curve (ECC) | | | | | | |
| | ECT_873 | Euler characteristic | Euler characteristic | | | | | | |
| | | curve (ECC) | curve (ECC) | | | | | | |
| | ECT_874 | Euler characteristic | Euler characteristic | | | | | | |
| | | curve (ECC) | curve (ECC) | | | | | | |
| | ECT_875 | Euler characteristic | Euler characteristic | | | | | | |
| | | curve (ECC) | curve (ECC) | | | | | | |
| | ECT_876 | Euler characteristic | Euler characteristic | | | | | | |
| | | curve (ECC) | curve (ECC) | | | | | | |
| | ECT_877 | Euler characteristic | Euler characteristic | | | | | | |
| | | curve (ECC) | curve (ECC) | | | | | | |
| | ECT_878 | Euler characteristic | Euler characteristic | | | | | | |
| | | curve (ECC) | curve (ECC) | | | | | | |
| | ECT_879 | Euler characteristic | Euler characteristic | | | | | | |
| | | curve (ECC) | curve (ECC) | | | | | | |
| | ECT_880 | Euler characteristic | Euler characteristic | | | | | | |
| | | curve (ECC) | curve (ECC) | | | | | | |
| | ECT_881 | Euler characteristic | Euler characteristic | | | | | | |
| | | curve (ECC) | curve (ECC) | | | | | | |
| | ECT_882 | Euler characteristic | Euler characteristic | | | | | | |
| | | curve (ECC) | curve (ECC) | | | | | | |
| | ECT_883 | Euler characteristic | Euler characteristic | | | | | | |
| | | curve (ECC) | curve (ECC) | | | | | | |
| | ECT_884 | Euler characteristic | Euler characteristic | | | | | | |
| | | curve (ECC) | curve (ECC) | | | | | | |
| | ECT_885 | Euler characteristic | Euler characteristic | | | | | | |
| | | curve (ECC) | curve (ECC) | | | | | | |
| | ECT_886 | Euler characteristic | Euler characteristic | | | | | | |
| | | curve (ECC) | curve (ECC) | | | | | | |
| | ECT_887 | Euler characteristic | Euler characteristic | | | | | | |
| | | curve (ECC) | curve (ECC) | | | | | | |
| | ECT_888 | Euler characteristic | Euler characteristic | | | | | | |
| | | curve (ECC) | curve (ECC) | | | | | | |
| | ECT_889 | Euler characteristic | Euler characteristic | | | | | | |
| | | curve (ECC) | curve (ECC) | | | | | | |
| | ECT_890 | Euler characteristic | Euler characteristic | | | | | | |
| | | curve (ECC) | curve (ECC) | | | | | | |
| | ECT_891 | Euler characteristic | Euler characteristic | | | | | | |
| | | curve (ECC) | curve (ECC) | | | | | | |
| | ECT_892 | Euler characteristic | Euler characteristic | | | | | | |
| | | curve (ECC) | curve (ECC) | | | | | | |
| | ECT_893 | Euler characteristic | Euler characteristic | | | | | | |
| | | curve (ECC) | curve (ECC) | | | | | | |
| | ECT_894 | Euler characteristic | Euler characteristic | | | | | | |
| | | curve (ECC) | curve (ECC) | | | | | | |
| | ECT_895 | Euler characteristic | Euler characteristic | | | | | | |
| | | curve (ECC) | curve (ECC) | | | | | | |
| | ECT_896 | Euler characteristic | Euler characteristic | | | | | | |
| | | curve (ECC) | curve (ECC) | | | | | | |
| | ECT_897 | Euler characteristic | Euler characteristic | | | | | | |
| | | curve (ECC) | curve (ECC) | | | | | | |
| | ECT_898 | Euler characteristic | Euler characteristic | | | | | | |
| | | curve (ECC) | curve (ECC) | | | | | | |
| | ECT_899 | Euler characteristic | Euler characteristic | | | | | | |
| | | curve (ECC) | curve (ECC) | | | | | | |
| | ECT_900 | Euler characteristic | Euler characteristic | | | | | | |
| | | curve (ECC) | curve (ECC) | | | | | | |
| | ECT_901 | Euler characteristic | Euler characteristic | | | | | | |
| | | curve (ECC) | curve (ECC) | | | | | | |
| | ECT_902 | Euler characteristic | Euler characteristic | | | | | | |
| | | curve (ECC) | curve (ECC) | | | | | | |
| | ECT_903 | Euler characteristic | Euler characteristic | | | | | | |
| | | curve (ECC) | curve (ECC) | | | | | | |
| | ECT_904 | Euler characteristic | Euler characteristic | | | | | | |
| | | curve (ECC) | curve (ECC) | | | | | | |
| | ECT_905 | Euler characteristic | Euler characteristic | | | | | | |
| | | curve (ECC) | curve (ECC) | | | | | | |
| | ECT_906 | Euler characteristic | Euler characteristic | | | | | | |
| | | curve (ECC) | curve (ECC) | | | | | | |
| | ECT_907 | Euler characteristic | Euler characteristic | | | | | | |
| | | curve (ECC) | curve (ECC) | | | | | | |
| | ECT_908 | Euler characteristic | Euler characteristic | | | | | | |
| | | curve (ECC) | curve (ECC) | | | | | | |
| | ECT_909 | Euler characteristic | Euler characteristic | | | | | | |
| | | curve (ECC) | curve (ECC) | | | | | | |
| | ECT_910 | Euler characteristic | Euler characteristic | | | | | | |
| | | curve (ECC) | curve (ECC) | | | | | | |
| | ECT_911 | Euler characteristic | Euler characteristic | | | | | | |
| | | curve (ECC) | curve (ECC) | | | | | | |
| | ECT_912 | Euler characteristic | Euler characteristic | | | | | | |
| | | curve (ECC) | curve (ECC) | | | | | | |
| | ECT_913 | Euler characteristic | Euler characteristic | | | | | | |
| | | curve (ECC) | curve (ECC) | | | | | | |
| | ECT_914 | Euler characteristic | Euler characteristic | | | | | | |
| | | curve (ECC) | curve (ECC) | | | | | | |
| | ECT_915 | Euler characteristic | Euler characteristic | | | | | | |
| | | curve (ECC) | curve (ECC) | | | | | | |
| | ECT_916 | Euler characteristic | Euler characteristic | | | | | | |
| | | curve (ECC) | curve (ECC) | | | | | | |
| | ECT_917 | Euler characteristic | Euler characteristic | | | | | | |
| | | curve (ECC) | curve (ECC) | | | | | | |
| | ECT_918 | Euler characteristic | Euler characteristic | | | | | | |
| | | curve (ECC) | curve (ECC) | | | | | | |
| | ECT_919 | Euler characteristic | Euler characteristic | | | | | | |
| | | curve (ECC) | curve (ECC) | | | | | | |
| | ECT_920 | Euler characteristic | Euler characteristic | | | | | | |
| | | curve (ECC) | curve (ECC) | | | | | | |
| | ECT_921 | Euler characteristic | Euler characteristic | | | | | | |
| | | curve (ECC) | curve (ECC) | | | | | | |
| | ECT_922 | Euler characteristic | Euler characteristic | | | | | | |
| | | curve (ECC) | curve (ECC) | | | | | | |
| | ECT_923 | Euler characteristic | Euler characteristic | | | | | | |
| | | curve (ECC) | curve (ECC) | | | | | | |
| | ECT_924 | Euler characteristic | Euler characteristic | | | | | | |
| | | curve (ECC) | curve (ECC) | | | | | | |
| | ECT_925 | Euler characteristic | Euler characteristic | | | | | | |
| | | curve (ECC) | curve (ECC) | | | | | | |
| | ECT_926 | Euler characteristic | Euler characteristic | | | | | | |
| | | curve (ECC) | curve (ECC) | | | | | | |
| | ECT_927 | Euler characteristic | Euler characteristic | | | | | | |
| | | curve (ECC) | curve (ECC) | | | | | | |
| | ECT_928 | Euler characteristic | Euler characteristic | | | | | | |
| | | curve (ECC) | curve (ECC) | | | | | | |
| | ECT_929 | Euler characteristic | Euler characteristic | | | | | | |
| | | curve (ECC) | curve (ECC) | | | | | | |
| | ECT_930 | Euler characteristic | Euler characteristic | | | | | | |
| | | curve (ECC) | curve (ECC) | | | | | | |
| | ECT_931 | Euler characteristic | Euler characteristic | | | | | | |
| | | curve (ECC) | curve (ECC) | | | | | | |
| | ECT_932 | Euler characteristic | Euler characteristic | | | | | | |
| | | curve (ECC) | curve (ECC) | | | | | | |
| | ECT_933 | Euler characteristic | Euler characteristic | | | | | | |
| | | curve (ECC) | curve (ECC) | | | | | | |
| | ECT_934 | Euler characteristic | Euler characteristic | | | | | | |
| | | curve (ECC) | curve (ECC) | | | | | | |
| | ECT_935 | Euler characteristic | Euler characteristic | | | | | | |
| | | curve (ECC) | curve (ECC) | | | | | | |
| | ECT_936 | Euler characteristic | Euler characteristic | | | | | | |
| | | curve (ECC) | curve (ECC) | | | | | | |
| | ECT_937 | Euler characteristic | Euler characteristic | | | | | | |
| | | curve (ECC) | curve (ECC) | | | | | | |
| | ECT_938 | Euler characteristic | Euler characteristic | | | | | | |
| | | curve (ECC) | curve (ECC) | | | | | | |
| | ECT_939 | Euler characteristic | Euler characteristic | | | | | | |
| | | curve (ECC) | curve (ECC) | | | | | | |
| | ECT_940 | Euler characteristic | Euler characteristic | | | | | | |
| | | curve (ECC) | curve (ECC) | | | | | | |
| | ECT_941 | Euler characteristic | Euler characteristic | | | | | | |
| | | curve (ECC) | curve (ECC) | | | | | | |
| | ECT_942 | Euler characteristic | Euler characteristic | | | | | | |
| | | curve (ECC) | curve (ECC) | | | | | | |
| | ECT_943 | Euler characteristic | Euler characteristic | | | | | | |
| | | curve (ECC) | curve (ECC) | | | | | | |
| | ECT_944 | Euler characteristic | Euler characteristic | | | | | | |
| | | curve (ECC) | curve (ECC) | | | | | | |
| | ECT_945 | Euler characteristic | Euler characteristic | | | | | | |
| | | curve (ECC) | curve (ECC) | | | | | | |
| | ECT_946 | Euler characteristic | Euler characteristic | | | | | | |
| | | curve (ECC) | curve (ECC) | | | | | | |
| | ECT_947 | Euler characteristic | Euler characteristic | | | | | | |
| | | curve (ECC) | curve (ECC) | | | | | | |
| | ECT_948 | Euler characteristic | Euler characteristic | | | | | | |
| | | curve (ECC) | curve (ECC) | | | | | | |
| | ECT_949 | Euler characteristic | Euler characteristic | | | | | | |
| | | curve (ECC) | curve (ECC) | | | | | | |
| | ECT_950 | Euler characteristic | Euler characteristic | | | | | | |
| | | curve (ECC) | curve (ECC) | | | | | | |
| | ECT_951 | Euler characteristic | Euler characteristic | | | | | | |
| | | curve (ECC) | curve (ECC) | | | | | | |
| | ECT_952 | Euler characteristic | Euler characteristic | | | | | | |
| | | curve (ECC) | curve (ECC) | | | | | | |
| | ECT_953 | Euler characteristic | Euler characteristic | | | | | | |
| | | curve (ECC) | curve (ECC) | | | | | | |
| | ECT_954 | Euler characteristic | Euler characteristic | | | | | | |
| | | curve (ECC) | curve (ECC) | | | | | | |
| | ECT_955 | Euler characteristic | Euler characteristic | | | | | | |
| | | curve (ECC) | curve (ECC) | | | | | | |
| | ECT_956 | Euler characteristic | Euler characteristic | | | | | | |
| | | curve (ECC) | curve (ECC) | | | | | | |
| | ECT_957 | Euler characteristic | Euler characteristic | | | | | | |
| | | curve (ECC) | curve (ECC) | | | | | | |
| | ECT_958 | Euler characteristic | Euler characteristic | | | | | | |
| | | curve (ECC) | curve (ECC) | | | | | | |
| | ECT_959 | Euler characteristic | Euler characteristic | | | | | | |
| | | curve (ECC) | curve (ECC) | | | | | | |
| | ECT_960 | Euler characteristic | Euler characteristic | | | | | | |
| | | curve (ECC) | curve (ECC) | | | | | | |
| | ECT_961 | Euler characteristic | Euler characteristic | | | | | | |
| | | curve (ECC) | curve (ECC) | | | | | | |
| | ECT_962 | Euler characteristic | Euler characteristic | | | | | | |
| | | curve (ECC) | curve (ECC) | | | | | | |
| | ECT_963 | Euler characteristic | Euler characteristic | | | | | | |
| | | curve (ECC) | curve (ECC) | | | | | | |
| | ECT_964 | Euler characteristic | Euler characteristic | | | | | | |
| | | curve (ECC) | curve (ECC) | | | | | | |
| | ECT_965 | Euler characteristic | Euler characteristic | | | | | | |
| | | curve (ECC) | curve (ECC) | | | | | | |
| | ECT_966 | Euler characteristic | Euler characteristic | | | | | | |
| | | curve (ECC) | curve (ECC) | | | | | | |
| | ECT_967 | Euler characteristic | Euler characteristic | | | | | | |
| | | curve (ECC) | curve (ECC) | | | | | | |
| | ECT_968 | Euler characteristic | Euler characteristic | | | | | | |
| | | curve (ECC) | curve (ECC) | | | | | | |
| | ECT_969 | Euler characteristic | Euler characteristic | | | | | | |
| | | curve (ECC) | curve (ECC) | | | | | | |
| | ECT_970 | Euler characteristic | Euler characteristic | | | | | | |
| | | curve (ECC) | curve (ECC) | | | | | | |
| | ECT_971 | Euler characteristic | Euler characteristic | | | | | | |
| | | curve (ECC) | curve (ECC) | | | | | | |
| | ECT_972 | Euler characteristic | Euler characteristic | | | | | | |
| | | curve (ECC) | curve (ECC) | | | | | | |
| | ECT_973 | Euler characteristic | Euler characteristic | | | | | | |
| | | curve (ECC) | curve (ECC) | | | | | | |
| | ECT_974 | Euler characteristic | Euler characteristic | | | | | | |
| | | curve (ECC) | curve (ECC) | | | | | | |
| | ECT_975 | Euler characteristic | Euler characteristic | | | | | | |
| | | curve (ECC) | curve (ECC) | | | | | | |
| | ECT_976 | Euler characteristic | Euler characteristic | | | | | | |
| | | curve (ECC) | curve (ECC) | | | | | | |
| | ECT_977 | Euler characteristic | Euler characteristic | | | | | | |
| | | curve (ECC) | curve (ECC) | | | | | | |
| | ECT_978 | Euler characteristic | Euler characteristic | | | | | | |
| | | curve (ECC) | curve (ECC) | | | | | | |
| | ECT_979 | Euler characteristic | Euler characteristic | | | | | | |
| | | curve (ECC) | curve (ECC) | | | | | | |
| | ECT_980 | Euler characteristic | Euler characteristic | | | | | | |
| | | curve (ECC) | curve (ECC) | | | | | | |
| | ECT_981 | Euler characteristic | Euler characteristic | | | | | | |
| | | curve (ECC) | curve (ECC) | | | | | | |
| | ECT_982 | Euler characteristic | Euler characteristic | | | | | | |
| | | curve (ECC) | curve (ECC) | | | | | | |
| | ECT_983 | Euler characteristic | Euler characteristic | | | | | | |
| | | curve (ECC) | curve (ECC) | | | | | | |
| | ECT_984 | Euler characteristic | Euler characteristic | | | | | | |
| | | curve (ECC) | curve (ECC) | | | | | | |
| | ECT_985 | Euler characteristic | Euler characteristic | | | | | | |
| | | curve (ECC) | curve (ECC) | | | | | | |
| | ECT_986 | Euler characteristic | Euler characteristic | | | | | | |
| | | curve (ECC) | curve (ECC) | | | | | | |
| | ECT_987 | Euler characteristic | Euler characteristic | | | | | | |
| | | curve (ECC) | curve (ECC) | | | | | | |
| | ECT_988 | Euler characteristic | Euler characteristic | | | | | | |
| | | curve (ECC) | curve (ECC) | | | | | | |
| | ECT_989 | Euler characteristic | Euler characteristic | | | | | | |
| | | curve (ECC) | curve (ECC) | | | | | | |
| | ECT_990 | Euler characteristic | Euler characteristic | | | | | | |
| | | curve (ECC) | curve (ECC) | | | | | | |
| | ECT_991 | Euler characteristic | Euler characteristic | | | | | | |
| | | curve (ECC) | curve (ECC) | | | | | | |
| | ECT_992 | Euler characteristic | Euler characteristic | | | | | | |
| | | curve (ECC) | curve (ECC) | | | | | | |
| | ECT_993 | Euler characteristic | Euler characteristic | | | | | | |
| | | curve (ECC) | curve (ECC) | | | | | | |
| | ECT_994 | Euler characteristic | Euler characteristic | | | | | | |
| | | curve (ECC) | curve (ECC) | | | | | | |
| | ECT_995 | Euler characteristic | Euler characteristic | | | | | | |
| | | curve (ECC) | curve (ECC) | | | | | | |
| | ECT_996 | Euler characteristic | Euler characteristic | | | | | | |
| | | curve (ECC) | curve (ECC) | | | | | | |
| | ECT_997 | Euler characteristic | Euler characteristic | | | | | | |
| | | curve (ECC) | curve (ECC) | | | | | | |
| | ECT_998 | Euler characteristic | Euler characteristic | | | | | | |
| | | curve (ECC) | curve (ECC) | | | | | | |
| | ECT_999 | Euler characteristic | Euler characteristic | | | | | | |
| | | curve (ECC) | curve (ECC) | | | | | | |
| | ECT_1000 | Euler characteristic | Euler characteristic | | | | | | |
| | | curve (ECC) | curve (ECC) | | | | | | |
| | ECT_1001 | Euler characteristic | Euler characteristic | | | | | | |
| | | curve (ECC) | curve (ECC) | | | | | | |
| | ECT_1002 | Euler characteristic | Euler characteristic | | | | | | |
| | | curve (ECC) | curve (ECC) | | | | | | |
| | ECT_1003 | Euler characteristic | Euler characteristic | | | | | | |
| | | curve (ECC) | curve (ECC) | | | | | | |
| | ECT_1004 | Euler characteristic | Euler characteristic | | | | | | |
| | | curve (ECC) | curve (ECC) | | | | | | |
| | ECT_1005 | Euler characteristic | Euler characteristic | | | | | | |
| | | curve (ECC) | curve (ECC) | | | | | | |
| | ECT_1006 | Euler characteristic | Euler characteristic | | | | | | |
| | | curve (ECC) | curve (ECC) | | | | | | |
| | ECT_1007 | Euler characteristic | Euler characteristic | | | | | | |
| | | curve (ECC) | curve (ECC) | | | | | | |
| | ECT_1008 | Euler characteristic | Euler characteristic | | | | | | |
| | | curve (ECC) | curve (ECC) | | | | | | |
| | ECT_1009 | Euler characteristic | Euler characteristic | | | | | | |
| | | curve (ECC) | curve (ECC) | | | | | | |
| | ECT_1010 | Euler characteristic | Euler characteristic | | | | | | |
| | | curve (ECC) | curve (ECC) | | | | | | |
| | ECT_1011 | Euler characteristic | Euler characteristic | | | | | | |
| | | curve (ECC) | curve (ECC) | | | | | | |
| | ECT_1012 | Euler characteristic | Euler characteristic | | | | | | |
| | | curve (ECC) | curve (ECC) | | | | | | |
| | ECT_1013 | Euler characteristic | Euler characteristic | | | | | | |
| | | curve (ECC) | curve (ECC) | | | | | | |
| | ECT_1014 | Euler characteristic | Euler characteristic | | | | | | |
| | | curve (ECC) | curve (ECC) | | | | | | |
| | ECT_1015 | Euler characteristic | Euler characteristic | | | | | | |
| | | curve (ECC) | curve (ECC) | | | | | | |
| | ECT_1016 | Euler characteristic | Euler characteristic | | | | | | |
| | | curve (ECC) | curve (ECC) | | | | | | |
| | ECT_1017 | Euler characteristic | Euler characteristic | | | | | | |
| | | curve (ECC) | curve (ECC) | | | | | | |
| | ECT_1018 | Euler characteristic | Euler characteristic | | | | | | |
| | | curve (ECC) | curve (ECC) | | | | | | |
| | ECT_1019 | Euler characteristic | Euler characteristic | | | | | | |
| | | curve (ECC) | curve (ECC) | | | | | | |
| | ECT_1020 | Euler characteristic | Euler characteristic | | | | | | |
| | | curve (ECC) | curve (ECC) | | | | | | |
| | ECT_1021 | Euler characteristic | Euler characteristic | | | | | | |
| | | curve (ECC) | curve (ECC) | | | | | | |
| | ECT_1022 | Euler characteristic | Euler characteristic | | | | | | |
| | | curve (ECC) | curve (ECC) | | | | | | |
| | ECT_1023 | Euler characteristic | Euler characteristic | | | | | | |
| | | curve (ECC) | curve (ECC) | | | | | | |
| | ECT_1024 | Euler characteristic | Euler characteristic | | | | | | |
| | | curve (ECC) | curve (ECC) | | | | | | |
| | ECT_1025 | Euler characteristic | Euler characteristic | | | | | | |
| | | curve (ECC) | curve (ECC) | | | | | | |
| | ECT_1026 | Euler characteristic | Euler characteristic | | | | | | |
| | | curve (ECC) | curve (ECC) | | | | | | |
| | ECT_1027 | Euler characteristic | Euler characteristic | | | | | | |
| | | curve (ECC) | curve (ECC) | | | | | | |
| | ECT_1028 | Euler characteristic | Euler characteristic | | | | | | |
| | | curve (ECC) | curve (ECC) | | | | | | |
| | ECT_1029 | Euler characteristic | Euler characteristic | | | | | | |
| | | curve (ECC) | curve (ECC) | | | | | | |
| | ECT_1030 | Euler characteristic | Euler characteristic | | | | | | |
| | | curve (ECC) | curve (ECC) | | | | | | |
| | ECT_1031 | Euler characteristic | Euler characteristic | | | | | | |
| | | curve (ECC) | curve (ECC) | | | | | | |
| | ECT_1032 | Euler characteristic | Euler characteristic | | | | | | |
| | | curve (ECC) | curve (ECC) | | | | | | |
| | ECT_1033 | Euler characteristic | Euler characteristic | | | | | | |
| | | curve (ECC) | curve (ECC) | | | | | | |
| | ECT_1034 | Euler characteristic | Euler characteristic | | | | | | |
| | | curve (ECC) | curve (ECC) | | | | | | |
| | ECT_1035 | Euler characteristic | Euler characteristic | | | | | | |
| | | curve (ECC) | curve (ECC) | | | | | | |
| | ECT_1036 | Euler characteristic | Euler characteristic | | | | | | |
| | | curve (ECC) | curve (ECC) | | | | | | |
| | ECT_1037 | Euler characteristic | Euler characteristic | | | | | | |
| | | curve (ECC) | curve (ECC) | | | | | | |
| | ECT_1038 | Euler characteristic | Euler characteristic | | | | | | |
| | | curve (ECC) | curve (ECC) | | | | | | |
| | ECT_1039 | Euler characteristic | Euler characteristic | | | | | | |
| | | curve (ECC) | curve (ECC) | | | | | | |
| | ECT_1040 | Euler characteristic | Euler characteristic | | | | | | |
| | | curve (ECC) | curve (ECC) | | | | | | |
| | ECT_1041 | Euler characteristic | Euler characteristic | | | | | | |
| | | curve (ECC) | curve (ECC) | | | | | | |
| | ECT_1042 | Euler characteristic | Euler characteristic | | | | | | |
| | | curve (ECC) | curve (ECC) | | | | | | |
| | ECT_1043 | Euler characteristic | Euler characteristic | | | | | | |
| | | curve (ECC) | curve (ECC) | | | | | | |
| | ECT_1044 | Euler characteristic | Euler characteristic | | | | | | |
| | | curve (ECC) | curve (ECC) | | | | | | |
| | ECT_1045 | Euler characteristic | Euler characteristic | | | | | | |
| | | curve (ECC) | curve (ECC) | | | | | | |
| | ECT_1046 | Euler characteristic | Euler characteristic | | | | | | |
| | | curve (ECC) | curve (ECC) | | | | | | |
| | ECT_1047 | Euler characteristic | Euler characteristic | | | | | | |
| | | curve (ECC) | curve (ECC) | | | | | | |
| | ECT_1048 | Euler characteristic | Euler characteristic | | | | | | |
| | | curve (ECC) | curve (ECC) | | | | | | |
| | ECT_1049 | Euler characteristic | Euler characteristic | | | | | | |
| | | curve (ECC) | curve (ECC) | | | | | | |
| | ECT_1050 | Euler characteristic | Euler characteristic | | | | | | |
| | | curve (ECC) | curve (ECC) | | | | | | |
| | ECT_1051 | Euler characteristic | Euler characteristic | | | | | | |
| | | curve (ECC) | curve (ECC) | | | | | | |
| | ECT_1052 | Euler characteristic | Euler characteristic | | | | | | |
| | | curve (ECC) | curve (ECC) | | | | | | |
| | ECT_1053 | Euler characteristic | Euler characteristic | | | | | | |
| | | curve (ECC) | curve (ECC) | | | | | | |
| | ECT_1054 | Euler characteristic | Euler characteristic | | | | | | |
| | | curve (ECC) | curve (ECC) | | | | | | |
| | ECT_1055 | Euler characteristic | Euler characteristic | | | | | | |
| | | curve (ECC) | curve (ECC) | | | | | | |
| | ECT_1056 | Euler characteristic | Euler characteristic | | | | | | |
| | | curve (ECC) | curve (ECC) | | | | | | |
| | ECT_1057 | Euler characteristic | Euler characteristic | | | | | | |
| | | curve (ECC) | curve (ECC) | | | | | | |
| | ECT_1058 | Euler characteristic | Euler characteristic | | | | | | |
| | | curve (ECC) | curve (ECC) | | | | | | |
| | ECT_1059 | Euler characteristic | Euler characteristic | | | | | | |
| | | curve (ECC) | curve (ECC) | | | | | | |
| | ECT_1060 | Euler characteristic | Euler characteristic | | | | | | |
| | | curve (ECC) | curve (ECC) | | | | | | |
| | ECT_1061 | Euler characteristic | Euler characteristic | | | | | | |
| | | curve (ECC) | curve (ECC) | | | | | | |
| | ECT_1062 | Euler characteristic | Euler characteristic | | | | | | |
| | | curve (ECC) | curve (ECC) | | | | | | |
| | ECT_1063 | Euler characteristic | Euler characteristic | | | | | | |
| | | curve (ECC) | curve (ECC) | | | | | | |
| | ECT_1064 | Euler characteristic | Euler characteristic | | | | | | |
| | | curve (ECC) | curve (ECC) | | | | | | |
| | ECT_1065 | Euler characteristic | Euler characteristic | | | | | | |
| | | curve (ECC) | curve (ECC) | | | | | | |
| | ECT_1066 | Euler characteristic | Euler characteristic | | | | | | |
| | | curve (ECC) | curve (ECC) | | | | | | |
| | ECT_1067 | Euler characteristic | Euler characteristic | | | | | | |
| | | curve (ECC) | curve (ECC) | | | | | | |
| | ECT_1068 | Euler characteristic | Euler characteristic | | | | | | |
| | | curve (ECC) | curve (ECC) | | | | | | |
| | ECT_1069 | Euler characteristic | Euler characteristic | | | | | | |
| | | curve (ECC) | curve (ECC) | | | | | | |
| | ECT_1070 | Euler characteristic | Euler characteristic | | | | | | |
| | | curve (ECC) | curve (ECC) | | | | | | |
| | ECT_1071 | Euler characteristic | Euler characteristic | | | | | | |
| | | curve (ECC) | curve (ECC) | | | | | | |
| | ECT_1072 | Euler characteristic | Euler characteristic | | | | | | |
| | | curve (ECC) | curve (ECC) | | | | | | |
| | ECT_1073 | Euler characteristic | Euler characteristic | | | | | | |
| | | curve (ECC) | curve (ECC) | | | | | | |
| | ECT_1074 | Euler characteristic | Euler characteristic | | | | | | |
| | | curve (ECC) | curve (ECC) | | | | | | |
| | ECT_1075 | Euler characteristic | Euler characteristic | | | | | | |
| | | curve (ECC) | curve (ECC) | | | | | | |
| | ECT_1076 | Euler characteristic | Euler characteristic | | | | | | |
| | | curve (ECC) | curve (ECC) | | | | | | |
| | ECT_1077 | Euler characteristic | Euler characteristic | | | | | | |
| | | curve (ECC) | curve (ECC) | | | | | | |
| | ECT_1078 | Euler characteristic | Euler characteristic | | | | | | |
| | | curve (ECC) | curve (ECC) | | | | | | |
| | ECT_1079 | Euler characteristic | Euler characteristic | | | | | | |
| | | curve (ECC) | curve (ECC) | | | | | | |
| | ECT_1080 | Euler characteristic | Euler characteristic | | | | | | |
| | | curve (ECC) | curve (ECC) | | | | | | |
| | ECT_1081 | Euler characteristic | Euler characteristic | | | | | | |
| | | curve (ECC) | curve (ECC) | | | | | | |
| | ECT_1082 | Euler characteristic | Euler characteristic | | | | | | |
| | | curve (ECC) | curve (ECC) | | | | | | |
| | ECT_1083 | Euler characteristic | Euler characteristic | | | | | | |
| | | curve (ECC) | curve (ECC) | | | | | | |
| | ECT_1084 | Euler characteristic | Euler characteristic | | | | | | |
| | | curve (ECC) | curve (ECC) | | | | | | |
| | ECT_1085 | Euler characteristic | Euler characteristic | | | | | | |
| | | curve (ECC) | curve (ECC) | | | | | | |
| | ECT_1086 | Euler characteristic | Euler characteristic | | | | | | |
| | | curve (ECC) | curve (ECC) | | | | | | |
| | ECT_1087 | Euler characteristic | Euler characteristic | | | | | | |
| | | curve (ECC) | curve (ECC) | | | | | | |
| | ECT_1088 | Euler characteristic | Euler characteristic | | | | | | |
| | | curve (ECC) | curve (ECC) | | | | | | |
| | ECT_1089 | Euler characteristic | Euler characteristic | | | | | | |
| | | curve (ECC) | curve (ECC) | | | | | | |
| | ECT_1090 | Euler characteristic | Euler characteristic | | | | | | |
| | | curve (ECC) | curve (ECC) | | | | | | |
| | ECT_1091 | Euler characteristic | Euler characteristic | | | | | | |
| | | curve (ECC) | curve (ECC) | | | | | | |
| | ECT_1092 | Euler characteristic | Euler characteristic | | | | | | |
| | | curve (ECC) | curve (ECC) | | | | | | |
| | ECT_1093 | Euler characteristic | Euler characteristic | | | | | | |
| | | curve (ECC) | curve (ECC) | | | | | | |
| | ECT_1094 | Euler characteristic | Euler characteristic | | | | | | |
| | | curve (ECC) | curve (ECC) | | | | | | |
| | ECT_1095 | Euler characteristic | Euler characteristic | | | | | | |
| | | curve (ECC) | curve (ECC) | | | | | | |
| | ECT_1096 | Euler characteristic | Euler characteristic | | | | | | |
| | | curve (ECC) | curve (ECC) | | | | | | |
| | ECT_1097 | Euler characteristic | Euler characteristic | | | | | | |
| | | curve (ECC) | curve (ECC) | | | | | | |
| | ECT_1098 | Euler characteristic | Euler characteristic | | | | | | |
| | | curve (ECC) | curve (ECC) | | | | | | |
| | ECT_1099 | Euler characteristic | Euler characteristic | | | | | | |
| | | curve (ECC) | curve (ECC) | | | | | | |
| | ECT_1100 | Euler characteristic | Euler characteristic | | | | | | |
| | | curve (ECC) | curve (ECC) | | | | | | |
| | ECT_1101 | Euler characteristic | Euler characteristic | | | | | | |
| | | curve (ECC) | curve (ECC) | | | | | | |
| | ECT_1102 | Euler characteristic | Euler characteristic | | | | | | |
| | | curve (ECC) | curve (ECC) | | | | | | |
| | ECT_1103 | Euler characteristic | Euler characteristic | | | | | | |
| | | curve (ECC) | curve (ECC) | | | | | | |
| | ECT_1104 | Euler characteristic | Euler characteristic | | | | | | |
| | | curve (ECC) | curve (ECC) | | | | | | |
| | ECT_1105 | Euler characteristic | Euler characteristic | | | | | | |
| | | curve (ECC) | curve (ECC) | | | | | | |
| | ECT_1106 | Euler characteristic | Euler characteristic | | | | | | |
| | | curve (ECC) | curve (ECC) | | | | | | |
| | ECT_1107 | Euler characteristic | Euler characteristic | | | | | | |
| | | curve (ECC) | curve (ECC) | | | | | | |
| | ECT_1108 | Euler characteristic | Euler characteristic | | | | | | |
| | | curve (ECC) | curve (ECC) | | | | | | |
| | ECT_1109 | Euler characteristic | Euler characteristic | | | | | | |
| | | curve (ECC) | curve (ECC) | | | | | | |
| | ECT_1110 | Euler characteristic | Euler characteristic | | | | | | |
| | | curve (ECC) | curve (ECC) | | | | | | |
| | ECT_1111 | Euler characteristic | Euler characteristic | | | | | | |
| | | curve (ECC) | curve (ECC) | | | | | | |
| | ECT_1112 | Euler characteristic | Euler characteristic | | | | | | |
| | | curve (ECC) | curve (ECC) | | | | | | |
| | ECT_1113 | Euler characteristic | Euler characteristic | | | | | | |
| | | curve (ECC) | curve (ECC) | | | | | | |
| | ECT_1114 | Euler characteristic | Euler characteristic | | | | | | |
| | | curve (ECC) | curve (ECC) | | | | | | |
| | ECT_1115 | Euler characteristic | Euler characteristic | | | | | | |
| | | curve (ECC) | curve (ECC) | | | | | | |
| | ECT_1116 | Euler characteristic | Euler characteristic | | | | | | |
| | | curve (ECC) | curve (ECC) | | | | | | |
| | ECT_1117 | Euler characteristic | Euler characteristic | | | | | | |
| | | curve (ECC) | curve (ECC) | | | | | | |
| | ECT_1118 | Euler characteristic | Euler characteristic | | | | | | |
| | | curve (ECC) | curve (ECC) | | | | | | |
| | ECT_1119 | Euler characteristic | Euler characteristic | | | | | | |
| | | curve (ECC) | curve (ECC) | | | | | | |
| | ECT_1120 | Euler characteristic | Euler characteristic | | | | | | |
| | | curve (ECC) | curve (ECC) | | | | | | |
| | ECT_1121 | Euler characteristic | Euler characteristic | | | | | | |
| | | curve (ECC) | curve (ECC) | | | | | | |
| | ECT_1122 | Euler characteristic | Euler characteristic | | | | | | |
| | | curve (ECC) | curve (ECC) | | | | | | |
| | ECT_1123 | Euler characteristic | Euler characteristic | | | | | | |
| | | curve (ECC) | curve (ECC) | | | | | | |
| | ECT_1124 | Euler characteristic | Euler characteristic | | | | | | |
| | | curve (ECC) | curve (ECC) | | | | | | |
| | ECT_1125 | Euler characteristic | Euler characteristic | | | | | | |
| | | curve (ECC) | curve (ECC) | | | | | | |
| | ECT_1126 | Euler characteristic | Euler characteristic | | | | | | |
| | | curve (ECC) | curve (ECC) | | | | | | |
| | ECT_1127 | Euler characteristic | Euler characteristic | | | | | | |
| | | curve (ECC) | curve (ECC) | | | | | | |
| | ECT_1128 | Euler characteristic | Euler characteristic | | | | | | |
| | | curve (ECC) | curve (ECC) | | | | | | |
| | ECT_1129 | Euler characteristic | Euler characteristic | | | | | | |
| | | curve (ECC) | curve (ECC) | | | | | | |
| | ECT_1130 | Euler characteristic | Euler characteristic | | | | | | |
| | | curve (ECC) | curve (ECC) | | | | | | |
| | ECT_1131 | Euler characteristic | Euler characteristic | | | | | | |
| | | curve (ECC) | curve (ECC) | | | | | | |
| | ECT_1132 | Euler characteristic | Euler characteristic | | | | | | |
| | | curve (ECC) | curve (ECC) | | | | | | |
| | ECT_1133 | Euler characteristic | Euler characteristic | | | | | | |
| | | curve (ECC) | curve (ECC) | | | | | | |
| | ECT_1134 | Euler characteristic | Euler characteristic | | | | | | |
| | | curve (ECC) | curve (ECC) | | | | | | |
| | ECT_1135 | Euler characteristic | Euler characteristic | | | | | | |
| | | curve (ECC) | curve (ECC) | | | | | | |
| | ECT_1136 | Euler characteristic | Euler characteristic | | | | | | |
| | | curve (ECC) | curve (ECC) | | | | | | |
| | ECT_1137 | Euler characteristic | Euler characteristic | | | | | | |
| | | curve (ECC) | curve (ECC) | | | | | | |
| | ECT_1138 | Euler characteristic | Euler characteristic | | | | | | |
| | | curve (ECC) | curve (ECC) | | | | | | |
| | ECT_1139 | Euler characteristic | Euler characteristic | | | | | | |
| | | curve (ECC) | curve (ECC) | | | | | | |
| | ECT_1140 | Euler characteristic | Euler characteristic | | | | | | |
| | | curve (ECC) | curve (ECC) | | | | | | |
| | ECT_1141 | Euler characteristic | Euler characteristic | | | | | | |
| | | curve (ECC) | curve (ECC) | | | | | | |
| | ECT_1142 | Euler characteristic | Euler characteristic | | | | | | |
| | | curve (ECC) | curve (ECC) | | | | | | |
| | ECT_1143 | Euler characteristic | Euler characteristic | | | | | | |
| | | curve (ECC) | curve (ECC) | | | | | | |
| | ECT_1144 | Euler characteristic | Euler characteristic | | | | | | |
| | | curve (ECC) | curve (ECC) | | | | | | |
| | ECT_1145 | Euler characteristic | Euler characteristic | | | | | | |
| | | curve (ECC) | curve (ECC) | | | | | | |
| | ECT_1146 | Euler characteristic | Euler characteristic | | | | | | |
| | | curve (ECC) | curve (ECC) | | | | | | |
| | ECT_1147 | Euler characteristic | Euler characteristic | | | | | | |
| | | curve (ECC) | curve (ECC) | | | | | | |
| | ECT_1148 | Euler characteristic | Euler characteristic | | | | | | |
| | | curve (ECC) | curve (ECC) | | | | | | |
| | ECT_1149 | Euler characteristic | Euler characteristic | | | | | | |
| | | curve (ECC) | curve (ECC) | | | | | | |
| | ECT_1150 | Euler characteristic | Euler characteristic | | | | | | |
| | | curve (ECC) | curve (ECC) | | | | | | |
| | ECT_1151 | Euler characteristic | Euler characteristic | | | | | | |
| | | curve (ECC) | curve (ECC) | | | | | | |
| | ECT_1152 | Euler characteristic | Euler characteristic | | | | | | |
| | | curve (ECC) | curve (ECC) | | | | | | |
| Shape context | Shape_Context_1 | Shape context | Shape context | 30 | Distri- | histogram | | Describe shape, concatenation the histogram | Belongie, S., |
| | Shape_Context_2 | Shape context | Shape context | | bution | | | of the relative angle and distance | Mori, G. and |
| | Shape_Context_3 | Shape context | Shape context | | | | | distribution of points around n uniform | Malik, J., |
| | Shape_Context_4 | Shape context | Shape context | | | | | sampled points on the organoid contour | 2006. |
| | Shape_Context_5 | Shape context | Shape context | | | | | | Matching |
| | Shape_Context_6 | Shape context | Shape context | | | | | | with shape |
| | Shape_Context_7 | Shape context | Shape context | | | | | | contexts. In |
| | Shape_Context_8 | Shape context | Shape context | | | | | | Statistics |
| | Shape_Context_9 | Shape context | Shape context | | | | | | and Analysis |
| | Shape_Context_10 | Shape context | Shape context | | | | | | of Shapes |
| | Shape_Context_11 | Shape context | Shape context | | | | | | (pp. 81-105). |
| | Shape_Context_12 | Shape context | Shape context | | | | | | Birkhäuser |
| | Shape_Context_13 | Shape context | Shape context | | | | | | Boston. |
| | Shape_Context_14 | Shape context | Shape context | | | | | | |
| | Shape_Context_15 | Shape context | Shape context | | | | | | |
| | Shape_Context_16 | Shape context | Shape context | | | | | | |
| | Shape_Context_17 | Shape context | Shape context | | | | | | |
| | Shape_Context_18 | Shape context | Shape context | | | | | | |
| | Shape_Context_19 | Shape context | Shape context | | | | | | |
| | Shape_Context_20 | Shape context | Shape context | | | | | | |
| | Shape_Context_21 | Shape context | Shape context | | | | | | |
| | Shape_Context_22 | Shape context | Shape context | | | | | | |
| | Shape_Context_23 | Shape context | Shape context | | | | | | |
| | Shape_Context_24 | Shape context | Shape context | | | | | | |
| | Shape_Context_25 | Shape context | Shape context | | | | | | |
| | Shape_Context_26 | Shape context | Shape context | | | | | | |
| | Shape_Context_27 | Shape context | Shape context | | | | | | |
| | Shape_Context_28 | Shape context | Shape context | | | | | | |
| | Shape_Context_29 | Shape context | Shape context | | | | | | |
| | Shape_Context_30 | Shape context | Shape context | | | | | | |
| | Shape_Context_31 | Shape context | Shape context | | | | | | |
| | Shape_Context_32 | Shape context | Shape context | | | | | | |
| | Shape_Context_33 | Shape context | Shape context | | | | | | |
| | Shape_Context_34 | Shape context | Shape context | | | | | | |
| | Shape_Context_35 | Shape context | Shape context | | | | | | |
| | Shape_Context_36 | Shape context | Shape context | | | | | | |
| | Shape_Context_37 | Shape context | Shape context | | | | | | |
| | Shape_Context_38 | Shape context | Shape context | | | | | | |
| | Shape_Context_39 | Shape context | Shape context | | | | | | |
| | Shape_Context_40 | Shape context | Shape context | | | | | | |
| | Shape_Context_41 | Shape context | Shape context | | | | | | |
| | Shape_Context_42 | Shape context | Shape context | | | | | | |
| | Shape_Context_43 | Shape context | Shape context | | | | | | |
| | Shape_Context_44 | Shape context | Shape context | | | | | | |
| | Shape_Context_45 | Shape context | Shape context | | | | | | |
| | Shape_Context_46 | Shape context | Shape context | | | | | | |
| | Shape_Context_47 | Shape context | Shape context | | | | | | |
| | Shape_Context_48 | Shape context | Shape context | | | | | | |
| | Shape_Context_49 | Shape context | Shape context | | | | | | |
| | Shape_Context_50 | Shape context | Shape context | | | | | | |
| | Shape_Context_51 | Shape context | Shape context | | | | | | |
| | Shape_Context_52 | Shape context | Shape context | | | | | | |
| | Shape_Context_53 | Shape context | Shape context | | | | | | |
| | Shape_Context_54 | Shape context | Shape context | | | | | | |
| | Shape_Context_55 | Shape context | Shape context | | | | | | |
| | Shape_Context_56 | Shape context | Shape context | | | | | | |
| | Shape_Context_57 | Shape context | Shape context | | | | | | |
| | Shape_Context_58 | Shape context | Shape context | | | | | | |
| | Shape_Context_59 | Shape context | Shape context | | | | | | |
| | Shape_Context_60 | Shape context | Shape context | | | | | | |
| | Shape_Context_61 | Shape context | Shape context | | | | | | |
| | Shape_Context_62 | Shape context | Shape context | | | | | | |
| | Shape_Context_63 | Shape context | Shape context | | | | | | |
| | Shape_Context_64 | Shape context | Shape context | | | | | | |
| | Shape_Context_65 | Shape context | Shape context | | | | | | |
| | Shape_Context_66 | Shape context | Shape context | | | | | | |
| | Shape_Context_67 | Shape context | Shape context | | | | | | |
| | Shape_Context_68 | Shape context | Shape context | | | | | | |
| | Shape_Context_69 | Shape context | Shape context | | | | | | |
| | Shape_Context_70 | Shape context | Shape context | | | | | | |
| | Shape_Context_71 | Shape context | Shape context | | | | | | |
| | Shape_Context_72 | Shape context | Shape context | | | | | | |
| | Shape_Context_73 | Shape context | Shape context | | | | | | |
| | Shape_Context_74 | Shape context | Shape context | | | | | | |
| | Shape_Context_75 | Shape context | Shape context | | | | | | |
| | Shape_Context_76 | Shape context | Shape context | | | | | | |
| | Shape_Context_77 | Shape context | Shape context | | | | | | |
| | Shape_Context_78 | Shape context | Shape context | | | | | | |
| | Shape_Context_79 | Shape context | Shape context | | | | | | |
| | Shape_Context_80 | Shape context | Shape context | | | | | | |
| | Shape_Context_81 | Shape context | Shape context | | | | | | |
| | Shape_Context_82 | Shape context | Shape context | | | | | | |
| | Shape_Context_83 | Shape context | Shape context | | | | | | |
| | Shape_Context_84 | Shape context | Shape context | | | | | | |
| | Shape_Context_85 | Shape context | Shape context | | | | | | |
| | Shape_Context_86 | Shape context | Shape context | | | | | | |
| | Shape_Context_87 | Shape context | Shape context | | | | | | |
| | Shape_Context_88 | Shape context | Shape context | | | | | | |
| | Shape_Context_89 | Shape context | Shape context | | | | | | |
| | Shape_Context_90 | Shape context | Shape context | | | | | | |
| | Shape_Context_91 | Shape context | Shape context | | | | | | |
| | Shape_Context_92 | Shape context | Shape context | | | | | | |
| | Shape_Context_93 | Shape context | Shape context | | | | | | |
| | Shape_Context_94 | Shape context | Shape context | | | | | | |
| | Shape_Context_95 | Shape context | Shape context | | | | | | |
| | Shape_Context_96 | Shape context | Shape context | | | | | | |
| | Shape_Context_97 | Shape context | Shape context | | | | | | |
| | Shape_Context_98 | Shape context | Shape context | | | | | | |
| | Shape_Context_99 | Shape context | Shape context | | | | | | |
| | Shape_Context_100 | Shape context | Shape context | | | | | | |
| | Shape_Context_101 | Shape context | Shape context | | | | | | |
| | Shape_Context_102 | Shape context | Shape context | | | | | | |
| | Shape_Context_103 | Shape context | Shape context | | | | | | |
| | Shape_Context_104 | Shape context | Shape context | | | | | | |
| | Shape_Context_105 | Shape context | Shape context | | | | | | |
| | Shape_Context_106 | Shape context | Shape context | | | | | | |
| | Shape_Context_107 | Shape context | Shape context | | | | | | |
| | Shape_Context_108 | Shape context | Shape context | | | | | | |
| | Shape_Context_109 | Shape context | Shape context | | | | | | |
| | Shape_Context_110 | Shape context | Shape context | | | | | | |
| | Shape_Context_111 | Shape context | Shape context | | | | | | |
| | Shape_Context_112 | Shape context | Shape context | | | | | | |
| | Shape_Context_113 | Shape context | Shape context | | | | | | |
| | Shape_Context_114 | Shape context | Shape context | | | | | | |
| | Shape_Context_115 | Shape context | Shape context | | | | | | |
| | Shape_Context_116 | Shape context | Shape context | | | | | | |
| | Shape_Context_117 | Shape context | Shape context | | | | | | |
| | Shape_Context_118 | Shape context | Shape context | | | | | | |
| | Shape_Context_119 | Shape context | Shape context | | | | | | |
| | Shape_Context_120 | Shape context | Shape context | | | | | | |
| | Shape_Context_121 | Shape context | Shape context | | | | | | |
| | Shape_Context_122 | Shape context | Shape context | | | | | | |
| | Shape_Context_123 | Shape context | Shape context | | | | | | |
| | Shape_Context_124 | Shape context | Shape context | | | | | | |
| | Shape_Context_125 | Shape context | Shape context | | | | | | |
| | Shape_Context_126 | Shape context | Shape context | | | | | | |
| | Shape_Context_127 | Shape context | Shape context | | | | | | |
| | Shape_Context_128 | Shape context | Shape context | | | | | | |
| | Shape_Context_129 | Shape context | Shape context | | | | | | |
| | Shape_Context_130 | Shape context | Shape context | | | | | | |
| | Shape_Context_131 | Shape context | Shape context | | | | | | |
| | Shape_Context_132 | Shape context | Shape context | | | | | | |
| | Shape_Context_133 | Shape context | Shape context | | | | | | |
| | Shape_Context_134 | Shape context | Shape context | | | | | | |
| | Shape_Context_135 | Shape context | Shape context | | | | | | |
| | Shape_Context_136 | Shape context | Shape context | | | | | | |
| | Shape_Context_137 | Shape context | Shape context | | | | | | |
| | Shape_Context_138 | Shape context | Shape context | | | | | | |
| | Shape_Context_139 | Shape context | Shape context | | | | | | |
| | Shape_Context_140 | Shape context | Shape context | | | | | | |
| | Shape_Context_141 | Shape context | Shape context | | | | | | |
| | Shape_Context_142 | Shape context | Shape context | | | | | | |
| | Shape_Context_143 | Shape context | Shape context | | | | | | |
| | Shape_Context_144 | Shape context | Shape context | | | | | | |
| | Shape_Context_145 | Shape context | Shape context | | | | | | |
| | Shape_Context_146 | Shape context | Shape context | | | | | | |
| | Shape_Context_147 | Shape context | Shape context | | | | | | |
| | Shape_Context_148 | Shape context | Shape context | | | | | | |
| | Shape_Context_149 | Shape context | Shape context | | | | | | |
| | Shape_Context_150 | Shape context | Shape context | | | | | | |
| | Shape_Context_151 | Shape context | Shape context | | | | | | |
| | Shape_Context_152 | Shape context | Shape context | | | | | | |
| | Shape_Context_153 | Shape context | Shape context | | | | | | |
| | Shape_Context_154 | Shape context | Shape context | | | | | | |
| | Shape_Context_155 | Shape context | Shape context | | | | | | |
| | Shape_Context_156 | Shape context | Shape context | | | | | | |
| | Shape_Context_157 | Shape context | Shape context | | | | | | |
| | Shape_Context_158 | Shape context | Shape context | | | | | | |
| | Shape_Context_159 | Shape context | Shape context | | | | | | |
| | Shape_Context_160 | Shape context | Shape context | | | | | | |
| | Shape_Context_161 | Shape context | Shape context | | | | | | |
| | Shape_Context_162 | Shape context | Shape context | | | | | | |
| | Shape_Context_163 | Shape context | Shape context | | | | | | |
| | Shape_Context_164 | Shape context | Shape context | | | | | | |
| | Shape_Context_165 | Shape context | Shape context | | | | | | |
| | Shape_Context_166 | Shape context | Shape context | | | | | | |
| | Shape_Context_167 | Shape context | Shape context | | | | | | |
| | Shape_Context_168 | Shape context | Shape context | | | | | | |
| | Shape_Context_169 | Shape context | Shape context | | | | | | |
| | Shape_Context_170 | Shape context | Shape context | | | | | | |
| | Shape_Context_171 | Shape context | Shape context | | | | | | |
| | Shape_Context_172 | Shape context | Shape context | | | | | | |
| | Shape_Context_173 | Shape context | Shape context | | | | | | |
| | Shape_Context_174 | Shape context | Shape context | | | | | | |
| | Shape_Context_175 | Shape context | Shape context | | | | | | |
| | Shape_Context_176 | Shape context | Shape context | | | | | | |
| | Shape_Context_177 | Shape context | Shape context | | | | | | |
| | Shape_Context_178 | Shape context | Shape context | | | | | | |
| | Shape_Context_179 | Shape context | Shape context | | | | | | |
| | Shape_Context_180 | Shape context | Shape context | | | | | | |
| | Shape_Context_181 | Shape context | Shape context | | | | | | |
| | Shape_Context_182 | Shape context | Shape context | | | | | | |
| | Shape_Context_183 | Shape context | Shape context | | | | | | |
| | Shape_Context_184 | Shape context | Shape context | | | | | | |
| | Shape_Context_185 | Shape context | Shape context | | | | | | |
| | Shape_Context_186 | Shape context | Shape context | | | | | | |
| | Shape_Context_187 | Shape context | Shape context | | | | | | |
| | Shape_Context_188 | Shape context | Shape context | | | | | | |
| | Shape_Context_189 | Shape context | Shape context | | | | | | |
| | Shape_Context_190 | Shape context | Shape context | | | | | | |
| | Shape_Context_191 | Shape context | Shape context | | | | | | |
| | Shape_Context_192 | Shape context | Shape context | | | | | | |
| | Shape_Context_193 | Shape context | Shape context | | | | | | |
| | Shape_Context_194 | Shape context | Shape context | | | | | | |
| | Shape_Context_195 | Shape context | Shape context | | | | | | |
| | Shape_Context_196 | Shape context | Shape context | | | | | | |
| | Shape_Context_197 | Shape context | Shape context | | | | | | |
| | Shape_Context_198 | Shape context | Shape context | | | | | | |
| | Shape_Context_199 | Shape context | Shape context | | | | | | |
| | Shape_Context_200 | Shape context | Shape context | | | | | | |
| | Shape_Context_201 | Shape context | Shape context | | | | | | |
| | Shape_Context_202 | Shape context | Shape context | | | | | | |
| | Shape_Context_203 | Shape context | Shape context | | | | | | |
| | Shape_Context_204 | Shape context | Shape context | | | | | | |
| | Shape_Context_205 | Shape context | Shape context | | | | | | |
| | Shape_Context_206 | Shape context | Shape context | | | | | | |
| | Shape_Context_207 | Shape context | Shape context | | | | | | |
| | Shape_Context_208 | Shape context | Shape context | | | | | | |
| | Shape_Context_209 | Shape context | Shape context | | | | | | |
| | Shape_Context_210 | Shape context | Shape context | | | | | | |
| | Shape_Context_211 | Shape context | Shape context | | | | | | |
| | Shape_Context_212 | Shape context | Shape context | | | | | | |
| | Shape_Context_213 | Shape context | Shape context | | | | | | |
| | Shape_Context_214 | Shape context | Shape context | | | | | | |
| | Shape_Context_215 | Shape context | Shape context | | | | | | |
| | Shape_Context_216 | Shape context | Shape context | | | | | | |
| | Shape_Context_217 | Shape context | Shape context | | | | | | |
| | Shape_Context_218 | Shape context | Shape context | | | | | | |
| | Shape_Context_219 | Shape context | Shape context | | | | | | |
| | Shape_Context_220 | Shape context | Shape context | | | | | | |
| | Shape_Context_221 | Shape context | Shape context | | | | | | |
| | Shape_Context_222 | Shape context | Shape context | | | | | | |
| | Shape_Context_223 | Shape context | Shape context | | | | | | |
| | Shape_Context_224 | Shape context | Shape context | | | | | | |
| | Shape_Context_225 | Shape context | Shape context | | | | | | |
| | Shape_Context_226 | Shape context | Shape context | | | | | | |
| | Shape_Context_227 | Shape context | Shape context | | | | | | |
| | Shape_Context_228 | Shape context | Shape context | | | | | | |
| | Shape_Context_229 | Shape context | Shape context | | | | | | |
| | Shape_Context_230 | Shape context | Shape context | | | | | | |
| | Shape_Context_231 | Shape context | Shape context | | | | | | |
| | Shape_Context_232 | Shape context | Shape context | | | | | | |
| | Shape_Context_233 | Shape context | Shape context | | | | | | |
| | Shape_Context_234 | Shape context | Shape context | | | | | | |
| | Shape_Context_235 | Shape context | Shape context | | | | | | |
| | Shape_Context_236 | Shape context | Shape context | | | | | | |
| | Shape_Context_237 | Shape context | Shape context | | | | | | |
| | Shape_Context_238 | Shape context | Shape context | | | | | | |
| | Shape_Context_239 | Shape context | Shape context | | | | | | |
| | Shape_Context_240 | Shape context | Shape context | | | | | | |
| | Shape_Context_241 | Shape context | Shape context | | | | | | |
| | Shape_Context_242 | Shape context | Shape context | | | | | | |
| | Shape_Context_243 | Shape context | Shape context | | | | | | |
| | Shape_Context_244 | Shape context | Shape context | | | | | | |
| | Shape_Context_245 | Shape context | Shape context | | | | | | |
| | Shape_Context_246 | Shape context | Shape context | | | | | | |
| | Shape_Context_247 | Shape context | Shape context | | | | | | |
| | Shape_Context_248 | Shape context | Shape context | | | | | | |
| | Shape_Context_249 | Shape context | Shape context | | | | | | |
| | Shape_Context_250 | Shape context | Shape context | | | | | | |
| | Shape_Context_251 | Shape context | Shape context | | | | | | |
| | Shape_Context_252 | Shape context | Shape context | | | | | | |
| | Shape_Context_253 | Shape context | Shape context | | | | | | |
| | Shape_Context_254 | Shape context | Shape context | | | | | | |
| | Shape_Context_255 | Shape context | Shape context | | | | | | |
| | Shape_Context_256 | Shape context | Shape context | | | | | | |
| | Shape_Context_257 | Shape context | Shape context | | | | | | |
| | Shape_Context_258 | Shape context | Shape context | | | | | | |
| | Shape_Context_259 | Shape context | Shape context | | | | | | |
| | Shape_Context_260 | Shape context | Shape context | | | | | | |
| | Shape_Context_261 | Shape context | Shape context | | | | | | |
| | Shape_Context_262 | Shape context | Shape context | | | | | | |
| | Shape_Context_263 | Shape context | Shape context | | | | | | |
| | Shape_Context_264 | Shape context | Shape context | | | | | | |
| | Shape_Context_265 | Shape context | Shape context | | | | | | |
| | Shape_Context_266 | Shape context | Shape context | | | | | | |
| | Shape_Context_267 | Shape context | Shape context | | | | | | |
| | Shape_Context_268 | Shape context | Shape context | | | | | | |
| | Shape_Context_269 | Shape context | Shape context | | | | | | |
| | Shape_Context_270 | Shape context | Shape context | | | | | | |
| | Shape_Context_271 | Shape context | Shape context | | | | | | |
| | Shape_Context_272 | Shape context | Shape context | | | | | | |
| | Shape_Context_273 | Shape context | Shape context | | | | | | |
| | Shape_Context_274 | Shape context | Shape context | | | | | | |
| | Shape_Context_275 | Shape context | Shape context | | | | | | |
| | Shape_Context_276 | Shape context | Shape context | | | | | | |
| | Shape_Context_277 | Shape context | Shape context | | | | | | |
| | Shape_Context_278 | Shape context | Shape context | | | | | | |
| | Shape_Context_279 | Shape context | Shape context | | | | | | |
| | Shape_Context_280 | Shape context | Shape context | | | | | | |
| | Shape_Context_281 | Shape context | Shape context | | | | | | |
| | Shape_Context_282 | Shape context | Shape context | | | | | | |
| | Shape_Context_283 | Shape context | Shape context | | | | | | |
| | Shape_Context_284 | Shape context | Shape context | | | | | | |
| | Shape_Context_285 | Shape context | Shape context | | | | | | |
| | Shape_Context_286 | Shape context | Shape context | | | | | | |
| | Shape_Context_287 | Shape context | Shape context | | | | | | |
| | Shape_Context_288 | Shape context | Shape context | | | | | | |
| | Shape_Context_289 | Shape context | Shape context | | | | | | |
| | Shape_Context_290 | Shape context | Shape context | | | | | | |
| | Shape_Context_291 | Shape context | Shape context | | | | | | |
| | Shape_Context_292 | Shape context | Shape context | | | | | | |
| | Shape_Context_293 | Shape context | Shape context | | | | | | |
| | Shape_Context_294 | Shape context | Shape context | | | | | | |
| | Shape_Context_295 | Shape context | Shape context | | | | | | |
| | Shape_Context_296 | Shape context | Shape context | | | | | | |
| | Shape_Context_297 | Shape context | Shape context | | | | | | |
| | Shape_Context_298 | Shape context | Shape context | | | | | | |
| | Shape_Context_299 | Shape context | Shape context | | | | | | |
| | Shape_Context_300 | Shape context | Shape context | | | | | | |

TABLE 2
SAM - A (Appearance phenome)

| | | | | # of | | |
|---|---|---|---|---|---|---|
| | | | | Features | | Measurement |
| Feature/Descriptor | Feature Names | Feature Category | Feature set | (dimension) | Scope | Type |
| Mean global intensity | mean_intensity | Mean Intensity | Intensity statistics | 1 | Global | scalar |
| | | Features | | | | |
| Standard deviation global intensity | stddev_intensity | Mean Intensity | Intensity statistics | 1 | Global | scalar |
| | | Features | | | | |
| Mean regional intensity (Region 1, | mean_intensity_{—} | Mean regional | Intensity statistics | 1 | Regional | scalar |
| object border) | region_001 | intensity features | | | | |
| Standard deviation of regional | stddev_intensity_{—} | Mean regional | Intensity statistics | 1 | Regional | scalar |
| (Region 1, object intensity border) | region_001 | intensity features | | | | |
| Mean regional intensity (Region 2, | mean_intensity_{—} | Mean regional | Intensity statistics | 1 | Regional | scalar |
| mid section) | region_002 | intensity features | | | | |
| Standard deviation of regional | stddev_intensity_{—} | Mean regional | Intensity statistics | 1 | Regional | scalar |
| intensity (Region 2, mid section) | region_002 | intensity features | | | | |
| Mean regional intensity (Region 3, | mean_intensity_{—} | Mean regional | Intensity statistics | 1 | Regional | scalar |
| inner region) | region_003 | intensity features | | | | |
| Standard deviation of regional | stddev_intensity_{—} | Mean regional | Intensity statistics | 1 | Regional | scalar |
| intensity (Region 3, inner region) | region_003 | intensity features | | | | |
| Haralick features | angular_second_{—} | Haralick Features | Haralick texture features | 13 | Distribution | vector |
| | moment | | | | | |
| | contrast | Haralick Features | Haralick texture features | | | |
| | correlation | Haralick Features | Haralick texture features | | | |
| | sum_squares_{—} | Haralick Features | Haralick texture features | | | |
| | variance | | | | | |
| | inv_diff_moment | Haralick Features | Haralick texture features | | | |
| | sum_average | Haralick Features | Haralick texture features | | | |
| | sum_variance | Haralick Features | Haralick texture features | | | |
| | sum_entropy | Haralick Features | Haralick texture features | | | |
| | entropy | Haralick Features | Haralick texture features | | | |
| | diff_variance | Haralick Features | Haralick texture features | | | |
| | diff_entropy | Haralick Features | Haralick texture features | | | |
| | info_measure_{—} | Haralick Features | Haralick texture features | | | |
| | correlation_1 | | | | | |
| | info_measure_{—} | Haralick Features | Haralick texture features | | | |
| | correlation_2 | | | | | |
| Scale invariant feature | sift_1_appear | SIFT_features | SIFT features | 128 | Distribution | Histogram |
| transform (SIFT) | sift_2_appear | SIFT_features | SIFT features | | | |
| | sift_3_appear | SIFT_features | SIFT features | | | |
| | sift_4_appear | SIFT_features | SIFT features | | | |
| | sift_5_appear | SIFT_features | SIFT features | | | |
| | sift_6_appear | SIFT_features | SIFT features | | | |
| | sift_7_appear | SIFT_features | SIFT features | | | |
| | sift_8_appear | SIFT_features | SIFT features | | | |
| | sift_9_appear | SIFT_features | SIFT features | | | |
| | sift_10_appear | SIFT_features | SIFT features | | | |
| | sift_11_appear | SIFT_features | SIFT features | | | |
| | sift_12_appear | SIFT_features | SIFT features | | | |
| | sift_13_appear | SIFT_features | SIFT features | | | |
| | sift_14_appear | SIFT_features | SIFT features | | | |
| | sift_15_appear | SIFT_features | SIFT features | | | |
| | sift_16_appear | SIFT_features | SIFT features | | | |
| | sift_17_appear | SIFT_features | SIFT features | | | |
| | sift_18_appear | SIFT_features | SIFT features | | | |
| | sift_19_appear | SIFT_features | SIFT features | | | |
| | sift_20_appear | SIFT_features | SIFT features | | | |
| | sift_21_appear | SIFT_features | SIFT features | | | |
| | sift_22_appear | SIFT_features | SIFT features | | | |
| | sift_23_appear | SIFT_features | SIFT features | | | |
| | sift_24_appear | SIFT_features | SIFT features | | | |
| | sift_25_appear | SIFT_features | SIFT features | | | |
| | sift_26_appear | SIFT_features | SIFT features | | | |
| | sift_27_appear | SIFT_features | SIFT features | | | |
| | sift_28_appear | SIFT_features | SIFT features | | | |
| | sift_29_appear | SIFT_features | SIFT features | | | |
| | sift_30_appear | SIFT_features | SIFT features | | | |
| | sift_31_appear | SIFT_features | SIFT features | | | |
| | sift_32_appear | SIFT_features | SIFT features | | | |
| | sift_33_appear | SIFT_features | SIFT features | | | |
| | sift_34_appear | SIFT_features | SIFT features | | | |
| | sift_35_appear | SIFT_features | SIFT features | | | |
| | sift_36_appear | SIFT_features | SIFT features | | | |
| | sift_37_appear | SIFT_features | SIFT features | | | |
| | sift_38_appear | SIFT_features | SIFT features | | | |
| | sift_39_appear | SIFT_features | SIFT features | | | |
| | sift_40_appear | SIFT_features | SIFT features | | | |
| | sift_41_appear | SIFT_features | SIFT features | | | |
| | sift_42_appear | SIFT_features | SIFT features | | | |
| | sift_43_appear | SIFT_features | SIFT features | | | |
| | sift_44_appear | SIFT_features | SIFT features | | | |
| | sift_45_appear | SIFT_features | SIFT features | | | |
| | sift_46_appear | SIFT_features | SIFT features | | | |
| | sift_47_appear | SIFT_features | SIFT features | | | |
| | sift_48_appear | SIFT_features | SIFT features | | | |
| | sift_49_appear | SIFT_features | SIFT features | | | |
| | sift_50_appear | SIFT_features | SIFT features | | | |
| | sift_51_appear | SIFT_features | SIFT features | | | |
| | sift_52_appear | SIFT_features | SIFT features | | | |
| | sift_53_appear | SIFT_features | SIFT features | | | |
| | sift_54_appear | SIFT_features | SIFT features | | | |
| | sift_55_appear | SIFT_features | SIFT features | | | |
| | sift_56_appear | SIFT_features | SIFT features | | | |
| | sift_57_appear | SIFT_features | SIFT features | | | |
| | sift_58_appear | SIFT_features | SIFT features | | | |
| | sift_59_appear | SIFT_features | SIFT features | | | |
| | sift_60_appear | SIFT_features | SIFT features | | | |
| | sift_61_appear | SIFT_features | SIFT features | | | |
| | sift_62_appear | SIFT_features | SIFT features | | | |
| | sift_63_appear | SIFT_features | SIFT features | | | |
| | sift_64_appear | SIFT_features | SIFT features | | | |
| | sift_65_appear | SIFT_features | SIFT features | | | |
| | sift_66_appear | SIFT_features | SIFT features | | | |
| | sift_67_appear | SIFT_features | SIFT features | | | |
| | sift_68_appear | SIFT_features | SIFT features | | | |
| | sift_69_appear | SIFT_features | SIFT features | | | |
| | sift_70_appear | SIFT_features | SIFT features | | | |
| | sift_71_appear | SIFT_features | SIFT features | | | |
| | sift_72_appear | SIFT_features | SIFT features | | | |
| | sift_73_appear | SIFT_features | SIFT features | | | |
| | sift_74_appear | SIFT_features | SIFT features | | | |
| | sift_75_appear | SIFT_features | SIFT features | | | |
| | sift_76_appear | SIFT_features | SIFT features | | | |
| | sift_77_appear | SIFT_features | SIFT features | | | |
| | sift_78_appear | SIFT_features | SIFT features | | | |
| | sift_79_appear | SIFT_features | SIFT features | | | |
| | sift_80_appear | SIFT_features | SIFT features | | | |
| | sift_81_appear | SIFT_features | SIFT features | | | |
| | sift_82_appear | SIFT_features | SIFT features | | | |
| | sift_83_appear | SIFT_features | SIFT features | | | |
| | sift_84_appear | SIFT_features | SIFT features | | | |
| | sift_85_appear | SIFT_features | SIFT features | | | |
| | sift_86_appear | SIFT_features | SIFT features | | | |
| | sift_87_appear | SIFT_features | SIFT features | | | |
| | sift_88_appear | SIFT_features | SIFT features | | | |
| | sift_89_appear | SIFT_features | SIFT features | | | |
| | sift_90_appear | SIFT_features | SIFT features | | | |
| | sift_91_appear | SIFT_features | SIFT features | | | |
| | sift_92_appear | SIFT_features | SIFT features | | | |
| | sift_93_appear | SIFT_features | SIFT features | | | |
| | sift_94_appear | SIFT_features | SIFT features | | | |
| | sift_95_appear | SIFT_features | SIFT features | | | |
| | sift_96_appear | SIFT_features | SIFT features | | | |
| | sift_97_appear | SIFT_features | SIFT features | | | |
| | sift_98_appear | SIFT_features | SIFT features | | | |
| | sift_99_appear | SIFT_features | SIFT features | | | |
| | sift_100_appear | SIFT_features | SIFT features | | | |
| | sift_101_appear | SIFT_features | SIFT features | | | |
| | sift_102_appear | SIFT_features | SIFT features | | | |
| | sift_103_appear | SIFT_features | SIFT features | | | |
| | sift_104_appear | SIFT_features | SIFT features | | | |
| | sift_105_appear | SIFT_features | SIFT features | | | |
| | sift_106_appear | SIFT_features | SIFT features | | | |
| | sift_107_appear | SIFT_features | SIFT features | | | |
| | sift_108_appear | SIFT_features | SIFT features | | | |
| | sift_109_appear | SIFT_features | SIFT features | | | |
| | sift_110_appear | SIFT_features | SIFT features | | | |
| | sift_111_appear | SIFT_features | SIFT features | | | |
| | sift_112_appear | SIFT_features | SIFT features | | | |
| | sift_113_appear | SIFT_features | SIFT features | | | |
| | sift_114_appear | SIFT_features | SIFT features | | | |
| | sift_115_appear | SIFT_features | SIFT features | | | |
| | sift_116_appear | SIFT_features | SIFT features | | | |
| | sift_117_appear | SIFT_features | SIFT features | | | |
| | sift_118_appear | SIFT_features | SIFT features | | | |
| | sift_119_appear | SIFT_features | SIFT features | | | |
| | sift_120_appear | SIFT_features | SIFT features | | | |
| | sift_121_appear | SIFT_features | SIFT features | | | |
| | sift_122_appear | SIFT_features | SIFT features | | | |
| | sift_123_appear | SIFT_features | SIFT features | | | |
| | sift_124_appear | SIFT_features | SIFT features | | | |
| | sift_125_appear | SIFT_features | SIFT features | | | |
| | sift_126_appear | SIFT_features | SIFT features | | | |
| | sift_127_appear | SIFT_features | SIFT features | | | |
| | sift_128_appear | SIFT_features | SIFT features | | | |

| Feature/Descriptor | Feature Names | Description | References |
|---|---|---|---|
| Mean global intensity | mean_intensity | Mean pixel intensity within the pixels | |
| | | enclosed by the object contour | |
| Standard deviation global intensity | stddev_intensity | Standard deviation of the pixel intensity | |
| | | within the pixels enclosed by the object contour | |
| Mean regional intensity (Region 1, | mean_intensity_{—} | Mean pixel intensity within the first region | |
| object border) | region_001 | (object border) after concentric equidistant | |
| | | partitioning of the total object area | |
| Standard deviation of regional | stddev_intensity_{—} | Standard deivation of pixel intensity within | |
| (Region 1, object intensity border) | region_001 | the first region (object border) after | |
| | | concentric equidistant partitioning of the | |
| | | total object area | |
| Mean regional intensity (Region 2, | mean_intensity_{—} | Mean pixel intensity within the second region | |
| mid section) | region_002 | (object middle region) after concentric | |
| | | equidistant partitioning of the total object area | |
| Standard deviation of regional | stddev_intensity_{—} | Standard deivation of pixel intensity within | |
| intensity (Region 2, mid section) | region_002 | second region (object middle region) after | |
| | | concentric equidistant partitioning of the | |
| | | total object area | |
| Mean regional intensity (Region 3, | mean_intensity_{—} | Mean pixel intensity within third region (object | |
| inner region) | region_003 | central region) after concentric equidistant | |
| | | partitioning of the total object area | |
| Standard deviation of regional | stddev_intensity_{—} | Standard deviation of pixel intensity within third | |
| intensity (Region 3, inner region) | region_003 | region (object central region) after concentric | |
| | | equidistant partitioning of the totalobject area | |
| Haralick features | angular_second_{—} | Set of statistic summaries of the gray level co- | Löfstedt, T., Brynolfsson, |
| | moment | occurrence matrix (GLCM), a matrix that counts | P., Asklund, T., Nyholm, |
| | contrast | the co-occurrence of neighbouring gray levels | T. and Garpebring, A., |
| | correlation | within a given distance threshold in the image. | 2019. Gray-level invariant |
| | sum_squares_{—} | | Haralick texture features. |
| | variance | | PloS one, 14(2), |
| | inv_diff_moment | | p.e0212110. |
| | sum_average | | |
| | sum_variance | | |
| | sum_entropy | | |
| | entropy | | |
| | diff_variance | | |
| | diff_entropy | | |
| | info_measure_{—} | | |
| | correlation_1 | | |
| | info_measure_{—} | | |
| | correlation_2 | | |
| Scale invariant feature | sift_1_appear | Histogram of spatially local image intensity | Lowe, D. G., 1999, |
| transform (SIFT) | sift_2_appear | gradient directions that is invariant to | September. Object |
| | sift_3_appear | translation, rotation and scaling | recognition from local |
| | sift_4_appear | | scale-invariant features. In |
| | sift_5_appear | | Proceedings of the seventh |
| | sift_6_appear | | IEEE international |
| | sift_7_appear | | conference on computer |
| | sift_8_appear | | vision (Vol. 2, pp. 1150- |
| | sift_9_appear | | 1157). leee. |
| | sift_10_appear | | |
| | sift_11_appear | | |
| | sift_12_appear | | |
| | sift_13_appear | | |
| | sift_14_appear | | |
| | sift_15_appear | | |
| | sift_16_appear | | |
| | sift_17_appear | | |
| | sift_18_appear | | |
| | sift_19_appear | | |
| | sift_20_appear | | |
| | sift_21_appear | | |
| | sift_22_appear | | |
| | sift_23_appear | | |
| | sift_24_appear | | |
| | sift_25_appear | | |
| | sift_26_appear | | |
| | sift_27_appear | | |
| | sift_28_appear | | |
| | sift_29_appear | | |
| | sift_30_appear | | |
| | sift_31_appear | | |
| | sift_32_appear | | |
| | sift_33_appear | | |
| | sift_34_appear | | |
| | sift_35_appear | | |
| | sift_36_appear | | |
| | sift_37_appear | | |
| | sift_38_appear | | |
| | sift_39_appear | | |
| | sift_40_appear | | |
| | sift_41_appear | | |
| | sift_42_appear | | |
| | sift_43_appear | | |
| | sift_44_appear | | |
| | sift_45_appear | | |
| | sift_46_appear | | |
| | sift_47_appear | | |
| | sift_48_appear | | |
| | sift_49_appear | | |
| | sift_50_appear | | |
| | sift_51_appear | | |
| | sift_52_appear | | |
| | sift_53_appear | | |
| | sift_54_appear | | |
| | sift_55_appear | | |
| | sift_56_appear | | |
| | sift_57_appear | | |
| | sift_58_appear | | |
| | sift_59_appear | | |
| | sift_60_appear | | |
| | sift_61_appear | | |
| | sift_62_appear | | |
| | sift_63_appear | | |
| | sift_64_appear | | |
| | sift_65_appear | | |
| | sift_66_appear | | |
| | sift_67_appear | | |
| | sift_68_appear | | |
| | sift_69_appear | | |
| | sift_70_appear | | |
| | sift_71_appear | | |
| | sift_72_appear | | |
| | sift_73_appear | | |
| | sift_74_appear | | |
| | sift_75_appear | | |
| | sift_76_appear | | |
| | sift_77_appear | | |
| | sift_78_appear | | |
| | sift_79_appear | | |
| | sift_80_appear | | |
| | sift_81_appear | | |
| | sift_82_appear | | |
| | sift_83_appear | | |
| | sift_84_appear | | |
| | sift_85_appear | | |
| | sift_86_appear | | |
| | sift_87_appear | | |
| | sift_88_appear | | |
| | sift_89_appear | | |
| | sift_90_appear | | |
| | sift_91_appear | | |
| | sift_92_appear | | |
| | sift_93_appear | | |
| | sift_94_appear | | |
| | sift_95_appear | | |
| | sift_96_appear | | |
| | sift_97_appear | | |
| | sift_98_appear | | |
| | sift_99_appear | | |
| | sift_100_appear | | |
| | sift_101_appear | | |
| | sift_102_appear | | |
| | sift_103_appear | | |
| | sift_104_appear | | |
| | sift_105_appear | | |
| | sift_106_appear | | |
| | sift_107_appear | | |
| | sift_108_appear | | |
| | sift_109_appear | | |
| | sift_110_appear | | |
| | sift_111_appear | | |
| | sift_112_appear | | |
| | sift_113_appear | | |
| | sift_114_appear | | |
| | sift_115_appear | | |
| | sift_116_appear | | |
| | sift_117_appear | | |
| | sift_118_appear | | |
| | sift_119_appear | | |
| | sift_120_appear | | |
| | sift_121_appear | | |
| | sift_122_appear | | |
| | sift_123_appear | | |
| | sift_124_appear | | |
| | sift_125_appear | | |
| | sift_126_appear | | |
| | sift_127_appear | | |
| | sift_128_appear | | |

TABLE 3
SAM - M (Motion phenome)

| | | | | | Measurement | Metric |
|---|---|---|---|---|---|---|
| Feature/Descriptor | Feature Names | Feature Category | Feature set | Scope | Type | Unit |
| Mean global speed | mean_speed_global | mean boundary | Speed features | Global | scalar | μm/h |
| | | speed statistics | | | | |
| Standard deviation | stddev_speed_global | mean boundary | Speed features | | scalar | μm/h |
| global speed | | speed statistics | | | | |
| Mean global optical flow | mean_speed_global_flow | mean optical flow | Speed features | Global | scalar | μm/h |
| speed | | speed statistics | | | | |
| Standard deviation | stddev_speed_global_flow | mean optical flow | Speed features | Global | scalar | μm/h |
| global optical flow speed | | speed statistics | | | | |
| Mean curl optical flow | mean_curl_global_flow | mean optical flow | Motion | Global | scalar | 1/h |
| | | speed statistics | directional | | | |
| Standard deviation curl | stddev_curl_global_flow | mean optical flow | Motion | Global | scalar | 1/h |
| optical flow | | speed statistics | directional | | | |
| Mean divergence optical | mean_divergence_global_flow | mean optical flow | Motion | Global | scalar | 1/h |
| flow | | speed statistics | directional | | | |
| Standard deviation | stddev_divergence_global_flow | mean optical flow | Motion | Global | scalar | 1/h |
| divergence optical flow | | speed statistics | directional | | | |
| Histogram of regional | hist_bin_1_region_001 | mean regional | Local texture | Regional | | |
| optical flow speeds | | optical flow speed | | | | |
| (Region 1, border | | histograms | | | | |
| region) | hist_bin_2_region_001 | mean regional | features | | | |
| | | optical flow speed | | | | |
| | | histograms | | | | |
| | hist_bin_3_region_001 | mean regional | | | | |
| | | optical flow speed | | | | |
| | | histograms | | | | |
| | hist_bin_4_region_001 | mean regional | | | | |
| | | optical flow speed | | | | |
| | | histograms | | | | |
| | hist_bin_5_region_001 | mean regional | | | | |
| | | optical flow speed | | | | |
| | | histograms | | | | |
| | hist_bin_6_region_001 | mean regional | | | | |
| | | optical flow speed | | | | |
| | | histograms | | | | |
| | hist_bin_7_region_001 | mean regional | | | | |
| | | optical flow speed | | | | |
| | | histograms | | | | |
| | hist_bin_8_region_001 | mean regional | | | | |
| | | optical flow speed | | | | |
| | | histograms | | | | |
| Mean regional optical | mean_speed_flow_region_1 | mean regional | Speed features | Regional | scalar | μm/h |
| flow speed (Region 1, | | optical flow speed | | | | |
| border region) | | statistics | | | | |
| Standard deviation | stddev_speed_flow_region1 | mean regional | Speed features | | scalar | μm/h |
| optical flow speed | | optical flow speed | | | | |
| (Region 1, border | | statistics | | | | |
| Histogram of regional | hist_bin_1_region_002 | mean regional | Local texture | Regional | | |
| optical flow speeds | | optical flow speed | features | | | |
| (Region 2, mid region) | | histograms | | | | |
| | hist_bin_2_region_002 | mean regional | | | | |
| | | optical flow speed | | | | |
| | | histograms | | | | |
| | hist_bin_3_region_002 | mean regional | | | | |
| | | optical flow speed | | | | |
| | | histograms | | | | |
| | hist_bin_4_region_002 | mean regional | | | | |
| | | optical flow speed | | | | |
| | | histograms | | | | |
| | hist_bin_5_region_002 | mean regional | | | | |
| | | optical flow speed | | | | |
| | | histograms | | | | |
| | hist_bin_6_region_002 | mean regional | | | | |
| | | optical flow speed | | | | |
| | | histograms | | | | |
| | hist_bin_7_region_002 | mean regional | | | | |
| | | optical flow speed | | | | |
| | | histograms | | | | |
| | hist_bin_8_region_002 | mean regional | | | | |
| | | optical flow speed | | | | |
| | | histograms | | | | |
| Mean regional optical | mean_speed_flow_region_2 | mean regional | Speed features | Regional | scalar | μm/h |
| flow speed (Region 2, | | optical flow speed | | | | |
| mid region) | | statistics | | | | |
| Standard deviation | stddev_speed_flow_region2 | mean regional | Speed features | | scalar | μm/h |
| optical flow speed | | optical flow speed | | | | |
| (Region 2, mid region) | | statistics | | | | |
| Histogram of regional | hist_bin_1_region_003 | mean regional | Local texture | Regional | | |
| optical flow speeds | | optical flow speed | features | | | |
| (Region 3, central | | histograms | | | | |
| region) | hist_bin_2_region_003 | mean regional | | | | |
| | | optical flow speed | | | | |
| | | histograms | | | | |
| | hist_bin_3_region_003 | mean regional | | | | |
| | | optical flow speed | | | | |
| | | histograms | | | | |
| | hist_bin_4_region_003 | mean regional | | | | |
| | | optical flow speed | | | | |
| | | histograms | | | | |
| | hist_bin_5_region_003 | mean regional | | | | |
| | | optical flow speed | | | | |
| | | histograms | | | | |
| | hist_bin_6_region_003 | mean regional | | | | |
| | | optical flow speed | | | | |
| | | histograms | | | | |
| | hist_bin_7_region003 | mean regional | | | | |
| | | optical flow speed | | | | |
| | | histograms | | | | |
| | hist_bin_8_region_003 | mean regional | | | | |
| | | optical flow speed | | | | |
| | | histograms | | | | |
| Mean regional optical | mean_speed_flow_region_3 | mean regional | Speed features | Regional | scalar | μm/h |
| flow speed (Region 3, | | optical flow speed | | | | |
| central region) | | statistics | | | | |
| Standard deviation | stddev_speed_flow_region3 | mean regional | Speed features | | scalar | μm/h |
| optical flow speed | | optical flow speed | | | | |
| (Region 3, central | | statistics | | | | |
| Scale invariant feature | sift_1_motion | speed SIFT features | Local texture | Distribution | histogram | |
| transform of optical flow | sift_2_motion | speed SIFT features | features | | | |
| speed | sift_3_motion | speed SIFT features | | | | |
| | sift_4_motion | speed SIFT features | | | | |
| | sift_5_motion | speed SIFT features | | | | |
| | sift_6_motion | speed SIFT features | | | | |
| | sift_7_motion | speed SIFT features | | | | |
| | sift_8_motion | speed SIFT features | | | | |
| | sift_9_motion | speed SIFT features | | | | |
| | sift_10_motion | speed SIFT features | | | | |
| | sift_11_motion | speed SIFT features | | | | |
| | sift_12_motion | speed SIFT features | | | | |
| | sift_13_motion | speed SIFT features | | | | |
| | sift_14_motion | speed SIFT features | | | | |
| | sift_15_motion | speed SIFT features | | | | |
| | sift_16_motion | speed SIFT features | | | | |
| | sift_17_motion | speed SIFT features | | | | |
| | sift_18_motion | speed SIFT features | | | | |
| | sift_19_motion | speed SIFT features | | | | |
| | sift_20_motion | speed SIFT features | | | | |
| | sift_21_motion | speed SIFT features | | | | |
| | sift_22_motion | speed SIFT features | | | | |
| | sift_23_motion | speed SIFT features | | | | |
| | sift_24_motion | speed SIFT features | | | | |
| | sift_25_motion | speed SIFT features | | | | |
| | sift_26_motion | speed SIFT features | | | | |
| | sift_27_motion | speed SIFT features | | | | |
| | sift_28_motion | speed SIFT features | | | | |
| | sift_29_motion | speed SIFT features | | | | |
| | sift_30_motion | speed SIFT features | | | | |
| | sift_31_motion | speed SIFT features | | | | |
| | sift_32_motion | speed SIFT features | | | | |
| | sift_33_motion | speed SIFT features | | | | |
| | sift_34_motion | speed SIFT features | | | | |
| | sift_35_motion | speed SIFT features | | | | |
| | sift_36_motion | speed SIFT features | | | | |
| | sift_37_motion | speed SIFT features | | | | |
| | sift_38_motion | speed SIFT features | | | | |
| | sift_39_motion | speed SIFT features | | | | |
| | sift_40_motion | speed SIFT features | | | | |
| | sift_41_motion | speed SIFT features | | | | |
| | sift_42_motion | speed SIFT features | | | | |
| | sift_43_motion | speed SIFT features | | | | |
| | sift_44_motion | speed SIFT features | | | | |
| | sift_45_motion | speed SIFT features | | | | |
| | sift_46_motion | speed SIFT features | | | | |
| | sift_47_motion | speed SIFT features | | | | |
| | sift_48_motion | speed SIFT features | | | | |
| | sift_49_motion | speed SIFT features | | | | |
| | sift_50_motion | speed SIFT features | | | | |
| | sift_51_motion | speed SIFT features | | | | |
| | sift_52_motion | speed SIFT features | | | | |
| | sift_53_motion | speed SIFT features | | | | |
| | sift_54_motion | speed SIFT features | | | | |
| | sift_55_motion | speed SIFT features | | | | |
| | sift_56_motion | speed SIFT features | | | | |
| | sift_57_motion | speed SIFT features | | | | |
| | sift_58_motion | speed SIFT features | | | | |
| | sift_59_motion | speed SIFT features | | | | |
| | sift_60_motion | speed SIFT features | | | | |
| | sift_61_motion | speed SIFT features | | | | |
| | sift_62_motion | speed SIFT features | | | | |
| | sift_63_motion | speed SIFT features | | | | |
| | sift_64_motion | speed SIFT features | | | | |
| | sift_65_motion | speed SIFT features | | | | |
| | sift_66_motion | speed SIFT features | | | | |
| | sift_67_motion | speed SIFT features | | | | |
| | sift_68_motion | speed SIFT features | | | | |
| | sift_69_motion | speed SIFT features | | | | |
| | sift_70_motion | speed SIFT features | | | | |
| | sift_71_motion | speed SIFT features | | | | |
| | sift_72_motion | speed SIFT features | | | | |
| | sift_73_motion | speed SIFT features | | | | |
| | sift_74_motion | speed SIFT features | | | | |
| | sift_75_motion | speed SIFT features | | | | |
| | sift_76_motion | speed SIFT features | | | | |
| | sift_77_motion | speed SIFT features | | | | |
| | sift_78_motion | speed SIFT features | | | | |
| | sift_79_motion | speed SIFT features | | | | |
| | sift_80_motion | speed SIFT features | | | | |
| | sift_81_motion | speed SIFT features | | | | |
| | sift_82_motion | speed SIFT features | | | | |
| | sift_83_motion | speed SIFT features | | | | |
| | sift_84_motion | speed SIFT features | | | | |
| | sift_85_motion | speed SIFT features | | | | |
| | sift_86_motion | speed SIFT features | | | | |
| | sift_87_motion | speed SIFT features | | | | |
| | sift_88_motion | speed SIFT features | | | | |
| | sift_89_motion | speed SIFT features | | | | |
| | sift_90_motion | speed SIFT features | | | | |
| | sift_91_motion | speed SIFT features | | | | |
| | sift_92_motion | speed SIFT features | | | | |
| | sift_93_motion | speed SIFT features | | | | |
| | sift_94_motion | speed SIFT features | | | | |
| | sift_95_motion | speed SIFT features | | | | |
| | sift_96_motion | speed SIFT features | | | | |
| | sift_97_motion | speed SIFT features | | | | |
| | sift_98_motion | speed SIFT features | | | | |
| | sift_99_motion | speed SIFT features | | | | |
| | sift_100_motion | speed SIFT features | | | | |
| | sift_101_motion | speed SIFT features | | | | |
| | sift_102_motion | speed SIFT features | | | | |
| | sift_103_motion | speed SIFT features | | | | |
| | sift_104_motion | speed SIFT features | | | | |
| | sift_105_motion | speed SIFT features | | | | |
| | sift_106_motion | speed SIFT features | | | | |
| | sift_107_motion | speed SIFT features | | | | |
| | sift_108_motion | speed SIFT features | | | | |
| | sift_109_motion | speed SIFT features | | | | |
| | sift_110_motion | speed SIFT features | | | | |
| | sift_111_motion | speed SIFT features | | | | |
| | sift_112_motion | speed SIFT features | | | | |
| | sift_113_motion | speed SIFT features | | | | |
| | sift_114_motion | speed SIFT features | | | | |
| | sift_115_motion | speed SIFT features | | | | |
| | sift_116_motion | speed SIFT features | | | | |
| | sift_117_motion | speed SIFT features | | | | |
| | sift_118_motion | speed SIFT features | | | | |
| | sift_119_motion | speed SIFT features | | | | |
| | sift_120_motion | speed SIFT features | | | | |
| | sift_121_motion | speed SIFT features | | | | |
| | sift_122_motion | speed SIFT features | | | | |
| | sift_123_motion | speed SIFT features | | | | |
| | sift_124_motion | speed SIFT features | | | | |
| | sift_125_motion | speed SIFT features | | | | |
| | sift_126_motion | speed SIFT features | | | | |
| | sift_127_motion | speed SIFT features | | | | |
| | sift_128_motion | speed SIFT features | | | | |
| Scale invariant feature | curl_sift_1 | Curl SIFT features | Local texture | Distribution | histogram | |
| transform of curl of | curl_sift_2 | Curl SIFT features | features | | | |
| optical flow speed | curl_sift_3 | Curl SIFT features | | | | |
| | curl_sift_4 | Curl SIFT features | | | | |
| | curl_sift_5 | Curl SIFT features | | | | |
| | curl_sift_6 | Curl SIFT features | | | | |
| | curl_sift_7 | Curl SIFT features | | | | |
| | curl_sift_8 | Curl SIFT features | | | | |
| | curl_sift_9 | Curl SIFT features | | | | |
| | curl_sift_10 | Curl SIFT features | | | | |
| | curl_sift_11 | Curl SIFT features | | | | |
| | curl_sift_12 | Curl SIFT features | | | | |
| | curl_sift_13 | Curl SIFT features | | | | |
| | curl_sift_14 | Curl SIFT features | | | | |
| | curl_sift_15 | Curl SIFT features | | | | |
| | curl_sift_16 | Curl SIFT features | | | | |
| | curl_sift_17 | Curl SIFT features | | | | |
| | curl_sift_18 | Curl SIFT features | | | | |
| | curl_sift_19 | Curl SIFT features | | | | |
| | curl_sift_20 | Curl SIFT features | | | | |
| | curl_sift_21 | Curl SIFT features | | | | |
| | curl_sift_22 | Curl SIFT features | | | | |
| | curl_sift_23 | Curl SIFT features | | | | |
| | curl_sift_24 | Curl SIFT features | | | | |
| | curl_sift_25 | Curl SIFT features | | | | |
| | curl_sift_26 | Curl SIFT features | | | | |
| | curl_sift_27 | Curl SIFT features | | | | |
| | curl_sift_28 | Curl SIFT features | | | | |
| | curl_sift_29 | Curl SIFT features | | | | |
| | curl_sift_30 | Curl SIFT features | | | | |
| | curl_sift_31 | Curl SIFT features | | | | |
| | curl_sift_32 | Curl SIFT features | | | | |
| | curl_sift_33 | Curl SIFT features | | | | |
| | curl_sift_34 | Curl SIFT features | | | | |
| | curl_sift_35 | Curl SIFT features | | | | |
| | curl_sift_36 | Curl SIFT features | | | | |
| | curl_sift_37 | Curl SIFT features | | | | |
| | curl_sift_38 | Curl SIFT features | | | | |
| | curl_sift_39 | Curl SIFT features | | | | |
| | curl_sift_40 | Curl SIFT features | | | | |
| | curl_sift_41 | Curl SIFT features | | | | |
| | curl_sift_42 | Curl SIFT features | | | | |
| | curl_sift_43 | Curl SIFT features | | | | |
| | curl_sift_44 | Curl SIFT features | | | | |
| | curl_sift_45 | Curl SIFT features | | | | |
| | curl_sift_46 | Curl SIFT features | | | | |
| | curl_sift_47 | Curl SIFT features | | | | |
| | curl_sift_48 | Curl SIFT features | | | | |
| | curl_sift_49 | Curl SIFT features | | | | |
| | curl_sift_50 | Curl SIFT features | | | | |
| | curl_sift_51 | Curl SIFT features | | | | |
| | curl_sift_52 | Curl SIFT features | | | | |
| | curl_sift_53 | Curl SIFT features | | | | |
| | curl_sift_54 | Curl SIFT features | | | | |
| | curl_sift_55 | Curl SIFT features | | | | |
| | curl_sift_56 | Curl SIFT features | | | | |
| | curl_sift_57 | Curl SIFT features | | | | |
| | curl_sift_58 | Curl SIFT features | | | | |
| | curl_sift_59 | Curl SIFT features | | | | |
| | curl_sift_60 | Curl SIFT features | | | | |
| | curl_sift_61 | Curl SIFT features | | | | |
| | curl_sift_62 | Curl SIFT features | | | | |
| | curl_sift_63 | Curl SIFT features | | | | |
| | curl_sift_64 | Curl SIFT features | | | | |
| | curl_sift_65 | Curl SIFT features | | | | |
| | curl_sift_66 | Curl SIFT features | | | | |
| | curl_sift_67 | Curl SIFT features | | | | |
| | curl_sift_68 | Curl SIFT features | | | | |
| | curl_sift_69 | Curl SIFT features | | | | |
| | curl_sift_70 | Curl SIFT features | | | | |
| | curl_sift_71 | Curl SIFT features | | | | |
| | curl_sift_72 | Curl SIFT features | | | | |
| | curl_sift_73 | Curl SIFT features | | | | |
| | curl_sift_74 | Curl SIFT features | | | | |
| | curl_sift_75 | Curl SIFT features | | | | |
| | curl_sift_76 | Curl SIFT features | | | | |
| | curl_sift_77 | Curl SIFT features | | | | |
| | curl_sift_78 | Curl SIFT features | | | | |
| | curl_sift_79 | Curl SIFT features | | | | |
| | curl_sift_80 | Curl SIFT features | | | | |
| | curl_sift_81 | Curl SIFT features | | | | |
| | curl_sift_82 | Curl SIFT features | | | | |
| | curl_sift_83 | Curl SIFT features | | | | |
| | curl_sift_84 | Curl SIFT features | | | | |
| | curl_sift_85 | Curl SIFT features | | | | |
| | curl_sift_86 | Curl SIFT features | | | | |
| | curl_sift_87 | Curl SIFT features | | | | |
| | curl_sift_88 | Curl SIFT features | | | | |
| | curl_sift_89 | Curl SIFT features | | | | |
| | curl_sift_90 | Curl SIFT features | | | | |
| | curl_sift_91 | Curl SIFT features | | | | |
| | curl_sift_92 | Curl SIFT features | | | | |
| | curl_sift_93 | Curl SIFT features | | | | |
| | curl_sift_94 | Curl SIFT features | | | | |
| | curl_sift_95 | Curl SIFT features | | | | |
| | curl_sift_96 | Curl SIFT features | | | | |
| | curl_sift_97 | Curl SIFT features | | | | |
| | curl_sift_98 | Curl SIFT features | | | | |
| | curl_sift_99 | Curl SIFT features | | | | |
| | curl_sift_100 | Curl SIFT features | | | | |
| | curl_sift_101 | Curl SIFT features | | | | |
| | curl_sift_102 | Curl SIFT features | | | | |
| | curl_sift_103 | Curl SIFT features | | | | |
| | curl_sift_104 | Curl SIFT features | | | | |
| | curl_sift_105 | Curl SIFT features | | | | |
| | curl_sift_106 | Curl SIFT features | | | | |
| | curl_sift_107 | Curl SIFT features | | | | |
| | curl_sift_108 | Curl SIFT features | | | | |
| | curl_sift_109 | Curl SIFT features | | | | |
| | curl_sift_110 | Curl SIFT features | | | | |
| | curl_sift_111 | Curl SIFT features | | | | |
| | curl_sift_112 | Curl SIFT features | | | | |
| | curl_sift_113 | Curl SIFT features | | | | |
| | curl_sift_114 | Curl SIFT features | | | | |
| | curl_sift_115 | Curl SIFT features | | | | |
| | curl_sift_116 | Curl SIFT features | | | | |
| | curl_sift_117 | Curl SIFT features | | | | |
| | curl_sift_118 | Curl SIFT features | | | | |
| | curl_sift_119 | Curl SIFT features | | | | |
| | curl_sift_120 | Curl SIFT features | | | | |
| | curl_sift_121 | Curl SIFT features | | | | |
| | curl_sift_122 | Curl SIFT features | | | | |
| | curl_sift_123 | Curl SIFT features | | | | |
| | curl_sift_124 | Curl SIFT features | | | | |
| | curl_sift_125 | Curl SIFT features | | | | |
| | curl_sift_126 | Curl SIFT features | | | | |
| | curl_sift_127 | Curl SIFT features | | | | |
| | curl_sift_128 | Curl SIFT features | | | | |
| Scale invariant feature | div_sift_1 | Divergence SIFT | Local texture | Distribution | Histogram | |
| transform of divergence | | features | features | | | |
| of optical flow speed | div_sift_2 | Divergence SIFT | | | | |
| | | features | | | | |
| | div_sift_3 | Divergence SIFT | | | | |
| | | features | | | | |
| | div_sift_4 | Divergence SIFT | | | | |
| | | features | | | | |
| | div_sift_5 | Divergence SIFT | | | | |
| | | features | | | | |
| | div_sift_6 | Divergence SIFT | | | | |
| | | features | | | | |
| | div_sift_7 | Divergence SIFT | | | | |
| | | features | | | | |
| | div_sift_8 | Divergence SIFT | | | | |
| | | features | | | | |
| | div_sift_9 | Divergence SIFT | | | | |
| | | features | | | | |
| | div_sift_10 | Divergence SIFT | | | | |
| | | features | | | | |
| | div_sift_11 | Divergence SIFT | | | | |
| | | features | | | | |
| | div_sift_12 | Divergence SIFT | | | | |
| | | features | | | | |
| | div_sift_13 | Divergence SIFT | | | | |
| | | features | | | | |
| | div_sift_14 | Divergence SIFT | | | | |
| | | features | | | | |
| | div_sift_15 | Divergence SIFT | | | | |
| | | features | | | | |
| | div_sift_16 | Divergence SIFT | | | | |
| | | features | | | | |
| | div_sift_17 | Divergence SIFT | | | | |
| | | features | | | | |
| | div_sift_18 | Divergence SIFT | | | | |
| | | features | | | | |
| | div_sift_19 | Divergence SIFT | | | | |
| | | features | | | | |
| | div_sift_20 | Divergence SIFT | | | | |
| | | features | | | | |
| | div_sift_21 | Divergence SIFT | | | | |
| | | features | | | | |
| | div_sift_22 | Divergence SIFT | | | | |
| | | features | | | | |
| | div_sift_23 | Divergence SIFT | | | | |
| | | features | | | | |
| | div_sift_24 | Divergence SIFT | | | | |
| | | features | | | | |
| | div_sift_25 | Divergence SIFT | | | | |
| | | features | | | | |
| | div_sift_26 | Divergence SIFT | | | | |
| | | features | | | | |
| | div_sift_27 | Divergence SIFT | | | | |
| | | features | | | | |
| | div_sift_28 | Divergence SIFT | | | | |
| | | features | | | | |
| | div_sift_29 | Divergence SIFT | | | | |
| | | features | | | | |
| | div_sift_30 | Divergence SIFT | | | | |
| | | features | | | | |
| | div_sift_31 | Divergence SIFT | | | | |
| | | features | | | | |
| | div_sift_32 | Divergence SIFT | | | | |
| | | features | | | | |
| | div_sift_33 | Divergence SIFT | | | | |
| | | features | | | | |
| | div_sift_34 | Divergence SIFT | | | | |
| | | features | | | | |
| | div_sift_35 | Divergence SIFT | | | | |
| | | features | | | | |
| | div_sift_36 | Divergence SIFT | | | | |
| | | features | | | | |
| | div_sift_37 | Divergence SIFT | | | | |
| | | features | | | | |
| | div_sift_38 | Divergence SIFT | | | | |
| | | features | | | | |
| | div_sift_39 | Divergence SIFT | | | | |
| | | features | | | | |
| | div_sift_40 | Divergence SIFT | | | | |
| | | features | | | | |
| | div_sift_41 | Divergence SIFT | | | | |
| | | features | | | | |
| | div_sift_42 | Divergence SIFT | | | | |
| | | features | | | | |
| | div_sift_43 | Divergence SIFT | | | | |
| | | features | | | | |
| | div_sift_44 | Divergence SIFT | | | | |
| | | features | | | | |
| | div_sift_45 | Divergence SIFT | | | | |
| | | features | | | | |
| | div_sift_46 | Divergence SIFT | | | | |
| | | features | | | | |
| | div_sift_47 | Divergence SIFT | | | | |
| | | features | | | | |
| | div_sift_48 | Divergence SIFT | | | | |
| | | features | | | | |
| | div_sift_49 | Divergence SIFT | | | | |
| | | features | | | | |
| | div_sift_50 | Divergence SIFT | | | | |
| | | features | | | | |
| | div_sift_51 | Divergence SIFT | | | | |
| | | features | | | | |
| | div_sift_52 | Divergence SIFT | | | | |
| | | features | | | | |
| | div_sift_53 | Divergence SIFT | | | | |
| | | features | | | | |
| | div_sift_54 | Divergence SIFT | | | | |
| | | features | | | | |
| | div_sift_55 | Divergence SIFT | | | | |
| | | features | | | | |
| | div_sift_56 | Divergence SIFT | | | | |
| | | features | | | | |
| | div_sift_57 | Divergence SIFT | | | | |
| | | features | | | | |
| | div_sift_58 | Divergence SIFT | | | | |
| | | features | | | | |
| | div_sift_59 | Divergence SIFT | | | | |
| | | features | | | | |
| | div_sift_60 | Divergence SIFT | | | | |
| | | features | | | | |
| | div_sift_61 | Divergence SIFT | | | | |
| | | features | | | | |
| | div_sift_62 | Divergence SIFT | | | | |
| | | features | | | | |
| | div_sift_63 | Divergence SIFT | | | | |
| | | features | | | | |
| | div_sift_64 | Divergence SIFT | | | | |
| | | features | | | | |
| | div_sift_65 | Divergence SIFT | | | | |
| | | features | | | | |
| | div_sift_66 | Divergence SIFT | | | | |
| | | features | | | | |
| | div_sift_67 | Divergence SIFT | | | | |
| | | features | | | | |
| | div_sift_68 | Divergence SIFT | | | | |
| | | features | | | | |
| | div_sift_69 | Divergence SIFT | | | | |
| | | features | | | | |
| | div_sift_70 | Divergence SIFT | | | | |
| | | features | | | | |
| | div_sift_71 | Divergence SIFT | | | | |
| | | features | | | | |
| | div_sift_72 | Divergence SIFT | | | | |
| | | features | | | | |
| | div_sift_73 | Divergence SIFT | | | | |
| | | features | | | | |
| | div_sift_74 | Divergence SIFT | | | | |
| | | features | | | | |
| | div_sift_75 | Divergence SIFT | | | | |
| | | features | | | | |
| | div_sift_76 | Divergence SIFT | | | | |
| | | features | | | | |
| | div_sift_77 | Divergence SIFT | | | | |
| | | features | | | | |
| | div_sift_78 | Divergence SIFT | | | | |
| | | features | | | | |
| | div_sift_79 | Divergence SIFT | | | | |
| | | features | | | | |
| | div_sift_80 | Divergence SIFT | | | | |
| | | features | | | | |
| | div_sift_81 | Divergence SIFT | | | | |
| | | features | | | | |
| | div_sift_82 | Divergence SIFT | | | | |
| | | features | | | | |
| | div_sift_83 | Divergence SIFT | | | | |
| | | features | | | | |
| | div_sift_84 | Divergence SIFT | | | | |
| | | features | | | | |
| | div_sift_85 | Divergence SIFT | | | | |
| | | features | | | | |
| | div_sift_86 | Divergence SIFT | | | | |
| | | features | | | | |
| | div_sift_87 | Divergence SIFT | | | | |
| | | features | | | | |
| | div_sift_88 | Divergence SIFT | | | | |
| | | features | | | | |
| | div_sift_89 | Divergence SIFT | | | | |
| | | features | | | | |
| | div_sift_90 | Divergence SIFT | | | | |
| | | features | | | | |
| | div_sift_91 | Divergence SIFT | | | | |
| | | features | | | | |
| | div_sift_92 | Divergence SIFT | | | | |
| | | features | | | | |
| | div_sift_93 | Divergence SIFT | | | | |
| | | features | | | | |
| | div_sift_94 | Divergence SIFT | | | | |
| | | features | | | | |
| | div_sift_95 | Divergence SIFT | | | | |
| | | features | | | | |
| | div_sift_96 | Divergence SIFT | | | | |
| | | features | | | | |
| | div_sift_97 | Divergence SIFT | | | | |
| | | features | | | | |
| | div_sift_98 | Divergence SIFT | | | | |
| | | features | | | | |
| | div_sift_99 | Divergence SIFT | | | | |
| | | features | | | | |
| | div_sift_100 | Divergence SIFT | | | | |
| | | features | | | | |
| | div_sift_101 | Divergence SIFT | | | | |
| | | features | | | | |
| | div_sift_102 | Divergence SIFT | | | | |
| | | features | | | | |
| | div_sift_103 | Divergence SIFT | | | | |
| | | features | | | | |
| | div_sift_104 | Divergence SIFT | | | | |
| | | features | | | | |
| | div_sift_105 | Divergence SIFT | | | | |
| | | features | | | | |
| | div_sift_106 | Divergence SIFT | | | | |
| | | features | | | | |
| | div_sift_107 | Divergence SIFT | | | | |
| | | features | | | | |
| | div_sift_108 | Divergence SIFT | | | | |
| | | features | | | | |
| | div_sift_109 | Divergence SIFT | | | | |
| | | features | | | | |
| | div_sift_110 | Divergence SIFT | | | | |
| | | features | | | | |
| | div_sift_111 | Divergence SIFT | | | | |
| | | features | | | | |
| | div_sift_112 | Divergence SIFT | | | | |
| | | features | | | | |
| | div_sift_113 | Divergence SIFT | | | | |
| | | features | | | | |
| | div_sift_114 | Divergence SIFT | | | | |
| | | features | | | | |
| | div_sift_115 | Divergence SIFT | | | | |
| | | features | | | | |
| | div_sift_116 | Divergence SIFT | | | | |
| | | features | | | | |
| | div_sift_117 | Divergence SIFT | | | | |
| | | features | | | | |
| | div_sift_118 | Divergence SIFT | | | | |
| | | features | | | | |
| | div_sift_119 | Divergence SIFT | | | | |
| | | features | | | | |
| | div_sift_120 | Divergence SIFT | | | | |
| | | features | | | | |
| | div_sift_121 | Divergence SIFT | | | | |
| | | features | | | | |
| | div_sift_122 | Divergence SIFT | | | | |
| | | features | | | | |
| | div_sift_123 | Divergence SIFT | | | | |
| | | features | | | | |
| | div_sift_124 | Divergence SIFT | | | | |
| | | features | | | | |
| | div_sift_125 | Divergence SIFT | | | | |
| | | features | | | | |
| | div_sift_126 | Divergence SIFT | | | | |
| | | features | | | | |
| | div_sift_127 | Divergence SIFT | | | | |
| | | features | | | | |
| | div_sift_128 | Divergence SIFT | | | | |
| | | features | | | | |

| Feature/Descriptor | Feature Names | Description | References |
|---|---|---|---|
| Mean global speed | mean_speed_global | Mean speed, computed as the average | GitHub |
| | | displacement of each boundary point | |
| | | between two successive frames divided | |
| | | by elapsed time | |
| Standard deviation | stddev_speed_global | Standard deviation of the displacement | GitHub |
| global speed | | of each boundary point between two | |
| | | successive frames divided by elapsed time | |
| Mean global optical flow | mean_speed_global_flow | Magnitude of the mean optical flow | Farnebäck, |
| speed | | velocity vector within the organoid area | Gunnar. “Two- |
| | | | frame motion |
| | | | estimation |
| | | | based on |
| | | | polynomial |
| | | | expansion.” |
| | | | Scandinavian |
| | | | conference on |
| | | | Image analysis. |
| | | | Springer, |
| | | | Berlin, |
| | | | Heidelberg, |
| | | | 2003. |
| Standard deviation | stddev_speed_global_flow | Standard deviation of the magnitude | GitHub |
| global optical flow speed | | of individual optical flow velocity | |
| | | vectors within the organoid area | |
| Mean curl optical flow | mean_curl_global_flow | mean of the curl of the optical flow, | GitHub |
| | | or vorticity within the organoid area | |
| Standard deviation curl | stddev_curl_global_flow | Standard deviation of the curl of the | GitHub |
| optical flow | | optical flow, or vorticity within the | |
| | | organoid area | |
| Mean divergence optical | mean_divergence_global_flow | mean of the divergence of the optical | GitHub |
| flow | | flow, or ‘outgoingness’ of vector | |
| | | directions within the organoid area | |
| Standard deviation | stddev_divergence_global_flow | Standard deviation of the divergence | GitHub |
| divergence optical flow | | of the optical flow, or ‘outgoingness’ | |
| | | of vector directions within the organoid area | |
| Histogram of regional | hist_bin_1_region_001 | | GitHub |
| optical flow speeds | hist_bin_2_region_001 | | |
| (Region 1, border | hist_bin_3_region_001 | | |
| region) | hist_bin_4_region_001 | | |
| | hist_bin_5_region_001 | | |
| | hist_bin_6_region_001 | | |
| | hist_bin_7_region_001 | | |
| | hist_bin_8_region_001 | | |
| Mean regional optical | mean_speed_flow_region_1 | Magnitude of the mean optical flow vector | GitHub |
| flow speed (Region 1, | | within each of 3 regions after concentric | |
| border region) | | equidistant partitioning of the total | |
| | | organoid area | |
| Standard deviation | stddev_speed_flow_region1 | Standard deviation of the magnitude of the | GitHub |
| optical flow speed | | optical flow vectors within each of the 3 | |
| (Region 1, border | | regions after concentric equidistant | |
| | | partitioning of the total organoid area | |
| Histogram of regional | hist_bin_1_region_002 | | GitHub |
| optical flow speeds | hist_bin_2_region_002 | | |
| (Region 2, mid region) | hist_bin_3_region_002 | | |
| | hist_bin_4_region_002 | | |
| | hist_bin_5_region_002 | | |
| | hist_bin_6_region_002 | | |
| | hist_bin_7_region_002 | | |
| | hist_bin_8_region_002 | | |
| Mean regional optical | mean_speed_flow_region_2 | Magnitude of the mean optical flow vector | GitHub |
| flow speed (Region 2, | | within each of 3 regions after concentric | |
| mid region) | | equidistant partitioning of the total | |
| | | organoid area | |
| Standard deviation | stddev_speed_flow_region2 | Standard deviation of the magnitude of the | GitHub |
| optical flow speed | | optical flow vectors within each of the 3 | |
| (Region 2, mid region) | | regions after concentric equidistant | |
| | | partitioning of the total organoid area | |
| Histogram of regional | hist_bin_1_region_003 | | GitHub |
| optical flow speeds | hist_bin_2_region_003 | | |
| (Region 3, central | hist_bin_3_region_003 | | |
| region) | hist_bin_4_region_003 | | |
| | hist_bin_5_region_003 | | |
| | hist_bin_6_region_003 | | |
| | hist_bin_7_region003 | | |
| | hist_bin_8_region_003 | | |
| Mean regional optical | mean_speed_flow_region_3 | Magnitude of the mean optical flow vector | GitHub |
| flow speed (Region 3, | | within each of 3 regions after concentric | |
| central region) | | equidistant partitioning of the total | |
| | | organoid area | |
| Standard deviation | stddev_speed_flow_region3 | Standard deviation of the magnitude of the | GitHub |
| optical flow speed | | optical flow vectors within each of the 3 | |
| (Region 3, central | | regions after concentric equidistant | |
| | | partitioning of the total organoid area | |
| Scale invariant feature | sift_1_motion | Histogram of spatially local optical flow | Lowe, D. G., |
| transform of optical flow | sift_2_motion | magnitude gradient directions that is | 1999, |
| speed | sift_3_motion | invariant to translation, rotation and scaling | September. |
| | sift_4_motion | | Object |
| | sift_5_motion | | recognition |
| | sift_6_motion | | from local scale- |
| | sift_7_motion | | invariant |
| | sift_8_motion | | features. In |
| | sift_9_motion | | Proceedings of |
| | sift_10_motion | | the seventh |
| | sift_11_motion | | IEEE |
| | sift_12_motion | | international |
| | sift_13_motion | | conference on |
| | sift_14_motion | | computer vision |
| | sift_15_motion | | (Vol. 2, pp. |
| | sift_16_motion | | 1150-1157). |
| | sift_17_motion | | Ieee. |
| | sift_18_motion | | |
| | sift_19_motion | | |
| | sift_20_motion | | |
| | sift_21_motion | | |
| | sift_22_motion | | |
| | sift_23_motion | | |
| | sift_24_motion | | |
| | sift_25_motion | | |
| | sift_26_motion | | |
| | sift_27_motion | | |
| | sift_28_motion | | |
| | sift_29_motion | | |
| | sift_30_motion | | |
| | sift_31_motion | | |
| | sift_32_motion | | |
| | sift_33_motion | | |
| | sift_34_motion | | |
| | sift_35_motion | | |
| | sift_36_motion | | |
| | sift_37_motion | | |
| | sift_38_motion | | |
| | sift_39_motion | | |
| | sift_40_motion | | |
| | sift_41_motion | | |
| | sift_42_motion | | |
| | sift_43_motion | | |
| | sift_44_motion | | |
| | sift_45_motion | | |
| | sift_46_motion | | |
| | sift_47_motion | | |
| | sift_48_motion | | |
| | sift_49_motion | | |
| | sift_50_motion | | |
| | sift_51_motion | | |
| | sift_52_motion | | |
| | sift_53_motion | | |
| | sift_54_motion | | |
| | sift_55_motion | | |
| | sift_56_motion | | |
| | sift_57_motion | | |
| | sift_58_motion | | |
| | sift_59_motion | | |
| | sift_60_motion | | |
| | sift_61_motion | | |
| | sift_62_motion | | |
| | sift_63_motion | | |
| | sift_64_motion | | |
| | sift_65_motion | | |
| | sift_66_motion | | |
| | sift_67_motion | | |
| | sift_68_motion | | |
| | sift_69_motion | | |
| | sift_70_motion | | |
| | sift_71_motion | | |
| | sift_72_motion | | |
| | sift_73_motion | | |
| | sift_74_motion | | |
| | sift_75_motion | | |
| | sift_76_motion | | |
| | sift_77_motion | | |
| | sift_78_motion | | |
| | sift_79_motion | | |
| | sift_80_motion | | |
| | sift_81_motion | | |
| | sift_82_motion | | |
| | sift_83_motion | | |
| | sift_84_motion | | |
| | sift_85_motion | | |
| | sift_86_motion | | |
| | sift_87_motion | | |
| | sift_88_motion | | |
| | sift_89_motion | | |
| | sift_90_motion | | |
| | sift_91_motion | | |
| | sift_92_motion | | |
| | sift_93_motion | | |
| | sift_94_motion | | |
| | sift_95_motion | | |
| | sift_96_motion | | |
| | sift_97_motion | | |
| | sift_98_motion | | |
| | sift_99_motion | | |
| | sift_100_motion | | |
| | sift_101_motion | | |
| | sift_102_motion | | |
| | sift_103_motion | | |
| | sift_104_motion | | |
| | sift_105_motion | | |
| | sift_106_motion | | |
| | sift_107_motion | | |
| | sift_108_motion | | |
| | sift_109_motion | | |
| | sift_110_motion | | |
| | sift_111_motion | | |
| | sift_112_motion | | |
| | sift_113_motion | | |
| | sift_114_motion | | |
| | sift_115_motion | | |
| | sift_116_motion | | |
| | sift_117_motion | | |
| | sift_118_motion | | |
| | sift_119_motion | | |
| | sift_120_motion | | |
| | sift_121_motion | | |
| | sift_122_motion | | |
| | sift_123_motion | | |
| | sift_124_motion | | |
| | sift_125_motion | | |
| | sift_126_motion | | |
| | sift_127_motion | | |
| | sift_128_motion | | |
| Scale invariant feature | curl_sift_1 | Histogram of spatially local optical flow | Lowe, D. G., |
| transform of curl of | curl_sift_2 | curl magnitude gradient directions that is | 1999, |
| optical flow speed | curl_sift_3 | invariant to translation, rotation and scaling | September. |
| | curl_sift_4 | | Object |
| | curl_sift_5 | | recognition |
| | curl_sift_6 | | from local scale- |
| | curl_sift_7 | | invariant |
| | curl_sift_8 | | features. In |
| | curl_sift_9 | | Proceedings of |
| | curl_sift_10 | | the seventh |
| | curl_sift_11 | | IEEE |
| | curl_sift_12 | | international |
| | curl_sift_13 | | conference on |
| | curl_sift_14 | | computer vision |
| | curl_sift_15 | | (Vol. 2, pp. |
| | curl_sift_16 | | 1150-1157). |
| | curl_sift_17 | | Ieee. |
| | curl_sift_18 | | |
| | curl_sift_19 | | |
| | curl_sift_20 | | |
| | curl_sift_21 | | |
| | curl_sift_22 | | |
| | curl_sift_23 | | |
| | curl_sift_24 | | |
| | curl_sift_25 | | |
| | curl_sift_26 | | |
| | curl_sift_27 | | |
| | curl_sift_28 | | |
| | curl_sift_29 | | |
| | curl_sift_30 | | |
| | curl_sift_31 | | |
| | curl_sift_32 | | |
| | curl_sift_33 | | |
| | curl_sift_34 | | |
| | curl_sift_35 | | |
| | curl_sift_36 | | |
| | curl_sift_37 | | |
| | curl_sift_38 | | |
| | curl_sift_39 | | |
| | curl_sift_40 | | |
| | curl_sift_41 | | |
| | curl_sift_42 | | |
| | curl_sift_43 | | |
| | curl_sift_44 | | |
| | curl_sift_45 | | |
| | curl_sift_46 | | |
| | curl_sift_47 | | |
| | curl_sift_48 | | |
| | curl_sift_49 | | |
| | curl_sift_50 | | |
| | curl_sift_51 | | |
| | curl_sift_52 | | |
| | curl_sift_53 | | |
| | curl_sift_54 | | |
| | curl_sift_55 | | |
| | curl_sift_56 | | |
| | curl_sift_57 | | |
| | curl_sift_58 | | |
| | curl_sift_59 | | |
| | curl_sift_60 | | |
| | curl_sift_61 | | |
| | curl_sift_62 | | |
| | curl_sift_63 | | |
| | curl_sift_64 | | |
| | curl_sift_65 | | |
| | curl_sift_66 | | |
| | curl_sift_67 | | |
| | curl_sift_68 | | |
| | curl_sift_69 | | |
| | curl_sift_70 | | |
| | curl_sift_71 | | |
| | curl_sift_72 | | |
| | curl_sift_73 | | |
| | curl_sift_74 | | |
| | curl_sift_75 | | |
| | curl_sift_76 | | |
| | curl_sift_77 | | |
| | curl_sift_78 | | |
| | curl_sift_79 | | |
| | curl_sift_80 | | |
| | curl_sift_81 | | |
| | curl_sift_82 | | |
| | curl_sift_83 | | |
| | curl_sift_84 | | |
| | curl_sift_85 | | |
| | curl_sift_86 | | |
| | curl_sift_87 | | |
| | curl_sift_88 | | |
| | curl_sift_89 | | |
| | curl_sift_90 | | |
| | curl_sift_91 | | |
| | curl_sift_92 | | |
| | curl_sift_93 | | |
| | curl_sift_94 | | |
| | curl_sift_95 | | |
| | curl_sift_96 | | |
| | curl_sift_97 | | |
| | curl_sift_98 | | |
| | curl_sift_99 | | |
| | curl_sift_100 | | |
| | curl_sift_101 | | |
| | curl_sift_102 | | |
| | curl_sift_103 | | |
| | curl_sift_104 | | |
| | curl_sift_105 | | |
| | curl_sift_106 | | |
| | curl_sift_107 | | |
| | curl_sift_108 | | |
| | curl_sift_109 | | |
| | curl_sift_110 | | |
| | curl_sift_111 | | |
| | curl_sift_112 | | |
| | curl_sift_113 | | |
| | curl_sift_114 | | |
| | curl_sift_115 | | |
| | curl_sift_116 | | |
| | curl_sift_117 | | |
| | curl_sift_118 | | |
| | curl_sift_119 | | |
| | curl_sift_120 | | |
| | curl_sift_121 | | |
| | curl_sift_122 | | |
| | curl_sift_123 | | |
| | curl_sift_124 | | |
| | curl_sift_125 | | |
| | curl_sift_126 | | |
| | curl_sift_127 | | |
| | curl_sift_128 | | |
| Scale invariant feature | div_sift_1 | Histogram of spatially local optical flow | Lowe, D. G., |
| transform of divergence | div_sift_2 | divergence gradient directions that is | 1999 |
| of optical flow speed | div_sift_3 | invariant to translation, rotation and | September. |
| | div_sift_4 | scaling | Object |
| | div_sift_5 | | recognition |
| | div_sift_6 | | from local scale- |
| | div_sift_7 | | invariant |
| | div_sift_8 | | features. In |
| | div_sift_9 | | Proceedings of |
| | div_sift_10 | | the seventh |
| | div_sift_11 | | IEEE |
| | div_sift_12 | | international |
| | div_sift_13 | | conference on |
| | div_sift_14 | | computer vision |
| | div_sift_15 | | (Vol. 2, pp. |
| | div_sift_16 | | 1150-1157). |
| | div_sift_17 | | Ieee. |
| | div_sift_18 | | |
| | div_sift_19 | | |
| | div_sift_20 | | |
| | div_sift_21 | | |
| | div_sift_22 | | |
| | div_sift_23 | | |
| | div_sift_24 | | |
| | div_sift_25 | | |
| | div_sift_26 | | |
| | div_sift_27 | | |
| | div_sift_28 | | |
| | div_sift_29 | | |
| | div_sift_30 | | |
| | div_sift_31 | | |
| | div_sift_32 | | |
| | div_sift_33 | | |
| | div_sift_34 | | |
| | div_sift_35 | | |
| | div_sift_36 | | |
| | div_sift_37 | | |
| | div_sift_38 | | |
| | div_sift_39 | | |
| | div_sift_40 | | |
| | div_sift_41 | | |
| | div_sift_42 | | |
| | div_sift_43 | | |
| | div_sift_44 | | |
| | div_sift_45 | | |
| | div_sift_46 | | |
| | div_sift_47 | | |
| | div_sift_48 | | |
| | div_sift_49 | | |
| | div_sift_50 | | |
| | div_sift_51 | | |
| | div_sift_52 | | |
| | div_sift_53 | | |
| | div_sift_54 | | |
| | div_sift_55 | | |
| | div_sift_56 | | |
| | div_sift_57 | | |
| | div_sift_58 | | |
| | div_sift_59 | | |
| | div_sift_60 | | |
| | div_sift_61 | | |
| | div_sift_62 | | |
| | div_sift_63 | | |
| | div_sift_64 | | |
| | div_sift_65 | | |
| | div_sift_66 | | |
| | div_sift_67 | | |
| | div_sift_68 | | |
| | div_sift_69 | | |
| | div_sift_70 | | |
| | div_sift_71 | | |
| | div_sift_72 | | |
| | div_sift_73 | | |
| | div_sift_74 | | |
| | div_sift_75 | | |
| | div_sift_76 | | |
| | div_sift_77 | | |
| | div_sift_78 | | |
| | div_sift_79 | | |
| | div_sift_80 | | |
| | div_sift_81 | | |
| | div_sift_82 | | |
| | div_sift_83 | | |
| | div_sift_84 | | |
| | div_sift_85 | | |
| | div_sift_86 | | |
| | div_sift_87 | | |
| | div_sift_88 | | |
| | div_sift_89 | | |
| | div_sift_90 | | |
| | div_sift_91 | | |
| | div_sift_92 | | |
| | div_sift_93 | | |
| | div_sift_94 | | |
| | div_sift_95 | | |
| | div_sift_96 | | |
| | div_sift_97 | | |
| | div_sift_98 | | |
| | div_sift_99 | | |
| | div_sift_100 | | |
| | div_sift_101 | | |
| | div_sift_102 | | |
| | div_sift_103 | | |
| | div_sift_104 | | |
| | div_sift_105 | | |
| | div_sift_106 | | |
| | div_sift_107 | | |
| | div_sift_108 | | |
| | div_sift_109 | | |
| | div_sift_110 | | |
| | div_sift_111 | | |
| | div_sift_112 | | |
| | div_sift_113 | | |
| | div_sift_114 | | |
| | div_sift_115 | | |
| | div_sift_116 | | |
| | div_sift_117 | | |
| | div_sift_118 | | |
| | div_sift_119 | | |
| | div_sift_120 | | |
| | div_sift_121 | | |
| | div_sift_122 | | |
| | div_sift_123 | | |
| | div_sift_124 | | |
| | div_sift_125 | | |
| | div_sift_126 | | |
| | div_sift_127 | | |
| | div_sift_128 | | |

TABLE 4
Unsupervised k-means clustering of MPEG-7 Shape Dataset with different possible SAM shape (SAM-S) descriptors MPEG-7 database is a standard
computer vision dataset for shape classification. 20 examples are provided from 70 shape categories as binary images. The contours of the
shapes discretized with equally spaced 200 boundary points were extracted from which we computed SAM shape features. Both the full SAM shape
descriptor (SAM-S) as well as its distinct component subset shape feature sets were comparedusing k-means clustering where k = 20, the
number of known shape clusters. A particular feature set, euler characteristic curves (ECC) are inherently high dimensional (1152), sparse,
categorical features which do not have much discriminative power as-is. Thus, dimensional reduction (kernel ECC, n = 100) was performed,
and included this in the separate and as part of the full SAM-S descriptor denoted SAM-S (kernel ECC) as opposed to simply using the raw
ECC, SAM-S (ECC). The clustering performance metrics: adjusted mutual information (AMI) and adjusted rand index (ARI) for validation, were used

| | k-means clustering to identify MPEG-7 shape categories (k = 70) | | | | | |
|---|---|---|---|---|---|---|
| | | | | ½*(AMI + | Average | |
| | Feature Descriptor | AMI | ARI | ARI) | Ranking | Feature Scope |
| Subset features of SAM- | Shape context, n = 300 | 0.652 | 0.435 | 0.544 | 3 | Distribution |
| A | Zernike moment, n = 25 | 0.638 | 0.413 | 0.526 | 4 | Regional |
| | Geometrical, n = 17 | 0.587 | 0.378 | 0.483 | 5 | Global |
| | chordal distance histograms, n = 8 | 0.555 | 0.327 | 0.441 | 7 | Distribution |
| | kernel Euler Characteristic Curve (kernel ECC), n = 100 ** | 0.528 | 0.320 | 0.424 | 8 | Distribution |
| | centroid statistics, n = 5 | 0.510 | 0.296 | 0.403 | 9 | Global |
| | Hu moments, n = 7 | 0.469 | 0.200 | 0.335 | 10 | Regional |
| | Euler Characteristic Curve (ECC), n = 1152 | 0.506 | 0.133 | 0.320 | 11 | Distribution |
| | Fourier features, n = 99 | 0.275 | 0.114 | 0.195 | 12 | Regional |
| Full SAM Appearance | SAM-S (ECC), n = 1614 | 0.634 | 0.327 | 0.481 | 6 | Combination |
| descriptor (SAM-A) | SAM-S (kernel ECC), n = 562 | 0.710 | 0.518 | 0.614 | 1 | Combination |
| Cellprofiler Shape | Cellprofiler shape features, n = 98 | 0.684 | 0.466 | 0.575 | 2 | Combination |
| features | | | | | | |
| Negative control | Random label permutation | 0.000 | 0.000 | 0.000 | 13 | n/a |

** kernel ECC is a dimensionality reduction of ECC features, based on comparing the similarity of ECC over multiple objects and therefore
AMI—adjusted mutual information score (upper-bounded by 1) the larger the better.
https://scikit-learn.org/stable/modules/generated/sklearn.metrics.adjusted_mutual_info score.html
ARI—adjusted rand index score (−0.5 to 1.0). −0.5 = especially discordant, 0.0 = random labelings, 1.0 = perfect match
https://scikit-learn.org/stable/modules/generated/sklearn.metrics.adjusted_rand_score.html#sklearn.metrics.adjusted_rand_score

TABLE 5
Unsupervised k-means clustering of Brodatz Texture Images with different possible SAM appearance
(SAM-A) descriptors The normalized Brodatz texture database contains 112 unique 512 × 512
pixel texture images. Normalized refers to the use of an intensity normalization process to eliminate
the original's grayscale background effect which provides a data bias that can overestimate
a method's performance. To test SAM appearance descriptor (SAM-A), a 14,336 dataset of 64 ×
64 pixel images were created by cropping each of the 512 × 512 texture images into 64 non-
overlapping 64 × 64 patches and then for each patch including additionally the patch rotated
by 90 degrees. Both the full SAM appearance descriptor (SAM-A) as well as its distinct component
subset appearance feature sets were compared using k-means clustering where k = 112, the
number of Brodatz texture images the patches originate from. The clustering performance metrics
used: adjusted mutual information (AMI) and adjusted rand index (ARI) for validation

| | k-means clustering to identify Brodatz texture images (k = 112) | | | | | |
|---|---|---|---|---|---|---|
| | | | | ½*(AMI + | Average | Scope |
| | Feature Descriptor | AMI | ARI | ARI) | Ranking | Feature |
| Subset features of SAM-A | Haralick texture | 0.446 | 0.126 | 0.286 | 3 | Distribution |
| | SIFT features, n = 128 | 0.408 | 0.111 | 0.260 | 4 | Distribution |
| | Intensity statistics, n = 8 | 0.195 | 0.025 | 0.110 | 5 | Global + |
| | | | | | | Regional |
| Full SAM Appearance | SAM-A, n = 149 | 0.464 | 0.141 | 0.303 | 2 | Combination |
| descriptor (SAM-A) | | | | | | |
| Cellprofiler Appearance | Cellprofiler | 0.487 | 0.143 | 0.315 | 1 | Combination |
| features | appearance features, | | | | | |
| Negative control | Random label | −0.001 | 0.000 | −0.001 | 6 | n/a |

AMI—adjusted mutual information score (upper-bounded by 1) the larger the better.
https://scikit-learn.org/stable/modules/generated/sklearn.metrics.adjusted_mutual_info
ARI—adjusted rand index score (−0.5 to 1.0). −0.5 = especially discordant, 0.0 = random labelings, 1.0 = perfect match
https://scikit-learn.org/stable/modules/generated/sklearn.metrics.adjusted_rand_score.html#sklearn.metrics.adjusted_rand_score

TABLE 6
Unsupervised k-means clustering of A2D Dataset using SAM
feature descriptor (full SAM (SAM-S + SAM-M + SAM-M))
There are 43 unique object-motion pairings in the A2D dataset, e.g.
adult-walking, tested using SAM-S, SAM-A, SAM-M descriptors separately
and together as the full SAM descriptor to correctly cluster the testing
dataset of A2D into the 43 pairings using k-means clustering algorithm.
The correct number of clusters was set as k = 43, and the clusters
were first fitted the k-means clusterer using the training dataset and
applied it to the testing dataset to compute the clustering performance
metrics; adjusted mutual information (AMI) and adjusted rand index (ARI).

| | k-means clustering to identify object-motion | | | |
|---|---|---|---|---|
| | Feature Descriptor | AMI | ARI | Ranking |
| Shape (S), Appearance | SAM-S (kernel ECC), n = 562 | 0.157 | 0.038 | 3 |
| (A), Motion(M) | SAM-A, n = 149 | 0.182 | 0.052 | 2 |
| components of full SAM | SAM-M, n = 422 | 0.135 | 0.038 | 4 |
| Full SAM feature | SAM (all, SAM-S + SAM-A + | 0.202 | 0.062 | 1 |
| descriptor | SAM-M), n = 1133 | | | |
| Negative control | Random label permutation | 0.000 | 0.000 | 5 |

AMI - adjusted mutual information score (upper-bounded by 1) the larger the better.
https://scikit-learn.org/stable/modules/generated/sklearn.metrics.adjusted_mutual_info_score.html
ARI - adjusted rand index score (−0.5 to 1.0). −0.5 = especially discordant, 0.0 = random labelings, 1.0 = perfect match
https://scikit-learn.org/stable/modules/generated/sklearn.metrics.adjusted_rand_score.html#sklearn.metrics.adjusted_rand_score

TABLE 7
Supervised classification of A2D Dataset with Support Vector Machines (SVM) using SAM feature descriptor
(full SAM (SAM-S + SAM-M + SAM-M)) There are 43 unique object-motion pairings in the A2D dataset
e.g. adult-walking, tested using SAM-S, SAM-A, SAM-M descriptors separately and together as the full SAM
descriptor to correctly train a machine learning classifier, a radial basis function support vector machine
(RBF-SVM) using the training dataset to correctly identify either the object, motion or object-motion pair
in the testing dataset of A2D. Since the number of object-motion pairings are large relative to the number
of images, we train independent RBF-SVM on object and motion classifiers and combine the results to generate
the object-motion pairing prediction. Perfomance was evaluated by computing the balanced accuracy (acc.)
the number of correctly predicted classes and F1 score, a harmonic average of precision and recall.

| | Training a RBF SVM to identify object/motion/object-motion | | | | | | | | | |
|---|---|---|---|---|---|---|---|---|---|---|
| | | Object (k = 7) | | | Motion (k = 9) | | | Object-Motion (k = 43) | | |
| | Feature | | | Ranking | | | Ranking | | | Ranking |
| | Descriptor | Acc | F1 | on object | Acc | F1 | on Motion | Acc | F1 | Both |
| Shape (S), | SAM-S | 0.574 | 0.579 | 3 | 0.376 | 0.358 | 4 | 0.201 | 0.246 | 4 |
| Appearance | (kernel | | | | | | | | | |
| (A), | ECC), | | | | | | | | | |
| Motion(M) | n = 562 | | | | | | | | | |
| components | SAM-A, | 0.591 | 0.608 | 2 | 0.422 | 0.405 | 3 | 0.289 | 0.273 | 3 |
| of | n = 149 | | | | | | | | | |
| | SAM-M, | 0.557 | 0.572 | 4 | 0.461 | 0.470 | 2 | 0.319 | 0.271 | 2 |
| | n = 422 | | | | | | | | | |
| Full SAM | SAM (all, | 0.686 | 0.693 | 1 | 0.538 | 0.535 | 1 | 0.341 | 0.410 | 1 |
| feature | SAM-S + | | | | | | | | | |
| descriptor | SAM-A + | | | | | | | | | |
| | SAM-M), | | | | | | | | | |
| | n = 1133 | | | | | | | | | |
| Negative | Random | 0.003 | 0.003 | 5 | 0.004 | 0.000 | 5 | 0.000 | 0.000 | 5 |
| control | label | | | | | | | | | |
| | permutation | | | | | | | | | |

As point of comparison to k-means clustering, the same
scores of AMI and ARI from the RBF-SVM can be reported
Object-Motion (k = 43)

| AMI | ARI | Ranking |
|---|---|---|
| 0.230 | 0.111 | 4 |
| 0.273 | 0.149 | 3 |
| 0.271 | 0.202 | 2 |
| 0.371 | 0.263 | 1 |
| 0.000 | 0.000 | 5 |

Acc.: balanced accuracy score. The fraction of correctly classified instances accounting for imbalance between classes (0-1). 0 = random, 1 = perfect match
https://scikit-learn.org/stable/modules/generated/sklearn.metrics.balanced_accuracy_score.html#sklearn.metrics.balanced_accuracy_score
F1: harmonic mean of the precision and recall 2*precision*recall/(precision + recall). (0-1) where 1 = perfect
https://scikit-learn.org/stable/modules/generated/sklearn.metrics.fl score.html#sklearn.metrics.fl_score

TABLE 8
Constituents of the organoid culture medium.

| Cell line | Medium and Supplements | Selectable Antibiotic |
|---|---|---|
| HEK293T Rspo1-Fc Cells | Advanced DMEM/F-12 | 0.3 mg/mL Zeocin |
| | 5% FBS | (ThermoFisher |
| | 1% 2 mM L-Glutamine | Scientific/R25001) |
| | 2 μL/mL 1M HEPES | |
| HEK293T Nog-Fc Cells | Advanced DMEM/F-12 | 10 μg/mL |
| | 5% FBS | Puromycin (ThermoFisher |
| | 1% 2 mM L-Glutamine | Scientific/A1113803) |
| | 2 μL/mL 1M HEPES | |
| HEK293T Wnt3A- | DMEM | 0.4 mg/mL G418 |
| Luciferase Reporter Cells | 10% FBS | (Sigma-Aldrich/A1720) |
| | 1% 2 mM L-Glutamine | |
| | 2 μlLmL 1M HEPES | |
| L Wnt3A Cells | DMEM | 0.4 mg/mL G418 |
| (CRL-2647TM) | 5% FBS | (Sigma-Aldrich/A1720) |
| | 1% 2 mM L-Glutamine | |
| | 2 μL/mL 1M HEPES | |

TABLE 9
Constituents of the human organoid medium (HOM) and reagent concentrations.

| Constituent | Company/Catalogue Number | Final Concentration | |
|---|---|---|---|
| A83-01 | StemCell Technologies/72024 | 500 | nM |
| Antibiotic Antimycotic | Sigma-Aldrich/A5955 | 1X | |
| Solution | | | |
| B27 | ThermoFisher | 1X | |
| | Scientific/17504001 | | |
| CHIR99021 | StemCell Technologies/72054 | 3 | μM |
| EGF | ThermoFisher | 50 | ng/mL |
| | Scientific/PHG0311 | | |
| FGF10 | Peprotech/100-26-25 | 100 | ng/mL |
| Gastrin | Sigma-Aldrich/G9145 | 10 | nM |
| N2 | ThermoFisher | 1X | |
| | Scientific/17502001 | | |
| N-Acetyl-Cysteine | Sigma-Aldrich/A9165-5G | 1 | mM |
| Nicotinamide | Sigma-Aldrich/N3376 | 10 | mM |
| Noggin | Conditioned Medium | 20% of final volume | |
| Penicillin/Streptomycin | Thermo Fisher | 200 units/mL | |
| | Scientific/15140122 | | |
| R-Spondin | Conditioned Medium | 30% of final volume | |
| SB202190 | StemCell Technologies/72632 | 10 | μM |
| Wnt3A | Conditioned Medium | 50% of final volume | |
| Y-27632 | StemCell Technologies/72308 | 10 | μM |

TABLE 10
PCR primers used for validating the genotype of mutant organoids.

| | Sequence (5′ to 3′), | SEQ |
|---|---|---|
| Genotype | F: forward, R: reverse | ID NO |
| APC wild type | F: GCC ATC CCT TCA CGT TAG | 1 |
| | R: TTC CAC TTT GGC ATA AGG C | 2 |
| APCmin | F: TTC TGA GAA AGA CAG AAG TTA | 3 |
| | R: TTC CAC TTT GGC ATA AGG C | 4 |
| KRAS wild type | F: ATG TCT TTC CCC AGC ACA GT | 5 |
| | R: TCC GAA TTC AGT GAC TAC AGA TG | 6 |
| KRAS G12D | F: CTA GCC ACC ATG GCT TGA GT | 7 |
| conditional allele | R: TCC GAA TTC AGT GAC TAC AGA TG | 8 |
| KRAS G12D | F: ATG TCT TTC CCC AGC ACA GT | 9 |
| recombined allele | R: TCC GAA TTC AGT GAC TAC AGA TG | 10 |
| p53 wild type | Mm_Trp53_1_SG QuantiTect Primer Assay | |
| | (Qiagen) | |
| | Mm_Trp53_1_SG QuantiTect Primer Assay | |
| | (Qiagen) | |
| p53 R172H | F: AGC TAG CCA CCA TGG CTT GAG TAA | 11 |
| conditional | GTC TGC A | |
| allele | R: CTT GGA GAC ATA GCC ACA CTG | 12 |
| p53 R172H | F: TTA CAC ATC CAG CCT CTG TGG | 13 |
| recombined | R: AGC TAG CCA CCA TGG CTT GAG TAA | 14 |
| allele | GTC TGC A | |

What is claimed is:

1. A method for characterizing morphodynamic profiles of one or more objects, comprising:
obtaining an image dataset comprising a plurality of images;
detecting a set of objects in each image of the image dataset;
segmenting each image in the image dataset to generate a plurality of image patches, each of the plurality of image patches comprising at least a portion of an object of the set of objects;
determining shape, appearance, and motion (SAM) features for each of the plurality of image patches, wherein the SAM features comprise a set of shape features, a set of appearance features, and a set of motion features;
generating SAM descriptors based on the SAM features; and
clustering the set of objects based on the SAM descriptors to provide one or more SAM phenotype clusters of objects having different morphodynamic profiles.

2. The method of claim 1, further comprising: after the step of determining the SAM features, performing dimensionality reduction for the SAM features to analyze the SAM features in a reduced- or two-dimensional space.

3. The method of claim 2, wherein the dimensional reduction is performed by Uniform Manifold Approximation and Projection (UMAP).

4. The method of any one of the preceding claims, comprising tracking the detected objects between consecutive images of an image sequence in the image dataset if the image dataset is derived from a video.

5. The method of any one of the preceding claims, comprising pre-processing the SAM features to remove zero-valued, noisy, or non-temporally varying features to select a subset of the SAM features.

6. The method of any one of the preceding claims, comprising computing a SAM phenotype trajectory over a period of time for each of the SAM phenotype clusters or user-defined sub-population of objects in a dataset that has been tagged with the same label to determine temporal evolution of phenotypic diversity in a given object population.

7. The method of any one of the preceding claims, comprising determining SAM phenotype frequency over a period of time and/or transition probability between the SAM phenotype clusters.

8. The method of claim 7, comprising determining the cluster transition probability using categorical hidden markov models (HMM).

9. The method of any one of the preceding claims, comprising automatically grouping the SAM features that exhibit the same covariation into one or more SAM modules.

10. The method of claim 9, further comprising automatic hierarchical clustering to automatically identifying the one or more SAM modules using a clustering metric.

11. The method of any one of the preceding claims, comprising identifying representative image exemplars to visualize a mean of the SAM phenotype clusters,

12. The method of any one of the preceding claims, comprising scoring the relative contribution of shape, appearance or motion or of spatial scale; global, local-regional and local-distribution to describe what type of features are most important in the dataset that has been analyzed.

13. The method of any one of the preceding claims, wherein the shape features comprise maximum curvature, minimum curvature, mean curvature, mean curvature magnitude, standard deviation curvature, skew curvature, kurtosis curvature, maximum centroid distance, mean centroid distance, standard deviation mean centroid distance ratio, maximum chordal distance, maximum of minimum centroid distance ratio, chordal distance histogram, area, convex hull area, solidity, extent, perimeter, equivalent circular diameter, major axis length, minor axis length, area perimeter aspect ratio, major over minor axis length ratio (eccentricity), moment of eccentricity, Hu moments, Zernike moments, Fourier features, Euler characteristic curves, shape context, a combination thereof, or transformations thereof.

14. The method of any one of the preceding claims, wherein the shape features comprise mean global intensity, standard deviation global intensity, mean regional intensity, standard deviation intensity, Haralick features, SIFT descriptor, a combination thereof, or transformations thereof.

15. The method of any one of the preceding claims, wherein the motion features comprise mean global speed, standard deviation global speed, mean global optical flow speed, standard deviation global optical flow speed, mean curl optical flow, standard deviation curl optical flow, mean divergence optical flow, standard deviation divergence optical flow, mean regional optical flow speed, standard deviation optical flow speed, histogram regional optical flow speeds, sift descriptor of optical flow speed, sift descriptor of curl of optical flow, SIFT descriptor of divergence of optical flow, a combination thereof, or transformations thereof.

16. The method of any one of the preceding claims, wherein the step of clustering comprises performing k-means clustering for the set of objects.

17. The method of any one of the preceding claims, wherein the step of clustering comprises performing an elbow method to select the number of the one or more SAM phenotype clusters of the objects.

18. The method of any one of the preceding claims, wherein the step of clustering comprises clustering temporal trajectories of the set of objects.

19. The method of claim 18, comprising generating a pairwise distance matrix using multidimensional dynamical time warping (DTW).

20. The method of any one of the preceding claims, wherein the step of clustering comprises performing hierarchical clustering for the set of objects.

21. The method of any one of the preceding claims, comprising determining a relationship between the one or more SAM phenotype clusters.

22. The method of claim 21, further comprising determining the relationship between the one or more SAM phenotype clusters using partition-based graph abstraction (PAGA).

23. The method of any one of the preceding claims, wherein the set of objects comprise biological entities.

24. The method of claim 23, wherein the morphodynamic profiles comprise a morphodynamic phenotype of the biological entities.

25. The method of any one of claims 23 to 24, further comprising characterizing a molecular profile of the biological entities.

26. The method of any one of claims 23 to 25, further comprising correlating the morphodynamic phenotype with a molecular profile of the biological entities.

27. The method of any one of claims 25 to 26, wherein the molecular profile is selected from genotype, transcription activity, transcriptomic profile, gene expression activity, genomic profile, protein expression activity, proteomic profile, protein interaction activity, cellular receptor expression activity, lipid profile, lipid activity, carbohydrate profile, microvesicle activity, glucose activity, metabolic profile, and combinations thereof.

28. The method of any one of claims 23 to 27, comprising correlating the morphodynamic phenotype of the biological entities with gene expression or transcription activities of the biological entities.

29. The method of any one of claims 25 to 28, comprising determining the molecular profile by a method selected from DNA analysis, RNA analysis, protein analysis, lipid analysis, metabolite analysis, mass spectrometry, and combinations thereof.

30. The method of any one of claims 25 to 29, comprising determining the molecular profile of the biological entities by single cell RNA sequencing.

31. The method of any one of claims 23 to 30, comprising correlating the morphodynamic phenotype of the biological entities with a clinical outcome of a treatment.

32. The method of any one of claims 23 to 31, wherein the biological entities comprise a cell, an organoid, an organelle, a virus particle, a biopolymer, a polypeptide, a nucleic acid, a lipid, an oligosaccharide, a biomarker, or a combination thereof.

33. The method of claim 32, wherein the cell is selected from a eukaryotic cell, a prokaryotic cell, a mammalian cell, a yeast cell, a tumor cell, a circulating tumor cell, a blood cell, a peripheral blood mononuclear cell, a cell of an immune system, a white blood cell, a T cell, a T helper cell, a lymphocyte, a CD4 lymphocyte, a progenitor cell, an endothelial progenitor cell, and a fetal cell.

34. The method of any one of the preceding claims, wherein the set of objects are detected by a trained object detection algorithm comprising a convolutional neural network.

35. The method of claim 34, wherein the trained object detection algorithm is YOLOv3.

36. The method of any one of the preceding claims, wherein the set of objects are tracked by a multi-object tracker.

37. The method of claim 36, wherein the multi-object tracker is an intersection-over-union bounding box tracker with optical flow guidance.

38. The method of any one of the preceding claims, wherein the set of objects are segmented by a trained object segmentation algorithm comprising a convolutional neural network.

39. The method of claim 38, wherein the trained object segmentation algorithm is an attention U-Net.

40. The method of any one of the preceding claims, wherein the images are obtained by timelapse microscopy.

41. The method of any one of claims 23 to 40, wherein the images are obtained for the biological entities under different conditions over a period of time.

42. The method of any one of the preceding claims, wherein the images are derived from a video or static images acquired over a period of time.

43. The method of any one of the preceding claims, wherein the images are label free images or fluorescent images.

44. The method of any one of the preceding claims, wherein the images comprise two-dimensional images and wherein the method comprises converting three-dimensional z-stack image frames into the two-dimensional images.

45. The method of any one of the preceding claims, comprising assembling videos of an object acquired from multi-part acquisitions into one long timelapse.

46. A system for characterizing morphodynamic profiles of one or more objects, comprising:
a processor, configured to:
obtain an image dataset comprising a plurality of images;
detect a set of objects in each image of the image dataset;
segment each image in the image dataset to generate a plurality of image patches, each of the plurality of image patches comprising at least a portion of an object of the set of objects;
determine shape, appearance, and motion (SAM) features for each of the plurality of image patches, wherein the SAM features comprise a set of shape features, a set of appearance features, and a set of motion features;
generate SAM descriptors based on the SAM features; and
cluster the set of objects based on the SAM descriptors to provide one or more SAM phenotype clusters of objects having different morphodynamic profiles.

47. The system of claim 46, wherein the processor is further configured to, after the step of determining the SAM features, perform dimensionality reduction for the SAM features to analyze the SAM features in a reduced- or two-dimensional space.

48. The system of claim 47, wherein the dimensional reduction is performed by Uniform Manifold Approximation and Projection (UMAP).

49. The system of any one of claims 46 to 48, wherein the processor is configured to track the detected objects between consecutive images of an image sequence in the image dataset if the image dataset is derived from a video.

50. The system of any one of claims 46 to 49, wherein the processor is configured to pre-process the SAM features to remove zero-valued, noisy, or non-temporally varying features to select a subset of the SAM features.

51. The system of any one of claims 46 to 50, wherein the processor is configured to compute a SAM phenotype trajectory over a period of time for each of the SAM phenotype clusters or user-defined sub-population of objects in a dataset that has been tagged with the same label to determine temporal evolution of phenotypic diversity in a given object population.

52. The system of any one of claims 46 to 51, wherein the processor is configured to determine SAM phenotype frequency over a period of time and/or transition probability between the SAM phenotype clusters.

53. The system of claim 52, wherein the processor is configured to determine the cluster transition probability using categorical hidden markov models (HMM).

54. The system method of any one of claims 46 to 53, wherein the processor is configured to automatically group the SAM features that exhibit the same covariation into one or more SAM modules.

55. The system of claims 46 to 54, wherein the processor is further configured to perform automatic hierarchical clustering to automatically identifying the one or more SAM modules using a cluster metric.

56. The system of claims 46 to 55, wherein the processor is further configured to identify representative image exemplars to visualize a mean of the SAM phenotype clusters,

57. The system of claims 46 to 55, wherein the processor is further configured to score relative contribution of shape, appearance or motion or of spatial scale; global, local-regional or local-distribution to describe what type of features are most important in the dataset that has been analyzed.

58. The system of any one of claims 46 to 57, wherein the shape features comprise maximum curvature, minimum curvature, mean curvature, mean curvature magnitude, standard deviation curvature, skew curvature, kurtosis curvature, maximum centroid distance, mean centroid distance, standard deviation mean centroid distance ratio, maximum chordal distance, maximum of minimum centroid distance ratio, chordal distance histogram, area, convex hull area, solidity, extent, perimeter, equivalent circular diameter, major axis length, minor axis length, area perimeter aspect ratio, major over minor axis length ratio (eccentricity), moment of eccentricity, Hu moments, Zernike moments, Fourier features, Euler characteristic curves, shape context, a combination thereof, or transformations thereof.

59. The system of any one of claims 46 to 58, wherein the shape features comprise mean global intensity, standard deviation global intensity, mean regional intensity, standard deviation intensity, Haralick features, SIFT descriptor, a combination thereof, and transformations thereof.

60. The system of any one of claims 46 to 59, wherein the motion features comprise mean global speed, standard deviation global speed, mean global optical flow speed, standard deviation global optical flow speed, mean curl optical flow, standard deviation curl optical flow, mean divergence optical flow, standard deviation divergence optical flow, mean regional optical flow speed, standard deviation optical flow speed, histogram regional optical flow speeds, sift descriptor of optical flow speed, sift descriptor of curl of optical flow, SIFT descriptor of divergence of optical flow, a combination thereof, or transformations thereof.

61. The system of any one of claims 46 to 60, wherein the step of clustering comprises performing k-means clustering for the set of objects.

62. The system of any one of claims 46 to 61, wherein the step of clustering comprises performing an elbow method to select the number of the one or more SAM phenotype clusters of the objects.

63. The system of any one of claims 46 to 62, wherein the step of clustering comprises clustering temporal trajectories of the set of objects.

64. The system of claim 63, wherein the processor is configured to generate a pairwise distance matrix using multidimensional dynamical time warping (DTW).

65. The system of any one of claims 46 to 64, wherein the step of clustering comprises performing hierarchical clustering for the set of objects.

66. The system of any one of claims 46 to 65, wherein the processor is configured to determine a relationship between the one or more SAM phenotype clusters.

67. The system of claim 66, wherein the processor is further configured to determine the relationship between the one or more SAM phenotype clusters using partition-based graph abstraction (PAGA).

68. The system of any one of claims 46 to 67, wherein the set of objects comprises biological entities.

69. The system of claim 68, wherein the morphodynamic profiles comprise a morphodynamic phenotype of the biological entities.

70. The system of any one of claims 68 to 69, wherein the processor is further configured to characterize a molecular profile of the biological entities.

71. The system of any one of claims 68 to 70, wherein the processor is further configured to correlate the morphodynamic phenotype with a molecular profile of the biological entities.

72. The system of any one of claims 70 to 71, wherein the molecular profile is selected from genotype, transcription activity, transcriptomic profile, gene expression activity, genomic profile, protein expression activity, proteomic profile, protein interaction activity, cellular receptor expression activity, lipid profile, lipid activity, carbohydrate profile, microvesicle activity, glucose activity, metabolic profile, and combinations thereof.

73. The system of any one of claims 68 to 72, wherein the processor is configured to correlate the morphodynamic phenotype of the biological entities with gene expression or transcription activities of the biological entities.

74. The system of any one of claims 70 to 73, wherein the processor is configured to determine the molecular profile by a system selected from DNA analysis, RNA analysis, protein analysis, lipid analysis, metabolite analysis, mass spectrometry, and combinations thereof.

75. The system of any one of claims 70 to 74, wherein the processor is configured to determine the molecular profile of the biological entities by single cell RNA sequencing.

76. The system of any one of claims 68 to 75, wherein the processor is configured to correlate the morphodynamic phenotype of the biological entities with a clinical outcome of a treatment.

77. The system of any one of claims 70 to 76, wherein the biological entities comprise a cell, an organoid, an organelle, a virus particle, a biopolymer, a polypeptide, a nucleic acid, a lipid, an oligosaccharide, a biomarker, or a combination thereof.

78. The system of claim 77, wherein the cell is selected from a eukaryotic cell, a prokaryotic cell, a mammalian cell, a yeast cell, a tumor cell, a circulating tumor cell, a blood cell, a peripheral blood mononuclear cell, a cell of an immune system, a white blood cell, a T cell, a T helper cell, a lymphocyte, a CD4 lymphocyte, a progenitor cell, an endothelial progenitor cell, and a fetal cell.

79. The system of any one of claims 46 to 78, wherein the set of objects is detected by a trained object detection algorithm comprising a convolutional neural network.

80. The system of claim 79, wherein the trained object detection algorithm is YOLOv3.

81. The system of any one of claims 46 to 80, wherein the set of objects are tracked by a multi-object tracker.

82. The system of claim 81, wherein the multi-object tracker is an intersection-over-union bounding box tracker with optical flow guidance.

83. The system of any one of claims 46 to 82, wherein the set of objects are segmented by a trained object segmentation algorithm comprising a convolutional neural network.

84. The system of claim 83, wherein the trained object segmentation algorithm is an attention U-Net.

85. The system of any one of claims 46 to 84, wherein the images are obtained by timelapse microscopy.

86. The system of any one of claims 68 to 85, wherein the images are obtained for the biological entities under different conditions over a period of time.

87. The system of any one of claims 46 to 86, wherein the images are derived from a video or static images acquired over a period of time.

88. The system of any one of claims 46 to 87, wherein the images are label free images or fluorescent images.

89. The system of any one of claims 46 to 88, wherein the images comprise two-dimensional images and wherein the processor is configured to convert three-dimensional z-stack image frames into two-dimensional images.

90. The system of any one of claims 46 to 89, wherein the processor is configured to assemble videos of an object acquired from multi-part acquisitions into one long timelapse.


SEQUENCE LISTING (14 sequences)

SEQ ID NO: 1 - DNA, 18 bp, synthetic construct
  source 1..18: mol_type = other DNA
gccatcccttcacgttag

SEQ ID NO: 2 - DNA, 19 bp, synthetic construct
  source 1..19: mol_type = other DNA
ttccactttggcataaggc

SEQ ID NO: 3 - DNA, 21 bp, synthetic construct
  source 1..21: mol_type = other DNA
ttctgagaaagacagaagtta

SEQ ID NO: 4 - DNA, 19 bp, synthetic construct
  source 1..19: mol_type = other DNA
ttccactttggcataaggc

SEQ ID NO: 5 - DNA, 20 bp, synthetic construct
  source 1..20: mol_type = other DNA
atgtctttccccagcacagt

SEQ ID NO: 6 - DNA, 23 bp, synthetic construct
  source 1..23: mol_type = other DNA
tccgaattcagtgactacagatg

SEQ ID NO: 7 - DNA, 20 bp, synthetic construct
  source 1..20: mol_type = other DNA
ctagccaccatggcttgagt

SEQ ID NO: 8 - DNA, 23 bp, synthetic construct
  source 1..23: mol_type = other DNA
tccgaattcagtgactacagatg

SEQ ID NO: 9 - DNA, 20 bp, synthetic construct
  source 1..20: mol_type = other DNA
atgtctttccccagcacagt

SEQ ID NO: 10 - DNA, 23 bp, synthetic construct
  source 1..23: mol_type = other DNA
tccgaattcagtgactacagatg

SEQ ID NO: 11 - DNA, 31 bp, synthetic construct
  source 1..31: mol_type = other DNA
agctagccaccatggcttgagtaagtctgca

SEQ ID NO: 12 - DNA, 21 bp, synthetic construct
  source 1..21: mol_type = other DNA
cttggagacatagccacactg

SEQ ID NO: 13 - DNA, 21 bp, synthetic construct
  source 1..21: mol_type = other DNA
ttacacatccagcctctgtgg

SEQ ID NO: 14 - DNA, 31 bp, synthetic construct; identical to SEQ ID NO: 11
  source 1..31: mol_type = other DNA